(19) United States
(12) Patent Application Publication
(10) Pub. No.: US 2024/0425856 A1
(43) Pub. Date: Dec. 26, 2024

(54) Compositions and Methods for Treating Cag Repeat Diseases

(71) Applicants: Iris Medicine, Inc., Berkeley, CA (US); Board of Trustees of Southern Illinois University, Springfield, IL (US)

(72) Inventors: Philip J. Jensik, Springfield, IL (US); Keith T. Gagnon, Springfield, IL (US); David Corey, Berkeley, CA (US); Eleonora de Klerk, Berkeley, CA (US); Megan Blewett, Berkeley, CA (US); Melissa A. Kotterman, Berkeley, CA (US)

(21) Appl. No.: 18/699,523

(22) PCT Filed: Oct. 5, 2022
(86) PCT No.: PCT/US2022/077572

§ 371 Date: Apr. 8, 2024

Related U.S. Application Data
(60) Provisional application No. 63/339,363, filed on May 6, 2022.
(60) Provisional application No. 63/253,070, filed on Oct. 6, 2021.

(51) Int. Cl. C12N 15/113; A61K 9/00; A61P 25/28; C12N 15/86

(52) U.S. Cl. CPC C12N 15/113; A61K 9/0019; A61K 9/0085; A61P 25/28; C12N 15/86; C12N 2310/14; C12N 2310/531; C12N 2750/14143

(57) ABSTRACT

The disclosure relates to compositions and methods for the production and therapeutic use of inhibitory double stranded RNAs.

### CROSS-REFERENCE

This application claims the benefit of U.S. Provisional Patent Application No. 63/253,070, filed Oct. 6, 2021, and U.S. Provisional Patent Application No. 63/339,363, filed May 6, 2022, which applications are incorporated herein by reference in their entirety.

### INCORPORATION-BY-REFERENCE OF MATERIAL ELECTRONICALLY SUBMITTED

A Sequence Listing is provided herewith as a Sequence Listing XML, “IRIS-001WO_SEQ_LIST” created on Oct. 4, 2022 and having a size of 1,298 KB. The contents of the Sequence Listing XML are incorporated by reference herein in their entirety.

### BACKGROUND

Repeat expansion disorders are autosomal dominant genetic disorders caused by expansion of DNA repeats. DNA repeats may be composed of single nucleotides to dodecamers or longer. The threshold at which repeat expansions become symptomatic varies with the particular disease. There are over 50 distinct diseases caused by repeat expansions. Repeat expansions may occur in coding or non-coding regions of genes. Repeat expansions may cause defects in a protein encoded by a gene; change the regulation of gene expression; produce a toxic RNA, or lead to chromosome instability.

Inhibition of both mutant and wild-type expression of a repeat-containing gene may induce significant side effects. Thus, suppression of the mutant repeat expansion allele is a desired therapeutic strategy for repeat expansion disorders. Current strategies for mutant allele-specific inhibition include targeting disease-associated single nucleotide polymorphisms (SNPs) or deletions with antisense oligonucleotides or RNA interference agents. However, identifying SNPs associated with repeat expansion mutations requires detailed population-specific genetic studies in large clinical cohorts. Furthermore, depending upon the frequency of the target SNPs or location on the mutant repeat expansion allele, certain affected individuals or populations may be excluded.

### BRIEF SUMMARY

The present disclosure provides a double-stranded RNA comprising: a) a first strand that hybridizes to a target CAG repeat region of a CAG repeat containing RNA; and b) a second strand that hybridizes to the first strand, wherein the first strand comprises: i) a first mismatch to the target CAG repeat region; and ii) at least a second mismatch to the target CAG repeat region. The present disclosure provides a DNA molecule comprising a nucleotide sequence encoding the first strand of the double-stranded RNA, where the nucleotide sequence is operably linked to a promoter that is functional in a eukaryotic cell. The present disclosure provides a recombinant nucleic acid comprising: a) a double-stranded RNA of the present disclosure; and b) a microRNA scaffold; the present disclosure also provides a recombinant expression vector comprising a nucleotide sequence encoding such a recombinant nucleic acid. The present disclosure provides a DNA molecule comprising a nucleotide sequence encoding a recombinant nucleic acid comprising: a) a double-stranded RNA of the present disclosure; and b) a microRNA scaffold; the present disclosure also provides a recombinant expression vector comprising such a DNA molecule. The present disclosure provides viral and non-viral delivery vehicles comprising a recombinant expression vector of the present disclosure; and pharmaceutical compositions comprising such delivery vehicles. The present disclosure provides methods for selectively reducing translation of a disease-associated CAG repeat-containing RNA.

In some embodiments, the present disclosure provides a double stranded RNA comprising from 5′ to 3′: (a) a 5′ leader sequence; (b) a 5′ stem comprising a passenger sequence or a guide sequence; (c) a 5′ linker of 1-6 bases; (d) a terminal loop; I a 3′ linker of 1-6 bases; (f) a 3′ stem comprising: (i) a guide sequence if the 5′ stem comprises the passenger sequence; or (ii) a passenger sequence if the 5′ stem comprises the guide sequence; and (g) a 3′ trailer sequence; wherein the guide sequence targets a CAG repeat region of a CAG repeat containing RNA (e.g., mRNA or pre-mRNA) and comprises 1-5 base mismatches relative to the CAG repeat region, wherein the base mismatches are located at positions 8-16 of the guide sequence.

### BRIEF DESCRIPTION OF THE SEVERAL VIEWS OF THE DRAWINGS

This patent or application file contains at least one drawing executed in color. Copies of this patent or patent application publication with color drawing(s) will be provided by the Office upon request and payment of the necessary fee.

FIGS. 1A-1D: Cloning and design of shRNAs expressed from a custom U6 promoter-driven construct. (FIG. 1A) Plasmid map for customized pxTRC-EGFP-puro. (FIG. 1B) Initial shRNA expression cassette (SEQ ID NO:760). (FIG. 1C) shRNA construct modifications to improve shRNA processing accuracy and abundance (SEQ ID NO:761). (FIG. 1D) shHD1L-1 design (SEQ ID NO:762). For FIGS. 1B-1D, green markings show Drosha cleavage on the 5′ leader and 3′ trailer sequences and Dicer cleavage at the 5′ upper stem and 3′ upper stem; and mismatch positions in guide strand are boxed in purple.

FIGS. 2A-2C: miRNA miR33 scaffold designs for shRNA expression. (FIG. 2A) miR33 miRNA structure and sequence elements (SEQ ID NO:763). (FIG. 2B) Generalized miR33 scaffold for shRNA cloning and expression (SEQ ID NO:764). (FIG. 2C) shHD-33full-mimic design (SEQ ID NO:765). For FIGS. 2A-2C, green markings show Drosha cleavage on the 5′ leader and 3′ trailer sequences and Dicer cleavage at the 5′ upper stem and 3′ upper stem; and mismatch positions in the guide strand are boxed in purple.

FIGS. 3A-3B: Cell based evaluation of select shRNAs targeting the CAG repeat expansion of HTT. (FIG. 3A) HEK 293T Luciferase assay results for several shRNAs targeting CAG repeats. Wild-type (wt) and mutant (mut) constructs are indicated and percent luciferase activity is normalized to scrambled shRNA control. Mean of two independent replicates is shown. Error is standard error of the mean (S.E.M.). Statistical significance is indicated by p-values less than 0.05 (*) or 0.01 (**). (FIG. 3B) Quantified knockdown of HTT wt and mut proteins in patient-derived cells assayed by Western blot and fit to Hill plot equation. Single replicate or mean of two replicates is shown. Error is S.E.M.

FIG. 4: is a graph comparing shRNA abundance for a virally encoded shRNA having a guide sequence perfectly matching the CAG repeat region of HTT mRNA vs. a virally encoded shRNA having a guide sequence comprising mismatch(es) relative to the CAG repeat region of HTT mRNA, wherein the mismatches are located at positions 8-16 of the guide sequence.

FIG. 5 is a graph showing that lentiviral constructs encoding allele selective shRNAs significantly decreased expression of the pathogenic HTT allele (mut-HTT) in fibroblast cells transduced with shHD-33full and shHD33-fullmimic compared to the normal HTT allele.

FIG. 6 depicts co-localization of DARP32 and GFP staining in zQ175 mouse striatum.

FIG. 7A-7B depicts western blot analysis of wild-type (WT) and mut HTT protein in striatum of zQ175 mice in the AAV9-shHD33-Full-Mimic treatment group relative to the AAV9-shScr treatment group.

FIG. 8 depicts dose-dependent AAV delivery and GFP expression in the striatum.

FIG. 9 depicts allele-selective knockdown in vivo with a small binding RNA (sbRNA) delivered via a recombinant AAV vector.

FIG. 10 depicts transduction efficiency in the cerebellum following ICV administration of recombinant AAV virions comprising a recombinant AAV encoding an sbRNA.

FIG. 11 depicts allele-selective knockdown in vivo with a small binding RNA (sbRNA) delivered via a recombinant AAV vector in a SCA2 mouse model (left panel) and partial restoration of expression of key cerebellar genes that are molecular markers of pathology in ATXN2-Q127 mice (right panel).

FIG. 12 depicts the effect of sbRNA on preservation of wild-type (WT) gene expression in ATXN2-Q127 mice. Protein levels were unchanged for non-target genes containing CAG repeats.

FIG. 13A-13E depict the effect of the registry on knockdown (FIG. 13(A) Guide Strand Column, SEQ ID Nos:406, 873, 406, 406, 406, 874, 875, from top to bottom respectively; Loop Column, SEQ ID Nos:876, 876, 877, 878, 876, 879, 876, from top to bottom respectively; Passenger Strand Column, SEQ ID Nos:881, 880, 880, 880, 880, 882, 883, 884, from top to bottom respectively) (FIG. 13(E) SEQ ID Nos:868, 869, 870, 871 and 872 from top to bottom respectively).

FIG. 14 is a schematic depiction of a guide sequence screening system.

FIG. 15 depicts knockdown and allele selectivity using various guide sequences.

FIG. 16 depicts guide sequence screening and allele selectivity.

FIG. 17 depicts the effect of the number of mismatches to the target CAG repeat region on knockdown and allele selectivity.

FIG. 18 depicts the effect of a single mismatch on knockdown and allele selectivity, where the mismatch is at position 8, 9, 10, or 11.

FIG. 19 depicts the effect, on knockdown and allele selectivity, of distance between a first mismatch and a second mismatch, when the first mismatch is at position 8, 9, 10, or 11.

FIG. 20 depicts the effect, on knockdown and allele selectivity, of distance between mismatches in guide sequences having three mismatches (left panel) or four mismatches (right panel) with a target CAG.

FIG. 21 depicts the effect, on knockdown and allele selectivity, of distance between mismatches in guide sequences having three mismatches with a target CAG, where the first mismatch is at position 9, 10, or 11.

FIG. 22 provides Table 6.

FIG. 23 provides Table 7.

FIG. 24 provides Table 8.

FIG. 25 provides nucleotide sequences of sbRNAs comprising miR451 5′ and 3′ flanking polynucleotides.

### DETAILED DESCRIPTION

Repeat expansion disorders present significant obstacles for selective inhibition of disease allele versus normal allele. The present disclosure provides double-stranded RNAs that can exploit differences in the number of repeats and achieve allele-selective inhibition of repeat-containing proteins. The double-stranded RNAs target the repeat region of a repeat-containing target RNA molecule (e.g., mRNA or pre-mRNA) and contains 1-5 (e.g., 1, 2, 3, 4, or 5) nucleobase mismatches relative to the repeat region in the target mRNA or pre-mRNA at positions 8-16 (e.g., 8, 9, 10, 11, 12, 13, 14, 15, or 16) of the guide sequence, which enhances the ability of double-stranded RNAs to selectively inhibit mutant protein expression versus wild-type. Standard design of vector encoded double-stranded RNAs targeting the repeat region of a repeat containing target mRNA or pre-mRNA and comprising 1-5 mismatches relative to the repeat region at positions 8-16 of the guide sequence revealed positional shifts in processing of the 5′ cleavage sites. Due to the shift in processing, the positions of the mismatch(es) in the guide sequence were also shifted, placing them at offset or undesirable positions. Double stranded RNAs having mismatches at offset or undesirable positions may not have desirable functions. The design of the double-stranded RNAs of the disclosure has been modified for vector expression, enhancing proper processing to place mismatches at desired positions and provide more predictable 5′ cleavage sites.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

As used herein, the term “nucleic acid” or “polynucleotide” refer to any nucleic acid polymer composed of covalently linked nucleotide subunits, such as polydeoxyribonucleotides or polyribonucleotides. Examples of nucleic acids include RNA and DNA.

As used herein, “RNA” refers to a molecule comprising one or more ribonucleotides and includes double-stranded RNA, single-stranded RNA, isolated RNA, synthetic RNA, recombinant RNA, as well as modified RNA that differs from naturally-occurring RNA by the addition, deletion, substitution, and/or alteration of one or more nucleotides. Nucleotides of RNA molecules may comprise standard nucleotides or non-standard nucleotides, such as non-naturally occurring nucleotides or chemically synthesized nucleotides.

As used herein, “DNA” refers to a molecule comprising one or more deoxyribonucleotides and includes double-stranded DNA, single-stranded DNA, isolated DNA, synthetic DNA, recombinant DNA, as well as modified DNA that differs from naturally-occurring DNA by the addition, deletion, substitution, and/or alteration of one or more nucleotides. Nucleotides of DNA molecules may comprise standard nucleotides or non-standard nucleotides, such as non-naturally occurring nucleotides or chemically synthesized nucleotides.

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety.

As used herein, “nucleotide” means a nucleoside further comprising a phosphate linking group. As used herein, “linked nucleosides” may or may not be linked by phosphate linkages and thus includes, but is not limited to “linked nucleotides.” As used herein, “linked nucleosides” are nucleosides that are connected in a continuous sequence (i.e. no additional nucleosides are present between those that are linked).

As used herein, “nucleobase” or “base” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

As used herein, “oligonucleotide” means a compound comprising a plurality of linked nucleosides. In some embodiments, an oligonucleotide comprises one or more unmodified ribonucleosides (RNA) and/or unmodified deoxyribonucleosides (DNA) and/or one or more modified nucleosides.

As used herein, “oligomeric compound” means a polymeric structure comprising two or more sub-structures. In certain embodiments, an oligomeric compound comprises an oligonucleotide. In certain embodiments, an oligomeric compound comprises one or more conjugate groups and/or terminal groups. In certain embodiments, an oligomeric compound consists of an oligonucleotide. Oligomeric compounds also include naturally occurring nucleic acids.

As used herein, “single-stranded” means an oligomeric compound that is not hybridized to its complement and which lacks sufficient self-complementarity to form a stable self-duplex.

As used herein, “double-stranded” means an oligomeric compound that is partially or completely hybridized to its complement to form a stable duplex molecule. A double-stranded oligomeric compound may be composed of two separate strands of complementary oligomeric compounds hybridized to each other or a single oligomeric compound which has sufficient self-complementarity to form a stable self-duplex. Stable self-duplexes may contain stem-loop structure(s) and/or bulge(s).

“Isolated” refers to a substance that has been isolated from its natural environment or artificially produced. As used herein with respect to a cell, “isolated” refers to a cell that has been isolated from its natural environment (e.g., from a subject, organ, tissue, or bodily fluid). As used herein with respect to a nucleic acid, “isolated” refers to a nucleic acid that has been isolated or purified from its natural environment (e.g., from a cell, cell organelle, or cytoplasm), recombinantly produced, amplified, or synthesized. In embodiments, an isolated nucleic acid includes a nucleic acid contained within a vector.

As used herein, the term “wild-type” or “non-mutant” form of a gene refers to a nucleic acid that encodes a protein associated with normal or non-pathogenic activity (e.g., a protein lacking a mutation, such as a repeat region expansion that results in higher risk of developing, onset, or progression of a neurodegenerative disease).

As used herein, the term “mutation” refers to any change in the structure of a gene, e.g., gene sequence, resulting in an altered form of the gene, which may be passed onto subsequent generations (hereditary mutation) or not (somatic mutation). Gene mutations include the substitution, insertion, or deletion of a single base in DNA or the substitution, insertion, deletion, or rearrangement of multiple bases or larger sections of genes or chromosomes, including repeat expansions.

As used herein, the term “inhibitory nucleic acid” refers to a nucleic acid that comprises a guide strand sequence that hybridizes to at least a portion of a target nucleic acid, e.g., target RNA, mRNA, or pre-mRNA, and inhibits its expression or activity. An inhibitory nucleic acid may target a protein coding region (e.g., exon) or non-coding region (e.g., 5′UTR, 3′UTR, intron, etc.) of a target nucleic acid. In some embodiments, an inhibitory nucleic acid is a single stranded or double stranded molecule. An inhibitory nucleic acid may further comprise a passenger strand sequence on a separate strand (e.g., double stranded duplex) or in the same strand (e.g., single stranded, self-annealing duplex structure). In some embodiments, an inhibitory nucleic acid is an RNA molecule, such as a siRNA, shRNA, pri-miRNA, pre-miRNA, or miRNA. In some embodiments, an inhibitory nucleic acid is double-stranded RNA (dsRNA), such as a pri-miRNA, pre-miRNA, miRNA, or shRNA.

As used herein, a “microRNA” or “miRNA” refers to a small non-coding RNA molecule capable of mediating silencing of a target gene by cleavage of the target mRNA, translational repression of the target mRNA, target mRNA degradation, or a combination thereof. Typically, miRNA is transcribed as a hairpin or stem-loop (e.g., having a self-complementary, single-stranded backbone) duplex structure, referred to as a primary miRNA (pri-miRNA), which is enzymatically processed (e.g., by Drosha, DGCR8, Pasha, etc.) into a pre-miRNA. Pre-miRNA is exported into the cytoplasm, where it is enzymatically processed by Dicer to produce a miRNA duplex with the passenger strand and then a single-stranded mature miRNA molecule, which is subsequently loaded into the RNA-induced silencing complex (RISC). Reference to a miRNA may include synthetic or artificial miRNAs.

As used herein, a “synthetic miRNA” or “artificial miRNA” or “amiRNA” or “small binding RNA” (sbRNA) refers to an endogenous, modified, or synthetic pri-miRNA or pre-miRNA (e.g., miRNA backbone or scaffold) in which the endogenous miRNA guide sequence and passenger sequence within the stem sequence have been replaced with a heterologous guide sequence and a heterologous passenger sequence that direct highly efficient RNA silencing of the targeted gene (see, e.g., Eamens et al. (2014), Methods Mol. Biol. 1062:211-224). In some embodiments, the nature of the complementarity of the guide and passenger sequences (e.g., number of bases, position of mismatches, types of bulges, etc.) can be similar or different from the nature of complementarity of the guide and passenger sequences in the endogenous miRNA backbone upon which the synthetic miRNA is constructed.

As used herein, the term “microRNA backbone,” “miR backbone,” “microRNA scaffold,” or “miR scaffold” refers to a pri-miRNA or pre-miRNA scaffold, with the stem sequence replaced by a heterologous RNA of interest, and is capable of producing a functional, mature miRNA that directs RNA silencing at the gene targeted by the miRNA of interest. In some cases, a miR backbone comprises a 5′ flanking region (also referred to herein as a “5′ flanking polynucleotide” or a “5′ leader”), a loop motif region (also referred to herein as a “loop polynucleotide”), and a 3′ flanking region (also referred to herein as a “3′ flanking polynucleotide” or a “3′ trailer”). In some cases, a miR backbone comprises a 5′ flanking region and a 3′ flanking region (and does not include a loop motif region). A miR backbone may be derived completely or partially from a wild type miRNA scaffold or be a completely artificial sequence.

As used herein, the term “short hairpin RNA” or “shRNA” includes a conventional stem-loop shRNA, which forms a precursor miRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, a conventional shRNA forms a primary miRNA (pri-miRNA) or a structure very similar to a natural pri-miRNA. The pri-miRNA is subsequently processed by Drosha and its cofactors into pre-miRNA. Therefore, the term “shRNA” includes pri-miRNA molecules and pre-miRNA molecules.

A “stem-loop structure” refers to a nucleic acid having a secondary structure that includes a region of nucleotides which are known or predicted to form a double strand or self-duplex (stem portion) that is linked on one side by a region of predominantly single-stranded nucleotides (terminal loop portion). The terms “hairpin”, “self-duplex” and “fold-back” structures are also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches.

As used herein, the term “guide strand sequence” of an inhibitory nucleic acid refers to a sequence that is substantially complementary (e.g., at least 45%, 50%, 55%, 60%, 65%, 70%, 75%, 80%, 85%, 90%, 95%, or 100% complementary) to a region of about 10-50 nucleotides (e.g., about 15-30, 16-25, 18-23, or 19-22 nucleotides) of the mRNA or pre-mRNA targeted for silencing. The guide sequence is sufficiently complementary to the target mRNA sequence to direct target-specific silencing, e.g., to trigger the destruction of the target mRNA by the RNAi machinery or process or to reduce translation of the target mRNA. In some embodiments, the guide strand sequence refers to the mature guide sequence remaining following cleavage by Dicer.

As used herein, the term “passenger strand sequence” of an inhibitory nucleic acid refers to a sequence that is homologous to the target mRNA or pre-mRNA, and partially or completely complementary to the guide strand sequence of an inhibitory nucleic acid. The guide strand sequence and passenger strand sequence of an inhibitory nucleic acid are hybridized to form a duplex structure (e.g., forming a double-stranded duplex or single-stranded self-annealing duplex structure). In some embodiments, the guide strand sequence and passenger strand sequence refers to the mature sequences remaining following cleavage by Dicer.

As used herein, the term “5′ arm” or “5′ stem” refers to a portion of a double stranded RNA (e.g., shRNA, pre-miRNA, pri-mRNA) that comprises the guide strand or passenger strand.

As used herein, the term “3′ arm” or “3′ stem” refers to a portion of a double stranded RNA that comprises the passenger strand to the 5′ stem's guide strand, or the guide strand to the 5′ stem's passenger strand.

As used herein, a “duplex,” when used in reference to an inhibitory nucleic acid, refers to two nucleic acid strands (e.g., a guide strand and passenger strand) hybridizing together to form a duplex structure. A duplex may be formed by two separate nucleic acid strands or by a single nucleic acid strand having a region of self-complementarity (e.g., hairpin or stem-loop).

As used herein, “target nucleic acid” means a nucleic acid molecule to which an antisense compound hybridizes. A target nucleic acid may be a mRNA (target mRNA) or pre-mRNA (target pre-mRNA) encoded by a target gene.

As used herein, “targeting” or “targeted to” means the association of an antisense compound to a particular target nucleic acid molecule or a particular region of a target nucleic acid molecule. A double-stranded RNA targets a target nucleic acid if it is sufficiently complementary to the target nucleic acid to allow hybridization under physiological conditions.

As used herein, the term “complementary” refers to the ability of polynucleotides to form base pairs with each other. Base pairs are typically formed by hydrogen bonds between nucleotide subunits in antiparallel polynucleotide strands or a single, self-annealing polynucleotide strand. Complementary polynucleotide strands can form base pairs in the Watson-Crick manner (e.g., A to T, A to U, C to G), or in any other manner that allows for the formation of duplexes. In some embodiments, complementary nucleotides include G and U (wobble base pair). As apparent to skilled persons in the art, when using RNA as opposed to DNA, uracil rather than thymine is the base that is considered to be complementary to adenosine. Furthermore, when a “U” is denoted in the context of the present invention, the ability to substitute a “T” is understood, unless otherwise stated. Complementarity also encompasses Watson-Crick base pairing between non-modified and modified nucleobases (e.g., 5-methyl cytosine substituted for cytosine). Full complementarity, perfect complementarity or 100% complementarity between two polynucleotide strands is where each nucleotide of one polynucleotide strand can form hydrogen bond with a nucleotide unit of a second polynucleotide strand. % complementarity refers to the number of nucleotides of a contiguous nucleotide sequence in a nucleic acid molecule that are complementary to an aligned reference sequence (e.g., a target mRNA, passenger strand), divided by the total number of nucleotides and multiplying by 100. In such an alignment, a nucleobase/nucleotide which does not form a base pair is called a mismatch. Insertions and deletions are not permitted in calculating % complementarity of a contiguous nucleotide sequence. It is understood by skilled persons in the art that in calculating complementarity, chemical modifications to nucleobases are not considered as long as the Watson-Crick base pairing capacity of the nucleobase is retained (e.g., 5-methyl cytosine is considered the same as cytosine for the purpose of calculating % complementarity).

As used herein, “non-complementary” in reference to nucleobases means a pair of nucleobases that do not form hydrogen bonds with one another.

As used herein, “mismatch” means a nucleobase of a first oligomeric compound that is not capable of pairing with a nucleobase at a corresponding position of a second oligomeric compound, when the first and second oligomeric compound are aligned. Either or both of the first and second oligomeric compounds may be oligonucleotides. Nucleotides that do not base pair include self-pairing nucleotides (A-A, T-T, U-U, C-C, and G-G), A and C, C and U, C and T, A and G. In some embodiments, a mismatch does not include G-U wobble base pairs.

The “percent identity” between two or more nucleic acid sequences refers to the proportion of nucleotides of a contiguous nucleotide sequence in a nucleic acid molecule that are shared by a reference sequence (i.e., % identity=number of identical nucleotides/total number of nucleotides in the aligned region (e.g., the contiguous nucleotide sequence)×100). Insertions and deletions are not permitted in the calculation of % identity of a contiguous nucleotide sequence. It is understood by skilled persons in the art that in calculating identity, chemical modifications to nucleobases are not considered as long as the Watson-Crick base pairing capacity of the nucleobase is retained (e.g., 5-methyl cytosine is considered the same as cytosine for the purpose of calculating % identity).

As used herein, the term “hybridizing” or “hybridizes” refers to two nucleic acid strands forming hydrogen bonds between base pairs on antiparallel strands, thereby forming a duplex. While not limited to a particular mechanism, the most common mechanism of pairing involves hydrogen bonding, which may be Watson-Crick, Hoogsteen or reversed Hoogsteen hydrogen bonding, between complementary nucleobases. The strength of hybridization between two nucleic acid strands may be described by the melting temperature (Tm), defined as at a given ionic strength and pH, the temperature at which 50% of a target sequence hybridizes to a complementary polynucleotide.

As used herein, “heterologous” refers to a nucleic acid that is not found in a native (naturally occurring) nucleic acid. For example, relative to a component of a microRNA (e.g., a 5′ flanking polynucleotide, a loop polynucleotide, a 3′ flanking polynucleotide) a heterologous guide sequence and a heterologous passenger sequence comprises a nucleotide sequence that is not associated with the microRNA in nature. As used herein, the “guide sequence” is interchangeable with “first strand” (or “targeting strand”, where the “targeting strand” hybridizes to a target RNA) of a double-stranded RNA, regardless of the orientation.

As used herein, “expression cassette” refers to any type of genetic construct containing a nucleic acid (e.g., transgene) in which part or all of the nucleic acid encoding sequence is capable of being transcribed. In some embodiments, expression includes transcription of the nucleic acid, for example, to generate a biologically-active polypeptide product or inhibitory RNA (e.g., siRNA, shRNA, miRNA) from a transcribed gene. In some embodiments, the transgene is operably linked to expression control sequences.

As used herein, the term “transgene” refers to an exogenous nucleic acid that has been transferred naturally or by genetic engineering means into another cell and is capable of being transcribed, and optionally translated.

As used herein, the term “gene expression” refers to the process by which a nucleic acid is transcribed from a nucleic acid molecule, and often, translated into a peptide or protein. The process can include transcription, post-transcriptional control, post-transcriptional modification, translation, post-translational control, post-translational modification, or any combination thereof. Reference to a measurement of “gene expression” may refer to measurement of the product of transcription (e.g., RNA or mRNA), or the product of translation (e.g., peptides or proteins).

As used herein, the term “inhibit expression of a gene” means to reduce, down-regulate, suppress, block, lower, or stop expression of the gene. The expression product of a gene can be an RNA molecule transcribed from the gene (e.g., an mRNA) or a polypeptide translated from an mRNA transcribed from the gene. A reduction in the level of an mRNA results in a reduction in the level of a polypeptide translated therefrom. In some embodiments, inhibition of expression reduces the level of a polypeptide without substantially affecting production of the encoding mRNA. The level of expression may be determined using standard techniques for measuring mRNA or protein.

As used herein, “vector” refers to a genetic construct that is capable of transporting a nucleic acid molecule (e.g., transgene encoding inhibitory nucleic acid) between cells and effecting expression of the nucleic acid molecule when operably-linked to suitable expression control sequences. Expression control sequences may include transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. The vector may be a plasmid, phage particle, transposon, cosmid, phagemid, chromosome, artificial chromosome, virus, virion, lipid nanoparticle, etc. Once transformed into a suitable host cell, the vector may replicate and function independently of the host genome, or may, in some instances, integrate into the genome itself.

As used herein, “host cell” refers to any cell that contains, or is capable of containing a composition of interest, e.g., an inhibitory nucleic acid. In embodiments, a host cell is a mammalian cell, such as a rodent cell (e.g., mouse or rat) or primate cell (e.g., monkey, chimpanzee, or human). In embodiments, a host cell may be in vitro or in vivo. In embodiments, a host cell may be from an established cell line or primary cells. In embodiments, a host cell may be obtained from a patient having or suspected of having a repeat expansion disease or disorder. In embodiments, a host cell is a non-CNS cell, such as a fibroblast. In embodiments, a host cell is a cell of the CNS, such as a neuron, a glial cell, an astrocyte, or a microglial cell.

As used herein, “expanded repeat containing gene” or “expanded repeat containing RNA” refers to a mutant gene or RNA molecule (e.g., pre-mRNA or mRNA) encoded by the mutant gene having a base sequence that includes a repeat region (e.g., CAG repeat) where the repeat region is expanded beyond a predetermined number or range of base repeats that are typically present in a “normal” expanded repeat containing gene or RNA encoded by the gene. The presence or length of the repeat region may affect normal processing, function or activity of the RNA or encoded protein and cause a “repeat expansion” or “expanded repeat” disease or disorder. Expanded repeats may be unstable (dynamic) mutations that change size in successive generations. An expanded repeat may be a dinucleotide repeat, a trinucleotide repeat, a tetranucleotide repeat, a pentanucleotide repeat, a hexanucleotide repeat, etc. In some embodiments, a repeat is a CAG repeat or polyglutamine. An expanded repeat containing gene or RNA encoded by the expanded repeat containing gene may also be referred to as a “pathologic allele” or “pathogenic allele.” In some embodiments, a pathologic or pathogenic allele of a CAG repeat containing gene or RNA encoded by the gene has ≥30 consecutive CAG repeats.

A “repeat expansion disease or disorder,” or “expanded repeat disease or disorder,” refers to a disease or disorder caused by the expansion of a base repeat sequence beyond a predetermined number or range of base repeats that are typically present in a “normal” expanded repeat containing gene or RNA encoded by the gene. A repeat expansion disease or disorder may manifest with markedly varied phenotypes depending on the size of the repeat expansion. Repeat expansion diseases or disorders are primarily neurodegenerative diseases. Some repeat expansion diseases are ophthalmologic diseases. In some embodiments, a repeat expansion disease or disorder is a polyglutamine disease.

As used herein, “neurodegenerative disease” or “neurodegenerative disorder” refers to diseases or disorders that exhibit neural cell death as a pathological state. A neurodegenerative disease may exhibit chronic neurodegeneration, e.g., slow, progressive neural cell death over a period of several years, or acute neurodegeneration, e.g., sudden onset or neural cell death. Examples of chronic, neurodegenerative diseases include Alzheimer's disease, Parkinson's disease, Huntington's disease, spinocerebellar ataxia types 1-8 (SCA1-8), frontotemporal dementia (FTD), and amyotrophic lateral sclerosis (ALS). A neurodegenerative disease may exhibit death of mainly one type of neuron or of multiple types of neurons.

As used herein, “subject,” “patient,” and “individual” are used interchangeably herein and refer to living organisms (e.g., mammals) selected for treatment or therapy. Examples of subjects include human and non-human mammals, such as primates (monkey, chimpanzee), cows, horses, sheep, dogs, cats, rats, mice, guinea pigs, pigs, and transgenic species thereof.

### A. Double-Stranded RNAs

The present disclosure provides artificial double stranded RNAs which function as artificial microRNAs or shRNAs. Double stranded RNAs of the disclosure modulate expression of a target RNA (e.g., mRNA or pre-mRNA) transcript. Double stranded RNAs include precursor molecules, which are processed inside the cell prior to modulation. Double stranded RNAs may be encoded in a plasmid, vector, genome, or other nucleic acid expression vector for delivery to a cell.

In some embodiments, artificial double stranded RNA comprise from 5′ to 3′: (a) a 5′ leader sequence; (b) a 5′ stem comprising or substantially comprising a passenger sequence or a guide sequence; (c) a 5′ linker of 1-6 bases; (d) a terminal loop; (e) a 3′ linker of 1-6 bases; (f) a 3′ stem comprising or substantially comprising: (i) a guide sequence if the 5′ stem comprises or substantially comprises the passenger sequence; or (ii) a passenger sequence if the 5′ stem comprises or substantially comprises the guide sequence; and (g) a 3′ trailer sequence; wherein the guide sequence targets a CAG repeat region of a CAG repeat containing mRNA or pre-mRNA and comprises 1-5 base mismatches relative to the CAG repeat region, wherein the base mismatches are located at positions 8-16 of the guide sequence.

In some embodiments, the double stranded RNA refers to a single RNA oligonucleotide compound having at least partial self-complementarity to form a stable self-duplex. Unless otherwise specified, numbering of the nucleotide positions in the double-stranded RNA counts from the 5′ to 3′ direction on the single RNA strand. Similarly, unless otherwise specified, nucleotide sequence is read from the 5′ to 3′ direction on the single RNA strand.

An artificial double stranded RNA of the present disclosure comprises a 5′ leader, also referred to as a 5′ flanking, sequence. The 5′ leader sequence may be derived from or obtained in whole or in part from wild type microRNA sequence or be in whole or in part artificial. In some embodiments, 5′ leader sequence is derived from or obtained in whole or in part from the flanking sequence of a wild type pre-miRNA scaffold or pri-miRNA scaffold.

The 5′ leader sequence is contiguously linked to the 5′ stem comprising or substantially comprising a passenger or a guide sequence. The 5′ leader sequence may be of any length. In some embodiments, the 5′ leader sequence is about 1 nucleotide to about 1,000 nucleotides in length, about 1 nucleotide to about 900 nucleotides, about 1 nucleotide to about 800 nucleotides, about 1 nucleotide to about 700 nucleotides, about 1 nucleotide to about 600 nucleotides, about 1 nucleotide to about 500 nucleotides, 1 nucleotide to about 400 nucleotides, about 1 nucleotide to about 300 nucleotides, about 1 nucleotide to about 200 nucleotides, about 1 nucleotide to about 100 nucleotides, about 1 nucleotide to about 75 nucleotides about 1 nucleotide to about 50 nucleotides, about 1 nucleotide to about 25 nucleotides, about 1 nucleotide to about 20 nucleotides, about 1 nucleotide to about 15 nucleotides, or about 1 nucleotide to about 10 nucleotides in length.

In some embodiments, the 5′ leader comprises a 5′ bulge sequence. As used herein, the term “bulge sequence” refers to a region of nucleic acid that is non-complementary to the nucleic acid opposite it in a duplex. For example, a duplex may contain a region of complementary nucleic acids, then a region of non-complementary nucleic acids, followed by a second region of complementary nucleic acids. The regions of complementary nucleic acids will bind to each other, whereas the central non-complementary region will not bind, thereby forming a “bulge.” In some embodiments, the two strands of nucleic acid positioned between the two complementary regions will be of different lengths, thereby forming a “bulge.”

An artificial double-stranded RNA of the present disclosure comprises a 3′ trailer, also referred to as a 3′ flanking sequence. The 3′ trailer sequence may be derived from or obtained in whole or in part from wild type microRNA sequence or be in whole or in part artificial. In some embodiments, the 3′ trailer sequence is derived from or obtained in whole or in part from the flanking sequence of a wild type pre-miRNA scaffold or pri-miRNA scaffold.

The 3′ trailer sequence is contiguously linked to the 3′ stem comprising or substantially comprising a guide or a passenger sequence. The 3′ trailer sequence may be of any length. In some embodiments, the 3′ trailer sequence is about 1 nucleotide to about 1,000 nucleotides in length, about 1 nucleotide to about 900 nucleotides, about 1 nucleotide to about 800 nucleotides, about 1 nucleotide to about 700 nucleotides, about 1 nucleotide to about 600 nucleotides, about 1 nucleotide to about 500 nucleotides, 1 nucleotide to about 400 nucleotides, about 1 nucleotide to about 300 nucleotides, about 1 nucleotide to about 200 nucleotides, about 1 nucleotide to about 100 nucleotides, about 1 nucleotide to about 75 nucleotides about 1 nucleotide to about 50 nucleotides, about 1 nucleotide to about 25 nucleotides, about 1 nucleotide to about 20 nucleotides, about 1 nucleotide to about 15 nucleotides, or about 1 nucleotide to about 10 nucleotides in length. In some embodiments, the 3′ trailer comprises a 3′ bulge sequence.

In some embodiments, the 3′ trailer comprises a polyU (polyuridine) tail. In some embodiments, the 3′ trailer comprises 3-6 uridines, e.g., 3 uridines, 4 uridines, 5 uridines or 6 uridines. In some embodiments, the polyU tail is immediately adjacent to the guide sequence or passenger sequence in the 3′ stem. In some embodiments, artificial double stranded RNA having a 3′ trailer comprising a polyU tail is expressed using a Pol III promoter.

In some embodiments, the 3′ trailer comprises a polyadenylation (pA) signal sequence. Suitable polyadenylation signals include, but are not limited to, an SV40 late pA signal, a BGH pA signal, and the like. In some embodiments, an artificial double stranded RNA having a 3′ trailer comprising a pA signal sequence is expressed using a Pol II promoter.

In some embodiments, the 5′ leader sequence and 3′ trailer sequence have the same number of nucleotides. In some embodiments, the 5′ leader sequence and 3′ trailer sequence have different lengths.

In some embodiments, the 5′ leader sequence and 3′ trailer sequence are obtained or derived from, in whole or in part, the same miRNA scaffold, for example the same wild type pre-miRNA scaffold or the same pri-miRNA scaffold. In some embodiments, the 5′ leader sequence and 3′ trailer sequence are both obtained from or derived from, in whole or in part, the miR-33 scaffold. In some embodiments, the 5′ leader sequence and 3′ trailer sequence are both obtained or derived from, in whole or in part, the pri-miR-33 scaffold. In some embodiments, the 5′ leader sequence and 3′ trailer sequence are both obtained or derived from, in whole or part, the pre-miR-33 scaffold. In some embodiments, the 5′ leader sequence and 3′ trailer sequence are selected from Table E.

In some embodiments, the 5′ leader sequence is not complementary to the 3′ trailer sequence. In some embodiments, the 5′ leader sequence is partially complementary to the 3′ trailer sequence. In some embodiments, the 5′ leader sequence contains one, two, or more C mismatches to the uridine(s) in the polyU tail in the 3′ trailer sequence (or C-T mismatch for a DNA sequence encoding the double stranded RNA).

In some embodiments, the 5′ leader and 3′ trailer sequences contain sequences that allow for recognition and cleavage by Drosha. The canonical pathway of miRNA biogenesis in mammals is initiated by the Drosha-DGCR8 (DiGeorge syndrome critical region gene 8) complex (the Microprocessor), which processes long primary miRNAs (pri-miRNAs) into ˜60-nt pre-miRNAs for further processing by Dicer into a duplex ˜22 nt long. In some embodiments, primary miRNA sequences used as, or as part of the 5′ leader sequence and/or 3′ trailer sequence may direct Drosha cleavage of the double-stranded RNA. Methods of using precursor miRNAs as scaffolds for selected expression of guide:passenger duplexes are provided in U.S. Patent Publication No. 2008/0226553 and Liu et al. (2008) Nucleic Acids Res. 36:2811-24, each of which is incorporated by reference in its entirety.

In some embodiments, the artificial double-stranded RNA is processed by a Drosha independent/Dicer dependent pathway. In some embodiments, splicing, 3′-5′ exoribonuclease, or pol III termination may substitute for Drosha cleavage.

In some embodiments, the artificial double-stranded RNA is processed by a Drosha dependent/Dicer independent pathway. An example of Drosha dependent/Dicer independent pathway processing is provided by pri-miR-451, which is processed by Drosha, resulting in a pre-miR-451, which is then cleaved by Ago2 (argonaute 2), ac-pre-mir-451, which is further resected by an as yet unknown mechanism to generate mature miR-451.

In some embodiments, the 5′ leader sequence and/or 3′ trailer sequence comprises or consists of a nucleotide sequence set forth in Table A. In some embodiments, an artificial double-stranded RNA comprises a 5′ leader sequence comprising or consisting of CCGG and a 3′ trailer sequence comprising or consisting of UUUUUG. In some embodiments, an artificial double-stranded RNA comprises a 5′ leader sequence comprising or consisting of CC and a 3′ trailer sequence comprising or consisting of UUUUUG. In some embodiments, an artificial double-stranded RNA comprises a 5′ leader sequence comprising or consisting of GCUG and a 3′ trailer sequence comprising or consisting of ga uuuuug. In some embodiments, an artificial double-stranded RNA comprises a 5′ leader sequence comprising or consisting of SEQ ID NO:7 and a 3′ trailer sequence comprising or consisting of SEQ ID NO:13.

TABLE A
Examples of 5′ Leader and 3′ Trailer Sequences

| 5′ Leader Sequences | | |
|---|---|---|
| Description | Sequence | SEQ ID NO: |
| | CCGG | NA |
| | CC | NA |
| | CACC | NA |
| miR-33 partial (DNA) | GCTG | NA |
| miR-33 partial (RNA) | GCUG | NA |
| miR-33 full (DNA) | tgcacacctcctgggggcagct ctg | 738 |
| miR-33 full (RNA) | ugcacaccuccugggggcagcu cug | 7 |
| 3′ Trailer Sequences | | |
| Description | Sequence | SEQ ID NO: |
| | TTTTTG | NA |
| | UUUUUG | NA |
| miR-33 partial (DNA) | ga tttttg | NA |
| miR-33 partial (RNA) | ga uuuuug | NA |
| miR-33 full (DNA) | gg aggcctgccctgactgcccac tttttg | 12 |
| miR-33 full (RNA) | gg aggccugcccugacugcccac uuuuug | 13 |

In some embodiments, the 5′ stem (or 5′ arm) of the double stranded RNA comprises a passenger sequence, also sometimes referred to as sense sequence. The passenger sequence has identity to the target mRNA transcript. The passenger sequence may be about 15-30 nucleotides in length, for example, 15, 16, 17, 18, 19, 20, 21, 22, 23, 24, 25, 26, 27, 28, 29 or 30 nucleotides in length. In some embodiments, the passenger sequence may be about 19-24 nucleotides in length.

In some embodiments, the 3′ stem (or 3′ arm) of the double stranded RNA comprises a guide sequence, also sometimes referred to as antisense sequence. The guide sequence has complementarity to the target mRNA transcript. The guide strand may be about 15-30 nucleotides in length, for example, 15, 16, 17, 18, 19, 20, 21, 22, 23, 24, 25, 26, 27, 28, 29 or 30 nucleotides in length. In some embodiments, the guide sequence may be about 19-24 nucleotides in length.

In some embodiments, the 5′ stem comprises the guide sequence while the 3′ stem comprises the passenger sequence of the double stranded RNA.

The guide sequence and passenger sequence have sufficient complementarity to form a double stranded siRNA molecule upon processing in a host cell, which acts as a suitable substrate for the RNA interference machinery such that the guide sequence derived from the 3′ stem (or 5′ stem) is recognized by the RISC complex and targets its specific mRNA transcript. In some embodiments, the guide sequence and passenger sequence have 100% complementarity. In some embodiments, the guide sequence and passenger sequence are substantially complementary to each other, e.g., about 70%, 75%, 80%, 85%, 90%, 95%, or 99% complementary. In some embodiments, the passenger sequence may comprise one to ten or one to five base mismatches or bulges.

The guide sequence may comprise a seed sequence, which has perfect or near-perfect Watson-Crick complementarity to the target mRNA sequence, located at positions 1-7, 2-7, 1-8, or 2-8, of the guide sequence relative to the first 5′ nucleotide of the guide strand. The seed region is important for efficient gene silencing by double stranded RNAs.

The guide sequence targets a CAG repeat region of a CAG repeat containing mRNA and comprises 1-5 mismatches relative to the CAG repeat region, wherein the base mismatches are located at positions 8-16 of the guide sequence. A mismatch includes self-pairing nucleotides (A-A, U-U, T-T, C-C, and G-G), A and C pairing, C and U pairing, C and T pairing, and A and G pairing. In some embodiments, the mismatch comprises a purine mismatch, such as introducing an adenosine base into the guide strand.

In some embodiments, the guide sequence targeting a CAG repeat region comprises about 1-5 base mismatches relative to the CAG repeat region of a CAG repeat containing mRNA. In some embodiments, the guide sequence targeting a CAG repeat region comprises about 1-4 base mismatches, about 1-3 base mismatches, about 1-2 base mismatches, about 2-5 base mismatches, about 3-4 base mismatches, about 3-5 base mismatches, or about 4-5 base mismatches relative to the CAG repeat region of a CAG repeat containing mRNA. In some embodiments, the guide sequence targeting a CAG repeat region comprises about one mismatch, about two mismatches, about three mismatches, about four mismatches, or about five mismatches relative to the CAG repeat region of a CAG repeat containing mRNA.

In some embodiments, at least one mismatch (1, 2, 3, 4, or 5) relative to the CAG repeat region may be located at positions 8-12 of the guide sequence. In some embodiments, at least one mismatch (1, 2, or 3) relative to the CAG repeat region may be located at positions 9-11 of the guide sequence. In some embodiments, two or more mismatches relative to the CAG repeat region located at positions 8-16 of the guide sequence are contiguous or adjacent to each other. In some embodiments, at least one mismatch relative to the CAG repeat region located at positions 8-16 is not adjacent to another mismatch located at positions 8-16.

In some embodiments, a single mismatch relative to the CAG repeat region is located at position 8 of the guide sequence. In some embodiments, a single mismatch relative to the CAG repeat region is located at position 9 of the guide sequence. In some embodiments, a single mismatch relative to the CAG repeat region is located at position 10 of the guide sequence. In some embodiments, a single mismatch relative to the CAG repeat region is located at position 11 of the guide sequence.

In some embodiments, one mismatch relative to the CAG repeat region is located at position 8 of the guide sequence and one mismatch relative to the CAG repeat region is located at any of positions 9-16 of the guide sequence. In some embodiments, one mismatch relative to the CAG repeat region is located at position 9 of the guide sequence and one mismatch relative to the CAG repeat region is located at any of positions 10-16 of the guide sequence. In some embodiments, one mismatch relative to the CAG repeat region is located at position 10 of the guide sequence and one mismatch relative to the CAG repeat region is located at any of positions 11-16 of the guide sequence. In some embodiments, one mismatch relative to the CAG repeat region is located at position 11 of the guide sequence and one mismatch relative to the CAG repeat region is located at any of positions 12-16 of the guide sequence.

In some embodiments, one mismatch relative to the CAG repeat region is located at position 8 of the guide sequence, one mismatch relative to the CAG repeat region is located at position 9 of the guide sequence, and one mismatch relative to the CAG repeat region is located at any of positions 10-16 of the guide sequence. In some embodiments, one mismatch relative to the CAG repeat region is located at position 9 of the guide sequence, one mismatch relative to the CAG repeat region is located at position 10 of the guide sequence, and one mismatch relative to the CAG repeat region is located at any of positions 11-16 of the guide sequence. In some embodiments, one mismatch relative to the CAG repeat region is located at position 10 of the guide sequence, one mismatch relative to the CAG repeat region is located position 11 of the guide sequence, and one mismatch relative to the CAG repeat region is located at any of positions 12-16 of the guide sequence. In some embodiments, one mismatch relative to the CAG repeat region is located at position 11 of the guide sequence, one mismatch relative to the CAG repeat region is located at position 12 of the guide sequence, and one mismatch relative to the CAG repeat region is located at any of positions 13-16 of the guide sequence.

In some embodiments, one mismatch relative to the CAG repeat region is located at position 8 of the guide sequence, one mismatch relative to the CAG repeat region is located position 9 of the guide sequence, one mismatch relative to the CAG repeat region is located at any of positions 12-16 of the guide sequence, and one mismatch relative to the CAG repeat region is located at position 14, 15, or 16. In some embodiments, one mismatch relative to the CAG repeat region is located at position 9 of the guide sequence, one mismatch relative to the CAG repeat region is located position 10 of the guide sequence, one mismatch relative to the CAG repeat region is located at any of positions 11-16 of the guide sequence, and one mismatch relative to the CAG repeat region is located at position 15 or 16. In some embodiments, one mismatch relative to the CAG repeat region is located at position 9 of the guide sequence, one mismatch relative to the CAG repeat region is located position 11 of the guide sequence, one mismatch relative to the CAG repeat region is located at any of positions 12-16 of the guide sequence, and one mismatch relative to the CAG repeat region is located at position 13, 14, 15 or 16.

In some embodiments, in a guide sequence targeting a CAG repeat region in a CAG register, one mismatch relative to the CAG repeat region is located at position 9 of the guide sequence, one mismatch relative to the CAG repeat region is located position 10 of the guide sequence, one mismatch relative to the CAG repeat region is located at position 11 of the guide sequence, one mismatch relative to the CAG repeat region is located at position 15 of the guide sequence, and one mismatch relative to the CAG repeat region is located at position 16 of the guide sequence. In some embodiments, the mismatches at positions 9, 10 and 11 are A, A, and A, respectively, and the mismatches at positions 15 and 16 are AA, AU, UA, or UU.

In some embodiments, a guide sequence targeting a CAG repeat region in a UGC register comprise one mismatch relative to the CAG repeat region is located at position 9 of the guide sequence, one mismatch relative to the CAG repeat region is located position 10 of the guide sequence, one mismatch relative to the CAG repeat region is located at position 12 of the guide sequence, one mismatch relative to the CAG repeat region is located at position 15 of the guide sequence, and one mismatch relative to the CAG repeat region is located at position 16 of the guide sequence. In some embodiments, the mismatches at positions 9, 10 and 12 are A, C, and A, respectively, and the mismatches at positions 15 and 16 are AA, UA, or UU.

In some embodiments, a guide sequence targeting a CAG repeat region in a GCU register comprise one mismatch relative to the CAG repeat region is located at position 9 of the guide sequence, one mismatch relative to the CAG repeat region is located position 10 of the guide sequence, one mismatch relative to the CAG repeat region is located at position 11 of the guide sequence, one mismatch relative to the CAG repeat region is located at position 15 of the guide sequence, and one mismatch relative to the CAG repeat region is located at position 16 of the guide sequence. In some embodiments, the mismatches at positions 9, 10 and 11 are A, U, and A, respectively, and the mismatches at positions 15 and 16 are AA or AU.

In some embodiments, the guide sequence targeting a CAG repeat region comprises or consists of any sequence selected from Tables B1-B2. In some embodiments, a double stranded RNA comprises a guide sequence selected from Tables B1-B2 and a corresponding passenger sequence that is perfectly complementary to the guide sequence or has one to ten or one to five mismatches or bulges compared to the selected guide sequence.

TABLE B1
CAG Repeat Targeting Oligonucleotides

| # | | |
|---|---|---|
| mismatches | Guide sequence (RNA) | SEQ ID NO: # |
| 1 | CUGCUGCUUCUGCUGCUGCUG | SEQ ID NO: 35 |
| | UGCUGCUGUUGCUGCUGCUGC | SEQ ID NO: 36 |
| | GCUGCUGCAGCUGCUGCUGCU | SEQ ID NO: 37 |
| 2 | CUGCUGCUUAUGCUGCUGCUG | SEQ ID NO: 38 |
| | UGCUGCUGUAUCUGCUGCUGC | SEQ ID NO: 39 |
| | CUGCUGCUUCUGAUGCUGCUG | SEQ ID NO: 41 |
| | CUGCUGCUUCUGCGGCUGCUG | SEQ ID NO: 42 |
| 3 | CUGCUGCUAGUACUGCUGCUG | SEQ ID NO: 43 |
| | GCUGCUGCAUAUGCUGCUGCU | SEQ ID NO: 40 |
| | CUGCUGCUAAUACUGCUGCUG | SEQ ID NO: 326 |
| | CUGCUGCUUGCGCUGCUGCUG | SEQ ID NO: 45 |
| | CUGCUGCUAGGGCUGCUGCUG | SEQ ID NO: 46 |
| 4 | CUGCUGCUAAAACUGCUGCUG | SEQ ID NO: 347 |
| | CUGCUGCUAAACCUGCUGCUG | SEQ ID NO: 48 |
| | CUGCUGCUUAAACUGCUGCUG | SEQ ID NO: 49 |
| | CUGCUGCUUAACCUGCUGCUG | SEQ ID NO: 50 |

Nucleotide mismatch(es) in guide sequence relative to the CAG repeat region of a CAG repeat containing mRNA is in bold and underlined.

TABLE B2
CAG Repeat Targeting

| Mismatch position & type | Guide sequence (RNA) | SEQ ID NO: # |
|---|---|---|
| 9: G > U | CUGCUGCUUCUGCUGCUGCUG | SEQ ID NO: 35 |
| 9: G > A | CUGCUGCUACUGCUGCUGCUG | SEQ ID |
| | | NO: 295 |
| 9: G > C | CUGCUGCUCCUGCUGCUGCUG | SEQ ID |
| | | NO: 220 |
| 9: C > A | UGCUGCUGAUGCUGCUGCUGC | SEQ ID |
| | | NO: 221 |
| 9: C > G | UGCUGCUGGUGCUGCUGCUGC | SEQ ID |
| | | NO: 222 |
| 9: U > A | GCUGCUGCAGCUGCUGCUGCU | SEQ ID NO: 37 |
| 9: U > C | GCUGCUGCCGCUGCUGCUGCU | SEQ ID |
| | | NO: 224 |
| 9: U > G | GCUGCUGCGGCUGCUGCUGCU | SEQ ID |
| | | NO: 225 |
| 9: G > U_10: C > A | CAGCAGCAGCAUAAGCAGCAG | SEQ ID NO: 75 |
| 9: G > U_11: U > G | CUGCUGCUUCGGCUGCUGCUG | SEQ ID NO: 76 |
| 9: G > U_12: G > A | CAGCAGCAUCAACAGCAGCGA | SEQ ID NO: 77 |
| 9: G > C_12: G > A | CUGCUGCUCCUACUGCUGCUG | SEQ ID NO: 78 |
| 9: G > A_13: C > G | CUGCUGCUACUGGUGCUGCUG | SEQ ID NO: 40 |
| 9: G > U_13: C > A | CUGCUGCUUCUGAUGCUGCUG | SEQ ID NO: 80 |
| 9: G > A_14: U > G | CUGCUGCUACUGCGGCUGCUG | SEQ ID NO: 81 |
| 9: G > C_14: U > C | CUGCUGCUCCUGCCGCUGCUG | SEQ ID NO: 82 |
| 9: G > U_14: U > C | CUGCUGCUUCUGCCGCUGCUG | SEQ ID NO: 83 |
| 9: G > U_14: U > G | CUGCUGCUUCUGCGGCUGCUG | SEQ ID NO: 41 |
| 9: G > C_15: G > A | CUGCUGCUCCUGCUACUGCUG | SEQ ID NO: 85 |
| 9: G > C_16: C > A | CUGCUGCUCCUGCUGAUGCUG | SEQ ID NO: 86 |
| 9: G > U_16: C > G | CUGCUGCUUCUGCUGGUGCUG | SEQ ID NO: 87 |
| 10: U > C_15: C > A | UGCUGCUGCCGCUGAUGCUGC | SEQ ID NO: 88 |
| 10: U > G_11: G > U | UGCUGCUGCGUCUGCUGCUGC | SEQ ID NO: 89 |
| 10: U > G_12: C > A | UGCUGCUGCGGAUGCUGCUGC | SEQ ID NO: 90 |
| 10: U > G_15: C > A | UGCUGCUGCGGCUGAUGCUGC | SEQ ID NO: 91 |
| 11: G > A_13: U > G | UGCUGCUGCUACGGCUGCUGC | SEQ ID NO: 92 |
| 11: G > A_15: C > A | UGCUGCUGCUACUGAUGCUGC | SEQ ID NO: 93 |
| 11: G > C_12: C > G | UGCUGCUGCUCGUGCUGCUGC | SEQ ID NO: 94 |
| 11: G > C_14: G > A | UGCUGCUGCUCCUACUGCUGC | SEQ ID NO: 95 |
| 11: G > U_12: C > A | UGCUGCUGCUUAUGCUGCUGC | SEQ ID NO: 96 |
| 11: G > U_13: U > G | UGCUGCUGCUUCGGCUGCUGC | SEQ ID NO: 97 |
| 11: G > U_14: G > A | UGCUGCUGCUUCUACUGCUGC | SEQ ID NO: 98 |
| 11: G > U_16: U > G | UGCUGCUGCUUCUGCGGCUGC | SEQ ID NO: 99 |
| 8: G > C_14: G > A | UGCUGCUCCUGCUACUGCUGC | SEQ ID |
| | | NO: 100 |
| 8: G > C_9: C > G | UGCUGCUCGUGCUGCUGCUGC | SEQ ID |
| | | NO: 101 |
| 8: G > U_10: U > G | UGCUGCUUCGGCUGCUGCUGC | SEQ ID |
| | | NO: 102 |
| 8: G > U_12: C > A | UGCUGCUUCUGAUGCUGCUGC | SEQ ID |
| | | NO: 103 |
| 8: G > U_13: U > C | UGCUGCUUCUGCCGCUGCUGC | SEQ ID |
| | | NO: 104 |
| 8: G > U_13: U > G | UGCUGCUUCUGCGGCUGCUGC | SEQ ID |
| | | NO: 105 |
| 8: G > U_9: C > A | UGCUGCUUAUGCUGCUGCUGC | SEQ ID |
| | | NO: 106 |
| 9: C > A 11: G > U | UGCUGCUGAUUCUGCUGCUGC | SEQ ID |
| | | NO: 107 |
| 9: C > A_16: U > C | UGCUGCUGAUGCUGCCGCUGC | SEQ ID |
| | | NO: 108 |
| 9: C > A_10: U > C | UGCUGCUGACGCUGCUGCUGC | SEQ ID |
| | | NO: 109 |
| 10: G > A_14: C > A | GCUGCUGCUACUGAUGCUGCU | SEQ ID |
| | | NO: 110 |
| 10: G > A_14:C > G | GCUGCUGCUACUGGUGCUGCU | SEQ ID |
| | | NO: 111 |
| 10: G > C_11: C > G | GCUGCUGCUCGUGCUGCUGCU | SEQ ID |
| | | NO: 112 |
| 10: G > C_13: G > A | GCUGCUGCUCCUACUGCUGCU | SEQ ID |
| | | NO: 113 |
| 10: G > U_11: C > A | GCUGCUGCUUAUGCUGCUGCU | SEQ ID |
| | | NO: 114 |
| 10: G > U_12: U > G | GCUGCUGCUUCGGCUGCUGCU | SEQ ID |
| | | NO: 115 |
| 10: G > U_14: C > A | GCUGCUGCUUCUGAUGCUGCU | SEQ ID |
| | | NO: 116 |
| 10: G > U_15: U > G | GCUGCUGCUUCUGCGGCUGCU | SEQ ID |
| | | NO: 117 |
| 11: C > A_13: G > A | GCUGCUGCUGAUACUGCUGCU | SEQ ID |
| | | NO: 118 |
| 11: C > A_13: G > C | GCUGCUGCUGAUCCUGCUGCU | SEQ ID |
| | | NO: 119 |
| 11: C > A_13: G > U | GCUGCUGCUGAUUCUGCUGCU | SEQ ID |
| | | NO: 120 |
| 11: C > A_15: U > A | GCUGCUGCUGAUGCAGCUGCU | SEQ ID |
| | | NO: 121 |
| 11: C > A_15: U > C | GCUGCUGCUGAUGCCGCUGCU | SEQ ID |
| | | NO: 122 |
| 11: C > U_14: C > A | GCUGCUGCUGUUGAUGCUGCU | SEQ ID |
| | | NO: 123 |
| 8: C > A_11: C > G | GCUGCUGAUGGUGCUGCUGCU | SEQ ID |
| | | NO: 124 |
| 8: C > A_12: U > G | GCUGCUGAUGCGGCUGCUGCU | SEQ ID |
| | | NO: 125 |
| 8: C > A_15: U > C | GCUGCUGAUGCUGCCGCUGCU | SEQ ID |
| | | NO: 126 |
| 8: C > A_16: G > C | GCUGCUGAUGCUGCUCCUGCU | SEQ ID |
| | | NO: 127 |
| 1_2-8: C > G_16: G > U | GCUGCUGGUGCUGCUUCUGCU | SEQ ID |
| | | NO: 128 |
| 2-9: U > C_14: C > A | GCUGCUGCCGCUGAUGCUGCU | SEQ ID |
| | | NO: 129 |
| 2-9: U > G_11: C > A | GCUGCUGCGGAUGCUGCUGCU | SEQ ID |
| | | NO: 130 |
| 9: U > G_14: C > A | GCUGCUGCGGCUGAUGCUGCU | SEQ ID |
| | | NO: 131 |
| 9: G > U_10: C > A_12: G > U | CUGCUGCUUAUUCUGCUGCUG | SEQ ID |
| | | NO: 132 |
| 9: G > C_10: C > A_13: C > A | CUGCUGCUCAUGAUGCUGCUG | SEQ ID |
| | | NO: 133 |
| 9: G > C_10: C > G_13: C > A | CUGCUGCUCGUGAUGCUGCUG | SEQ ID |
| | | NO: 134 |
| 9: G > C_10: C > G_13: C > G | CUGCUGCUCGUGGUGCUGCUG | SEQ ID |
| | | NO: 135 |
| 9: G > U_10: C > A_13: C > G | CUGCUGCUUAUGGUGCUGCUG | SEQ ID |
| | | NO: 136 |
| 9: G > U_10: C > G_13: C > A | CUGCUGCUUGUGAUGCUGCUG | SEQ ID |
| | | NO: 137 |
| 9: G > U_10: C > G_13: C > G | CUGCUGCUUGUGGUGCUGCUG | SEQ ID |
| | | NO: 138 |
| 9: G > A_10: C > G_14: U > G | CUGCUGCUAGUGCGGCUGCUG | SEQ ID |
| | | NO: 139 |
| 9: G > C_10: C > G_14: U > A | CUGCUGCUCGUGCAGCUGCUG | SEQ ID |
| | | NO: 140 |
| 9: G > C_10: C > G_14: U > G | CUGCUGCUCGUGCGGCUGCUG | SEQ ID |
| | | NO: 141 |
| 9: G > U_10: C > A_14: U > C | CUGCUGCUUAUGCCGCUGCUG | SEQ ID |
| | | NO: 142 |
| 9: G > U_10: C > A_14: U > G | CUGCUGCUUAUGCGGCUGCUG | SEQ ID |
| | | NO: 143 |
| 9: G > C_10: C > A_15: G > U | CUGCUGCUCAUGCUUCUGCUG | SEQ ID |
| | | NO: 144 |
| 9: G > U_10: C > A_15: G > A | CUGCUGCUUAUGCUACUGCUG | SEQ ID |
| | | NO: 145 |
| 9: G > U_10: C > A_15: G > C | CUGCUGCUUAUGCUCCUGCUG | SEQ ID |
| | | NO: 146 |
| 9: G > C_10: C > G_16: C > A | CUGCUGCUCGUGCUGAUGCUG | SEQ ID |
| | | NO: 147 |
| 9: G > C_10: C > G_16: C > G | CUGCUGCUCGUGCUGGUGCUG | SEQ ID |
| | | NO: 148 |
| 9: G > U_10: C > A_16: C > G | CUGCUGCUUAUGCUGGUGCUG | SEQ ID |
| | | NO: 149 |
| 9: G > U_10: C > G_16: C > A | CUGCUGCUUGUGCUGAUGCUG | SEQ ID |
| | | NO: 150 |
| 9: G > A_10: C > A_11: U > A | CUGCUGCUAAAGCUGCUGCUG | SEQ ID |
| | | NO: 325 |
| 9: G > A_10: C > A_11: U > C | CUGCUGCUAACGCUGCUGCUG | SEQ ID |
| | | NO: 152 |
| 9: G > A_10: C > G_11: U > G | CUGCUGCUAGGGCUGCUGCUG | SEQ ID NO: 46 |
| 9: G > C_10: C > A_11: U > C | CUGCUGCUCACGCUGCUGCUG | SEQ ID |
| | | NO: 154 |
| 9: G > C_10: C > G_11: U > C | CUGCUGCUCGCGCUGCUGCUG | SEQ ID |
| | | NO: 155 |
| 9: G > U_10: C > A_11: U > C | CUGCUGCUUACGCUGCUGCUG | SEQ ID |
| | | NO: 156 |
| 9: G > U_10: C > A_11: U > G | CUGCUGCUUAGGCUGCUGCUG | SEQ ID |
| | | NO: 157 |
| 9: G > U_10: C > G_11: U > C | CUGCUGCUUGCGCUGCUGCUG | SEQ ID NO: 45 |
| 9: C > A_10: U > A_14: G > U | UGCUGCUGAAGCUUCUGCUGC | SEQ ID |
| | | NO: 159 |
| 9: C > A_10: U > C_12: C > A | UGCUGCUGACGAUGCUGCUGC | SEQ ID |
| | | NO: 160 |
| 9: C > A_10: U > C_12: C > G | UGCUGCUGACGGUGCUGCUGC | SEQ ID |
| | | NO: 161 |
| 9: C > A_10: U > C_13: U > G | UGCUGCUGACGCGGCUGCUGC | SEQ ID |
| | | NO: 162 |
| 9: C > A_10: U > C_14: G > U | UGCUGCUGACGCUUCUGCUGC | SEQ ID |
| | | NO: 163 |
| 9: C > G_10: U > C_14: G > C | UGCUGCUGGCGCUCCUGCUGC | SEQ ID |
| | | NO: 164 |
| 9: U > A_10: G > C_14: C > G | GCUGCUGCACCUGGUGCUGCU | SEQ ID |
| | | NO: 165 |
| 9: U > G_10: G > A_14: C > A | GCUGCUGCGACUGAUGCUGCU | SEQ ID |
| | | NO: 166 |
| 9: U > G_10: G > A_16: G > U | GCUGCUGCGACUGCUUCUGCU | SEQ ID |
| | | NO: 167 |
| 9: U > G_10: G > C_11: C > A | GCUGCUGCGCAUGCUGCUGCU | SEQ ID |
| | | NO: 168 |
| 9: U > G_10: G > C_11: C > G | GCUGCUGCGCGUGCUGCUGCU | SEQ ID |
| | | NO: 169 |
| 9: U > G_10: G > C_14: C > A | GCUGCUGCGCCUGAUGCUGCU | SEQ ID |
| | | NO: 170 |
| 9: U > G_10: G > C_14: C > G | GCUGCUGCGCCUGGUGCUGCU | SEQ ID |
| | | NO: 171 |
| 9: U > G_10: G > U_11: C > A | GCUGCUGCGUAUGCUGCUGCU | SEQ ID |
| | | NO: 172 |
| 9: U > G_10: G > U_13: G > U | GCUGCUGCGUCUUCUGCUGCU | SEQ ID |
| | | NO: 173 |
| 9: U > G_10: G > U_14: C > A | GCUGCUGCGUCUGAUGCUGCU | SEQ ID |
| | | NO: 174 |
| 9: U > G_10: G > U_14: C > G | GCUGCUGCGUCUGGUGCUGCU | SEQ ID |
| | | NO: 175 |
| 9: U > G_10: G > U_15: U > A | GCUGCUGCGUCUGCAGCUGCU | SEQ ID |
| | | NO: 176 |
| 9: U > A_10: G > U_11: C > A | GCUGCUGCAUAUGCUGCUGCU | SEQ ID NO: 43 |
| 9: G > A_10: C > G_12: | CUGCUGCUAGUCCUUCUGCUG | SEQ ID |
| G > C_15: G > U | | NO: 178 |
| 9: G > C_10: C > A_12: | CUGCUGCUCAUUCUACUGCUG | SEQ ID |
| G > U_15: G > A | | NO: 179 |
| 9: G > C_10: C > G_12: | CUGCUGCUCGUACUGAUGCUG | SEQ ID |
| G > A_16: C > A | | NO: 180 |
| 9: G > C_10: C > G_12: | CUGCUGCUCGUUCUGGUGCUG | SEQ ID |
| G > U_16: C > G | | NO: 181 |
| 9: G > A_10: C > G_13: | CUGCUGCUAGUGAUCCUGCUG | SEQ ID |
| C > A_15: G > C | | NO: 182 |
| 9: G > A_10: C > G_13: | CUGCUGCUAGUGGUUCUGCUG | SEQ ID |
| C > G_15: G > U | | NO: 183 |
| 9: G > A_10: C > A_14: | CUGCUGCUAAUGCGUCUGCUG | SEQ ID |
| U > G_15: G > U | | NO: 184 |
| 9: G > C_10: C > A_14: | CUGCUGCUCAUGCGUCUGCUG | SEQ ID |
| U > G_15: G > U | | NO: 185 |
| 9: G > C_10: C > G_14: | CUGCUGCUCGUGCGCCUGCUG | SEQ ID |
| U > G_15: G > C | | NO: 186 |
| 9: G > C_10: C > G_14: | CUGCUGCUCGUGCGUCUGCUG | SEQ ID |
| U > G_15: G > U | | NO: 187 |
| 9: G > U_10: C > A_14: | CUGCUGCUUAUGCGUCUGCUG | SEQ ID |
| U > G_15: G > U | | NO: 188 |
| 9: G > A_10: C > A_14: | CUGCUGCUAAUGCGGUUGCUG | SEQ ID |
| U > G_16: C > U | | NO: 189 |
| 9: G > C_10: C > G_14: | CUGCUGCUCGUGCAGAUGCUG | SEQ ID |
| U > A_16: C > A | | NO: 190 |
| 9: G > C_10: C > G_14: | CUGCUGCUCGUGCGGAUGCUG | SEQ ID |
| U > G_16: C > A | | NO: 191 |
| 9: G > A_10: C > G_15: | CUGCUGCUAGUGCUUAUGCUG | SEQ ID |
| G > U_16: C > A | | NO: 192 |
| 9: G > C_10: C > G_15: | CUGCUGCUCGUGCUCAUGCUG | SEQ ID |
| G > C_16: C > A | | NO: 193 |
| 9: G > C_10: C > G_15: | CUGCUGCUCGUGCUUAUGCUG | SEQ ID |
| G > U_16: C > A | | NO: 194 |
| 9: G > A_10: C > A_11: | CUGCUGCUAAGGCUCCUGCUG | SEQ ID |
| U > G_15: G > C | | NO: 195 |
| 9: G > U_10: C > A_11: | CUGCUGCUUAGGCUCCUGCUG | SEQ ID |
| U > G_15: G > C | | NO: 196 |
| 9: G > A_10: C > A_11: | CUGCUGCUAACGCUGUUGCUG | SEQ ID |
| U > C_16: C > U | | NO: 197 |
| 9: G > A_10: C > G_11: | CUGCUGCUAGCGCUGUUGCUG | SEQ ID |
| U > C_16: C > U | | NO: 198 |
| 9: G > U_10: C > A_11: | CUGCUGCUUAGGCUGUUGCUG | SEQ ID |
| U > G_16: C > U | | NO: 199 |
| 9: C > A_10: U > C_14: | UGCUGCUGACGCUUUUGCUGC | SEQ ID |
| G > U_15: C > U | | NO: 200 |
| 9: C > A_10: U > C_14: | UGCUGCUGACGCUUCGGCUGC | SEQ ID |
| G > U_16: U > G | | NO: 201 |
| 9: C > G_10: U > C_14: | UGCUGCUGGCGCUUUUGCUGC | SEQ ID |
| G > U_15: C > U | | NO: 202 |
| 9: C > U_10: U > C_14: | UGCUGCUGUCGCUUCGGCUGC | SEQ ID |
| G > U_16: U > G | | NO: 203 |
| 9: U > A_10: G > U_11: | GCUGCUGCAUAUACUGCUGCU | SEQ ID |
| C > A_13: G > A | | NO: 204 |
| 9: U > G_10: G > C_11: | GCUGCUGCGCAUGCUUCUGCU | SEQ ID |
| C > A_16: G > U | | NO: 205 |
| 9: U > G_10: G > C_11: | GCUGCUGCGCGUGAUGCUGCU | SEQ ID |
| C > G_14: C > A | | NO: 206 |
| 9: U > G_10: G > C_11: | GCUGCUGCGCGUGCUUCUGCU | SEQ ID |
| C > G_16: G > U | | NO: 207 |
| 9: U > G_10: G > U_11: | GCUGCUGCGUAUGCCGCUGCU | SEQ ID |
| C > A_15: U > C | | NO: 208 |
| 9: U > G_10: G > U_11: | GCUGCUGCGUAUGCUUCUGCU | SEQ ID |
| C > A_16: G > U | | NO: 209 |
| 9: G > A_10: C > A_11: | CUGCUGCUAAAGCUAAUGCUG | SEQ ID |
| U > A_15: G > A_16: C > A | | NO: 210 |
| 9: G > A_10: C > A_11: | CUGCUGCUAAAGCUAUUGCUG | SEQ ID |
| U > A_15: G > A_16: C > U | | NO: 211 |
| 9: G > A_10: C > A_11: | CUGCUGCUAAAGCUUAUGCUG | SEQ ID |
| U > A_15: G > U_16: C > A | | NO: 212 |
| 9: G > A_10:C > A_11: | CUGCUGCUAAAGCUUUUGCUG | SEQ ID |
| U > A_15: G > U_16: C > U | | NO: 213 |
| 9:C > A_10: U > C_12: | UGCUGCUGACGAUGAAGCUGC | SEQ ID |
| C > A_15: C > A 16: U > A | | NO: 214 |
| 9: C > A_10:U > C_12: | UGCUGCUGACGAUGUAGCUGC | SEQ ID |
| C > A_15: C > U_16: U > A | | NO: 215 |
| 9: U > A_10: G > U_11: | GCUGCUGCAUAUGCAACUGCU | SEQ ID |
| C > A_15: U > A_16: G > A | | NO: 216 |
| 9: U > A_10: G > U_11: | | SEQ ID |
| C > A_15: U > A_16: G > U | GCUGCUGCAUAUGCAUCUGCU | NO: 217 |

In some embodiments, specificity or selectivity of a guide sequence targeting a CAG repeat region of a CAG repeat containing mRNA or pre-mRNA increases with the number of base mismatches relative to the CAG repeat region, wherein the base mismatches are located at positions 8-16 of the guide sequence. Specificity (or off-target activity) of a guide sequence may be determined by detecting potential off-target matches in the human unspliced transcriptome (Ensembl database, release 100). For example, for a 21mer guide sequence targeting CAG repeat having perfect complementarity to CAG repeat (or AGC or GCA if using guide sequence targeting the repeat in a different register) in seed sequence (nucleotides 1-7 from 5′ end) and mismatch at position 8, 9, 10, or 11 of the guide sequence; the following steps may be used to measure off-target activity: measure frequency of off-target genes with perfect matches to the guide sequence; measure frequency of off-target genes with perfect 17mer match to guide sequence within positions 1-21; measure frequency of off-target genes matching the guide sequence having 0, 1, 2, 3, or 4 mismatches between positions 8 and 21 inclusive. Off-target frequencies of perfect matches, perfect 17mer match over positions 1-21, matches to guide with 0 mismatch, matches to guide with 1 mismatch, off-target match to guide with 2 mismatches, off-target match to guide with 3 mismatches, off-target match to guide with 4 mismatches may be tallied.

In some embodiments, specificity of a guide sequence targeting a CAG repeat region of a CAG repeat containing mRNA or pre-mRNA increases with 2-5 base mismatches relative to the CAG repeat region, wherein the base mismatches are located at positions 8-16 of the guide sequence. In some embodiments, specificity of a guide sequence targeting a CAG repeat region of a CAG repeat containing mRNA or pre-mRNA increases with 3-5 base mismatches relative to the CAG repeat region, wherein the base mismatches are located at positions 8-16 of the guide sequence.

In some embodiments, a guide sequence targeting a CAG repeat region of a CAG repeat containing mRNA or pre-mRNA having 1, 2, 3, 4, or 5 base mismatches relative to the CAG repeat region, wherein the base mismatches are located at positions 8-16 of the guide sequence, has 0 predicted perfectly matching off-target transcripts.

In some embodiments, a guide sequence targeting a CAG repeat region of a CAG repeat containing mRNA or pre-mRNA having 1, 2, 3, 4, or 5 base mismatches relative to the CAG repeat region, wherein the base mismatches are located at positions 8-16 of the guide sequence, 0-1 predicted off-target transcripts having perfect 17mer match within positions 1-21.

In some embodiments, a guide sequence targeting a CAG repeat region of a CAG repeat containing mRNA or pre-mRNA having 1, 2, 3, 4, or 5 base mismatches relative to the CAG repeat region, wherein the base mismatches are located at positions 8-16 of the guide sequence, 0-2 predicted off-target transcripts with one mismatch.

In some embodiments, a guide sequence targeting a CAG repeat region of a CAG repeat containing mRNA or pre-mRNA having 1, 2, 3, 4, or 5 base mismatches relative to the CAG repeat region, wherein the base mismatches are located at positions 8-16 of the guide sequence, 0-2 predicted off-target transcripts having perfect 17mer match within positions 1-21.

In some embodiments, a guide sequence targeting a CAG repeat region of a CAG repeat containing mRNA or pre-mRNA having 1, 2, 3, 4, or 5 base mismatches relative to the CAG repeat region, wherein the base mismatches are located at positions 8-16 of the guide sequence, 0-66 predicted off-target transcripts with one mismatch.

TABLE B3
Predicted Off-Target Profile of CAG Repeat Targeting Oligonucleotide

| | | | Frequency | | | | | |
|---|---|---|---|---|---|---|---|---|
| | | | perfect | | | | | |
| | | | 17 mer | off- | off- | off- | off- | off- |
| | | | match | target | target | target | target | target |
| Mismatch | | SEQ | within | genes | genes | genes | genes | genes |
| position & | | ID | positions | w/ 0 | w/ 1 | w/ 2 | w/ 3 | w/ 4 |
| type | sequence (5′-3′) | NO: | 1-21 | mm | mm | mm | mm | mm |
| 9: G > U_10: U > G | CAGCAGCAGCAUAAGC | 75 | 0 | 0 | 4 | 219 | 490 | 1233 |
| | AGCAG | | | | | | | |
| 9: G > U_11: U > G | CUGCUGCUUCGGCUGC | 76 | 0 | 0 | 5 | 229 | 512 | 1300 |
| | UGCUG | | | | | | | |
| 9: G > U_12: G > A | CAGCAGCAGUAGAAGC | 741 | 1 | 0 | 14 | 224 | 500 | 1316 |
| | AGCAG | | | | | | | |
| 9: G > C_12: G > A | CUGCUGCUCCUACUGC | 78 | 1 | 0 | 16 | 219 | 540 | 1353 |
| | UGCUG | | | | | | | |
| 9: G > A_13: C > G | CUGCUGCUACUGGUGC | 79 | 1 | 0 | 15 | 217 | 517 | 1272 |
| | UGCUG | | | | | | | |
| 9: G > U_13: C > A | CUGCUGCUUCUGAUGC | 40 | 0 | 0 | 3 | 221 | 523 | 1367 |
| | UGCUG | | | | | | | |
| 9: G > A_14: U > G | CUGCUGCUACUGCGGC | 81 | 1 | 0 | 16 | 214 | 504 | 1197 |
| | UGCUG | | | | | | | |
| 9: G > C_14: U > C | CUGCUGCUCCUGCCGC | 82 | 1 | 0 | 19 | 233 | 621 | 1585 |
| | UGCUG | | | | | | | |
| 9: G > U_14: U > C | CUGCUGCUUCUGCCGC | 83 | 1 | 0 | 17 | 228 | 589 | 1447 |
| | UGCUG | | | | | | | |
| 9: G > U_14: U > G | CUGCUGCUUCUGCGGC | 41 | 0 | 0 | 4 | 221 | 507 | 1301 |
| | UGCUG | | | | | | | |
| 9: G > C_15: G > A | CUGCUGCUCCUGCUAC | 85 | 0 | 0 | 18 | 211 | 557 | 1471 |
| | UGCUG | | | | | | | |
| 9: G > C_16: C > A | CUGCUGCUCCUGCUGA | 86 | 1 | 0 | 5 | 210 | 525 | 1333 |
| | UGCUG | | | | | | | |
| 9: G > U_16: C > G | CUGCUGCUUCUGCUGG | 87 | 1 | 0 | 3 | 221 | 552 | 1531 |
| | UGCUG | | | | | | | |
| 9: G > U_10: C > A_ | CUGCUGCUUAUUCUGC | 132 | 0 | 0 | 0 | 11 | 280 | 875 |
| 12: G > U | UGCUG | | | | | | | |
| 9: G > C_10: C > A_ | CUGCUGCUCAUGAUGC | 133 | 0 | 0 | 0 | 10 | 266 | 928 |
| 13: C > A | UGCUG | | | | | | | |
| 9: G > C_10: C > G_ | CUGCUGCUCGUGAUGC | 134 | 0 | 0 | 0 | 11 | 256 | 832 |
| 13: C > A | UGCUG | | | | | | | |
| 9: G > C_10: C > G_ | CUGCUGCUCGUGGUGC | 135 | 0 | 0 | 0 | 11 | 274 | 869 |
| 13: C > G | UGCUG | | | | | | | |
| 9: G > U_10: C > A_ | CUGCUGCUUAUGGUGC | 136 | C | 0 | 0 | 8 | 295 | 792 |
| 13: C > G | UGCUG | | | | | | | |
| 9: G > U_10: C > G_ | CUGCUGCUUGUGAUGC | 137 | 0 | 0 | 0 | 13 | 286 | 970 |
| 13: C > A | UGCUG | | | | | | | |
| 9: G > U_10: C > G_ | CUGCUGCUUGUGGUGC | 138 | 0 | 0 | 0 | 14 | 314 | 929 |
| 13: C > G | UGCUG | | | | | | | |
| 9: G > A_10: C > G_ | CUGCUGCUAGUGCGGC | 139 | 0 | 0 | 1 | 20 | 273 | 752 |
| 14: U > G | UGCUG | | | | | | | |
| 9: G > C_10: C > G_ | CUGCUGCUCGUGCAGC | 140 | 0 | 0 | 0 | 13 | 317 | 967 |
| 14: U > A | UGCUG | | | | | | | |
| 9: G > C_10: C > G_ | CUGCUGCUCGUGCGGC | 141 | 0 | 0 | 0 | 15 | 260 | 809 |
| 14: U > G | UGCUG | | | | | | | |
| 9: G > U_10: C > A_ | CUGCUGCUUAUGCCGC | 142 | 0 | 0 | 0 | 20 | 280 | 807 |
| 14: U > C | UGCUG | | | | | | | |
| 9: G > U_10: C > A_ | CUGCUGCUUAUGCGGC | 143 | 0 | 0 | 0 | 8 | 265 | 712 |
| 14: U > G | UGCUG | | | | | | | |
| 9: G > C_10: C > A_ | CUGCUGCUCAUGCUUC | 144 | 0 | 0 | 0 | 14 | 283 | 961 |
| 15: G > U | UGCUG | | | | | | | |
| 9: G > U_10: C > A_ | CUGCUGCUUAUGCUAC | 145 | 0 | 0 | 2 | 20 | 265 | 781 |
| 15: G > A | UGCUG | | | | | | | |
| 9: G > U_10: C > A_ | CUGCUGCUUAUGCUCC | 146 | 0 | 0 | 1 | 14 | 279 | 843 |
| 15: G > C | UGCUG | | | | | | | |
| 9: G > C_10: C > G_ | CUGCUGCUCGUGCUGA | 147 | 0 | 0 | 0 | 11 | 271 | 803 |
| 16: C > A | UGCUG | | | | | | | |
| 9: G > C_10: C > G_ | CUGCUGCUCGUGCUGG | 148 | 0 | 0 | 0 | 16 | 290 | 879 |
| 16: C > G | UGCUG | | | | | | | |
| 9: G > U_10: C > A_ | CUGCUGCUUAUGCUGG | 149 | 0 | 0 | 0 | 10 | 299 | 864 |
| 16: C > G | UGCUG | | | | | | | |
| 9: G > U_10: C > G_ | CUGCUGCUUGUGCUGA | 150 | 0 | 0 | 0 | 13 | 295 | 884 |
| 16: C > A | UGCUG | | | | | | | |
| 9: G > A_10: C > A_ | CUGCUGCUAAAGCUGC | 325 | 1 | 0 | 0 | 32 | 303 | 996 |
| 11: U > A | UGCUG | | | | | | | |
| 9: G > A_10: C > A_ | CUGCUGCUAACGCUGC | 152 | 1 | 0 | 1 | 34 | 315 | 845 |
| 11: U > C | UGCUG | | | | | | | |
| 9: G > A_10: C > G_ | CUGCUGCUAGGGCUGC | 46 | 0 | 0 | 2 | 31 | 339 | 1005 |
| 11: U > G | UGCUG | | | | | | | |
| 9: G > C_10: C > A_ | CUGCUGCUCACGCUGC | 154 | 1 | 0 | 0 | 37 | 332 | 1043 |
| 11: U > C | UGCUG | | | | | | | |
| 9: G > C_10: C > G_ | CUGCUGCUCGCGCUGC | 155 | 1 | 0 | 1 | 39 | 319 | 983 |
| 11: U > C | UGCUG | | | | | | | |
| 9: G > U_10: C > A_ | CUGCUGCUUACGCUGC | 156 | 0 | 0 | 1 | 21 | 310 | 883 |
| 11: U > C | UGCUG | | | | | | | |
| 9: G > U_10: C > A_ | CUGCUGCUUAGGCUGC | 157 | 1 | 0 | 0 | 21 | 326 | 999 |
| 11: U > G | UGCUG | | | | | | | |
| 9: G > U_10: C > G_ | CUGCUGCUUGCGCUGC | 45 | 0 | 0 | 3 | 32 | 325 | 1007 |
| 11: U > C | UGCUG | | | | | | | |
| 9: G > A_10: C > G_ | CUGCUGCUAGUCCUUC | 178 | 0 | 0 | 0 | 0 | 62 | 582 |
| 12: G > C_15: G > U | UGCUG | | | | | | | |
| 9: G > C_10: C > A_ | CUGCUGCUCAUUCUAC | 179 | 0 | 0 | 0 | 0 | 63 | 600 |
| 12: G > U_15: G > A | UGCUG | | | | | | | |
| 9: G > C_10: C > G_ | CUGCUGCUCGUACUGA | 180 | 0 | 0 | 0 | 0 | 47 | 456 |
| 12: G > A_16: C > A | UGCUG | | | | | | | |
| 9: G > C_10: C > G_ | CUGCUGCUCGUUCUGG | 181 | 0 | 0 | 0 | 0 | 51 | 519 |
| 12: G > U_16: C > G | UGCUG | | | | | | | |
| 9: G > A 10:C > G_ | CUGCUGCUAGUGAUCC | 182 | 0 | 0 | 0 | 0 | 47 | 544 |
| 13: C > A_15: G > C | UGCUG | | | | | | | |
| 9: G > A_10: C > G_ | CUGCUGCUAGUGGUUC | 183 | 0 | 0 | 0 | 0 | 58 | 547 |
| 13: C > G_15: G > U | UGCUG | | | | | | | |
| 9: G > A_10: C > A_ | CUGCUGCUAAUGCGUC | 184 | 0 | 0 | 0 | 0 | 39 | 429 |
| 14: U > G_15: G > U | UGCUG | | | | | | | |
| 9: G > C_10: C > A_ | CUGCUGCUCAUGCGUC | 185 | 0 | 0 | 0 | 0 | 32 | 506 |
| 14: U > G_15: G > U | UGCUG | | | | | | | |
| 9: G > C_10: C > G_ | CUGCUGCUCGUGCGCC | 186 | 0 | 0 | 0 | 0 | 50 | 504 |
| 14: U > G_15: G > C | UGCUG | | | | | | | |
| 9: G > C_10: C > G_ | CUGCUGCUCGUGCGUC | 187 | 0 | 0 | 0 | 0 | 28 | 464 |
| 14: U > G_15: G > U | UGCUG | | | | | | | |
| 9: G > U_10: C > A_ | CUGCUGCUUAUGCGUC | 188 | 0 | 0 | 0 | 0 | 27 | 464 |
| 14: U > G_15: G > U | UGCUG | | | | | | | |
| 9: G > A_10: C > A_ | CUGCUGCUAAUGCGGU | 189 | 0 | 0 | 0 | 0 | 79 | 446 |
| 14: U > G_16: C > U | UGCUG | | | | | | | |
| 9: G > C_10: C > G_ | CUGCUGCUCGUGCAGA | 190 | 0 | 0 | 0 | 0 | 50 | 662 |
| 14: U > A_16: C > A | UGCUG | | | | | | | |
| 9: G > C_10: C > G_ | CUGCUGCUCGUGCGGA | 191 | 0 | 0 | 0 | 0 | 27 | 408 |
| 14: U > G_16: C > A | UGCUG | | | | | | | |
| 9: G > A_10: C > G_ | CUGCUGCUAGUGCUUA | 192 | 0 | 0 | 0 | 0 | 40 | 447 |
| 15: G > U_16: C > A | UGCUG | | | | | | | |
| 9: G > C_10: C > G_ | CUGCUGCUCGUGCUCA | 193 | 0 | 0 | 0 | 0 | 47 | 516 |
| 15: G > C_16: C > A | UGCUG | | | | | | | |
| 9: G > C_10: C > G_ | CUGCUGCUCGUGCUUA | 194 | 0 | 0 | 0 | 0 | 27 | 429 |
| 15: G > U_16: C > A | UGCUG | | | | | | | |
| 9: G > A_10: C > A_ | CUGCUGCUAAGGCUCC | 195 | 0 | 0 | 0 | 1 | 77 | 616 |
| 11: U > G_15: G > C | UGCUG | | | | | | | |
| 9: G > U_10: C > A_ | CUGCUGCUUAGGCUCC | 196 | 0 | 0 | 0 | 1 | 68 | 664 |
| 11: U > G_15: G > C | UGCUG | | | | | | | |
| 9: G > A_10: C > A_ | CUGCUGCUAACGCUGU | 197 | 0 | 0 | 0 | 1 | 101 | 511 |
| 11: U > C_16: C > U | UGCUG | | | | | | | |
| 9: G > A_10: C > G_ | CUGCUGCUAGCGCUGU | 198 | 0 | 0 | 0 | 1 | 109 | 501 |
| 11: U > C_16: C > U | UGCUG | | | | | | | |
| 9: G > U_10: C > A_ | CUGCUGCUUAGGCUGU | 199 | 0 | 0 | 0 | 1 | 103 | 642 |
| 11: U > G_16: C > U | UGCUG | | | | | | | |
| 9: G > A_10: C > A_ | CUGCUGCUAAAGCUAA | 210 | NA | NA | NA | NA | NA | NA |
| 11: U > A_15: G > A_ | UGCUG | | | | | | | |
| 16: C > A | | | | | | | | |
| 9: G > A_10: C > A_ | CUGCUGCUAAAGCUAU | 211 | NA | NA | NA | NA | NA | NA |
| 11: U > A_15: G > A_ | UGCUG | | | | | | | |
| 16: C > U | | | | | | | | |
| 9: G > A_10: C > A_ | CUGCUGCUAAAGCUUA | 212 | NA | NA | NA | NA | NA | NA |
| 11: U > A_15: G > U_ | UGCUG | | | | | | | |
| 16: C > A | | | | | | | | |
| 9:G > A_10: C > A_ | CUGCUGCUAAAGCUUU | 213 | NA | NA | NA | NA | NA | NA |
| 11: U > A_15: G > U_ | UGCUG | | | | | | | |
| 16: C > U | | | | | | | | |
| 10: U > C_15: C > A | UGCUGCUGCCGCUGAU | 88 | 0 | 0 | 11 | 237 | 520 | 1235 |
| | GCUGC | | | | | | | |
| 10: U > G_11: G > U | UGCUGCUGCGUCUGCU | 89 | 0 | 0 | 2 | 239 | 509 | 1339 |
| | GCUGC | | | | | | | |
| 10: U > G_12: C > A | UGCUGCUGCGGAUGCU | 90 | 0 | 0 | 4 | 232 | 502 | 1188 |
| | GCUGC | | | | | | | |
| 10: U > G_15: C > A | UGCUGCUGCGGCUGAU | 91 | 0 | 0 | 1 | 240 | 475 | 1131 |
| | GCUGC | | | | | | | |
| 11: G > A_13: U > G | UGCUGCUGCUACGGCU | 92 | 0 | 0 | 14 | 226 | 519 | 1150 |
| | GCUGC | | | | | | | |
| 11: G > A_15: C > A | UGCUGCUGCUACUGAU | 93 | 0 | 0 | 11 | 229 | 485 | 1132 |
| | GCUGC | | | | | | | |
| 11: G > C_12: C > G | UGCUGCUGCUCGUGCU | 94 | 0 | 0 | 6 | 224 | 487 | 1171 |
| | GCUGC | | | | | | | |
| 11: G > C_14: G > A | UGCUGCUGCUCCUACU | 95 | 0 | 0 | 22 | 218 | 519 | 1349 |
| | GCUGC | | | | | | | |
| 11: G > U_12: C > A | UGCUGCUGCUUAUGCU | 96 | 0 | 0 | 4 | 215 | 474 | 1122 |
| | GCUGC | | | | | | | |
| 11: G > U_13: U > G | UGCUGCUGCUUCGGCU | 97 | 0 | 0 | 4 | 223 | 501 | 1269 |
| | GCUGC | | | | | | | |
| 11: G > U_14: G > A | UGCUGCUGCUUCUACU | 98 | 0 | 0 | 20 | 215 | 505 | 1346 |
| | GCUGC | | | | | | | |
| 11: G > U_16: U > G | UGCUGCUGCUUCUGCG | 99 | 0 | 0 | 2 | 211 | 519 | 1293 |
| | GCUGC | | | | | | | |
| 8: G > C_14: G > A | UGCUGCUCCUGCUACU | 100 | 0 | 0 | 19 | 216 | 545 | 1387 |
| | GCUGC | | | | | | | |
| 8: G > C_9: C > G | UGCUGCUCGUGCUGCU | 101 | 0 | 0 | 3 | 224 | 485 | 1241 |
| | GCUGC | | | | | | | |
| 8: G > U_10: U > G | UGCUGCUUCGGCUGCU | 102 | 0 | 0 | 4 | 228 | 500 | 1231 |
| | GCUGC | | | | | | | |
| 8: G > U_12: C > A | UGCUGCUUCUGAUGCU | 103 | 0 | 0 | 5 | 218 | 498 | 1204 |
| | GCUGC | | | | | | | |
| 8: G > U_13: U > C | UGCUGCUUCUGCCGCU | 104 | 0 | 0 | 20 | 232 | 557 | 1405 |
| | GCUGC | | | | | | | |
| 8: G > U_13: U > G | UGCUGCUUCUGCGGCU | 105 | 0 | 0 | 7 | 218 | 508 | 1244 |
| | GCUGC | | | | | | | |
| 8: G > U_9: C > A | UGCUGCUUAUGCUGCU | 106 | 0 | 0 | 9 | 215 | 473 | 1216 |
| | GCUGC | | | | | | | |
| 9: C > A 11: G > U | UGCUGCUGAUUCUGCU | 107 | 0 | 0 | 7 | 228 | 476 | 1256 |
| | GCUGC | | | | | | | |
| 9: C > A_16: U > C | UGCUGCUGAUGCUGCC | 108 | 0 | 0 | 20 | 241 | 573 | 1318 |
| | GCUGC | | | | | | | |
| 9: C > A_10: U > C | UGCUGCUGACGCUGCU | 109 | 3 | 0 | 14 | 241 | 543 | 1441 |
| | GCUGC | | | | | | | |
| 9: C > A_10: U > A_ | UGCUGCUGAAGCUUCU | 159 | 0 | 0 | 0 | 25 | 301 | 1043 |
| 14: G > U | GCUGC | | | | | | | |
| 9: C > A_10: U > C_ | UGCUGCUGACGAUGCU | 160 | 0 | 0 | 0 | 16 | 287 | 812 |
| 12: C > A | GCUGC | | | | | | | |
| 9: C > A_10: U > C_ | UGCUGCUGACGGUGCU | 161 | 0 | 0 | 0 | 25 | 291 | 854 |
| 12: C > G | GCUGC | | | | | | | |
| 9: C > A_10: U > C_ | UGCUGCUGACGCGGCU | 162 | 0 | 0 | 0 | 27 | 301 | 854 |
| 13: U > G | GCUGC | | | | | | | |
| 9: C > A_10: U > C_ | UGCUGCUGACGCUUCU | 163 | 0 | 0 | 0 | 20 | 280 | 845 |
| 14: G > U | GCUGC | | | | | | | |
| 9: C > G_10: U > C_ | UGCUGCUGGCGCUCCU | 164 | 0 | 0 | 0 | 24 | 322 | 972 |
| 14: G > C | GCUGC | | | | | | | |
| 9: C > A_10: U > C_ | UGCUGCUGACGCUUUU | 200 | 0 | 0 | 0 | 0 | 102 | 504 |
| 14: G > U_15: C > U | GCUGC | | | | | | | |
| 9: C > A_10: U > C_ | UGCUGCUGACGCUUCG | 201 | 0 | 0 | 0 | 0 | 46 | 476 |
| 14: G > U_16: U > G | GCUGC | | | | | | | |
| 9: C > G_10: U > C_ | UGCUGCUGGCGCUUUU | 202 | 0 | 0 | 0 | 0 | 98 | 519 |
| 14: G > U_15: C > U | GCUGC | | | | | | | |
| 9: C > U_10: U > C_ | UGCUGCUGUCGCUUCG | 203 | 0 | 0 | 0 | 0 | 97 | 524 |
| 14: G > U_16: U > G | GCUGC | | | | | | | |
| 9: C > A_10: U > C_ | UGCUGCUGACGAUGAA | 214 | NA | NA | NA | NA | NA | NA |
| 12: C > A_15: C > A_ | GCUGC | | | | | | | |
| 16: U > A | | | | | | | | |
| 9: C > A_10: U > C_ | UGCUGCUGACGAUGUA | 215 | NA | NA | NA | NA | NA | NA |
| 12: C > A_15: C > U_ | GCUGC | | | | | | | |
| 16: U > A | | | | | | | | |
| 10: G > A_14: C > A | GCUGCUGCUACUGAUG | 110 | 0 | 0 | 13 | 230 | 484 | 1137 |
| | CUGCU | | | | | | | |
| 10: G > A_14: C > G | GCUGCUGCUACUGGUG | 111 | 0 | 0 | 15 | 228 | 505 | 1190 |
| | CUGCU | | | | | | | |
| 10: G > C_11: C > G | GCUGCUGCUCGUGCUG | 112 | 0 | 0 | 2 | 237 | 515 | 1207 |
| | CUGCU | | | | | | | |
| 10: G > C_13: G > A | GCUGCUGCUCCUACUG | 113 | 0 | 0 | 17 | 236 | 543 | 1307 |
| | CUGCU | | | | | | | |
| 10: G > U_11: C > A | GCUGCUGCUUAUGCUG | 114 | 0 | 0 | 5 | 226 | 483 | 1239 |
| | CUGCU | | | | | | | |
| 10: G > U_12: U > G | GCUGCUGCUUCGGCUG | 115 | 0 | 0 | 7 | 236 | 505 | 1267 |
| | CUGCU | | | | | | | |
| 10: G > U_14: C > A | GCUGCUGCUUCUGAUG | 116 | 0 | 0 | 3 | 227 | 474 | 1228 |
| | CUGCU | | | | | | | |
| 10: G > U_15: U > G | GCUGCUGCUUCUGCGG | 117 | 0 | 0 | 4 | 225 | 503 | 1250 |
| | CUGCU | | | | | | | |
| 11: C > A_13: G > A | GCUGCUGCUGAUACUG | 118 | 0 | 0 | 17 | 236 | 541 | 1362 |
| | CUGCU | | | | | | | |
| 11: C > A_13: G > C | GCUGCUGCUGAUCCUG | 119 | 0 | 0 | 3 | 234 | 495 | 1358 |
| | CUGCU | | | | | | | |
| 11: C > A_13: G > U | GCUGCUGCUGAUUCUG | 120 | 0 | 0 | 4 | 232 | 513 | 1308 |
| | CUGCU | | | | | | | |
| 11: C > A_15: U > A | GCUGCUGCUGAUGCAG | 121 | 0 | 0 | 5 | 235 | 542 | 1418 |
| | CUGCU | | | | | | | |
| 11: C > A_15: U > C | GCUGCUGCUGAUGCCG | 122 | 0 | 0 | 13 | 247 | 540 | 1356 |
| | CUGCU | | | | | | | |
| 11: C > U_14: C > A | GCUGCUGCUGUUGAUG | 123 | 0 | 0 | 44 | 253 | 528 | 1283 |
| | CUGCU | | | | | | | |
| 8: C > A_11: C > G | GCUGCUGAUGGUGCUG | 124 | 0 | 0 | 6 | 227 | 507 | 1219 |
| | CUGCU | | | | | | | |
| 8: C > A_12: U > G | GCUGCUGAUGCGGCUG | 125 | 0 | 0 | 8 | 241 | 478 | 1215 |
| | CUGCU | | | | | | | |
| 8: C > A_15: U > C | GCUGCUGAUGCUGCCG | 126 | 0 | 0 | 16 | 255 | 537 | 1260 |
| | CUGCU | | | | | | | |
| 8: C > A_16: G > C | GCUGCUGAUGCUGCUC | 127 | 0 | 0 | 6 | 238 | 500 | 1260 |
| | CUGCU | | | | | | | |
| 8: C > G_16: G > U | GCUGCUGGUGCUGCUU | 128 | 0 | 0 | 8 | 232 | 507 | 1335 |
| | CUGCU | | | | | | | |
| 9: U > C_14: C > A | GCUGCUGCCGCUGAUG | 129 | 0 | 0 | 11 | 246 | 504 | 1182 |
| | CUGCU | | | | | | | |
| 9: U > G_11: C > A | GCUGCUGCGGAUGCUG | 130 | 0 | 0 | 6 | 235 | 517 | 1267 |
| | CUGCU | | | | | | | |
| 9: U > G_14: C > A | GCUGCUGCGGCUGAUG | 131 | 0 | 0 | 4 | 236 | 488 | 1132 |
| | CUGCU | | | | | | | |
| 9: U > A_10: G > C_ | GCUGCUGCACCUGGUG | 165 | 0 | 0 | 0 | 15 | 317 | 976 |
| 14: C > G | CUGCU | | | | | | | |
| 9: U > G_10: G > A_ | GCUGCUGCGACUGAUG | 166 | 0 | 0 | 0 | 20 | 306 | 801 |
| 14: C > A | CUGCU | | | | | | | |
| 9: U > G_10: G > A_ | GCUGCUGCGACUGCUU | 167 | 0 | 0 | 0 | 20 | 308 | 844 |
| 16: G > U | CUGCU | | | | | | | |
| 9: U > G_10: G > C_ | GCUGCUGCGCAUGCUG | 168 | 0 | 0 | 0 | 13 | 298 | 869 |
| 11: C > A | CUGCU | | | | | | | |
| 9: U > G_10: G > C_ | GCUGCUGCGCGUGCUG | 169 | 0 | 0 | 0 | 16 | 307 | 815 |
| 11: C > G | CUGCU | | | | | | | |
| 9: U > G_10: G > C_ | GCUGCUGCGCCUGAUG | 170 | 0 | 0 | 0 | 11 | 293 | 755 |
| 14: C > A | CUGCU | | | | | | | |
| 9: U > G_10: G > C_ | GCUGCUGCGCCUGGUG | 171 | 0 | 0 | 0 | 17 | 310 | 909 |
| 14: C > G | CUGCU | | | | | | | |
| 9: U > G_10: G > U_ | GCUGCUGCGUAUGCUG | 172 | 0 | 0 | 0 | 13 | 282 | 784 |
| 11: C > A | CUGCU | | | | | | | |
| 9: U > G_10: G > U_ | GCUGCUGCGUCUUCUG | 173 | 0 | 0 | 1 | 13 | 293 | 864 |
| 13: G > U | CUGCU | | | | | | | |
| 9: U > G_10: G > U_ | GCUGCUGCGUCUGAUG | 174 | 0 | 0 | 0 | 8 | 276 | 743 |
| 14: C > A | CUGCU | | | | | | | |
| 9: U > G_10: G > U_ | GCUGCUGCGUCUGGUG | 175 | 0 | 0 | 0 | 12 | 288 | 825 |
| 14: C > G | CUGCU | | | | | | | |
| 9: U > G_10: G > U_ | GCUGCUGCGUCUGCAG | 176 | 0 | 0 | 0 | 19 | 326 | 1045 |
| 15: U > A | CUGCU | | | | | | | |
| 9: U > A_10: G > U_ | GCUGCUGCAUAUGCUG | 43 | 0 | 0 | 0 | 23 | 302 | 884 |
| 11: C > A | CUGCU | | | | | | | |
| 9: U > A_10: G > U_ | GCUGCUGCAUAUACUG | 204 | 0 | 0 | 0 | 0 | 69 | 576 |
| 11: C > A_13: G > A | CUGCU | | | | | | | |
| 9: U > G_10: G > C_ | GCUGCUGCGCAUGCUU | 205 | 0 | 0 | 0 | 0 | 37 | 507 |
| 11: C > A_16: G > U | CUGCU | | | | | | | |
| 9: U > G_10: G > C_ | GCUGCUGCGCGUGAUG | 206 | 0 | 0 | 0 | 0 | 33 | 437 |
| 11: C > G_14: C > A | CUGCU | | | | | | | |
| 9: U > G_10: G > C_ | GCUGCUGCGCGUGCUU | 207 | 0 | 0 | 0 | 0 | 33 | 490 |
| 11: C > G_16: G > U | CUGCU | | | | | | | |
| 9: U > G_10: G > U_ | GCUGCUGCGUAUGCCG | 208 | 0 | 0 | 0 | 0 | 45 | 458 |
| 11: C > A_15: U > C | CUGCU | | | | | | | |
| 9: U > G_10: G > U_ | GCUGCUGCGUAUGCUU | 209 | 0 | 0 | 0 | 0 | 30 | 482 |
| 11: C > A_16: G > U | CUGCU | | | | | | | |
| 9: U > A_10: G > U_ | GCUGCUGCAUAUGCAA | 216 | NA | NA | NA | NA | NA | NA |
| 11: C > A_15: U > A_ | CUGCU | | | | | | | |
| 16: G > A | | | | | | | | |
| 9: U > A_10: G > U_ | GCUGCUGCAUAUGCAU | 217 | NA | NA | NA | NA | NA | NA |
| 11: C > A_15: U > A_ | CUGCU | | | | | | | |
| 16: G > U | | | | | | | | |

mm = mismatch

Table B4 shows exemplary filtering criteria for off-target activity of the guide sequences targeting a CAG repeat region of a CAG repeat containing mRNA or pre-mRNA from Table B3. In some embodiments, specificity of a guide sequence targeting a CAG repeat region of a CAG repeat containing mRNA or pre-mRNA is according to any one of the thresholds set in Table B4, or a combination thereof.

TABLE B4
Filtering Methods for Guide Sequences for Off-Target Activity

| # | | | |
|---|---|---|---|
| Mismatches | CAG registry | UGC registry | GCU registry |
| 1 | No sequences with | One sequence | No sequences |
| | zero perfect | with zero | with zero |
| | match | perfect match | perfect match |
| | | (mm0), but | |
| | | excluded due to | |
| | | 27 perfect 17-mer | |
| | | match within | |
| | | position 1-21 | |
| 2 | 0 perfect match | 0 perfect match | 0 perfect match |
| | 0-1 off targets | 0-1 off targets | 0-1 off targets |
| | with perfect 17- | with perfect 17- | with perfect 17- |
| | mer match within | mer match within | mer match within |
| | position 1-21 | position 1-21 | position 1-21 |
| | Position 9 | Position 9 | Position 9 |
| | mismatch is kept | mismatch is kept | mismatch is kept |
| | static, second | static, second | static, second |
| | mutation is | mutation is | mutation is |
| | introduced | introduced | introduced |
| | anywhere from | anywhere from | anywhere from |
| | position 10 to | position 10 to | position 10 to |
| | 16 | 16 | 16 |
| | Genes checked | Genes checked | Genes checked |
| | only if | only if | only if |
| | expression is in | expression is in | expression is in |
| | brain and within | brain and within | brain and within |
| | Q4 | Q4 | Q4 |
| 3 | 0 perfect match | 0 perfect match | 0 perfect match |
| | 0-1 off targets | 0-1 off targets | 0-1 off targets |
| | with perfect 17- | with perfect 17- | with perfect 17- |
| | mer match within | mer match within | mer match within |
| | position 1-21 | position 1-21 | position 1-21 |
| | Up to 2 off | Up to 2 off | Up to 2 off |
| | targets with 1 | targets with 1 | targets with 1 |
| | mismatch | mismatch | mismatch |
| | Less than 20 off | Less than 40 off | Less than 20 off |
| | target with 2 | target with 2 | target with 2 |
| | mismatches | mismatches | mismatches |
| | | (relaxed filter, | |
| | | due to absence | |
| | | of sequences | |
| | | passing the 20 | |
| | | filter) | |
| | Position 9 and | Position 9 and | Position 9 and |
| | 10 mismatches | 10 mismatches | 10 mismatches |
| | are kept static, | are kept static, | are kept static, |
| | third mutation | third mutation | third mutation |
| | introduced | introduced | introduced |
| | anywhere from | anywhere from | anywhere from |
| | position 11 to | position 11 to | position 11 to |
| | 16 | 16 | 16 |
| | To force | To force | To force |
| | sequences with | sequences with | sequences with |
| | mutations in | mutations in | mutations in |
| | positions 9, 10, | positions 9, 10, | positions 9, 10, |
| | and 11, off | and 11, off | and 11, off |
| | targets with | targets with | targets with |
| | perfect 17-mer | perfect 17-mer | perfect 17-mer |
| | match within | match within | match within |
| | position 1-21 | position 1-21 | position 1-21 |
| | are relaxed to 1 | are relaxed to 1 | are relaxed to 1 |
| | and off target | and off target | and off target |
| | with 2 | with 2 | with 2 |
| | mismatches are | mismatches are | mismatches are |
| | relaxed to 40 | relaxed to 40 | relaxed to 40 |
| | Genes checked | Genes checked | Genes checked |
| | only if | only if | only if |
| | expression is in | expression is in | expression is in |
| | brain and within | brain and within | brain and within |
| | Q4 | Q4 | Q4 |
| 4 | 0 perfect match | 0 perfect match | 0 perfect match |
| | 0 off targets | 0 off targets | 0 off targets |
| | with perfect 17- | with perfect 17- | with perfect 17- |
| | mer match within | mer match within | mer match within |
| | position 1-21 | position 1-21 | position 1-21 |
| | 0 off targets | 0 off targets | 0 off targets |
| | with 1 mismatch | with 1 mismatch | with 1 mismatch |
| | 0-1 off targets | 0-1 off targets | 0-1 off targets |
| | with 2 | with 2 | with 2 |
| | mismatches | mismatches | mismatches |
| | Position 9-10 | Position 9-10 | Position 9-10 |
| | mismatches are | mismatches are | mismatches are |
| | kept static, | kept static, | kept static, |
| | third mutation | third mutation | third mutation |
| | is added | is added | is added |
| | anywhere between | anywhere between | anywhere between |
| | 11-16, fourth | 11-16, fourth | 11-16, fourth |
| | mutation in | mutation in | mutation in |
| | added in | added in | added in |
| | position 15 or | position 15 or | position 15 or |
| | 16 | 16 | 16 |
| | To maintain 9, | Mutations in 9, | To maintain 9, |
| | 10, 11t static, | 10, 11 cannot be | 10, 11t static, |
| | and introduce | kept static | and introduce |
| | fourth mutation | | fourth mutation |
| | in position 15 | | in position 15 |
| | or 16, off | | or 16, off |
| | target for 2 | | target for 2 |
| | mismatches is | | mismatches is |
| | relaxed to 0-1 | | relaxed to 0-1 |
| | Genes checked | Genes checked | Genes checked |
| | only if | only if | only if |
| | expression is in | expression is in | expression is in |
| | brain and within | brain and within | brain and within |
| | Q4 | Q4 | Q4 |

In some embodiments, a linker is present in the artificial double stranded RNA, joining the stem and the loop of the artificial double stranded RNA. In some embodiments, a 5′ linker joins the 5′ stem and the loop of the artificial double stranded RNA. In some embodiments, a 3′ linker joins the 3′ stem and the loop of the artificial double stranded RNA. In some embodiments, a 5′ linker joins the 5′ stem and the loop of the artificial double stranded RNA, and a 3′ linker joins the 3′ stem and the loop of the artificial double stranded RNA. In some embodiments, the 5′ linker and/or 3′ linker has about 1-6 nucleotides, 1-5 nucleotides, 1-4 nucleotides, 1-3 nucleotides, 1-2 nucleotides, 2-6 nucleotides, 3-6 nucleotides, 4-6 nucleotides, 5-6 nucleotides, 2-5 nucleotides, or 2-4 nucleotides. In some embodiments, the 5′ linker and/or 3′ linker has about 1 nucleotide, 2 nucleotides, 3 nucleotides, 4 nucleotides, 5 nucleotides, or 6 nucleotides. In some embodiments, the 5′ linker the same number of nucleotides as the 3′ linker. In some embodiments, the 5′ linker is 100% complementary to the 3′ linker. In some embodiments, the 5′ linker comprises or consists of the nucleotide sequence CAGC and/or the 3′ linker comprises or consists of the nucleotide sequence of GCUG. In some cases, a sbRNA of the present disclosure does not include linkers.

In some embodiments, the 5′ linker and 3′ linker each comprise at least 4 nucleotides, optionally wherein at least 75% of the 5′ linker nucleotides are complementary to the 3′ linker nucleotides.

Examples of 5′ and 3′ linkers are provided in Table C. In some embodiments, a double stranded RNA has a 5′ linker of SEQ ID NO:15 and a 3′ linker of SEQ ID NO:23.

TABLE C
5′ Linkers and 3′ Linkers
5′ Linkers Sequence
T
CAGC
CTGC
CUGC
3′ Linkers Sequence
GCA
GNN
GTG (DNA)
GUG (RNA)
GCTG (DNA)
5′ Linkers Sequence
GCUG (RNA)
GCT (DNA)
GCU (RNA)
GCA (DNA)

In some embodiments, a terminal loop separates the 5′ linker and the 3′ linker of the artificial double stranded RNA. The terminal loop sequence may be of any length or have about 4 nucleotides to about 1,000 nucleotides, about 4 nucleotides to about 900 nucleotides, about 4 nucleotides to about 800 nucleotides, about 4 nucleotides to about 700 nucleotides, about 4 nucleotides to about 600 nucleotides, about 4 nucleotides to about 500 nucleotides, about 4 nucleotides to about 400 nucleotides, about 4 nucleotides to about 300 nucleotides, about 4 nucleotides to about 200 nucleotides, about 4 nucleotides to about 100 nucleotides, about 4 nucleotides to about 90 nucleotides about 4 nucleotides to about 80 nucleotides, about 4 nucleotides to about 70 nucleotides, about 4 nucleotides to about 50 nucleotides, about 4 nucleotides to about 40 nucleotides, about 4 nucleotides to about 30 nucleotides, about 4 nucleotides to about 20 nucleotides, about 4 nucleotides to about 15 nucleotides, or about 4 nucleotides to about 10 nucleotides. In some embodiments, the terminal loop sequence has about 4 nucleotides, 5 nucleotides, 6 nucleotides, 7, nucleotides, 8 nucleotides, 9 nucleotides, 10 nucleotides, 11 nucleotides, 12, nucleotides, 13 nucleotides, 14 nucleotides, or 15 nucleotides. In some embodiments, the terminal loop comprises a palindromic sequence. In some embodiments, the terminal loop comprises an asymmetric sequence. In some embodiments, the terminal loop comprises or consists of the nucleotide sequence of ACCUGC. Examples of loop sequences are provided in Table D.

TABLE D
Terminal Loop Sequences

| Description | Sequence | SEQ ID NO: |
|---|---|---|
| Palindromic loop (DNA) | CTCGAG | NA |
| Palindromic loop (RNA) | CUCGAG | NA |
| Asymmetric loop (DNA) | ACCTGC | NA |
| Asymmetric loop (RNA) | ACCUGC | NA |
| miR-30 loop (DNA) | Gtgaagccacagatg | SEQ ID NO: 31 |
| miR-30 loop (RNA) | Gugaagccacagaug | SEQ ID NO: 32 |
| miR-33 loop (DNA) | gttctggcaatacct | SEQ ID NO: 33 |
| miR-33 loop (RNA) | guucuggcaauaccu | SEQ ID NO: 34 |

Additional embodiments of 5′ leader sequences, 3′ trailer sequences, and terminal loop sequences are provided in Table E. In some embodiments, a double stranded RNA is an artificial miRNA comprising from 5′ to 3′: a 5′ leader sequence, a passenger sequence or guide sequence, a terminal loop, a guide sequence or passenger sequence, and a 3′ trailer sequence, wherein the guide sequence targets a CAG repeat region of a CAG repeat containing RNA and comprises 1-5 base mismatches relative to the CAG repeat region, wherein the base mismatches are located at positions 8-16 of the guide sequence. In some embodiments, the 5′ leader sequence, 3′ trailer sequence, and terminal loop of the artificial miRNA are selected from Table E. In some embodiments, the guide sequence is selected from Tables B1-B2.

TABLE E
Exemplary 5′ leader, 3′ trailer, and Terminal Loop Sequences

| | 5′ leader | 3′ trailer | |
|---|---|---|---|
| Description | sequence | sequence | Loop sequence |
| hsa-mir-26a- | GUGGCCUCG | GGGACGC | GUGCAGGUCCCAAU |
| 1 | | | GGG |
| | | | [SEQ ID |
| | | | NO: 274] |
| hsa-mir-26a- | GGCUGUGGCUGGA | UGGAGGCAGCU | GUUUCCAUCUGUGA |
| 2 | [SEQ ID NO: 227] | [SEQ ID NO: 251] | GG |
| | | | [SEQ ID |
| | | | NO: 275] |
| hsa-mir-33a | CUGUG | AG | UGUUCUGGUGGUAC |
| | | | CCAUG |
| | | | [SEQ ID |
| | | | NO: 276] |
| hsa-mir-451a | CUUGGGAAUGGCAAGG | UCUUGCUAUACCCAGA | N/A |
| | [SEQ ID NO: 229] | [SEQ ID NO: 253] | |
| hsa-mir-22 | GGCUGAGCCGCAGU | UGCCCUCUGCC | UGUCCUGACCCAGC |
| | [SEQ ID NO: 230] | [SEQ ID NO: 254] | UA |
| | | | [SEQ ID |
| | | | NO: 277] |
| hsa-mir-375 | CCCC | GGC | GGACCUGAGCGU |
| | | | [SEQ ID |
| | | | NO: 278] |
| hsa-mir-21 | UGUCGGG | CUGACA | CUGUUGAAUCUCAU |
| | | | GG |
| | | | [SEQ ID |
| | | | NO: 279] |
| hsa-mir-99a | CCCAUUGGCAUA | UGUCAGUGUG | GUGAAGUGGACCGC |
| | [SEQ ID NO: 233] | [SEQ ID NO: 257] | A |
| | | | [SEQ ID |
| | | | NO: 280] |
| hsa-mir-199a- | GCCAA | GGC | AGGAGGCUCUCAAU |
| 1 | | | GUGU |
| | | | [SEQ ID |
| | | | NO: 281] |
| hsa-mir-199a- | AGGAAGCUUCUGGAGA | GACUGGGCAAGGGAGAG | AGGACAAUGCCGUU |
| 2 | UCCUGCUCCGUCGC | CA | GU |
| | [SEQ ID NO: 235] | [SEQ ID NO: 259] | [SEQ ID |
| | | | NO: 282] |
| hsa-mir-30a | GCGAC | UGC | CUGUGAAGCCACAG |
| | | | AUGGG |
| | | | [SEQ ID |
| | | | NO: 283] |
| mmu-mir-26a- | AAGGCCGUGGCCUCG | GGGACGCGGGCCUG | GUGCAGGUCCCAAG |
| 1 | [SEQ ID NO: 237] | [SEQ ID NO: 261] | GGG |
| | | | [SEQ ID |
| | | | NO: 284] |
| mmu-mir-26a- | GGCUGCGGCUGGA | UGGAGGCAGCG | GUGUCCGUCCAUGA |
| 2 | [SEQ ID NO: 238] | [SEQ ID NO: 262] | GG |
| | | | [SEQ ID |
| | | | NO: 285] |
| mmu-mir-33 | CUGUG | GG | UGUUCUGGCAAUAC |
| | | | CUGUG |
| | | | [SEQ ID |
| | | | NO: 286] |
| mmu-mir-451a | CUUGGGAAUGGCGAGG | UCUUGCUGCUCCCACA | N/A |
| | [SEQ ID NO: 240] | [SEQ ID NO: 264] | |
| mmu-mir-22 | ACCUGGCUGAGCCGCA | UGCCCUCUGCCCCUGGC | UGUCCUGACCCAGC |
| | GU | [SEQ ID NO: 265] | UA [SEQ ID |
| | [SEQ ID NO: 241] | | NO: 277] |
| mmu-mir-375 | CCCC | GGC | CGGACCUGAGCGU |
| | | | [SEQ ID |
| | | | NO: 287] |
| mmu-mir-21a | UGUACCACCUUGUCGG | UGACAUUUUGGUAUC | CUGUUGAAUCUCAU |
| | A | [SEQ ID NO: 267] | GG |
| | [SEQ ID NO: 243] | | [SEQ ID |
| | | | NO: 279] |
| mmu-mir-99a | CAUA | GUG | GUGAAGUGGACCGC |
| | | | [SEQ ID |
| | | | NO: 289] |
| mmu-mir-99b | GGCACC | UGUC | GGGCCUUCGCCGCA |
| | | | CA |
| | | | [SEQ ID |
| | | | NO: 290] |
| mmu-mir-199a- | GCCAU | GGC | AGGAGGCUGGGACA |
| 1 | | | UGU |
| | | | [SEQ ID |
| | | | NO: 291] |
| mmu-mir-199a- | UGGAAGCUUCAGGAGA | GACUGGGCAAGGGCCAG | AGGACAAUGCCGUU |
| 2 | UCCUGCUCCGUCGC | [SEQ ID NO: 271] | GU |
| | [SEQ ID NO: 247] | | [SEQ ID |
| | | | NO: 292] |
| mmu-mir-30a | GCGAC | UGC | CUGUGAAGCCACAA |
| | | | AUGGG |
| | | | [SEQ ID |
| | | | NO: 282] |
| eSIBR | CTGGAGGCTTGCTTTG | CAGGAAGGACACAAGGC | UUUUGGCCUCUGAC |
| (miR155- | GGCTGTATUGCUG | CCTTTATCAGCACTCAC | UGA |
| based) | [SEQ ID NO: 249] | ATGGAACAAATGGCCAC | [SEQ ID |
| | | CGTGGGAGGATGACAA | NO: 294] |
| | | [SEQ ID NO: 273] | |
| hsa-min-pri- | ACUGUCCUUUU [SEQ | GAAUGGUGGU [SEQ | UGUAUAUCUGAAAG |
| miR101-2 | ID NO: 885] | ID NO: 886] | G [SEQ ID |
| | | | NO: 887] |
| hsa-min-pri- | GUGUUGGGGACUCGCG | ACCCGGCGGGCGCGGCG | CUGUAGCGCAAUU |
| miR203a | CGCUGGGUCC [SEQ | ACAGCGA [SEQ ID | [SEQ ID |
| | ID NO: 888] | NO: 889] | NO: 890] |
| hsa-min-pri- | UGUUUUGAGCGGGGG | UCCUCUCAUUUGCUAUA | UUGUCAUAAACCG |
| miR335 | [SEQ ID NO: 891] | UUCA [SEQ ID | [SEQ ID |
| | | NO: 892] | NO: 893] |
| hsa-min-pri- | CUGACUAUGCCUCCC | GGGUUGUAGUC [SEQ | UAAAGCUGGAGA |
| miR324 | [SEQ ID NO: 894] | ID NO: 895] | [SEQ ID |
| | | | NO: 896] |
| hsa-min-pri- | AGGCCUCGCUGUUCUC | CACGGCGGGGACA | UUCUACUGCUCACU |
| miR135a-1 | [SEQ ID NO: 897] | [SEQ ID NO: 898] | CA [SEQ ID |
| | | | NO: 899] |

In some embodiments, a 5′ leader of Table E is extended at the 5′ terminus with 4 nucleotides and/or the corresponding 3′ trailer of Table E is extended at the 3′ terminus with 4

nucleotides that are non-complementary to the 4 nucleotides of the 5′ terminus of the corresponding 5′ leader sequence for improved processing. In some embodiments, a 5′ leader of Table E is extended at the 5′ terminus with 4 U's and/or the corresponding 3′ trailer of Table E is extended at the 3′ terminus with 4 U's for improved processing.

It should be understood that the double stranded RNAs of the disclosure comprising guide sequences targeting a CAG repeat region of a CAG repeat containing mRNA, also includes double stranded RNAs comprising guide sequences targeting a different frame, also referred to as register, of the CAG repeat region of the CAG repeat containing mRNA. Thus, targeting CAG repeats includes guide sequence having a +1 shift in frame to target AGC repeats or a +2 shift in frame to target GCA repeats and still target the same CAG repeat containing mRNA transcript.

In some embodiments, cleavage by Drosha and/or Dicer define the sequence and function of the siRNA produced from the double stranded RNA. In some embodiments, the double stranded RNA is cleaved by Drosha within the 5′ leader sequence and/or the 3′ trailer sequence to produce shRNA comprising the guide sequence and passenger sequence. In some embodiments, the shRNA is cleaved by Dicer to produce siRNA. The siRNA are loaded onto the RNA induced silencing complex (RISC). In some embodiments, the double stranded RNA is a pri-miRNA-like molecule that is cleaved by Drosha to produce pre-miRNA. The pre-miRNA molecule is a shRNA-like molecule that can subsequently be processed by Dicer to result in an siRNA-like duplex. In some embodiments, cleavage of the double stranded RNA is Dicer independent (e.g., cleaved by Ago2). In some embodiments, the shRNA produced from the double stranded RNA has a 5′ overhang and/or 3′ overhang, e.g., 1-6 nucleotides. In some embodiments, the shRNA produced from the double stranded RNA has a 2-3 nucleotide overhang at the 5′ end and/or the 3′ end. In some embodiments, the shRNA produced from the double stranded RNA has a dinucleotide overhang at the 5′ end and/or the 3′ end. In some embodiments, the shRNA produced from the double stranded RNA has about 38 to 300 nucleotides, 38 to 250 nucleotides, 38 to 200 nucleotides, 38 to 150 nucleotides, 38 to 100 nucleotides, 38 to 75 nucleotides or 38 to 50 nucleotides.

In some embodiments, the siRNA produced or processed from the double stranded RNA in the mammalian cell comprise the 1-5 base mismatches relative to the CAG repeat region at their predicted positions within positions 8-16 of the guide sequence. The presence of the 1-5 base mismatches relative to the CAG repeat region at their predicted positions within positions 8-16 of the guide sequence may be reflective of correct 5′ processing of the guide strand. In some embodiments, the siRNA produced or processed from the double stranded RNA in the mammalian cell and having the 1-5 base mismatches relative to the CAG repeat region at their predicted positions within positions 8-16 of the guide sequence are at an abundance of at least 10%, 15%, 20%, 25%, 30%, 35%, 40%, 45%, 50%, 55%, 60%, 65%, 70%, 75%, 80%, 85%, 90%, 95%, 97%, or 99% or more in the mammalian cell (in vitro or in vivo) compared to other siRNAs produced or processed from the same double stranded RNA.

The guide strand leads RISC to cognate target mRNAs in a sequence specific manner. In some embodiments, the guide strand induces cleavage of the target mRNA transcript. In some embodiments, the guide strand induces translational repression and/or posterior repression through mRNA decay.

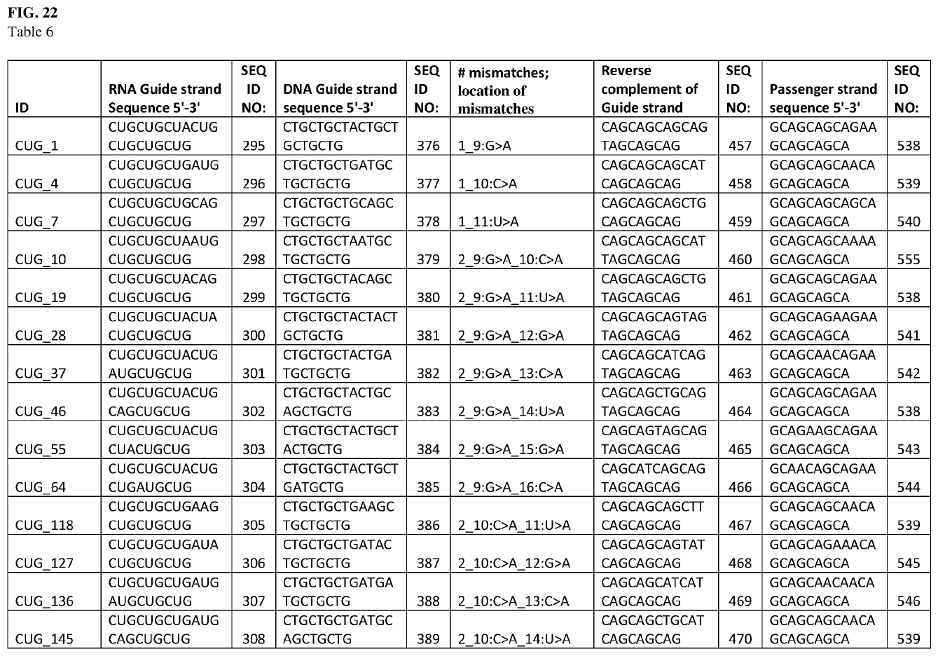

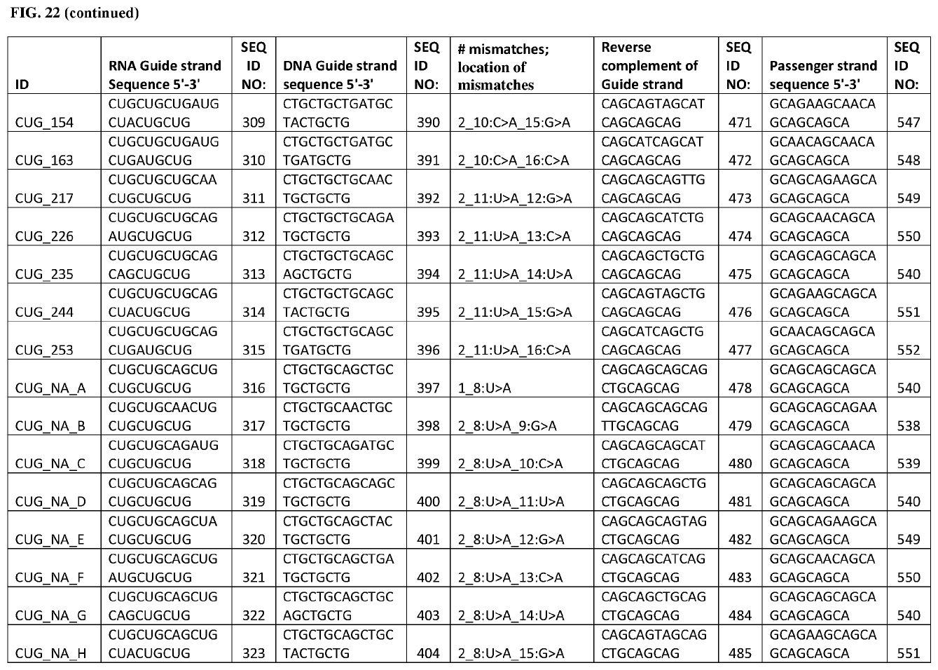

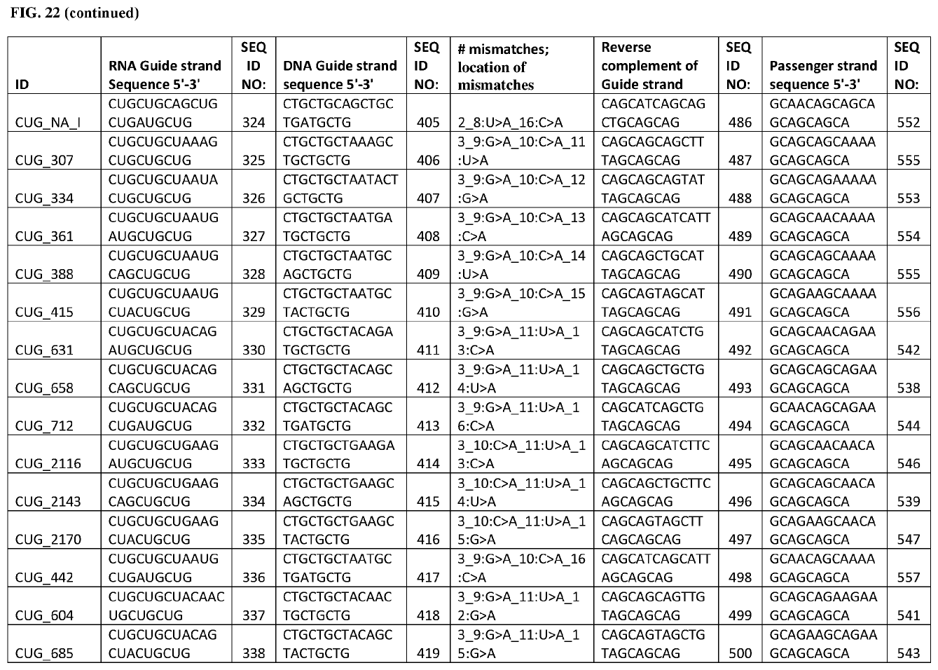

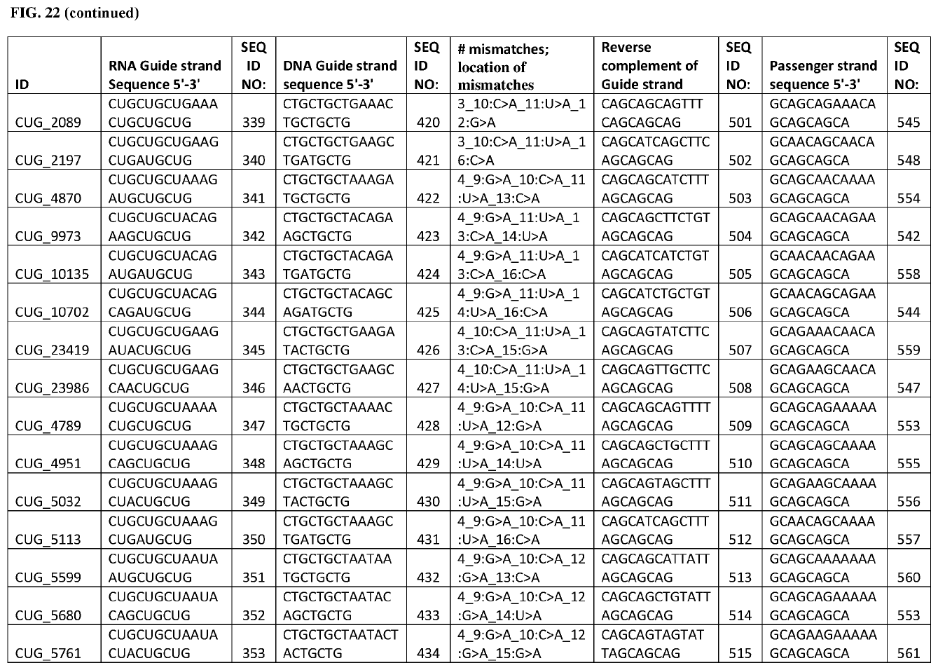

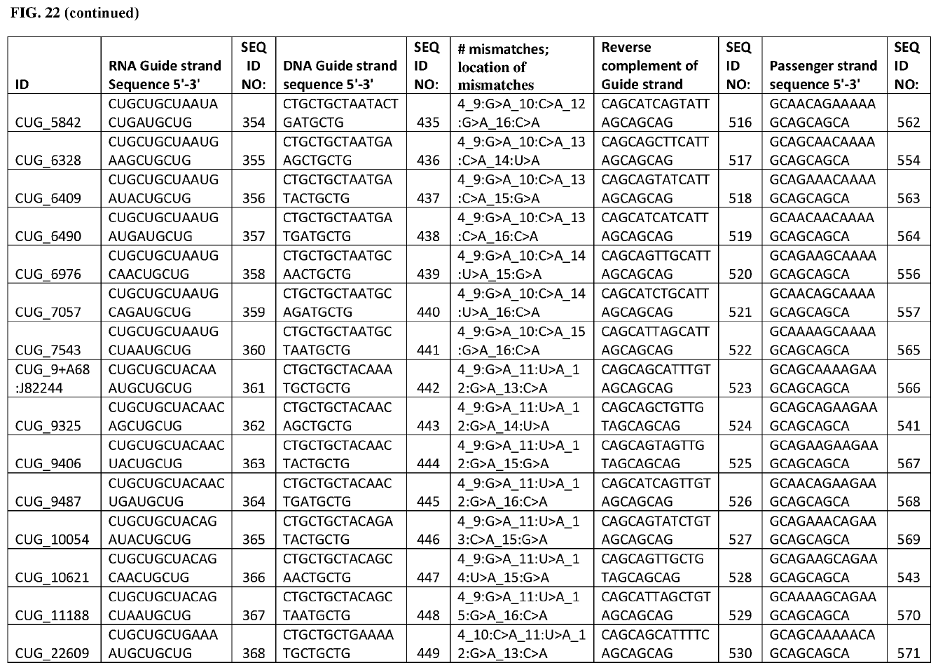

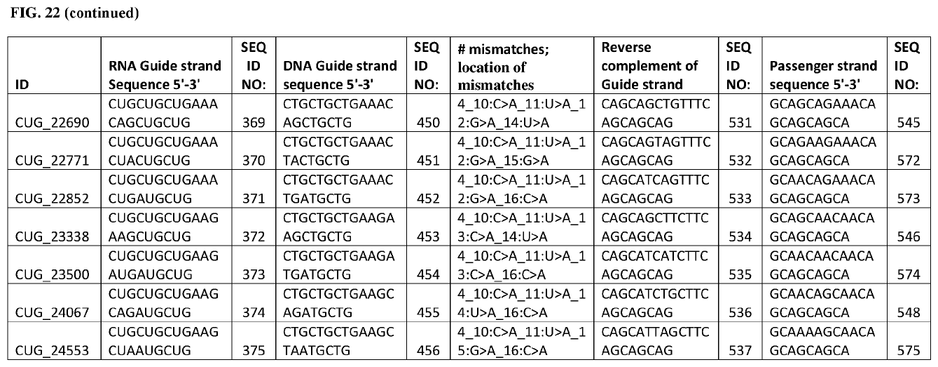

In some cases, a double-stranded RNA of the present disclosure comprises: a) a first strand that hybridizes to a target CAG repeat region of a CAG repeat-containing RNA; and b) a second strand that hybridizes to the first strand, wherein the first strand comprises: i) a first mismatch to the target CAG repeat region; and ii) at least a second mismatch to the target CAG repeat region, wherein: 1) when the first mismatch is at position 8 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), SEQ ID NO:745 (UGCUGCUGCUGCUGCUGCUGCUG (SEQ ID NO:745)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867)), the second mismatch is from is from 1 to 8 bases 3′ of the first mismatch; 2) when the first mismatch is at position 9 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), or SEQ ID NO:745 (UGCUGCUGCUGCUGCUGCUGCUG (SEQ ID NO:745), the second mismatch is from 1 to 7 bases 3′ of the first mismatch; 3) when the first mismatch is at position 10 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), or (UGCUGCUGCUGCUGCUGCUGCUG (SEQ ID NO:745)), the second mismatch is from 1 to 6 bases 3′ of the first mismatch; and 4) when the first mismatch is at position 11 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), or (UGCUGCUGCUGCUGCUGCUGCUG (SEQ ID NO:745)), the second mismatch is from 1 to 5 bases 3′ of the first mismatch. In some cases, the first strand comprises no more than 2 mismatches with the target CAG repeat region. In some cases, the first strand comprises no more than 3 mismatches with the target CAG repeat region. In some cases, the first strand comprises no more than 4 mismatches with the target CAG repeat region. In some cases, the second strand is 100% complementary to the first strand. In some cases, the second strand comprises from 1 to 10 mismatches (e.g., from 1 to 4, from 3 to 5, from 5 to 7, or from 5 to 10 mismatches) to the first strand. In some cases, the second strand comprises from 1 to 4 mismatches to the first strand. In some cases, the second strand comprises no more than 1, 2, 3, or 4 mismatches to the first strand. In some cases, the second strand comprises no more than 1 mismatch to the first strand. In some cases, the second strand comprises no more than 2 mismatches to the first strand. In some cases, the second strand comprises no more than 3 mismatches to the first strand. In some cases, the second strand comprises no more than 4 mismatches to the first strand. In some cases, the second strand comprises no more than 5 mismatches to the first strand. In some cases, the double-stranded RNA has a length of from 18 nucleotides to 25 nucleotides. In some cases, the double-stranded RNA has a length of 20 nucleotides. In some cases, the double-stranded RNA has a length of 21 nucleotides. In some cases, the double-stranded RNA has a length of 22 nucleotides. In some cases, the double-stranded RNA has a length of 23 nucleotides. In some cases, the double-stranded RNA has a length of 24 nucleotides. In some cases, the double-stranded RNA has a length of 25 nucleotides. In some cases, the guide sequence comprises any one of SEQ ID NOs:298-375 (see Table 6; FIG. 22) and has a length of 21 nucleotides. In some cases, each mismatch is generated by substituting a nucleotide (e.g., a nucleotide present in CUGCUGCUGCUGCUGCUGCUG (SEQ ID NO:743), a nucleotide present in GCUGCUGCUGCUGCUGCUGCU (SEQ ID NO:866), or a nucleotide present in UGCUGCUGCUGCUGCUGCUGC (SEQ ID NO:867)) with a different nucleotide. In some cases, each mismatch is generated by a substitution independently selected from: a) a substitution of a G with an A, a U, or a C; b) a substitution of a U with an A, a G, or a C; and c) a substitution of a C with an A, a U, or a G.

### First Mismatch at Position 8; 2 Mismatches

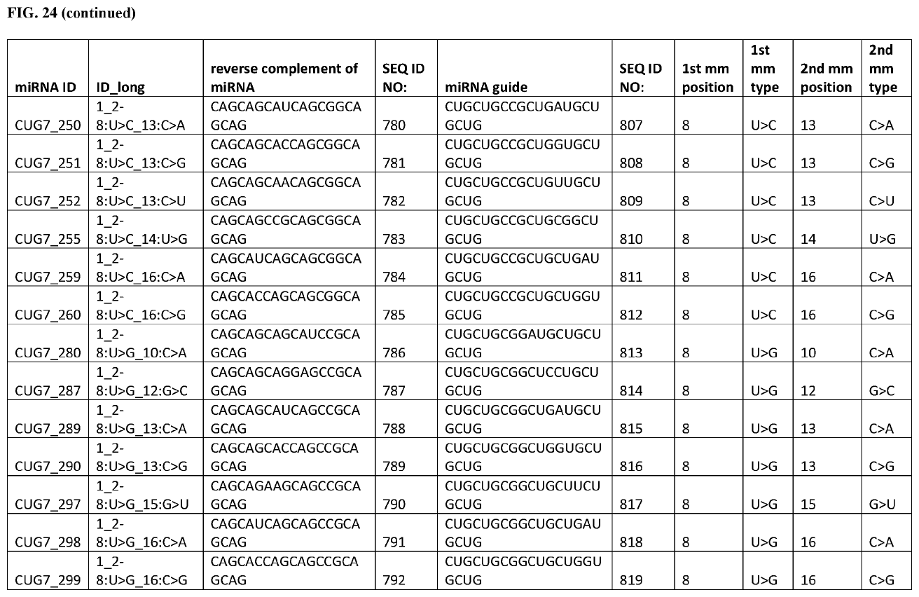

In some cases, a double-stranded RNA of the present disclosure comprises: a) a first strand that hybridizes to a target CAG repeat region of a CAG repeat-containing RNA; and b) a second strand that hybridizes to the first strand, wherein the first strand comprises: i) a first mismatch to the target CAG repeat region, where the first mismatch is at position 8 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), or SEQ ID NO:745 (UGCUGCUGCUGCUGCUGCUGCUG (SEQ ID NO:745); and ii) a second mismatch to the target CAG repeat region, wherein the second mismatch is from 1 to 8 bases 3′ of the first mismatch. In some cases, the first strand is a variant comprising at least a first and a second substitution of the nucleotide sequence: CUGCUGCUGCUGCUGCUGCUG (SEQ ID NO:743) wherein the first substitution generates the first mismatch and is at position 8 based on the numbering of CUGCUGCUGCUGCUGCUGCUG (SEQ ID NO:743) and the second substitution generates the second mismatch and is from 1 to 8 bases 3′ of the first mismatch. In some cases, the first strand is a variant comprising at least a first and a second substitution of the nucleotide sequence: GCUGCUGCUGCUGCUGCUGCU (SEQ ID NO:866), wherein the first substitution generates the first mismatch and is at position 8 based on the numbering of GCUGCUGCUGCUGCUGCUGCU (SEQ ID NO:866), and the second substitution generates the second mismatch and is from 1 to 8 bases 3′ of the first mismatch. In some cases, the first strand is a variant comprising at least a first and a second substitution of the nucleotide sequence: UGCUGCUGCUGCUGCUGCUGC (SEQ ID NO:867), wherein the first substitution generates the first mismatch and is at position 8 based on the numbering of UGCUGCUGCUGCUGCUGCUGC (SEQ ID NO:867), and the second substitution generates the second mismatch and is from 1 to 8 bases 3′ of the first mismatch. In some cases, the first strand comprises no more than 2 mismatches with the target CAG repeat region. In some cases, the first strand comprises no more than 3 mismatches with the target CAG repeat region. In some cases, the first strand comprises no more than 4 mismatches with the target CAG repeat region. In some cases, the second strand is 100% complementary to the first strand. In some cases, the second strand comprises from 1 to 10 mismatches (e.g., from 1 to 4, from 3 to 5, from 5 to 7, or from 5 to 10 mismatches) to the first strand. In some cases, the second strand comprises from 1 to 4 mismatches to the first strand. In some cases, the second strand comprises no more than 1, 2, 3, or 4 mismatches to the first strand. In some cases, the second strand comprises no more than 1 mismatch to the first strand. In some cases, the second strand comprises no more than 2 mismatches to the first strand. In some cases, the second strand comprises no more than 3 mismatches to the first strand. In some cases, the second strand comprises no more than 4 mismatches to the first strand. In some cases, the second strand comprises no more than 5 mismatches to the first strand. In some cases, the double-stranded RNA has a length of from 18 nucleotides to 25 nucleotides. In some cases, the double-stranded RNA has a length of 20 nucleotides. In some cases, the double-stranded RNA has a length of 21 nucleotides. In some cases, the double-stranded RNA has a length of 22 nucleotides. In some cases, the double-stranded RNA has a length of 23 nucleotides. In some cases, the double-stranded RNA has a length of 24 nucleotides. In some cases, the double-stranded RNA has a length of 25 nucleotides. In some cases, each mismatch is generated by a substitution independently selected from: a) a substitution of a G with an A, a U, or a C; b) a substitution of a U with an A, a G, or a C; and c) a substitution of a C with an A, a U, or a G. In some cases, the first strand comprises a nucleotide sequence selected from SEQ ID NOs:317-324. In some cases, the first strand comprises the following nucleotide sequence: CUGCUGCAACUGCUGCUGCUG (SEQ ID NO:317; RNA guide strand sequence of “CUG_NA_B” in Table 6). In some cases, the first strand comprises the following nucleotide sequence: CUGCUGCAGAUGCUGCUGCUG (SEQ ID NO:318; RNA guide strand sequence of “CUG_NA_C” in Table 6). In some cases, the first strand comprises the following nucleotide sequence: CUGCUGCAGCAGCUGCUGCUG (SEQ ID NO:319; RNA guide strand sequence of “CUG_NA_D” in Table 6). In some cases, the first strand comprises the following nucleotide sequence: CUGCUGCAGCUACUGCUGCUG (SEQ ID NO:320; RNA guide strand sequence of “CUG_NA_E” in Table 6). In some cases, the first strand comprises the following nucleotide sequence: CUGCUGCAGCUGAUGCUGCUG (SEQ ID NO:321; RNA guide strand sequence of “CUG_NA_F” in Table 6). In some cases, the first strand comprises the following nucleotide sequence: CUGCUGCAGCUGCAGCUGCUG (SEQ ID NO:322; RNA guide strand sequence of “CUG_NA_G” in Table 6). In some cases, the first strand comprises the following nucleotide sequence: CUGCUGCAGCUGCUACUGCUG (SEQ ID NO:323; RNA guide strand sequence of “CUG_NA_H” in Table 6). In some cases, the first strand comprises the following nucleotide sequence: CUGCUGCAGCUGCUGAUGCUG (SEQ ID NO:324; RNA guide strand sequence of “CUG_NA_I” in Table 6). In some cases, the first strand comprises a nucleotide sequence selected from SEQ ID NOs:804-819 (as depicted in Table 8; FIG. 24).

### First Mismatch at Position 8; 3 Mismatches

In some cases, a double-stranded RNA of the present disclosure comprises a) a first strand that hybridizes to a target CAG repeat region of a CAG repeat-containing RNA, wherein the first strand comprises a first mismatch to the target CAG repeat region, wherein the first mismatch is at position 8 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867)); and b) a second strand that hybridizes to the first strand, wherein the first strand comprises a second mismatch and a third mismatch to the target CAG repeat region, and wherein the second and third mismatches are from 1 to 8 bases 3′ of the first mismatch. In some cases, the first strand is a variant comprising at least a first, a second, and a third substitution of the nucleotide sequence CUGCUGCUGCUGCUGCUGCUG (SEQ ID NO:743), wherein the first substitution generates the first mismatch and is at position 8 based on the numbering of CUGCUGCUGCUGCUGCUGCUG (SEQ ID NO:743), wherein the second substitution generates the second mismatch and the third substitution generates the third mismatch, and wherein the second substitution and the third substitution are from 1 to 8 bases 3′ of the first mismatch. In some cases, the first strand is a variant comprising at least a first, a second, and a third substitution of the nucleotide sequence GCUGCUGCUGCUGCUGCUGCU (SEQ ID NO:866), and wherein the first substitution generates the first mismatch and is at position 8 based on the numbering of GCUGCUGCUGCUGCUGCUGCU (SEQ ID NO:866), wherein the second substitution generates the second mismatch and the third substitution generates the third mismatch, and wherein the second substitution and the third substitution are from 1 to 8 bases 3′ of the first mismatch. In some cases, the first strand is a variant comprising at least a first, a second, and a third substitution of the nucleotide sequence UGCUGCUGCUGCUGCUGCUGC (SEQ ID NO:867), and wherein the first substitution generates the first mismatch and is at position 8 based on the numbering of UGCUGCUGCUGCUGCUGCUGC (SEQ ID NO:867), wherein the second substitution generates the second mismatch and the third substitution generates the third mismatch, and wherein the second substitution and the third substitution are from 1 to 8 bases 3′ of the first mismatch. In some cases, the first strand comprises no more than 3 mismatches with the target CAG repeat region. In some cases, the first strand comprises no more than 4 mismatches with the target CAG repeat region. In some cases, the second strand is 100% complementary to the first strand. In some cases, the second strand comprises from 1 to 10 mismatches (e.g., from 1 to 4, from 3 to 5, from 5 to 7, or from 5 to 10 mismatches) to the first strand. In some cases, the second strand comprises from 1 to 4 mismatches to the first strand. In some cases, the second strand comprises no more than 1, 2, 3, or 4 mismatches to the first strand. In some cases, the second strand comprises no more than 1 mismatch to the first strand. In some cases, the second strand comprises no more than 2 mismatches to the first strand. In some cases, the second strand comprises no more than 3 mismatches to the first strand. In some cases, the second strand comprises no more than 4 mismatches to the first strand. In some cases, the second strand comprises no more than 5 mismatches to the first strand. In some cases, the double-stranded RNA has a length of from 18 nucleotides to 25 nucleotides. In some cases, the double-stranded RNA has a length of 20 nucleotides. In some cases, the double-stranded RNA has a length of 21 nucleotides. In some cases, the double-stranded RNA has a length of 22 nucleotides. In some cases, the double-stranded RNA has a length of 23 nucleotides. In some cases, the double-stranded RNA has a length of 24 nucleotides. In some cases, the double-stranded RNA has a length of 25 nucleotides. In some cases, each mismatch is generated by a substitution independently selected from: a) a substitution of a G with an A, a U, or a C; b) a substitution of a U with an A, a G, or a C; and c) a substitution of a C with an A, a U, or a G.

### First Mismatch at Position 8; 4 Mismatches

In some cases, a double-stranded RNA of the present disclosure comprises a) a first strand that hybridizes to a target CAG repeat region of a CAG repeat-containing RNA, wherein the first strand comprises a first mismatch to the target CAG repeat region, wherein the first mismatch is at position 8 based on the numbering of SEQ ID NO:743 (CUGCUGCUGCUGCUGCUGCUG (SEQ ID NO:743)), SEQ ID NO:867 (GCUGCUGCUGCUGCUGCUGCU (SEQ ID NO:867)), or SEQ ID NO:866 (UGCUGCUGCUGCUGCUGCUGC (SEQ ID NO:866)); and b) a second strand that hybridizes to the first strand, wherein the first strand comprises a second mismatch, a third mismatch, and a fourth mismatch to the target CAG repeat region, and wherein the second, third, and fourth mismatches are from 1 to 8 bases 3′ of the first mismatch. In some cases, the first strand is a variant comprising at least a first, a second, a third, and a fourth substitution of the nucleotide sequence CUGCUGCUGCUGCUGCUGCUG (SEQ ID NO:743), wherein the first substitution generates the first mismatch and is at position 8 based on the numbering of CUGCUGCUGCUGCUGCUGCUG (SEQ ID NO:743), wherein the second substitution generates the second mismatch, the third substitution generates the third mismatch, and the fourth substitution generates the fourth mismatch, and wherein the second, third, and fourth substitutions are from 1 to 8 bases 3′ of the first mismatch. In some cases, the first strand is a variant comprising at least a first, a second, a third, and a fourth substitution of the nucleotide sequence GCUGCUGCUGCUGCUGCUGCU (SEQ ID NO:866), wherein the first substitution generates the first mismatch and is at position 8 based on the numbering of GCUGCUGCUGCUGCUGCUGCU (SEQ ID NO:866), wherein the second substitution generates the second mismatch, the third substitution generates the third mismatch, and the fourth substitution generates the fourth mismatch, and wherein the second, third, and fourth substitutions are from 1 to 8 bases 3′ of the first mismatch. In some cases, the first strand is a variant comprising at least a first, a second, a third, and a fourth substitution of the nucleotide sequence UGCUGCUGCUGCUGCUGCUGC (SEQ ID NO:867), wherein the first substitution generates the first mismatch and is at position 8 based on the numbering of UGCUGCUGCUGCUGCUGCUGC (SEQ ID NO:867), wherein the second substitution generates the second mismatch, the third substitution generates the third mismatch, and the fourth substitution generates the fourth mismatch, and wherein the second, third, and fourth substitutions are from 1 to 8 bases 3′ of the first mismatch. In some cases, the first strand comprises no more than 4 mismatches with the target CAG repeat region. In some cases, the second strand is 100% complementary to the first strand. In some cases, the second strand comprises from 1 to 10 mismatches (e.g., from 1 to 4, from 3 to 5, from 5 to 7, or from 5 to 10 mismatches) to the first strand. In some cases, the second strand comprises from 1 to 4 mismatches to the first strand. In some cases, the second strand comprises no more than 1, 2, 3, or 4 mismatches to the first strand. In some cases, the second strand comprises no more than 1 mismatch to the first strand. In some cases, the second strand comprises no more than 2 mismatches to the first strand. In some cases, the second strand comprises no more than 3 mismatches to the first strand. In some cases, the second strand comprises no more than 4 mismatches to the first strand. In some cases, the second strand comprises no more than 5 mismatches to the first strand. In some cases, the double-stranded RNA has a length of from 18 nucleotides to 25 nucleotides. In some cases, the double-stranded RNA has a length of 20 nucleotides. In some cases, the double-stranded RNA has a length of 21 nucleotides. In some cases, the double-stranded RNA has a length of 22 nucleotides. In some cases, the double-stranded RNA has a length of 23 nucleotides. In some cases, the double-stranded RNA has a length of 24 nucleotides. In some cases, the double-stranded RNA has a length of 25 nucleotides. In some cases, each mismatch is generated by a substitution independently selected from: a) a substitution of a G with an A, a U, or a C; b) a substitution of a U with an A, a G, or a C; and c) a substitution of a C with an A, a U, or a G.

### First Mismatch at Position 9; 2 Mismatches

In some cases, a double-stranded RNA of the present disclosure comprises: a) a first strand that hybridizes to a target CAG repeat region of a CAG repeat-containing RNA; and b) a second strand that hybridizes to the first strand, wherein the first strand comprises: i) a first mismatch to the target CAG repeat region, where the first mismatch is at position 9 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), SEQ ID NO:745 (UGCUGCUGCUGCUGCUGCUGCUG (SEQ ID NO:745), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867); and ii) a second mismatch to the target CAG repeat region, wherein the second mismatch is from 1 to 7 bases 3′ of the first mismatch. In some cases, the first strand is a variant comprising at least a first and a second substitution of the nucleotide sequence CUGCUGCUGCUGCUGCUGCUG (SEQ ID NO:743), wherein the first substitution generates the first mismatch and is at position 9 based on the numbering of CUGCUGCUGCUGCUGCUGCUG (SEQ ID NO:743), and the second substitution generates the second mismatch and is from 1 to 7 bases 3′ of the first mismatch. In some cases, the first strand is a variant comprising at least a first and a second substitution of the nucleotide sequence GCUGCUGCUGCUGCUGCUGCU (SEQ ID NO:866), wherein the first substitution generates the first mismatch and is at position 9 based on the numbering of GCUGCUGCUGCUGCUGCUGCU (SEQ ID NO:866), and the second substitution generates the second mismatch and is from 1 to 7 bases 3′ of the first mismatch. In some cases, the first strand is a variant comprising at least a first and a second substitution of the nucleotide sequence UGCUGCUGCUGCUGCUGCUGC (SEQ ID NO:867), wherein the first substitution generates the first mismatch and is at position 9 based on the numbering of UGCUGCUGCUGCUGCUGCUGC (SEQ ID NO:867), and the second substitution generates the second mismatch and is from 1 to 7 bases 3′ of the first mismatch. In some cases, the first strand comprises no more than 2 mismatches with the target CAG repeat region. In some cases, the second strand is 100% complementary to the first strand. In some cases, the second strand comprises from 1 to 10 mismatches (e.g., from 1 to 4, from 3 to 5, from 5 to 7, or from 5 to 10 mismatches) to the first strand. In some cases, the second strand comprises from 1 to 4 mismatches to the first strand. In some cases, the second strand comprises no more than 1, 2, 3, or 4 mismatches to the first strand. In some cases, the second strand comprises no more than 1 mismatch to the first strand. In some cases, the second strand comprises no more than 2 mismatches to the first strand. In some cases, the second strand comprises no more than 3 mismatches to the first strand. In some cases, the second strand comprises no more than 4 mismatches to the first strand. In some cases, the second strand comprises no more than 5 mismatches to the first strand. In some cases, the double-stranded RNA has a length of from 18 nucleotides to 25 nucleotides. In some cases, the double-stranded RNA has a length of 20 nucleotides. In some cases, the double-stranded RNA has a length of 21 nucleotides. In some cases, the double-stranded RNA has a length of 22 nucleotides. In some cases, the double-stranded RNA has a length of 23 nucleotides. In some cases, the double-stranded RNA has a length of 24 nucleotides. In some cases, the double-stranded RNA has a length of 25 nucleotides. In some cases, each mismatch is generated by a substitution independently selected from: a) a substitution of a G with an A, a U, or a C; b) a substitution of a U with an A, a G, or a C; and c) a substitution of a C with an A, a U, or a G. In some cases, the first strand comprises a nucleotide sequence selected from SEQ ID NOs:298-304.

### First Mismatch at Position 9; 3 Mismatches

In some cases, a double-stranded RNA of the present disclosure comprises: a) a first strand that hybridizes to a target CAG repeat region of a CAG repeat-containing RNA; and b) a second strand that hybridizes to the first strand, where the first mismatch is at position 9 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), SEQ ID NO:745 (UGCUGCUGCUGCUGCUGCUGCUG (SEQ ID NO:745), (GCUGCUGCUGCUGCUGCUGCU (SEQ ID NO:866)), or SEQ ID NO:867 (UGCUGCUGCUGCUGCUGCUGC (SEQ ID NO:867), where the first strand comprises a second mismatch and a third mismatch to the target CAG repeat region, and where the second and third mismatches are from 1 to 7 bases 3′ of the first mismatch. In some cases, the first strand is a variant comprising at least a first, a second, and a third substitution of the nucleotide sequence CUGCUGCUGCUGCUGCUGCUG (SEQ ID NO:743), wherein the first substitution generates the first mismatch and is at position 9 based on the numbering of CUGCUGCUGCUGCUGCUGCUG (SEQ ID NO:743), wherein the second substitution generates the second mismatch and the third substitution generates the third mismatch, and wherein the second substitution and the third substitution are from 1 to 7 bases 3′ of the first mismatch. In some cases, the first strand is a variant comprising at least a first, a second, and a third substitution of the nucleotide sequence GCUGCUGCUGCUGCUGCUGCU (SEQ ID NO:866), wherein the first substitution generates the first mismatch and is at position 9 based on the numbering of GCUGCUGCUGCUGCUGCUGCU (SEQ ID NO:866), wherein the second substitution generates the second mismatch and the third substitution generates the third mismatch, and wherein the second substitution and the third substitution are from 1 to 7 bases 3′ of the first mismatch. In some cases, the first strand is a variant comprising at least a first, a second, and a third substitution of the nucleotide sequence UGCUGCUGCUGCUGCUGCUGC (SEQ ID NO:867), wherein the first substitution generates the first mismatch and is at position 9 based on the numbering of UGCUGCUGCUGCUGCUGCUGC (SEQ ID NO:867), wherein the second substitution generates the second mismatch and the third substitution generates the third mismatch, and wherein the second substitution and the third substitution are from 1 to 7 bases 3′ of the first mismatch. In some cases, the first strand comprises no more than 3 mismatches with the target CAG repeat region. In some cases, the first strand comprises no more than 4 mismatches with the target CAG repeat region. In some cases, the second strand is 100% complementary to the first strand. In some cases, the second strand comprises from 1 to 10 mismatches (e.g., from 1 to 4, from 3 to 5, from 5 to 7, or from 5 to 10 mismatches) to the first strand. In some cases, the second strand comprises from 1 to 4 mismatches to the first strand. In some cases, the second strand comprises no more than 1, 2, 3, or 4 mismatches to the first strand. In some cases, the second strand comprises no more than 1 mismatch to the first strand. In some cases, the second strand comprises no more than 2 mismatches to the first strand. In some cases, the second strand comprises no more than 3 mismatches to the first strand. In some cases, the second strand comprises no more than 4 mismatches to the first strand. In some cases, the second strand comprises no more than 5 mismatches to the first strand. In some cases, the double-stranded RNA has a length of from 18 nucleotides to 25 nucleotides. In some cases, the double-stranded RNA has a length of 20 nucleotides. In some cases, the double-stranded RNA has a length of 21 nucleotides. In some cases, the double-stranded RNA has a length of 22 nucleotides. In some cases, the double-stranded RNA has a length of 23 nucleotides. In some cases, the double-stranded RNA has a length of 24 nucleotides. In some cases, the double-stranded RNA has a length of 25 nucleotides. In some cases, each mismatch is generated by a substitution independently selected from: a) a substitution of a G with an A, a U, or a C; b) a substitution of a U with an A, a G, or a C; and c) a substitution of a C with an A, a U, or a G. In some cases, the first strand comprises a nucleotide sequence selected from SEQ ID NOs:325-332 and 336-338. In some cases, the first strand comprises the following nucleotide sequence: CUGCUGCUAAAGCUGCUGCUG (SEQ ID NO:325; RNA guide strand sequence of “CUG_307” in Table 6). In some cases, the first strand comprises the following nucleotide sequence: CUGCUGCUAAUACUGCUGCUG (SEQ ID NO:326; RNA guide strand sequence of “CUG_334” in Table 6). In some cases, the first strand comprises the following nucleotide sequence: CUGCUGCUAAUGAUGCUGCUG (SEQ ID NO:327; RNA guide strand sequence of “CUG_361” in Table 6). In some cases, the first strand comprises the following nucleotide sequence: CUGCUGCUAAUGCAGCUGCUG (SEQ ID NO:328; RNA guide strand sequence of “CUG_388” in Table 6). In some cases, the first strand comprises the following nucleotide sequence: CUGCUGCUAAUGCUACUGCUG (SEQ ID NO:329; RNA guide strand sequence of “CUG_415” in Table 6). In some cases, the first strand comprises the following nucleotide sequence: CUGCUGCUACAGAUGCUGCUG (SEQ ID NO:330; RNA guide strand sequence of “CUG_631” in Table 6). In some cases, the first strand comprises the following nucleotide sequence: CUGCUGCUACAGCAGCUGCUG (SEQ ID NO:331; RNA guide strand sequence of “CUG_658” in Table 6). In some cases, the first strand comprises the following nucleotide sequence: CUGCUGCUACAGCUGAUGCUG (SEQ ID NO:332; RNA guide strand sequence of “CUG_712” in Table 6). In some cases, the first strand comprises the following nucleotide sequence: CUGCUGCUAAUGCUGAUGCUG (SEQ ID NO:336; RNA guide strand sequence of “CUG_442” in Table 6). In some cases, the first strand comprises the following nucleotide sequence: CUGCUGCUACAACUGCUGCUG (SEQ ID NO:337; RNA guide strand sequence of “CUG_604” in Table 6). In some cases, the first strand comprises the following nucleotide sequence: CUGCUGCUACAGCUACUGCUG (SEQ ID NO:338; RNA guide strand sequence of “CUG_685” in Table 6).

### First Mismatch at Position 9; 4 Mismatches

In some cases, a double-stranded RNA of the present disclosure comprises: a) a first strand that hybridizes to a target CAG repeat region of a CAG repeat-containing RNA; and b) a second strand that hybridizes to the first strand, where the first mismatch is at position 9 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), SEQ ID NO:745 (UGCUGCUGCUGCUGCUGCUGCUG (SEQ ID NO:745), (GCUGCUGCUGCUGCUGCUGCU (SEQ ID NO:866)), or SEQ ID NO:867 (UGCUGCUGCUGCUGCUGCUGC (SEQ ID NO:867), wherein the first strand comprises a second mismatch, a third mismatch, and a fourth mismatch to the target CAG repeat region, and wherein the second, third, and fourth mismatches are from 1 to 7 bases 3′ of the first mismatch. In some cases, the first strand is a variant comprising at least a first, a second, a third, and a fourth substitution of the nucleotide sequence CUGCUGCUGCUGCUGCUGCUG (SEQ ID NO:743), wherein the first substitution generates the first mismatch and is at position 9 based on the numbering of CUGCUGCUGCUGCUGCUGCUG (SEQ ID NO:743), wherein the second substitution generates the second mismatch, the third substitution generates the third mismatch, and the fourth substitution generates the fourth mismatch, and wherein the second, third, and fourth substitutions are from 1 to 7 bases 3′ of the first mismatch. In some cases, the first strand is a variant comprising at least a first, a second, a third, and a fourth substitution of the nucleotide sequence GCUGCUGCUGCUGCUGCUGCU (SEQ ID NO:866), wherein the first substitution generates the first mismatch and is at position 9 based on the numbering of GCUGCUGCUGCUGCUGCUGCU (SEQ ID NO:866), wherein the second substitution generates the second mismatch, the third substitution generates the third mismatch, and the fourth substitution generates the fourth mismatch, and wherein the second, third, and fourth substitutions are from 1 to 7 bases 3′ of the first mismatch. In some cases, the first strand is a variant comprising at least a first, a second, a third, and a fourth substitution of the nucleotide sequence UGCUGCUGCUGCUGCUGCUGC (SEQ ID NO:867), wherein the first substitution generates the first mismatch and is at position 9 based on the numbering of UGCUGCUGCUGCUGCUGCUGC (SEQ ID NO:867), wherein the second substitution generates the second mismatch, the third substitution generates the third mismatch, and the fourth substitution generates the fourth mismatch, and wherein the second, third, and fourth substitutions are from 1 to 7 bases 3′ of the first mismatch. In some cases, the first strand comprises no more than 4 mismatches with the target CAG repeat region. In some cases, the second strand is 100% complementary to the first strand. In some cases, the second strand comprises from 1 to 10 mismatches (e.g., from 1 to 4, from 3 to 5, from 5 to 7, or from 5 to 10 mismatches) to the first strand. In some cases, the second strand comprises from 1 to 4 mismatches to the first strand. In some cases, the second strand comprises no more than 1, 2, 3, or 4 mismatches to the first strand. In some cases, the second strand comprises no more than 1 mismatch to the first strand. In some cases, the second strand comprises no more than 2 mismatches to the first strand. In some cases, the second strand comprises no more than 3 mismatches to the first strand. In some cases, the second strand comprises no more than 4 mismatches to the first strand. In some cases, the second strand comprises no more than 5 mismatches to the first strand. In some cases, the double-stranded RNA has a length of from 18 nucleotides to 25 nucleotides. In some cases, the double-stranded RNA has a length of 20 nucleotides. In some cases, the double-stranded RNA has a length of 21 nucleotides. In some cases, the double-stranded RNA has a length of 22 nucleotides. In some cases, the double-stranded RNA has a length of 23 nucleotides. In some cases, the double-stranded RNA has a length of 24 nucleotides. In some cases, the double-stranded RNA has a length of 25 nucleotides. In some cases, each mismatch is generated by a substitution independently selected from: a) a substitution of a G with an A, a U, or a C; b) a substitution of a U with an A, a G, or a C; and c) a substitution of a C with an A, a U, or a G. In some cases, the first strand comprises a nucleotide sequence selected from SEQ ID NOs:341-344 and 347-367.

### First Mismatch at Position 10; 2 Mismatches

In some cases, a double-stranded RNA of the present disclosure comprises: a) a first strand that hybridizes to a target CAG repeat region of a CAG repeat-containing RNA; and b) a second strand that hybridizes to the first strand, wherein the first strand comprises: i) a first mismatch to the target CAG repeat region, where the first mismatch is at position 10 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), SEQ ID NO:745 (UGCUGCUGCUGCUGCUGCUGCUG (SEQ ID NO:745), (GCUGCUGCUGCUGCUGCUGCU (SEQ ID NO:866)), or SEQ ID NO:867 (UGCUGCUGCUGCUGCUGCUGC (SEQ ID NO:867); and ii) a second mismatch to the target CAG repeat region, wherein the second mismatch is from 1 to 6 bases 3′ of the first mismatch. In some cases, the first strand is a variant comprising at least a first and a second substitution of the nucleotide sequence CUGCUGCUGCUGCUGCUGCUG (SEQ ID NO:743), wherein the first substitution generates the first mismatch and is at position 10 based on the numbering of CUGCUGCUGCUGCUGCUGCUG (SEQ ID NO:743), and wherein the second substitution generates the second mismatch and is from 1 to 6 bases 3′ of the first mismatch. In some cases, the first strand is a variant comprising at least a first and a second substitution of the nucleotide sequence GCUGCUGCUGCUGCUGCUGCU (SEQ ID NO:866), wherein the first substitution generates the first mismatch and is at position 10 based on the numbering of GCUGCUGCUGCUGCUGCUGCU (SEQ ID NO:866), and wherein the second substitution generates the second mismatch and is from 1 to 6 bases 3′ of the first mismatch. In some cases, the first strand is a variant comprising at least a first and a second substitution of the nucleotide sequence UGCUGCUGCUGCUGCUGCUGC (SEQ ID NO:867), wherein the first substitution generates the first mismatch and is at position 10 based on the numbering of UGCUGCUGCUGCUGCUGCUGC (SEQ ID NO:867), and wherein the second substitution generates the second mismatch and is from 1 to 6 bases 3′ of the first mismatch. In some cases, the first strand comprises no more than 2 mismatches with the target CAG repeat region. In some cases, the first strand comprises no more than 3 mismatches with the target CAG repeat region. In some cases, the first strand comprises no more than 4 mismatches with the target CAG repeat region. In some cases, the second strand is 100% complementary to the first strand. In some cases, the second strand comprises from 1 to 10 mismatches (e.g., from 1 to 4, from 3 to 5, from 5 to 7, or from 5 to 10 mismatches) to the first strand. In some cases, the second strand comprises from 1 to 4 mismatches to the first strand. In some cases, the second strand comprises no more than 1, 2, 3, or 4 mismatches to the first strand. In some cases, the second strand comprises no more than 1 mismatch to the first strand. In some cases, the second strand comprises no more than 2 mismatches to the first strand. In some cases, the second strand comprises no more than 3 mismatches to the first strand. In some cases, the second strand comprises no more than 4 mismatches to the first strand. In some cases, the second strand comprises no more than 5 mismatches to the first strand. In some cases, the double-stranded RNA has a length of from 18 nucleotides to 25 nucleotides. In some cases, the double-stranded RNA has a length of 20 nucleotides. In some cases, the double-stranded RNA has a length of 21 nucleotides. In some cases, the double-stranded RNA has a length of 22 nucleotides. In some cases, the double-stranded RNA has a length of 23 nucleotides. In some cases, the double-stranded RNA has a length of 24 nucleotides. In some cases, the double-stranded RNA has a length of 25 nucleotides. In some cases, each mismatch is generated by a substitution independently selected from: a) a substitution of a G with an A, a U, or a C; b) a substitution of a U with an A, a G, or a C; and c) a substitution of a C with an A, a U, or a G. In some cases, the first strand comprises a nucleotide sequence selected from SEQ ID NOs:305-310.

### First Mismatch at Position 10; 3 Mismatches

In some cases, a double-stranded RNA of the present disclosure comprises: a) a first strand that hybridizes to a target CAG repeat region of a CAG repeat-containing RNA; and b) a second strand that hybridizes to the first strand, where the first mismatch is at position 10 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), or (UGCUGCUGCUGCUGCUGCUGCUG (SEQ ID NO:745)), where the first strand comprises a second mismatch and a third mismatch to the target CAG repeat region, and where the second and third mismatches are from 1 to 6 bases 3′ of the first mismatch. In some cases, the first strand is a variant comprising at least a first, a second, and a third substitution of the nucleotide sequence CUGCUGCUGCUGCUGCUGCUG (SEQ ID NO:743), wherein the first substitution generates the first mismatch and is at position 10 based on the numbering of CUGCUGCUGCUGCUGCUGCUG (SEQ ID NO:743), wherein the second substitution generates the second mismatch and the third substitution generates the third mismatch, and wherein the second substitution and the third substitution are from 1 to 6 bases 3′ of the first mismatch. In some cases, the first strand is a variant comprising at least a first, a second, and a third substitution of the nucleotide sequence GCUGCUGCUGCUGCUGCUGCU (SEQ ID NO:866), wherein the first substitution generates the first mismatch and is at position 10 based on the numbering of GCUGCUGCUGCUGCUGCUGCU (SEQ ID NO:866), wherein the second substitution generates the second mismatch and the third substitution generates the third mismatch, and wherein the second substitution and the third substitution are from 1 to 6 bases 3′ of the first mismatch. In some cases, the first strand is a variant comprising at least a first, a second, and a third substitution of the nucleotide sequence UGCUGCUGCUGCUGCUGCUGC (SEQ ID NO:867), wherein the first substitution generates the first mismatch and is at position 10 based on the numbering UGCUGCUGCUGCUGCUGCUGC (SEQ ID NO:867), wherein the second substitution generates the second mismatch and the third substitution generates the third mismatch, and wherein the second substitution and the third substitution are from 1 to 6 bases 3′ of the first mismatch. In some cases, the first strand comprises no more than 3 mismatches with the target CAG repeat region. In some cases, the first strand comprises no more than 4 mismatches with the target CAG repeat region. In some cases, the second strand is 100% complementary to the first strand. In some cases, the second strand comprises from 1 to 10 mismatches (e.g., from 1 to 4, from 3 to 5, from 5 to 7, or from 5 to 10 mismatches) to the first strand. In some cases, the second strand comprises from 1 to 4 mismatches to the first strand. In some cases, the second strand comprises no more than 1, 2, 3, or 4 mismatches to the first strand. In some cases, the second strand comprises no more than 1 mismatch to the first strand. In some cases, the second strand comprises no more than 2 mismatches to the first strand. In some cases, the second strand comprises no more than 3 mismatches to the first strand. In some cases, the second strand comprises no more than 4 mismatches to the first strand. In some cases, the second strand comprises no more than 5 mismatches to the first strand. In some cases, the double-stranded RNA has a length of from 18 nucleotides to 25 nucleotides. In some cases, the double-stranded RNA has a length of 20 nucleotides. In some cases, the double-stranded RNA has a length of 21 nucleotides. In some cases, the double-stranded RNA has a length of 22 nucleotides. In some cases, the double-stranded RNA has a length of 23 nucleotides. In some cases, the double-stranded RNA has a length of 24 nucleotides. In some cases, the double-stranded RNA has a length of 25 nucleotides. In some cases, each mismatch is generated by a substitution independently selected from: a) a substitution of a G with an A, a U, or a C; b) a substitution of a U with an A, a G, or a C; and c) a substitution of a C with an A, a U, or a G. In some cases, the first strand comprises a nucleotide sequence selected from SEQ ID NO:333-335, 339, and 340. In some cases, the first strand comprises the following nucleotide sequence: CUGCUGCUGAAGAUGCUGCUG (SEQ ID NO:333; RNA guide strand sequence of “CUG_2116” in Table 6). In some cases, the first strand comprises the following nucleotide sequence: CUGCUGCUGAAGCAGCUGCUG (SEQ ID NO:334; RNA guide strand sequence of “CUG_2143” in Table 6). In some cases, the first strand comprises the following nucleotide sequence: CUGCUGCUGAAGCUACUGCUG (SEQ ID NO:335; RNA guide strand sequence of “CUG_2170” in Table 6). In some cases, the first strand comprises the following nucleotide sequence: CUGCUGCUGAAACUGCUGCUG (SEQ ID NO:339; RNA guide strand sequence of “CUG_2089” in Table 6). In some cases, the first strand comprises the following nucleotide sequence: CUGCUGCUGAAGCUGAUGCUG (SEQ ID NO:340; RNA guide strand sequence of “CUG_2197” in Table 6).

### First Mismatch at Position 10; 4 Mismatches

In some cases, a double-stranded RNA of the present disclosure comprises: a) a first strand that hybridizes to a target CAG repeat region of a CAG repeat-containing RNA; and b) a second strand that hybridizes to the first strand, where the first mismatch is at position 11 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), SEQ ID NO:745 (UGCUGCUGCUGCUGCUGCUGCUG (SEQ ID NO:745), (GCUGCUGCUGCUGCUGCUGCU (SEQ ID NO:866)), or SEQ ID NO:867 (UGCUGCUGCUGCUGCUGCUGC (SEQ ID NO:867), wherein the first strand comprises a second mismatch, a third mismatch, and a fourth mismatch to the target CAG repeat region, and wherein the second, third, and fourth mismatches are from 1 to 6 bases 3′ of the first mismatch. In some cases, the first strand is a variant comprising at least a first, a second, a third, and a fourth substitution of the nucleotide sequence CUGCUGCUGCUGCUGCUGCUG (SEQ ID NO:743), wherein the first substitution generates the first mismatch and is at position 10 based on the numbering of CUGCUGCUGCUGCUGCUGCUG (SEQ ID NO:743), wherein the second substitution generates the second mismatch, the third substitution generates the third mismatch, and the fourth substitution generates the fourth mismatch, and wherein the second, third, and fourth substitutions are from 1 to 6 bases 3′ of the first mismatch. In some cases, the first strand is a variant comprising at least a first, a second, a third, and a fourth substitution of the nucleotide sequence GCUGCUGCUGCUGCUGCUGCU (SEQ ID NO:866), wherein the first substitution generates the first mismatch and is at position 10 based on the numbering of GCUGCUGCUGCUGCUGCUGCU (SEQ ID NO:866), wherein the second substitution generates the second mismatch, the third substitution generates the third mismatch, and the fourth substitution generates the fourth mismatch, and wherein the second, third, and fourth substitutions are from 1 to 6 bases 3′ of the first mismatch. In some cases, the first strand is a variant comprising at least a first, a second, a third, and a fourth substitution of the nucleotide sequence UGCUGCUGCUGCUGCUGCUGC (SEQ ID NO:867), wherein the first substitution generates the first mismatch and is at position 10 based on the numbering of UGCUGCUGCUGCUGCUGCUGC (SEQ ID NO:867), wherein the second substitution generates the second mismatch, the third substitution generates the third mismatch, and the fourth substitution generates the fourth mismatch, and wherein the second, third, and fourth substitutions are from 1 to 6 bases 3′ of the first mismatch. In some cases, the first strand comprises no more than 4 mismatches with the target CAG repeat region. In some cases, the second strand is 100% complementary to the first strand. In some cases, the second strand comprises from 1 to 10 mismatches (e.g., from 1 to 4, from 3 to 5, from 5 to 7, or from 5 to 10 mismatches) to the first strand. In some cases, the second strand comprises from 1 to 4 mismatches to the first strand. In some cases, the second strand comprises no more than 1, 2, 3, or 4 mismatches to the first strand. In some cases, the second strand comprises no more than 1 mismatch to the first strand. In some cases, the second strand comprises no more than 2 mismatches to the first strand. In some cases, the second strand comprises no more than 3 mismatches to the first strand. In some cases, the second strand comprises no more than 4 mismatches to the first strand. In some cases, the second strand comprises no more than 5 mismatches to the first strand. In some cases, the double-stranded RNA has a length of from 18 nucleotides to 25 nucleotides. In some cases, the double-stranded RNA has a length of 20 nucleotides. In some cases, the double-stranded RNA has a length of 21 nucleotides. In some cases, the double-stranded RNA has a length of 22 nucleotides. In some cases, the double-stranded RNA has a length of 23 nucleotides. In some cases, the double-stranded RNA has a length of 24 nucleotides. In some cases, the double-stranded RNA has a length of 25 nucleotides. In some cases, each mismatch is generated by a substitution independently selected from: a) a substitution of a G with an A, a U, or a C; b) a substitution of a U with an A, a G, or a C; and c) a substitution of a C with an A, a U, or a G. In some cases, the first strand comprises a nucleotide sequence selected from SEQ ID NOs:345, 346, and 368-375.

### First Mismatch at Position 11; 2 Mismatches

In some cases, a double-stranded RNA of the present disclosure comprises: a) a first strand that hybridizes to a target CAG repeat region of a CAG repeat-containing RNA; and b) a second strand that hybridizes to the first strand, where the first strand comprises: i) a first mismatch to the target CAG repeat region, wherein the first mismatch is at position 11 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), (UGCUGCUGCUGCUGCUGCUGCUG (SEQ ID NO:745), (GCUGCUGCUGCUGCUGCUGCU (SEQ ID NO:866), or (UGCUGCUGCUGCUGCUGCUGC (SEQ ID NO:867); and ii) a second mismatch to the target CAG repeat region, wherein the second mismatch is from 1 to 5 bases 3′ of the first mismatch. In some cases, the first strand is a variant comprising at least a first and a second substitution of the nucleotide sequence CUGCUGCUGCUGCUGCUGCUG (SEQ ID NO:743), wherein the first substitution generates the first mismatch and is at position 11 based on the numbering of CUGCUGCUGCUGCUGCUGCUG (SEQ ID NO:743), and the second substitution generates the second mismatch and is from 1 to 5 bases 3′ of the first mismatch. In some cases, the first strand is a variant comprising at least a first and a second substitution of the nucleotide sequence GCUGCUGCUGCUGCUGCUGCU (SEQ ID NO:866), wherein the first substitution generates the first mismatch and is at position 11 based on the numbering of GCUGCUGCUGCUGCUGCUGCU (SEQ ID NO:866), and the second substitution generates the second mismatch and is from 1 to 5 bases 3′ of the first mismatch. the first strand is a variant comprising at least a first and a second substitution of the nucleotide sequence UGCUGCUGCUGCUGCUGCUGC (SEQ ID NO:743), wherein the first substitution generates the first mismatch and is at position 11 based on the numbering of UGCUGCUGCUGCUGCUGCUGC (SEQ ID NO:743), and the second substitution generates the second mismatch and is from 1 to 5 bases 3′ of the first mismatch. In some cases, the first strand comprises no more than 2 mismatches with the target CAG repeat region. In some cases, the first strand comprises no more than 3 mismatches with the target CAG repeat region. In some cases, the first strand comprises no more than 4 mismatches with the target CAG repeat region. In some cases, the second strand is 100% complementary to the first strand. In some cases, the second strand comprises from 1 to 10 mismatches (e.g., from 1 to 4, from 3 to 5, from 5 to 7, or from 5 to 10 mismatches) to the first strand. In some cases, the second strand comprises from 1 to 4 mismatches to the first strand. In some cases, the second strand comprises no more than 1, 2, 3, or 4 mismatches to the first strand. In some cases, the second strand comprises no more than 1 mismatch to the first strand. In some cases, the second strand comprises no more than 2 mismatches to the first strand. In some cases, the second strand comprises no more than 3 mismatches to the first strand. In some cases, the second strand comprises no more than 4 mismatches to the first strand. In some cases, the second strand comprises no more than 5 mismatches to the first strand. In some cases, the double-stranded RNA has a length of from 18 nucleotides to 25 nucleotides. In some cases, the double-stranded RNA has a length of 20 nucleotides. In some cases, the double-stranded RNA has a length of 21 nucleotides. In some cases, the double-stranded RNA has a length of 22 nucleotides. In some cases, the double-stranded RNA has a length of 23 nucleotides. In some cases, the double-stranded RNA has a length of 24 nucleotides. In some cases, the double-stranded RNA has a length of 25 nucleotides. In some cases, each mismatch is generated by a substitution independently selected from: a) a substitution of a G with an A, a U, or a C; b) a substitution of a U with an A, a G, or a C; and c) a substitution of a C with an A, a U, or a G. In some cases, the first strand comprises a nucleotide sequence selected from SEQ ID NOs:311-315. In some cases, the first strand comprises a nucleotide sequence selected from SEQ ID NOs:793-803 (as depicted in Table 8; FIG. 24). In some cases, the first strand comprises the following nucleotide sequence: CUGCUGCUGCAACUGCUGCUG (SEQ ID NO:311); RNA guide strand sequence of “CUG_217” in Table 6). In some cases, the first strand comprises the following nucleotide sequence: CUGCUGCUGCAGAUGCUGCUG (SEQ ID NO:312; RNA guide strand sequence of “CUG_226” in Table 6). In some cases, the first strand comprises the following nucleotide sequence: CUGCUGCUGCAGCAGCUGCUG (SEQ ID NO:313; RNA guide strand sequence of “CUG_235” in Table 6). In some cases, the first strand comprises the following nucleotide sequence: CUGCUGCUGCAGCUACUGCUG (SEQ ID NO:314; RNA guide strand sequence of “CUG_244” in Table 6). In some cases, the first strand comprises the following nucleotide sequence: CUGCUGCUGCAGCUGAUGCUG (SEQ ID NO:315; RNA guide strand sequence of “CUG_253” in Table 6).

### First Mismatch at Position 11; 3 Mismatches

In some cases, a double-stranded RNA of the present disclosure comprises: a) a first strand that hybridizes to a target CAG repeat region of a CAG repeat-containing RNA; and b) a second strand that hybridizes to the first strand, where the first mismatch is at position 11 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), or (UGCUGCUGCUGCUGCUGCUGCUG (SEQ ID NO:745)), where the first strand comprises a second mismatch and a third mismatch to the target CAG repeat region, and where the second and third mismatches are from 1 to 5 bases 3′ of the first mismatch. In some cases, the first strand is a variant comprising at least a first, a second, and a third substitution of the nucleotide sequence CUGCUGCUGCUGCUGCUGCUG (SEQ ID NO:743), wherein the first substitution generates the first mismatch and is at position 11 based on the numbering of CUGCUGCUGCUGCUGCUGCUG (SEQ ID NO:743), wherein the second substitution generates the second mismatch and the third substitution generates the third mismatch, and wherein the second substitution and the third substitution are from 1 to 5 bases 3′ of the first mismatch. In some cases, the first strand is a variant comprising at least a first, a second, and a third substitution of the nucleotide sequence GCUGCUGCUGCUGCUGCUGCU (SEQ ID NO:866), wherein the first substitution generates the first mismatch and is at position 11 based on the numbering of GCUGCUGCUGCUGCUGCUGCU (SEQ ID NO:866), wherein the second substitution generates the second mismatch and the third substitution generates the third mismatch, and wherein the second substitution and the third substitution are from 1 to 5 bases 3′ of the first mismatch. In some cases, the first strand is a variant comprising at least a first, a second, and a third substitution of the nucleotide sequence UGCUGCUGCUGCUGCUGCUGC (SEQ ID NO:867), wherein the first substitution generates the first mismatch and is at position 11 based on the numbering of UGCUGCUGCUGCUGCUGCUGC (SEQ ID NO:867), wherein the second substitution generates the second mismatch and the third substitution generates the third mismatch, and wherein the second substitution and the third substitution are from 1 to 5 bases 3′ of the first mismatch. In some cases, the first strand comprises no more than 3 mismatches with the target CAG repeat region. In some cases, the first strand comprises no more than 4 mismatches with the target CAG repeat region. In some cases, the second strand is 100% complementary to the first strand. In some cases, the second strand comprises from 1 to 10 mismatches (e.g., from 1 to 4, from 3 to 5, from 5 to 7, or from 5 to 10 mismatches) to the first strand. In some cases, the second strand comprises from 1 to 4 mismatches to the first strand. In some cases, the second strand comprises no more than 1, 2, 3, or 4 mismatches to the first strand. In some cases, the second strand comprises no more than 1 mismatch to the first strand. In some cases, the second strand comprises no more than 2 mismatches to the first strand. In some cases, the second strand comprises no more than 3 mismatches to the first strand. In some cases, the second strand comprises no more than 4 mismatches to the first strand. In some cases, the second strand comprises no more than 5 mismatches to the first strand. In some cases, the double-stranded RNA has a length of from 18 nucleotides to 25 nucleotides. In some cases, the double-stranded RNA has a length of 20 nucleotides. In some cases, the double-stranded RNA has a length of 21 nucleotides. In some cases, the double-stranded RNA has a length of 22 nucleotides. In some cases, the double-stranded RNA has a length of 23 nucleotides. In some cases, the double-stranded RNA has a length of 24 nucleotides. In some cases, the double-stranded RNA has a length of 25 nucleotides. In some cases, each mismatch is generated by a substitution independently selected from: a) a substitution of a G with an A, a U, or a C; b) a substitution of a U with an A, a G, or a C; and c) a substitution of a C with an A, a U, or a G.

### First Mismatch at Position 11; 4 Mismatches

In some cases, a double-stranded RNA of the present disclosure comprises: a) a first strand that hybridizes to a target CAG repeat region of a CAG repeat-containing RNA; and b) a second strand that hybridizes to the first strand, where the first mismatch is at position 11 based on the numbering of SEQ ID NO:743 (CUGCUGCUGCUGCUGCUGCUG (SEQ ID NO:743)), SEQ ID NO:744 (GCUGCUGCUGCUGCUGCUGCUG (SEQ ID NO:744)), or (UGCUGCUGCUGCUGCUGCUGCUG (SEQ ID NO:745)), where the first strand comprises a second mismatch, a third mismatch, and a fourth mismatch to the target CAG repeat region, and where the second, third, and fourth mismatches are from 1 to 5 bases 3′ of the first mismatch. In some cases, the first strand is a variant comprising at least a first, a second, a third, and a fourth substitution of the nucleotide sequence CUGCUGCUGCUGCUGCUGCUG (SEQ ID NO:743), wherein the first substitution generates the first mismatch and is at position 11 based on the numbering of CUGCUGCUGCUGCUGCUGCUG (SEQ ID NO:743), wherein the second substitution generates the second mismatch, the third substitution generates the third mismatch, and the fourth substitution generates the fourth mismatch, and wherein the second, third, and fourth substitutions are from 1 to 5 bases 3′ of the first mismatch. In some cases, the first strand is a variant comprising at least a first, a second, a third, and a fourth substitution of the nucleotide sequence GCUGCUGCUGCUGCUGCUGCU (SEQ ID NO:866), wherein the first substitution generates the first mismatch and is at position 11 based on the numbering of GCUGCUGCUGCUGCUGCUGCU (SEQ ID NO:866), wherein the second substitution generates the second mismatch, the third substitution generates the third mismatch, and the fourth substitution generates the fourth mismatch, and wherein the second, third, and fourth substitutions are from 1 to 5 bases 3′ of the first mismatch. In some cases, the first strand is a variant comprising at least a first, a second, a third, and a fourth substitution of the nucleotide sequence UGCUGCUGCUGCUGCUGCUGC (SEQ ID NO:867), wherein the first substitution generates the first mismatch and is at position 11 based on the numbering of UGCUGCUGCUGCUGCUGCUGC (SEQ ID NO:867), wherein the second substitution generates the second mismatch, the third substitution generates the third mismatch, and the fourth substitution generates the fourth mismatch, and wherein the second, third, and fourth substitutions are from 1 to 5 bases 3′ of the first mismatch. In some cases, the first strand comprises no more than 4 mismatches with the target CAG repeat region. In some cases, the second strand is 100% complementary to the first strand. In some cases, the second strand comprises from 1 to 10 mismatches (e.g., from 1 to 4, from 3 to 5, from 5 to 7, or from 5 to 10 mismatches) to the first strand. In some cases, the second strand comprises from 1 to 4 mismatches to the first strand. In some cases, the second strand comprises no more than 1, 2, 3, or 4 mismatches to the first strand. In some cases, the second strand comprises no more than 1 mismatch to the first strand. In some cases, the second strand comprises no more than 2 mismatches to the first strand. In some cases, the second strand comprises no more than 3 mismatches to the first strand. In some cases, the second strand comprises no more than 4 mismatches to the first strand. In some cases, the second strand comprises no more than 5 mismatches to the first strand. In some cases, the double-stranded RNA has a length of from 18 nucleotides to 25 nucleotides. In some cases, the double-stranded RNA has a length of 20 nucleotides. In some cases, the double-stranded RNA has a length of 21 nucleotides. In some cases, the double-stranded RNA has a length of 22 nucleotides. In some cases, the double-stranded RNA has a length of 23 nucleotides. In some cases, the double-stranded RNA has a length of 24 nucleotides. In some cases, the double-stranded RNA has a length of 25 nucleotides. In some cases, each mismatch is generated by a substitution independently selected from: a) a substitution of a G with an A, a U, or a C; b) a substitution of a U with an A, a G, or a C; and c) a substitution of a C with an A, a U, or a G.

### B. Target Nucleic Acids

The double stranded RNAs of the present disclosure may be targeted to any gene or nucleic acid construct containing the targeted repeat region. In some embodiments, genes (DNA or mRNA) that encode human or primate proteins are targeted. In some embodiments, non-coding genes are targeted. In some embodiments, coding regions of a gene are targeted. In some embodiments, non-coding regions of a gene are targeted.

In some embodiments, double stranded RNAs of the present disclosure target CAG-repeat containing (polyglutamine) genes. In some embodiments, the CAG repeat containing gene is selected from HTT, Ataxin 1, Ataxin 2, Ataxin 3, CACNA1A, Ataxin 7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, and MAB21L1. Expansion of the CAG-repeat is associated with a number of dominant, genetic disorders referred to as polyglutamine (polyQ) diseases. While CAG-repeat containing proteins are ubiquitously expressed throughout the body, the pathology of polyglutamine diseases primarily appears in, but is not limited to, neuronal tissue. Thus, as used herein, the term polyglutamine disease refers to any disease or disorder associated with CAG-repeat expansion, including, but not limited to neurodegenerative diseases.

Huntingtin (HIT), also known as interesting transcript 15 (IT15), refers to a gene encoding huntingtin protein. The exact function of huntingtin is unknown but is involved in axonal transport. An example of a huntingtin transcript sequence is provided by NCBI Reference Sequence NM_002111.8 (SEQ ID NO:820). Typically, the polyglutamine tract of huntingtin has 10-35 CAG repeats. Expansion of the polyglutamine tract to 36 to more than 120 CAG repeats causes Huntington's disease. Early signs and symptoms can include irritability, depression, small involuntary movements, poor coordination, and trouble learning new information or making decisions. Many people with Huntington disease develop involuntary jerking or twitching movements known as chorea. As the disease progresses, these movements become more pronounced. Affected individuals may have trouble walking, speaking, and swallowing. People with this disorder also experience changes in personality and a decline in thinking and reasoning abilities.

Ataxin 1 (ATXN1), also known as spinocerebellar ataxia type 1 (SCA1), refers to a gene encoding a polyglutamine containing protein expressed primarily in the nucleus where it binds chromatin and functions as a transcriptional repressor. An example of a ATXN1 transcript sequence is provided by NCBI Reference Sequence NM_001128164.2 (SEQ ID NO:821). Mutant forms of ataxin-1 containing expansion of the polyglutamine tract to typically about 40-83 repeats cause the movement disorder spinocerebellar ataxia type 1 (SCA1) through a toxic gain-of-function mechanism in the cerebellum. The cerebellar dysfunction is progressive and permanent. People with this condition initially experience problems with coordination and balance (ataxia). Other signs and symptoms of SCA1 include speech and swallowing difficulties, muscle stiffness (spasticity), and weakness in the muscles that control eye movement (ophthalmoplegia). Eye muscle weakness leads to rapid, involuntary eye movements (nystagmus). Individuals with SCA1 may have difficulty processing, learning, and remembering information (cognitive impairment). Over time, individuals with SCA1 may develop numbness, tingling, or pain in the arms and legs (sensory neuropathy); uncontrolled muscle tensing (dystonia); muscle wasting (atrophy); and muscle twitches (fasciculations). Rarely, rigidity, tremors, and involuntary jerking movements (chorea) have been reported in people who have been affected for many years.

Ataxin 2 (ATXN2), also known as spinocerebellar ataxia type 2 (SCA2), refers to a gene encoding a polyglutamine containing, RNA-binding protein that targets cis-regulatory elements in 3′ UTRs to stabilize a subset of mRNAs and increase protein expression. An example of an ATXN2 transcript sequence is provided by NCBI Reference Sequence: NM_001372574.1 (SEQ ID NO:822). Polyglutamine repeat expansion in ATXN2 (e.g., typically ˜33 or more repeats) can cause signs and symptoms of spinocerebellar ataxia type 2 (SCA2). People with SCA2 initially experience problems with coordination and balance (ataxia). Other early signs and symptoms of SCA2 include additional movement problems, speech and swallowing difficulties, and weakness in the muscles that control eye movement (ophthalmoplegia). Eye muscle weakness leads to involuntary back-and-forth eye movements (nystagmus) and a decreased ability to make rapid eye movements (saccadic slowing). Over time, individuals with SCA2 may develop loss of sensation and weakness in the limbs (peripheral neuropathy), muscle wasting (atrophy), uncontrolled muscle tensing (dystonia), and involuntary jerking movements (chorea). Some people with SCA2 develop a group of movement abnormalities known as parkinsonism, which includes unusually slow movement (bradykinesia), involuntary trembling (tremor), and muscle stiffness (rigidity). Individuals with SCA2 may have problems with short term memory, planning, and problem solving, or experience an overall decline in intellectual function (dementia). Intermediate polyglutamine expansion (27-33 CAG repeats) in ATXN2 also increases the risk of amyotrophic lateral sclerosis (ALS). ALS is a neurodegenerative neuromuscular disease that results in the progressive loss of motor neurons that control voluntary muscles. Early symptoms of ALS include stiff muscles, muscle twitches, and gradual increasing weakness and muscle wasting. Limb-onset ALS begins with weakness in the arms or legs, while bulbar-onset ALS begins with difficulty speaking or swallowing. Half of the people with ALS develop at least mild difficulties with thinking and behavior, and about 15% develop frontotemporal dementia. Most people experience pain. Motor neuron loss continues until the ability to eat, speak, move, and finally the ability to breathe is lost. ALS eventually causes paralysis and early death, usually from respiratory failure.

Ataxin 3 (ATXN3), also known as spinocerebellar ataxia type 3 (SCA3), refers to a gene encoding a polyglutamine containing, deubiquitinating enzyme. An example of an ATXN3 transcript sequence is provided by NCBI Reference Sequence NM_004993.6 (SEQ ID NO:823). Expansion of the polyglutamine repeat from the normal 13-36 to more than 50 causes Machado-Joseph disease (MJD), also known as Machado-Joseph Azorean disease, Machado's disease, Joseph's disease or spinocerebellar ataxia type 3 (SCA3). People with this condition initially experience problems with coordination and balance (ataxia). Other early signs and symptoms of SCA3 include speech difficulties, uncontrolled muscle tensing (dystonia), muscle stiffness (spasticity), rigidity, tremors, bulging eyes, and double vision. People with this condition may experience sleep disorders such as restless leg syndrome or REM sleep behavior disorder. Over time, individuals with SCA3 may develop loss of sensation and weakness in the limbs (peripheral neuropathy), muscle cramps, muscle twitches (fasciculations), and swallowing difficulties. Individuals with SCA3 may have problems with memory, planning, and problem solving.

Calcium voltage-gated channel subunit alpha1 A (CACNA1A), also known as spinocerebellar ataxia type 6 (SCA6), encodes the α1A pore-forming subunit of the neuronal calcium channel P/Q. An example of a CACNA1A transcript sequence is provided by NCBI reference sequence NM_000068.4 (SEQ ID NO:824). Expansion of the polyglutamine tract in the CACNA1A gene to typically 19-33 repeats causes spinocerebellar ataxia type 6 (SCA6). People with this condition initially experience problems with coordination and balance (ataxia). Other early signs and symptoms of SCA6 include speech difficulties, involuntary eye movements (nystagmus), and double vision. Over time, individuals with SCA6 may develop loss of coordination in their arms, tremors, and uncontrolled muscle tensing (dystonia).

Ataxin 7 (ATXN7), also known as spinocerebellar ataxia type 7 (SCA7), encodes a polyglutamine containing protein that is an integral subunit of GCN5 (general control of amino acid synthesis-5; KAT2A)-containing SAGA family of histone acetyltransferase (HAT) complexes. An example of an ATXN7 transcript sequence is provided by NCBI Reference Sequence NM_001377405.1 (SEQ ID NO:825). Polyglutamine expansion in ATXN7 causes spinocerebellar ataxia type 7 (SCA7), which is characterized by progressive cerebellar ataxia, retinal degeneration or blindness due to cone-rod dystrophy, and mild changes in sensation or reflexes. Later symptoms include loss of motor control, unclear speech (dysarthria), and difficulty swallowing (dysphagia).

Protein Phosphatase 2 Regulatory Subunit Bbeta (PPP2R2B), also known as spinocerebellar ataxia type 12 (SCA12), encodes the B regulatory subunit of Protein Phosphatase 2, a serine/threonine phosphatase. An example of a PPP2R2B transcript sequence is provided by NCBI Reference Sequence NM_181674.3 (SEQ ID NO:826). Expansion of the polyglutamine repeat from the typically normal range of about 7-28 to about 55-78 causes spinocerebellar ataxia type 12 (SCA12). The age of onset of symptoms of SCA12 ranges from 8 to 55 years, though most commonly occurs in the fourth decade. Symptoms typically begin with tremors and progress to cerebellar ataxia. Signs of dementia have also been reported as associated with SCA12.

TATA-Box Binding Protein (TBP), also known as spinocerebellar ataxia type 17 (SCAJ7), encodes the TATA-binding protein, a component of transcription factor IID (TFIID). An example of a TBP transcript sequence is provided by NCBI Reference Sequence NM_003194.5 (SEQ ID NO:827). TBP typically has 25-42 polyglutamine repeats, expansion to 45-66 repeats is associated with spinocerebellar ataxia type 17 (SCA17). People with this condition typically experience symptoms such as ataxia, dementia, and involuntary movements such as chorea and dystonia, rigidity, and pyramidal signs such as spasticity, weakness, slowing of rapid alternating movements, and hyperreflexia.

Androgen Receptor (AR) encodes a steroid-hormone activated transcription factor. An example of an AR transcript sequence is provided by NCBI Reference Sequence NM_000044.6 (SEQ ID NO:828). Expansion of the polyglutamine repeats from the typically 9-34 repeats to 38-62 repeats causes spinal bulbar muscular atrophy (SBMA), also known as Kennedy's disease. SBMA is characterized by muscle weakness and atrophy that worsens over time, resulting in cramping and difficulty with walking, swallowing, and speech. SBMA may also result in gynecomastia and infertility.

Atrophin 1 (ATN1) encodes a protein that is hypothesized to be a transcriptional co-repressor that recruits Nuclear Receptor Subfamily 2 Group E Member 1 (NR2E1) to repress transcription. An example of an ATN1 transcript sequence is provided by NCBI Reference Sequence NM_001007026.2 (SEQ ID NO:829). Dentatorubral pallidoluysian atrophy (DRPLA) is a rare neurodegenerative disorder related to the expansion of the polyglutamine repeat in ATN1 from the typical 7-35 copies to 49-93 copies. When DRPLA manifests before about age 20 it is typically associated with myoclonus, ataxia, seizures, behavioral changes, and intellectual disability. When it manifests after about age 20 it is associated with ataxia, choreoathetosis, delusions, and dementia.

Myeloid/Lymphoid Or Mixed-Lineage Leukemia Translocated To Chromosome 3 (MLLT3), also known as AF-9, encodes a component of the super elongation complex (SEC), which is necessary to increase the catalytic rate of RNA polymerase II transcription. An example of a MLLT3 transcript sequence is provided by NCBI Reference Sequence NM_004529.4 (SEQ ID NO:830). MLLT3 includes an unstable polyglutamine repeat and genetic aberrations involving MLLT3 have been associated with leukemias and neuromotor development delay, cerebellar ataxia, and epilepsy.

Bone Morphogenic Protein 2 Inducible Kinase (BMP2K) encodes a protein related to skeletal development and patterning. An example of a BMP2K transcript sequence is provided by NCBI Reference Sequence NM_198892.2 (SEQ ID NO:831). BMP2K includes a polyglutamine repeat and is associated with myopia and cancer, specifically gene mis-regulation associated with cancer.

THAP Domain Containing 11 (THAP11) encodes a transcriptional repressor associated with embryogenesis. An example of a THAP11 transcript sequence is provided by NCBI Reference Sequence NM_020457.3 (SEQ ID NO:832). THAPI1 includes a polyglutamine repeat of typically about 29 copies, but ranges from 20 to over 40 copies. An increased number of polyglutamine repeats, for example 38 copies, is associated with neurodegenerative disease. Expansion of the polyglutamine in THAPI1 is also associated with intracellular aggregation of THAPI1, cellular toxicity, growth inhibition, GO/GI arrest, and inhibition of transcription activity.

Zinc Finger Homeobox 3 (ZFHX3) encodes a transcription factor that regulates myogenic and neuronal differentiation. It also functions as a tumor suppressor in several cancers and is associated with atrial fibrillation. An example of a ZFHX3 transcript sequence is provided by NCBI Reference Sequence NM_006885.4 (SEQ ID NO:833). ZFHX3 includes a polyglutamine repeat. Individuals with an expanded polyglutamine repeat, e.g. having 19 copies, is associated with coronary heart disease, hypertension, diabetes mellitus, or dyslipidemia as compared to those with fewer repeats, e.g. 17 copies.

POU Class 3 Homeobox 2 (POU3F2) encodes a transcription factor that is related to neuronal differentiation. An example of a POU3F2 transcript sequence is provided by NCBI Reference Sequence NM_005604.4 (SEQ ID NO:834). POU3F2 includes a polyglutamine tract and is associated with bipolar disorder, obesity, developmental delay, and intellectual disability.

Mastermind Like Transcriptional Coactivator 2 (MAML2) encodes a transcriptional coactivator for NOTCH proteins and promotes β-catenin turnover. An example of a MAML2 transcript sequence is provided by NCBI Reference Sequence NM_032427.4 (SEQ ID NO:835). MAML2 includes a polyglutamine tract with observed variability and is associated with cancers such as mucoepidermoid carcinomas, hidradenoma, B cell-derived lymphomas, and chronic lymphocytic leukemia.

Mastermind Like Transcriptional Coactivator 3 (MAML3) encodes a transcriptional coactivator for NOTCH proteins. An example of a MAML3 transcript sequence is provided by NCBI Reference Sequence NM_018717.5 (SEQ ID NO:836). MAML3 includes a polyglutamine tract and is associated with cancers such as schneiderian carcinoma and ossifying fibromyxoid tumor.

SWI/SNF Related, Matrix Associated, Actin Dependent Regulator Of Chromatin, Subfamily A, Member 2 (SMARCA2) encodes a component of the SWI/SNF complex that is involved in transcriptional regulation by chromatin remodeling. SMARCA2 is also involved in neural development. An example of a SMARCA2 transcript sequence is provided by NCBI Reference Sequence NM_003070.5 (SEQ ID NO:837). SMARCA2 includes a polymorphic polyglutamine tract and is associated conditions such as Nicolaides-Baraitser syndrome and blepharophimosis-impaired intellectual development syndrome. The SMARCA2 gene is also located on a chromosomal region that is linked to schizophrenia and bipolar disorder.

Origin Recognition Complex Subunit 4 (ORC4) encodes a component of the six subunit origin recognition complex (ORC) that is necessary for the initiation of DNA replication. An example of an ORC4 transcript sequence is provided by NCBI Reference Sequence NM_001190879.3 (SEQ ID NO:838). ORC4 includes a region of polymorphic trinucleotide CAG repeats located upstream of the coding sequence and is associated with Meier-Gorlin syndrome 1 and Meier-Gorlin syndrome 2

RUNX Family Transcription Factor 2 (RUNX2) encodes a nuclear protein involved osteoblastic differentiation and skeletal morphogenesis. An example of a RUNX2 transcript sequence is provided by NCBI Reference Sequence NM_001024630.4 (SEQ ID NO:839). RUNX2 includes a polyglutamine tract and a polyalanine tract. Expansion of the polyglutamine tract from, e.g., typical 23 residues to, e.g., 27-30 residues causes cleidocranial dysplasia, decreased bone mineral density, and decreases RUNX2 transactivation capacity. Cleidocranial dysplasia (CCD) is a disorder affecting the skull, bones and teeth. Signs and symptoms include absent or underdeveloped collar bones, delayed closing of fontanels in the skull, dental abnormalities, short stature, decreased bone density, hearing loss, and other bone abnormalities.

Mediator Complex Subunit 12 (MED12) encodes a component of the preinitiation complex that is involved in the control of initiation of transcription. An example of a MED12 transcript sequence is provided by NCBI Reference Sequence NM_005120.3 (SEQ ID NO:840). MED12 has a polyglutamine tract and is associated with Opitz-Kaveggia syndrome, Lujan-Fryns syndrome, Ohdo syndrome, X-linked, and tumor formation, e.g., in uterine leiomyomas.

E1A Binding Protein P400 (EP400) encodes a component of the NuA4 histone acetyltransferase complex that is involved in transcriptional activation. An example of an EP400 transcript sequence is provided by NCBI Reference Sequence NM_015409.5 (SEQ ID NO:841). EP400 normally contains about 32 CAG repeats. EP400 is involved in ossifying fibromyxoid tumor and epilepsy, familial temporal lobe, 1.

Membrane Associated Guanylate Kinase, WW And PDZ Domain Containing 1 (MAGI1) encodes a protein involved in the assembly of multiprotein complexes at regions of cell to cell contact. An example of a MAGI1 transcript sequence is provided by NCBI Reference Sequence NM_015520.2 (SEQ ID NO:842). MAGI1 contains a polymorphic polyglutamine tract and is associated with conditions such as cervical large cell neuroendocrine carcinoma and microscopic colitis.

UBAP1-MVB12-Associated (UMA) Domain Containing 1 (UMAD1) is a protein coding gene. An example of a UMAD1 transcript sequence is provided by NCBI Reference Sequence NM_001302348.2 (SEQ ID NO:843). UMAD1 includes a region of polymorphic trinucleotide CAG repeats upstream of the start codon and is associated with retinitis pigmentosa.

DM1 Locus Antisense RNA (DM1-AS) is an RNA gene. An example of a DM1-AS RNA sequence is provided by NCBI Reference Sequence NR_147193.1 (SEQ ID NO:844). DM1-AS includes a region of polymorphic trinucleotide CAG repeats in an intron and is associated with myotonic dystrophy 1 and branchiootorenal syndrome 2.

AC007161.3 also known as ENSG00000283549 is an RNA gene and contains CAG repeats.

Interferon Regulatory Factor 2 Binding Protein Like (IRF2BPL) encodes a transcription factor associated with the development of the central nervous system and in neuronal maintenance and with regulating female reproductive function. An example of an IRF2BPL transcript sequence is provided by NCBI Reference Sequence NM_024496.4 (SEQ ID NO:845). IRF2BPL includes a polyglutamine tract and is associated with neurological problems such as neurodevelopmental disorder with regression, abnormal movements, loss of speech, and seizures and Irf2bpl-related regressive neurodevelopmental disorder-dystonia-seizures syndrome.

Mab-21 Like 1 (MAB21L1) encodes a protein associated with eye and cerebellum development. An example of a MAB21L1 transcript sequence is provided by NCBI Reference Sequence NM_005584.5 (SEQ ID NO:846). MAB21L1 is associated with cerebellar, ocular, craniofacial, and genital Syndrome and hydrophthalmos. MAB21L1 includes polymorphic trinucleotide CAG repeats in the 5′ untranslated portion of the gene that are associated with psychiatric conditions such as bipolar disorder.

In some embodiments, a pathogenic or pathologic allele of a CAG repeat containing gene or RNA encoded by the CAG repeat containing gene contains at least about 30 consecutive CAG repeats.

### C. Expression Cassettes and Vectors

In some embodiments, double stranded RNAs of the disclosure are encoded by a nucleic acid molecule, for example a DNA sequence. Double stranded RNA sequences provided herein can be converted to DNA format by replacing each uracil base “U” with a thymine “T” base.

In some embodiments, nucleic acid molecule (e.g., DNA) encoding the double stranded RNA is contained within an expression cassette.

In some embodiments, the expression cassette further comprises one or more expression control sequences (regulatory sequences) operably linked with the transgene. “Operably linked” sequences include expression control sequences that are contiguous with the transgene or act in trans or at a distance from the transgene to control its expression. Examples of expression control sequences include transcription initiation sequences, termination sequences, promoter sequences, enhancer sequences, repressor sequences, splice site sequences, polyadenylation (polyA) signal sequences, or any combination thereof.

In some embodiments, a promoter is an endogenous promoter, synthetic promoter, hybrid promoter, constitutive promoter, inducible promoter, tissue-specific promoter (e.g., CNS-specific), or cell-specific promoter (neurons, glial cells, or astrocytes). Examples of constitutive promoters include, Rous sarcoma virus (RSV) LTR promoter (optionally with the RSV enhancer), cytomegalovirus (CMV) promoter (optionally with the CMV enhancer), SV40 promoter, and dihydrofolate reductase promoter. Examples of inducible promoters include zinc-inducible sheep metallothionine (MT) promoter, dexamethasone (Dex)-inducible mouse mammary tumor virus (MMTV) promoter, T7 polymerase promoter system, the ecdysone insect promoter, tetracycline-repressible system, tetracycline-inducible system, RU486-inducible system, and the rapamycin-inducible system. Further examples of promoters that may be used include, for example, chicken beta-actin promoter (CBA promoter), a CAG promoter, an H1 promoter, a CD68 promoter, a JeT promoter, synapsin promoter, RNA pol II promoter, or an RNA pol III promoter (e.g., U6, H1, etc.).

In some embodiments, a promoter is an RNA pol II promoter. Examples of pol II promoters include PGK, CBA, U1, CMV, EIF1α, EF1α, CAG, or synaptophysin promoters. In some embodiments, the promoter is a tissue-specific RNA pol II promoter. In some embodiments, the tissue-specific RNA pol II promoter is derived from a gene that exhibits neuron-specific expression. In some embodiments, an expression cassette comprises a pol II promoter and a poly(A) tail, e.g., with the DNA sequence encoding the double stranded RNA flanked on the 5′ end by the pol II promoter and on the 3′ end by the poly(A) tail.

In some embodiments, a promoter is a neuron specific promoter. Examples of neuron-specific promoters include those from neuron specific enolase (NSE), human synapsin 1, human synapsin 2 promoter, caMK kinase, and tubuline.

In some embodiments, a promoter is an RNA pol III promoter. Examples of pol III promoters include U6, H1, 7SK, Y, RPR, MRP, and selenocysteine tRNA. In some embodiments, an expression cassette comprises a pol III promoter and a poly(T) tail, e.g., with the DNA sequence encoding the double stranded RNA flanked on the 5′ end by the pol III promoter and on the 3′ end by the poly(T) tail.

In some embodiments, a promoter is a RNA pol I promoter. In some embodiments, an expression cassette comprises a pol I promoter and a 3′-box, e.g. with the DNA sequence encoding the double stranded RNA flanked on the 5′ end by the pol I promoter and on the 3′ end by the 3′-box.

Expression cassettes for double stranded RNAs are known in the art, see, e.g., ter Brake et al. Mol. Ther. (2008) 16:557; Maczuga et al., BMC Biotechnol. (2012) 12:42; and Bofill-De Ros and Gu (2016) 103:157.

In some embodiments, the DNA sequence encoding the double stranded RNA of the disclosure is positioned in an untranslated region of an expression cassette. In some embodiments, the sequence encoding the inhibitory nucleic acid of the present disclosure is positioned in an intron, a 5′ untranslated region (5 ′UTR), or a 3′ untranslated region (3′UTR) of the expression cassette. In some embodiments, the sequence encoding the inhibitory nucleic acid of the present disclosure is positioned in an intron downstream of the promoter and upstream of an expressed gene.

In some embodiments, the DNA sequence encoding a double stranded RNA of the disclosure is flanked by two AAV inverted terminal repeats (ITRs) (e.g., 5′ ITR and 3′ ITR) within the expression cassette. In some embodiments, each AAV ITR is a full length ITR (e.g., approximately 145 bp in length, and containing functional Rep binding site (RBS) and terminal resolution site (trs)). In some embodiments, one of the ITRs is truncated (e.g., shortened or not full-length). In some embodiments, a truncated ITR lacks a functional terminal resolution site (trs) and is used for production of self-complementary AAV vectors (scAAV vectors).

In some embodiments, double-stranded RNAs described herein can be encoded by vectors, such as plasmids, non-viral vectors, or viral vectors. The use of vectors for expressing double-stranded RNAs of the present disclosure may allow for continual or controlled expression of the double-stranded RNAs in the subject, rather than multiple doses of the double-stranded RNAs to the subject. The present disclosure provides a vector comprising an isolated nucleic acid comprising an expression cassette encoding a double-stranded RNA described herein.

Viral vectors include, but are not limited to, herpesvirus (HSV) vectors, retroviral vectors, adenoviral vectors, adeno-associated viral (AAV) vectors, lentiviral vectors, baculoviral vectors, and the like.

In some embodiments, the vector encoding a double stranded RNA of the disclosure is a retroviral vector. In some embodiments, a retroviral vector is a mouse stem cell virus, murine leukemia virus (e.g., Moloney murine leukemia virus vector), feline leukemia virus, feline sarcoma virus, or avian reticuloendotheliosis virus vector. In some embodiments, the vector encoding a double stranded RNA of the disclosure is a lentivirus or lentiviral based vector. In some embodiments, a lentiviral vector is a HIV (human immunodeficiency virus, including HIV type 1 and HIV type 2, equine infectious anemia virus, feline immunodeficiency virus (FIV), bovine immune deficiency virus (BIV), and simian immunodeficiency virus (SIV), equine infectious anemia virus, or Maedi-Visna viral vector. Methods for expressing shRNAs using lentivirus engineered cells are known in the art, for example, Stegmeier et al. Proc. Natl. Acad. Sci. USA (2005) 102:13212-13217; Klinghoffer et al. RNA (2010) 16:879-884. Production of replication-incompetent recombinant lentivirus may be achieved, for example, by co-transfection of expression vectors and packaging plasmids using commercially available packaging cell lines, such as TLA-HEK293TM, and packaging plasmids (Thermo Scientific/Open Biosystems, Huntsville, AL).

In some embodiments, the vector encoding a double stranded RNA of the disclosure is an adeno-associated virus (AAV) vector, such as a recombinant rAAV vector, which is produced by recombinant methods. AAV is a single-stranded, non-enveloped DNA virus having a genome that encodes proteins for replication (rep) and the capsid (Cap), flanked by two ITRs, which serve as the origin of replication of the viral genome. AAV also contains a packaging sequence, allowing packaging of the viral genome into an AAV capsid. In some embodiments, the AAV vector comprises an expression cassette encoding a double stranded RNA of the present disclosure flanked by two cis-acting AAV ITRs (5′ ITR and 3′ ITR). Functional ITR sequences are used for the rescue, replication and packaging of the AAV viral particle. Thus, an AAV vector is defined herein to include at least those sequences required in cis for replication and packaging (e.g., one or two functional ITRs and packaging sequence) of the virus. In some embodiments, each AAV ITR is a full length ITR (e.g., approximately 145 bp in length, and containing functional Rep binding site (RBS) and terminal resolution site (trs)). In some embodiments, one or both of the ITRs is modified, e.g., by insertion, deletion, or substitution, provided that the ITRs provide for functional rescue, replication, and packaging. In some embodiments, a modified ITR lacks a functional terminal resolution site (trs) and is used for production of self-complementary AAV vectors (scAAV vectors). In some embodiments, the ITRs are selected from any one of serotypes AAV1, AAV2, AAV3, AAV4, AAV5, AAV6, AAV7, AAV8, AAV9, AAV.Rh10, AAV11 and variants thereof. In some cases, the ITRs are from AAV2.

Other expression control sequences may be present in the rAAV vector operably linked to the DNA sequence encoding the double stranded RNA, including one or more of transcription initiation sequences, termination sequences, promoter sequences, enhancer sequences, repressor sequences, splice site sequences, polyadenylation (polyA) signal sequences, or any combination thereof.

rAAV vectors may have one or more AAV wild type genes deleted in whole or in part. In some embodiments the rAAV vector is replication defective. In some embodiments, the rAAV vector lacks a functional Rep protein and/or capsid protein.

Methods of packaging recombinant AAV vector into AAV capsids using host cell culture are known in the art. In some embodiments, one or more of the required components for packaging the rAAV vector, (e.g., Rep sequence, cap sequence, and/or accessory functions) may be provided by a stable host cell that has been engineered to contain the one or more required components (e.g., by a vector). Expression of the required components for AAV packaging may be under control of an inducible or constitutive promoter in the host packaging cell. AAV helper vectors are commonly used to provide transient expression of AAV rep and/or cap genes, which function in trans, to complement missing AAV functions that are necessary for AAV replication. In some embodiments, AAV helper vectors lack AAV ITRs and can neither replicate nor package themselves. AAV helper vectors can be in the form of a plasmid, phage, transposon, cosmid, virus, or virion.

Recombinant AAV vectors of the present disclosure may be encapsidated by an AAV capsid to form a rAAV particle. A “rAAV particle” or “rAAV virion” refers to an infectious, replication-defective virus including an AAV protein shell, encapsidating a transgene of interest which is flanked on both sides by AAV ITRs. A rAAV particle is produced in a suitable host cell which has sequences specifying a rAAV vector, AAV helper functions and accessory functions introduced therein to render the host cell capable of encoding AAV polypeptides that are required for packaging the rAAV vector (containing the transgene sequence of interest) into infectious rAAV particles for subsequent gene delivery to a target cell.

In some embodiments, rAAV particles may be produced using the triple transfection method (see, e.g., U.S. Pat. No. 6,001,650, incorporated herein by reference in its entirety). In this approach, the rAAV particles are produced by transfecting a host cell with a rAAV vector (comprising a transgene) to be packaged into rAAV particles, an AAV helper vector, and an accessory function vector. In some embodiments, the AAV helper function vector supports efficient AAV vector production without generating any detectable wild-type AAV virions (e.g., AAV virions containing functional rep and cap genes). The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus. In some embodiments, a double transfection method, wherein the AAV helper function and accessory function are cloned on a single vector, is used to generate rAAV particles.

The AAV capsid is an important element in determining the tissue-specificity of the rAAV particle. Thus, a rAAV particle having a particular capsid tissue specificity can be selected. In some embodiments, the rAAV particle comprises a capsid selected from an AAV serotype selected from AAV1, AAV2, AAV3, AAV4, AAV5, AAV6, AAV7, AAV8, AAV9, AAV.Rh10, AAV11, and variants thereof. In some embodiments, the AAV capsid is selected from a serotype that is capable of crossing the blood-brain barrier, e.g., AAV9, AAVrh.10, or a variant thereof. In some embodiments, the AAV capsid is a chimeric AAV capsid.

In some embodiments, the rAAV vector is a mammalian serotype AAV vector (e.g., AAV genome and ITRs derived from mammalian serotype AAV), including a primate serotype AAV vector or human serotype AAV vector. In some embodiments, the AAV vector is derived from any one of serotypes AAV1, AAV2, AAV3, AAV4, AAV5, AAV6, AAV7, AAV8, AAV9, AAV.Rh10, AAV11, and variants thereof. In some embodiments, the AAV vector is a chimeric AAV vector. In some embodiments, rAAV vectors may be vectors comprising an AAV genome and AAV capsid derived from the same AAV serotype. In some embodiments, rAAV vectors are pseudotyped, meaning the rAAV vectors comprise an AAV genome derived from one AAV serotype and an AAV capsid derived at least in part from a different AAV serotype.

In some embodiments, the rAAV vector is AAV9 serotype. In some embodiments, the rAAV comprises an AAV9 capsid protein (e.g., SEQ ID NO:2 of U.S. Pat. No. 7,198,951), an AAV9 rep protein (e.g., SEQ ID NO:3 of U.S. Pat. No. 7,198,951), or both. In some embodiments, the rAAV comprises: (i) an AAV9 capsid protein (e.g., SEQ ID NO:2 of U.S. Pat. No. 7,198,951), and (ii) AAV2 ITRs.

In some embodiments, the rAAV particle is capable of transducing cells of the central nervous system (CNS). In some embodiments, the rAAV particle is capable of transducing non-neuronal cells or neuronal cells of the CNS. In some embodiments, the CNS cell is a neuron, glial cell, astrocyte, or microglial cell.

In some embodiments, the rAAV vector is a self-complementary AAV (scAAV) vector. scAAV vectors contain two complementary DNA strands in the form of a dimeric inverted repeat genome. The two complementary strands within the dimeric inverted repeat genome anneal together to form one double stranded DNA that is ready for immediate replication and transcription, thus bypassing the requirement for host cell DNA synthesis. Self-complementary AAV vectors are described in U.S. Pat. Nos. 7,465,583; 7,790,154; 8,361,457; and 8,784,799.

The present disclosure also provides host cells transfected with the rAAV comprising a DNA sequence encoding the double stranded RNAs described herein. In some embodiments, the host cell is a prokaryotic cell or a eukaryotic cell. In some embodiments, the host cell is a mammalian cell (e.g., HEK293T, COS cells, HeLa cells, KB cells), bacterial cell (E. coli), yeast cell, insect cell (Sf9, Sf21, Drosophila, mosquito), etc. In some embodiments, the host cell is obtained or derived from a human subject. In some embodiments, the host cell is a fibroblast.

### DNA Molecule Encoding One or Both Strands of a Double-Stranded RNA

The present disclosure provides a DNA molecule comprising a nucleotide sequence encoding the first strand of a double-stranded RNA of the present disclosure. In some cases, the nucleotide sequence encoding the first strand is operably linked to a promoter. In some cases, the nucleotide sequence encoding the first strand is operably linked to a promoter that is functional in a eukaryotic cell. The present disclosure provides a DNA molecule comprising a nucleotide sequence encoding: i) the first strand of a double-stranded RNA of the present disclosure; and ii) the second strand of a double-stranded RNA of the present disclosure. In some cases, the nucleotide sequence encoding the first strand and the second strand is operably linked to a promoter. In some cases, the promoter is a PolII promoter. In some cases, the promoter is a U6 promoter. In some cases, the promoter is a CAG promoter. In some cases, the promoter is a CBA promoter. In some cases, the promoter is a CMV promoter. In some cases, the promoter is an EF1α promoter. In some cases, the promoter is an H1 promoter. In some cases, a DNA molecule of the present disclosure comprises a nucleotide sequence that encodes any one of SEQ ID NOs:295-375.

### Recombinant RNA Molecules

The present disclosure provides a recombinant nucleic acid (e.g., a recombinant RNA; which may be referred to as an “artificial microRNA” or a “small binding RNA” (sbRNA)) comprising: a) a double-stranded RNA of the present disclosure; and b) a microRNA scaffold comprising a 5′ flanking polynucleotide (also referred to herein as a “5′ leader”), a loop polynucleotide, and a 3′ flanking polynucleotide (also referred to herein as a “3′ trailer”), wherein the recombinant nucleic acid comprises: i) the 5′ flanking polynucleotide; ii) the first strand of the double-stranded RNA; iii) the loop polynucleotide; (iv) the second strand of the double-stranded RNA; and iii) the 3′ trailer polynucleotide; and wherein at least one of the 5′ flanking polynucleotide, the loop polynucleotide, and the 3′ flanking polynucleotide is heterologous to the first and/or the second strand of the double-stranded RNA. The present disclosure provides a recombinant nucleic acid (e.g., a recombinant RNA) comprising: a) a double-stranded RNA of the present disclosure; and b) a microRNA scaffold comprising a 5′ flanking polynucleotide, a loop polynucleotide, and a 3′ flanking polynucleotide, wherein the recombinant nucleic acid comprises: i) the 5′ flanking polynucleotide; ii) the second strand of the double-stranded RNA; iii) the loop polynucleotide; (iv) the first strand of the double-stranded RNA; and iii) the 3′ flanking polynucleotide; and wherein at least one of the 5′ flanking polynucleotide, the loop polynucleotide, and the 3′ flanking polynucleotide is heterologous to the first and/or the second strand of the double-stranded RNA. In some cases, the 5′ flanking polynucleotide, the loop polynucleotide, and the 3′ flanking polynucleotide are derived from miR33.

The present disclosure provides a recombinant nucleic acid (e.g., a recombinant RNA; which may be referred to as an “artificial microRNA” or a “small binding RNA” (sbRNA)) comprising: a) a double-stranded RNA of the present disclosure; and b) a microRNA scaffold comprising a 5′ flanking polynucleotide (also referred to herein as a “5′ leader”) and a 3′ flanking polynucleotide (also referred to herein as a “3′ trailer”), wherein the recombinant nucleic acid comprises: i) the 5′ flanking polynucleotide; ii) the first strand of the double-stranded RNA; iii) the second strand of the double-stranded RNA; and iv) the 3′ trailer polynucleotide; and wherein one or both of the 5′ flanking polynucleotide and the 3′ flanking polynucleotide is heterologous to the first and/or the second strand of the double-stranded RNA. The present disclosure provides a recombinant nucleic acid (e.g., a recombinant RNA) comprising: a) a double-stranded RNA of the present disclosure; and b) a microRNA scaffold comprising a 5′ flanking polynucleotide and a 3′ flanking polynucleotide, wherein the recombinant nucleic acid comprises: i) the 5′ flanking polynucleotide; ii) the second strand of the double-stranded RNA; iii) the first strand of the double-stranded RNA; and iv) the 3′ flanking polynucleotide; and wherein one or both of 5′ flanking polynucleotide and the 3′ flanking polynucleotide is heterologous to the first and/or the second strand of the double-stranded RNA. In some cases, the 5′ flanking polynucleotide and the 3′ flanking polynucleotide are derived from miR451.

### Cassettes Encoding a Recombinant RNA Molecule

The present disclosure provides a DNA molecule (e.g, a “cassette”, which can be inserted into an expression vector to generate a recombinant expression vector) comprising a nucleotide sequence encoding a recombinant RNA molecule of the present disclosure (where the recombinant RNA molecule may be referred to as an “artificial microRNA” or an “sbRNA”), where the recombinant RNA molecule comprises: a) a double-stranded RNA of the present disclosure; and b) a microRNA scaffold comprising a 5′ flanking polynucleotide (also referred to herein as a “5′ leader”), a loop polynucleotide, and a 3′ flanking polynucleotide (also referred to herein as a “3′ trailer”), wherein the recombinant nucleic acid comprises: i) the 5′ flanking polynucleotide; ii) the first strand of the double-stranded RNA; iii) the loop polynucleotide; (iv) the second strand of the double-stranded RNA; and iii) the 3′ trailer polynucleotide; and wherein at least one of the 5′ flanking polynucleotide, the loop polynucleotide, and the 3′ flanking polynucleotide is heterologous to the first and/or the second strand of the double-stranded RNA. The present disclosure provides a DNA molecule (e.g, a “cassette”, which can be inserted into an expression vector to generate a recombinant expression vector) comprising a nucleotide sequence encoding a recombinant RNA molecule of the present disclosure (where the recombinant RNA molecule may be referred to as an “artificial microRNA” or “sbRNA”), where the recombinant RNA molecule comprises: a) a double-stranded RNA of the present disclosure; and b) a microRNA scaffold comprising a 5′ flanking polynucleotide, a loop polynucleotide, and a 3′ flanking polynucleotide, wherein the recombinant nucleic acid comprises: i) the 5′ flanking polynucleotide; ii) the second strand of the double-stranded RNA; iii) the loop polynucleotide; (iv) the first strand of the double-stranded RNA; and iii) the 3′ flanking polynucleotide; and wherein at least one of the 5′ flanking polynucleotide, the loop polynucleotide, and the 3′ flanking polynucleotide is heterologous to the first and/or the second strand of the double-stranded RNA. In some cases, the 5′ flanking polynucleotide, the loop polynucleotide, and the 3′ flanking polynucleotide are derived from miR33. In some cases, the cassette includes a Pol3 transcription sequence; for example, in some cases, the cassette includes the nucleotide sequence TTTTTG 3′ of the nucleotide sequence encoding the 3′ trailer polynucleotide. In some cases, the cassette includes the nucleotide sequence Tₙ, where n is an integer from 5 to 10 (e.g., n is 5, 6, 7, 8, 9, or 10), 3′ of the nucleotide sequence encoding the 3′ trailer polynucleotide. In some cases, a cassette has a length of from about 110 nucleotides to about 150 nucleotides. In some cases, the cassette includes a Pol II transcription sequence; for example, in some cases, the cassette includes a polyadenylation sequence 3′ of the nucleotide sequence encoding the 3′ flanking polynucleotide.

The present disclosure provides a DNA molecule (e.g, a “cassette”, which can be inserted into an expression vector to generate a recombinant expression vector) comprising a nucleotide sequence encoding a recombinant RNA molecule of the present disclosure (where the recombinant RNA molecule may be referred to as an “artificial microRNA” or “sbRNA”), where the recombinant RNA molecule comprises: a) a double-stranded RNA of the present disclosure; and b) a microRNA scaffold comprising a 5′ flanking polynucleotide (also referred to herein as a “5′ leader”) and a 3′ flanking polynucleotide (also referred to herein as a “3′ trailer”), wherein the recombinant nucleic acid comprises: i) the 5′ flanking polynucleotide; ii) the first strand of the double-stranded RNA; iii) the second strand of the double-stranded RNA; and iv) the 3′ trailer polynucleotide; and wherein one or both of the 5′ flanking polynucleotide and the 3′ flanking polynucleotide is heterologous to the first and/or the second strand of the double-stranded RNA. The present disclosure provides a DNA molecule (e.g, a “cassette”, which can be inserted into an expression vector to generate a recombinant expression vector) comprising a nucleotide sequence encoding a recombinant RNA molecule of the present disclosure (where the recombinant RNA molecule may be referred to as an “artificial microRNA” or “sbRNA”), where the recombinant RNA molecule comprises: a) a double-stranded RNA of the present disclosure; and b) a microRNA scaffold comprising a 5′ flanking polynucleotide and a 3′ flanking polynucleotide, wherein the recombinant nucleic acid comprises: i) the 5′ flanking polynucleotide; ii) the second strand of the double-stranded RNA; iii) the first strand of the double-stranded RNA; and iv) the 3′ flanking polynucleotide; and wherein one or both of 5′ flanking polynucleotide and the 3′ flanking polynucleotide is heterologous to the first and/or the second strand of the double-stranded RNA. In some cases, the 5′ flanking polynucleotide and the 3′ flanking polynucleotide are derived from miR451. In some cases, the cassette includes a Pol3 transcription sequence; for example, in some cases, the cassette includes the nucleotide sequence TTTTTG 3′ of the nucleotide sequence encoding the 3′ trailer polynucleotide. In some cases, the cassette includes the nucleotide sequence Tₙ, where n is an integer from 5 to 10 (e.g., n is 5, 6, 7, 8, 9, or 10), 3′ of the nucleotide sequence encoding the 3′ trailer polynucleotide. In some cases, a cassette has a length of from about 110 nucleotides to about 650 nucleotides (e.g., from 110 nucleotides (nt) to 115 nt, from 115 nt to 120 nt, from 500 nt to 600 nt, or from 600 nt to 610 nt). In some cases, the cassette includes a Pol II transcription sequence; for example, in some cases, the cassette includes a polyadenylation sequence 3′ of the nucleotide sequence encoding the 3′ flanking polynucleotide.

In some cases, the portion of the cassette encoding the 5′ flanking polynucleotide comprises the nucleotide sequence tgcacacctcctggcgggcagctctg (SEQ ID NO:738). In some cases, the portion of the cassette encoding the loop polynucleotide comprises the nucleotide sequence tgttctggcaatacctg (SEQ ID NO:739). In some cases, the portion of the cassette encoding the 3′ flanking polynucleotide comprises the nucleotide sequence gggaggcctgccctgactgcccac (SEQ ID NO:740). In some cases, the cassette includes a Pol3 transcription sequence; for example, in some cases, the cassette includes the nucleotide sequence TTTTTG 3′ of the nucleotide sequence encoding the 3′ trailer polynucleotide. In some cases, a cassette has a length of from about 110 nucleotides to about 150 nucleotides. In some cases, the cassette includes a Pol II transcription sequence; for example, in some cases, the cassette includes a polyadenylation sequence 3′ of the nucleotide sequence encoding the 3′ flanking polynucleotide.

In some cases, the portion of the cassette encoding the 5′ flanking polynucleotide comprises the nucleotide sequence acctactgactgccagggcacttgggaatggcaagg (SEQ ID NO:854). In some cases, the portion of the cassette encoding the 3′ flanking polynucleotide comprises the nucleotide sequence tcttgctatacccagaaaacgtgccaggaagagaac (SEQ ID NO:855). In some cases, the portion of the cassette encoding the 5′ flanking polynucleotide comprises the nucleotide sequence acctactgactgccagggcacttgggaatggcaagg (SEQ ID NO:854); and the portion of the cassette encoding the 3′ flanking polynucleotide comprises the nucleotide sequence tcttgctatacccagaaaacgtgccaggaagagaac (SEQ ID NO:855).

In some cases, the portion of the cassette encoding the 5′ flanking polynucleotide comprises the nucleotide sequence gctcctgggcaacgtgctggttattgtgctgtctcatcattttggcaaagaattaagggcgaattcgagctcggtacctcgcgaatgcatctag atatcggcgctatgcttcctgtgcccccagtggggccctggctgggatTtcatcatatactgtaagtttgcgatgagacactacagtatagatg atgtactagtccgggcacccccagctctggagcctgacaaggaggacaggagagatgctgcaagcccaagaagctctctgctcagcctgtc acaacctactgactgccagggcacttgggaatggcaagg (SEQ ID NO:856). In some cases, the portion of the cassette encoding the 3′ flanking polynucleotide comprises the nucleotide sequence tcttgctatacccagaaaacgtgccaggaagagaactcaggaccctgaagcagactactggaagggagactccagctcaaacaaggcagg ggtgggggcgtgggattgggggtaggggagggaatagatacattttctctttcctgttgtaaagaaataaagataagccaggcacagtggct cacgcctgtaatcccaccactttcagaggccaaggcgctggatccagatctcgagcggccgcccg (SEQ ID NO:857). In some cases, the portion of the cassette encoding the 5′ flanking polynucleotide comprises the nucleotide sequence gctcctgggcaacgtgctggttattgtgctgtctcatcattttggcaaagaattaagggcgaattcgagctcggtacctcgcgaatgcatctag atatcggcgctatgcttcctgtgcccccagtggggccctggctgggatTtcatcatatactgtaagtttgcgatgagacactacagtatagatg atgtactagtccgggcacccccagctctggagcctgacaaggaggacaggagagatgctgcaagcccaagaagctctctgctcagcctgtc acaacctactgactgccagggcacttgggaatggcaagg (SEQ ID NO:856); and the portion of the cassette encoding the 3′ flanking polynucleotide comprises the nucleotide sequence tcttgctatacccagaaaacgtgccaggaagagaactcaggaccctgaagcagactactggaagggagactccagctcaaacaaggcagg ggtgggggcgtgggattgggggtaggggagggaatagatacattttctctttcctgttgtaaagaaataaagataagccaggcacagtggct cacgcctgtaatcccaccactttcagaggccaaggcgctggatccagatctcgagcggccgcccg (SEQ ID NO:857).

In some cases, the portion of the cassette encoding the 5′ flanking polynucleotide comprises the nucleotide sequence gctcctgggcaacgtgctggttattgtgctgtctcatcattttggcaaagaattaagggcgaattcgagctcggtacctcgcgaatgcatctag atatcggcgctatgcttcctgtgcccccagtggggccctggctgggatAtcatcatatactgtaagtttgcgatgagacactacagtatagatg atgtactagtccgggcacccccagctctggagcctgacaaggaggacaggagagatgctgcaagcccaagaagctctctgctcagcctgtc acaacctactgactgccagggcacttgggaatggcaagg (SEQ ID NO:858). In some cases, the portion of the cassette encoding the 3′ flanking polynucleotide comprises the nucleotide sequence tcttgctatacccagaaaacgtgccaggaagagaactcaggaccctgaagcagactactggaagggagactccagctcaaacaaggcagg ggtgggggcgtgggattgggggtaggggagggaatagatacattttctctttcctgttgtaaagaaataaagataagccaggcacagtggct cacgcctgtaatcccaccactttcagaggccaaggcgctggatccagatctcgagcggccgccc (SEQ ID NO:859). In some cases, the portion of the cassette encoding the 5′ flanking polynucleotide comprises the nucleotide sequence gctcctgggcaacgtgctggttattgtgctgtctcatcattttggcaaagaattaagggcgaattcgagctcggtacctcgcgaatgcatctag atatcggcgctatgcttcctgtgcccccagtggggccctggctgggatAtcatcatatactgtaagtttgcgatgagacactacagtatagatg atgtactagtccgggcacccccagctctggagcctgacaaggaggacaggagagatgctgcaagcccaagaagctctctgctcagcctgtc acaacctactgactgccagggcacttgggaatggcaagg (SEQ ID NO:858); and the portion of the cassette encoding the 3′ flanking polynucleotide comprises the nucleotide sequence tcttgctatacccagaaaacgtgccaggaagagaactcaggaccctgaagcagactactggaagggagactccagctcaaacaaggcagg ggtgggggcgtgggattgggggtaggggagggaatagatacattttctctttcctgttgtaaagaaataaagataagccaggcacagtggct cacgcctgtaatcccaccactttcagaggccaaggcgctggatccagatctcgagcggccgccc (SEQ ID NO:859).

The following are non-limiting examples of cassettes. In the following cassettes: (i) tgcacacctcctggcgggcagctctg (SEQ ID NO:738) encodes the 5′ leader polynucleotide; (ii) the first upper case sequence encodes the first strand of the double-stranded RNA; (iii) tgttctggcaatacctg (SEQ ID NO:739) encodes the loop polynucleotide; (iv) the second upper case sequence encodes the second strand of the double-stranded RNA; (v) gggaggcctgccctgactgcccac (SEQ ID NO:740) encodes the 3′ trailer polynucleotide; and (vi) TTTTTG is the Pol3 transcription termination sequence. In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette includes the nucleotide sequence TTTTT in place of the 3′ TTTTTG sequence.

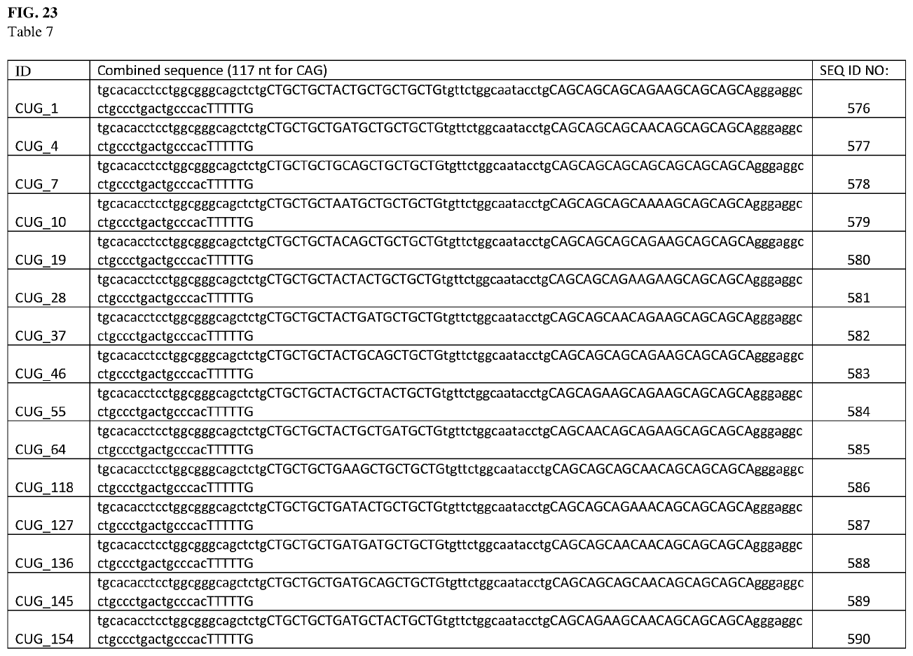

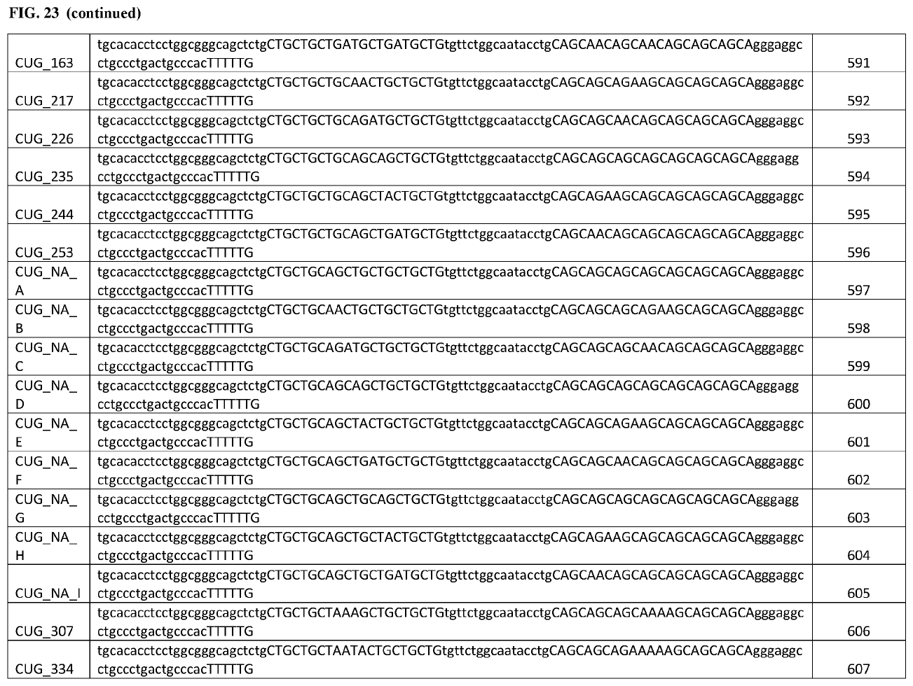

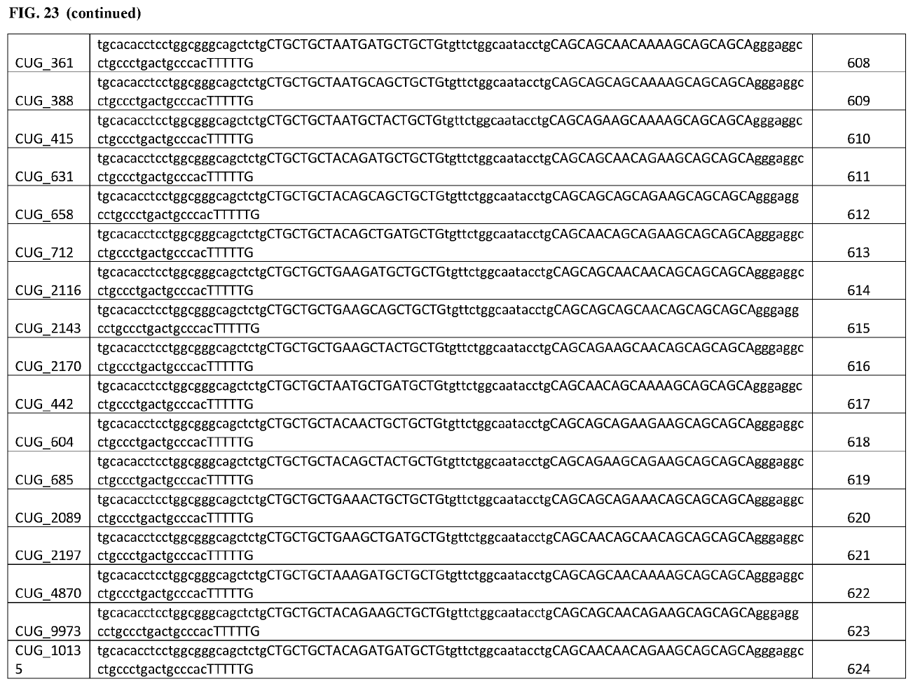

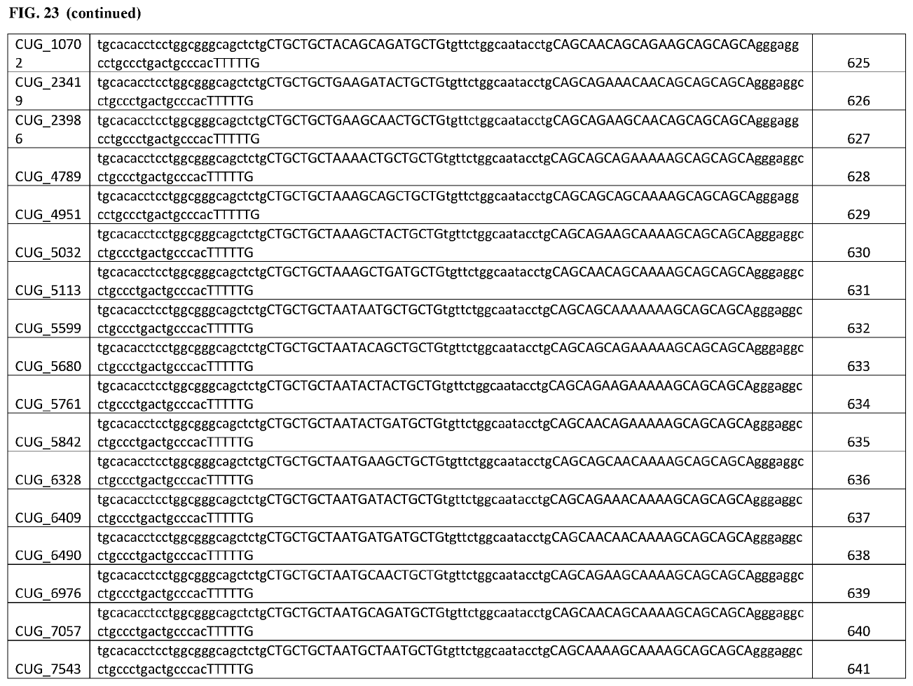

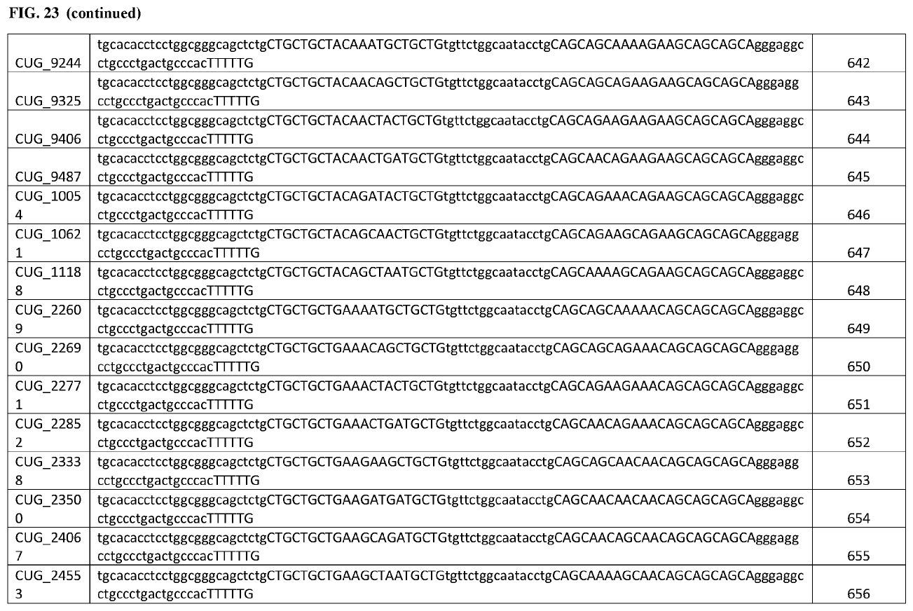

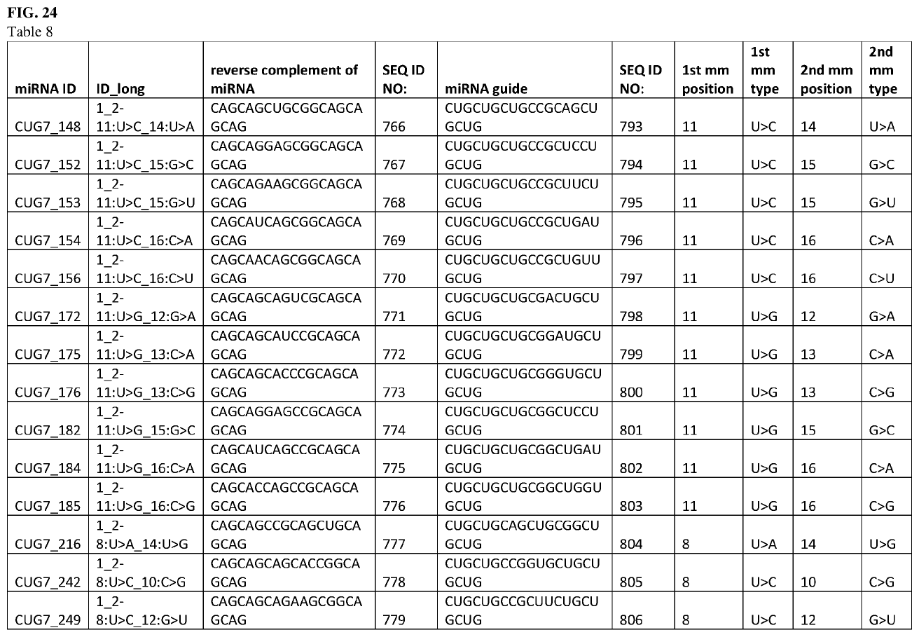

1) In some cases, a cassette comprises the nucleotide sequence: tgcacacctcctggcgggcagctctgCTGCTGCTAATGCTGCTGCTGtgttctggcaatacctgCAGCAGCAGCA AAAGCAGCAGCAgggaggcctgccctgactgcccacTTTTTG (SEQ ID NO:579; “CUG-10” in Table 7; FIG. 23). In some cases, the cassette does not include the 3′ TTTTTG sequence.
2) In some cases, a cassette comprises the nucleotide sequence: tgcacacctcctggcgggcagctctgCTGCTGCTACAGCTGCTGCTGtgttctggcaatacctgCAGCAGCAGCAGAAGCA GCAGCAgggaggcctgccctgactgcccacTTTTTG (SEQ ID NO:580; “CUG_19” in Table 7; FIG. 23). In some cases, the cassette does not include the 3′ TTTTTG sequence;
3) In some cases, a cassette comprises the nucleotide sequence: tgcacacctcctggcgggcagctctgCTGCTGCTACTACTGCTGCTGtgttctggcaatacctgCAGCAGCAGAAGAAGCA GCAGCAgggaggcctgccctgactgcccacTTTTTG (SEQ ID NO:581; “CUG_28” in Table 7; FIG. 23). In some cases, the cassette does not include the 3′ TTTTTG sequence;
4) In some cases, a cassette comprises the nucleotide sequence: tgcacacctcctggcgggcagctctgCTGCTGCTACTGATGCTGCTGtgttctggcaatacctgCAGCAGCAACAGAAGCA GCAGCAgggaggcctgccctgactgcccacTTTTTG (SEQ ID NO:582; “CUG_37” in Table 7; FIG. 23). In some cases, the cassette does not include the 3′ TTTTTG sequence;
5) In some cases, a cassette comprises the nucleotide sequence: tgcacacctcctggcgggcagctctgCTGCTGCTACTGCAGCTGCTGtgttctggcaatacctgCAGCAGCAGCAGAAGCA GCAGCAgggaggcctgccctgactgcccacTTTTTG (SEQ ID NO:583; “CUG_46” in Table 7; FIG. 23). In some cases, the cassette does not include the 3′ TTTTTG sequence;
6) In some cases, a cassette comprises the nucleotide sequence: tgcacacctcctggcgggcagctctgCTGCTGCTACTGCTACTGCTGtgttctggcaatacctgCAGCAGAAGCAGAAGCA GCAGCAgggaggcctgccctgactgcccacTTTTTG (SEQ ID NO:584; CUG_55” in Table 7; FIG. 23). In some cases, the cassette does not include the 3′ TTTTTG sequence;
7) In some cases, a cassette comprises the nucleotide sequence: tgcacacctcctggcgggcagctctgCTGCTGCTACTGCTGATGCTGtgttctggcaatacctgCAGCAACAGCAGAAGCA GCAGCAgggaggcctgccctgactgcccacTTTTTG (SEQ ID NO:585; “CUG_64” in Table 7; FIG. 23). In some cases, the cassette does not include the 3′ TTTTTG sequence;
8) In some cases, a cassette comprises the nucleotide sequence: tgcacacctcctggcgggcagctctgCTGCTGCTGAAGCTGCTGCTGtgttctggcaatacctgCAGCAGCAGCAACAGCA GCAGCAgggaggcctgccctgactgcccacTTTTTG (SEQ ID NO:586; “CUG_118” in Table 7; FIG. 23). In some cases, the cassette does not include the 3′ TTTTTG sequence;
9) In some cases, a cassette comprises the nucleotide sequence: tgcacacctcctggcgggcagctctgCTGCTGCTGATACTGCTGCTGtgttctggcaatacctgCAGCAGCAGAAACAGCA GCAGCAgggaggcctgccctgactgcccacTTTTTG (SEQ ID NO:587; “CUG_127” in Table 7; FIG. 23). In some cases, the cassette does not include the 3′ TTTTTG sequence;
10) In some cases, a cassette comprises the nucleotide sequence: tgcacacctcctggcgggcagctctgCTGCTGCTGATGATGCTGCTGtgttctggcaatacctgCAGCAGCAACAACAGCA GCAGCAgggaggcctgccctgactgcccacTTTTTG (SEQ ID NO:588; “CUG_136” in Table 7; FIG. 23). In some cases, the cassette does not include the 3′ TTTTTG sequence;
11) In some cases, a cassette comprises the nucleotide sequence: tgcacacctcctggcgggcagctctgCTGCTGCTGATGCAGCTGCTGtgttctggcaatacctgCAGCAGCAGCAACAGCA GCAGCAgggaggcctgccctgactgcccacTTTTTG (SEQ ID NO:589; “CUG_145” in Table 7; FIG. 23). In some cases, the cassette does not include the 3′ TTTTTG sequence;
12) In some cases, a cassette comprises the nucleotide sequence: tgcacacctcctggcgggcagctctgCTGCTGCTGATGCTACTGCTGtgttctggcaatacctgCAGCAGAAGCAACAGCA GCAGCAgggaggcctgccctgactgcccacTTTTTG (SEQ ID NO:590; “CUG_154” in Table 7; FIG. 23). In some cases, the cassette does not include the 3′ TTTTTG sequence;
13) In some cases, a cassette comprises the nucleotide sequence: tgcacacctcctggcgggcagctctgCTGCTGCTGATGCTGATGCTGtgttctggcaatacctgCAGCAACAGCAACAGCA GCAGCAgggaggcctgccctgactgcccacTTTTTG (SEQ ID NO:591; “CUG_163” in Table 7; FIG. 23). In some cases, the cassette does not include the 3′ TTTTTG sequence;
14) In some cases, a cassette comprises the nucleotide sequence: tgcacacctcctggcgggcagctctgCTGCTGCTGCAACTGCTGCTGtgttctggcaatacctgCAGCAGCAGAAGCAGCA GCAGCAgggaggcctgccctgactgcccacTTTTTG (SEQ ID NO:592; “CUG_217” in Table 7; FIG. 23). In some cases, the cassette does not include the 3′ TTTTTG sequence;
15) In some cases, a cassette comprises the nucleotide sequence: tgcacacctcctggcgggcagctctgCTGCTGCTGCAGATGCTGCTGtgttctggcaatacctgCAGCAGCAACAGCAGCA GCAGCAgggaggcctgccctgactgcccacTTTTTG (SEQ ID NO:593; “CUG_226” in Table 7; FIG. 23). In some cases, the cassette does not include the 3′ TTTTTG sequence;
16) In some cases, a cassette comprises the nucleotide sequence: tgcacacctcctggcgggcagctctgCTGCTGCTGCAGCAGCTGCTGtgttctggcaatacctgCAGCAGCAGCAGCAGCA GCAGCAgggaggcctgccctgactgcccacTTTTTG (SEQ ID NO:594; “CUG_235” in Table 7; FIG. 23). In some cases, the cassette does not include the 3′ TTTTTG sequence;
17) In some cases, a cassette comprises the nucleotide sequence: tgcacacctcctggcgggcagctctgCTGCTGCTGCAGCTACTGCTGtgttctggcaatacctgCAGCAGAAGCAGCAGCA GCAGCAgggaggcctgccctgactgcccacTTTTTG (SEQ ID NO:595; “CUG_244” in Table 7; FIG. 23). In some cases, the cassette does not include the 3′ TTTTTG sequence;
18) In some cases, a cassette comprises the nucleotide sequence: tgcacacctcctggcgggcagctctgCTGCTGCTGCAGCTGATGCTGtgttctggcaatacctgCAGCAACAGCAGCAGCA GCAGCAgggaggcctgccctgactgcccacTTTTTG (SEQ ID NO:596; “CUG_253” in Table 7; FIG. 23). In some cases, the cassette does not include the 3′ TTTTTG sequence;
19) In some cases, a cassette comprises the nucleotide sequence: tgcacacctcctggcgggcagctctgCTGCTGCAGCTGCTGCTGCTGtgttctggcaatacctgCAGCAGCAGCAGCAGCA GCAGCAgggaggcctgccctgactgcccacTTTTTG (SEQ ID NO:597; “CUG_NA-A” in Table 7; FIG. 23). In some cases, the cassette does not include the 3′ TTTTTG sequence;
20) In some cases, a cassette comprises the nucleotide sequence: tgcacacctcctggcgggcagctctgCTGCTGCAACTGCTGCTGCTGtgttctggcaatacctgCAGCAGCAGCAGAAGCA GCAGCAgggaggcctgccctgactgcccacTTTTTG (SEQ ID NO:598; “CUG_NA_B” in Table 7; FIG. 23). In some cases, the cassette does not include the 3′ TTTTTG sequence;
21) In some cases, a cassette comprises the nucleotide sequence: tgcacacctcctggcgggcagctctgCTGCTGCAGATGCTGCTGCTGtgttctggcaatacctgCAGCAGCAGCAACAGCA GCAGCAgggaggcctgccctgactgcccacTTTTTG (SEQ ID NO:599; “CUG_NA_C” in Table 7; FIG. 23). In some cases, the cassette does not include the 3′ TTTTTG sequence. In some cases, the cassette does not include the 3′ TTTTTG sequence;
22) In some cases, a cassette comprises the nucleotide sequence: tgcacacctcctggcgggcagctctgCTGCTGCAGCAGCTGCTGCTGtgttctggcaatacctgCAGCAGCAGCAGCAGCA GCAGCAgggaggcctgccctgactgcccacTTTTTG (SEQ ID NO:600; “CUG_NA_D” in Table 7; FIG. 23). In some cases, the cassette does not include the 3′ TTTTTG sequence;
23) In some cases, a cassette comprises the nucleotide sequence: tgcacacctcctggcgggcagctctgCTGCTGCAGCTACTGCTGCTGtgttctggcaatacctgCAGCAGCAGAAGCAGCA GCAGCAgggaggcctgccctgactgcccacTTTTTG (SEQ ID NO:601; “CUG_NA_E” in Table 7; FIG. 23). In some cases, the cassette does not include the 3′ TTTTTG sequence;
24) In some cases, a cassette comprises the nucleotide sequence: tgcacacctcctggcgggcagctctgCTGCTGCAGCTGATGCTGCTGtgttctggcaatacctgCAGCAGCAACAGCAGCA GCAGCAgggaggcctgccctgactgcccacTTTTTG (SEQ ID NO:602; “CUG_NA_F” in Table 7; FIG. 23). In some cases, the cassette does not include the 3′ TTTTTG sequence;
25) In some cases, a cassette comprises the nucleotide sequence: tgcacacctcctggcgggcagctctgCTGCTGCAGCTGCAGCTGCTGtgttctggcaatacctgCAGCAGCAGCAGCAGCA GCAGCAgggaggcctgccctgactgcccacTTTTTG (SEQ ID NO:603; “CUG_NA_G” in Table 7; FIG. 23). In some cases, the cassette does not include the 3′ TTTTTG sequence;
26) In some cases, a cassette comprises the nucleotide sequence: tgcacacctcctggcgggcagctctgCTGCTGCAGCTGCTACTGCTGtgttctggcaatacctgCAGCAGAAGCAGCAGCA GCAGCAgggaggcctgccctgactgcccacTTTTTG (SEQ ID NO:604; “CUG_NA_H” in Table 7; FIG. 23). In some cases, the cassette does not include the 3′ TTTTTG sequence;
27) In some cases, a cassette comprises the nucleotide sequence: tgcacacctcctggcgggcagctctgCTGCTGCAGCTGCTGATGCTGtgttctggcaatacctgCAGCAACAGCAGCAGCA GCAGCAgggaggcctgccctgactgcccacTTTTTG (SEQ ID NO:605; “CUG_NA_I” in Table 7; FIG. 23). In some cases, the cassette does not include the 3′ TTTTTG sequence;
28) In some cases, a cassette comprises the nucleotide sequence: tgcacacctcctggcgggcagctctgCTGCTGCTAAAGCTGCTGCTGtgttctggcaatacctgCAGCAGCAGCAAAAGCA GCAGCAgggaggcctgccctgactgcccacTTTTTG (SEQ ID NO:606; “CUG_307” in Table 7; FIG. 23). In some cases, the cassette does not include the 3′ TTTTTG sequence;
29) In some cases, a cassette comprises the nucleotide sequence: tgcacacctcctggcgggcagctctgCTGCTGCTAATACTGCTGCTGtgttctggcaatacctgCAGCAGCAGAAAAAGCA GCAGCAgggaggcctgccctgactgcccacTTTTTG (SEQ ID NO:607; “CUG_334” in Table 7; FIG. 23). In some cases, the cassette does not include the 3′ TTTTTG sequence;
30) In some cases, a cassette comprises the nucleotide sequence: tgcacacctcctggcgggcagctctgCTGCTGCTAATGATGCTGCTGtgttctggcaatacctgCAGCAGCAACAAAAGCA GCAGCAgggaggcctgccctgactgcccacTTTTTG (SEQ ID NO:608; “CUG_361” in Table 7; FIG. 23). In some cases, the cassette does not include the 3′ TTTTTG sequence;
31) In some cases, a cassette comprises the nucleotide sequence: tgcacacctcctggcgggcagctctgCTGCTGCTAATGCAGCTGCTGtgttctggcaatacctgCAGCAGCAGCAAAAGCA GCAGCAgggaggcctgccctgactgcccacTTTTTG (SEQ ID NO:609; “CUG_388” in Table 7; FIG. 23). In some cases, the cassette does not include the 3′ TTTTTG sequence;
32) In some cases, a cassette comprises the nucleotide sequence: tgcacacctcctggcgggcagctctgCTGCTGCTAATGCTACTGCTGtgttctggcaatacctgCAGCAGAAGCAAAAGCA GCAGCAgggaggcctgccctgactgcccacTTTTTG (SEQ ID NO:610; “CUG_415” in Table 7; FIG. 23). In some cases, the cassette does not include the 3′ TTTTTG sequence;
33) In some cases, a cassette comprises the nucleotide sequence: tgcacacctcctggcgggcagctctgCTGCTGCTACAGATGCTGCTGtgttctggcaatacctgCAGCAGCAACAGAAGCA GCAGCAgggaggcctgccctgactgcccacTTTTTG (SEQ ID NO:611; “CUG_631” in Table 7; FIG. 23). In some cases, the cassette does not include the 3′ TTTTTG sequence;
34) In some cases, a cassette comprises the nucleotide sequence: tgcacacctcctggcgggcagctctgCTGCTGCTACAGCAGCTGCTGtgttctggcaatacctgCAGCAGCAGCAGAAGCA GCAGCAgggaggcctgccctgactgcccacTTTTTG (SEQ ID NO:612; “CUG_658” in Table 7; FIG. 23). In some cases, the cassette does not include the 3′ TTTTTG sequence;
35) In some cases, a cassette comprises the nucleotide sequence: tgcacacctcctggcgggcagctctgCTGCTGCTACAGCTGATGCTGtgttctggcaatacctgCAGCAACAGCAGAAGCA GCAGCAgggaggcctgccctgactgcccacTTTTTG (SEQ ID NO:613; “CUG_712” in Table 7; FIG. 23). In some cases, the cassette does not include the 3′ TTTTTG sequence;
36) In some cases, a cassette comprises the nucleotide sequence: tgcacacctcctggcgggcagctctgCTGCTGCTGAAGATGCTGCTGtgttctggcaatacctgCAGCAGCAACAACAGCA GCAGCAgggaggcctgccctgactgcccacTTTTTG (SEQ ID NO:614; “CUG_2116” in Table 7; FIG. 23). In some cases, the cassette does not include the 3′ TTTTTG sequence;
37) In some cases, a cassette comprises the nucleotide sequence: tgcacacctcctggcgggcagctctgCTGCTGCTGAAGCAGCTGCTGtgttctggcaatacctgCAGCAGCAGCAACAGCA GCAGCAgggaggcctgccctgactgcccacTTTTTG (SEQ ID NO:615; “CUG_2143” in Table 7; FIG. 23). In some cases, the cassette does not include the 3′ TTTTTG sequence;
38) In some cases, a cassette comprises the nucleotide sequence: tgcacacctcctggcgggcagctctgCTGCTGCTGAAGCTACTGCTGtgttctggcaatacctgCAGCAGAAGCAACAGCA GCAGCAgggaggcctgccctgactgcccacTTTTTG (SEQ ID NO:616; “CUG_2170” in Table 7; FIG. 23). In some cases, the cassette does not include the 3′ TTTTTG sequence;
39) In some cases, a cassette comprises the nucleotide sequence: tgcacacctcctggcgggcagctctgCTGCTGCTAATGCTGATGCTGtgttctggcaatacctgCAGCAACAGCAAAAGCA GCAGCAgggaggcctgccctgactgcccacTTTTTG (SEQ ID NO:617; “CUG_442” in Table 7; FIG. 23). In some cases, the cassette does not include the 3′ TTTTTG sequence;
40) In some cases, a cassette comprises the nucleotide sequence: tgcacacctcctggcgggcagctctgCTGCTGCTACAACTGCTGCTGtgttctggcaatacctgCAGCAGCAGAAGAAGCA GCAGCAgggaggcctgccctgactgcccacTTTTTG (SEQ ID NO:618; “CUG_604” in Table 7; FIG. 23). In some cases, the cassette does not include the 3′ TTTTTG sequence;
41) In some cases, a cassette comprises the nucleotide sequence: tgcacacctcctggcgggcagctctgCTGCTGCTACAGCTACTGCTGtgttctggcaatacctgCAGCAGAAGCAGAAGCA GCAGCAgggaggcctgccctgactgcccacTTTTTG (SEQ ID NO:619; “CUG_685” in Table 7; FIG. 23). In some cases, the cassette does not include the 3′ TTTTTG sequence;
42) In some cases, a cassette comprises the nucleotide sequence: tgcacacctcctggcgggcagctctgCTGCTGCTGAAACTGCTGCTGtgttctggcaatacctgCAGCAGCAGAAACAGCA GCAGCAgggaggcctgccctgactgcccacTTTTTG (SEQ ID NO:620; “CUG_2089” in Table 7; FIG. 23). In some cases, the cassette does not include the 3′ TTTTTG sequence;
43) In some cases, a cassette comprises the nucleotide sequence: tgcacacctcctggcgggcagctctgCTGCTGCTGAAGCTGATGCTGtgttctggcaatacctgCAGCAACAGCAACAGCA GCAGCAgggaggcctgccctgactgcccacTTTTTG (SEQ ID NO:621; “CUG_2197” in Table 7; FIG. 23). In some cases, the cassette does not include the 3′ TTTTTG sequence;
44) In some cases, a cassette comprises the nucleotide sequence: tgcacacctcctggcgggcagctctgCTGCTGCTAAAGATGCTGCTGtgttctggcaatacctgCAGCAGCAACAAAAGCA GCAGCAgggaggcctgccctgactgcccacTTTTTG (SEQ ID NO:622; “CUG_4870” in Table 7; FIG. 23). In some cases, the cassette does not include the 3′ TTTTTG sequence;
45) In some cases, a cassette comprises the nucleotide sequence: tgcacacctcctggcgggcagctctgCTGCTGCTACAGAAGCTGCTGtgttctggcaatacctgCAGCAGCAACAGAAGCA GCAGCAgggaggcctgccctgactgcccacTTTTTG (SEQ ID NO:623; “CUG_9973” in Table 7; FIG. 23). In some cases, the cassette does not include the 3′ TTTTTG sequence;
46) In some cases, a cassette comprises the nucleotide sequence: tgcacacctcctggcgggcagctctgCTGCTGCTACAGATGATGCTGtgttctggcaatacctgCAGCAACAACAGAAGCA GCAGCAgggaggcctgccctgactgcccacTTTTTG (SEQ ID NO:624; “CUG_1013” in Table 7; FIG. 23). In some cases, the cassette does not include the 3′ TTTTTG sequence;
47) In some cases, a cassette comprises the nucleotide sequence: tgcacacctcctggcgggcagctctgCTGCTGCTACAGCAGATGCTGtgttctggcaatacctgCAGCAACAGCAGAAGCA GCAGCAgggaggcctgccctgactgcccacTTTTTG (SEQ ID NO:625; “CUG_1070” in Table 7; FIG. 23). In some cases, the cassette does not include the 3′ TTTTTG sequence;
48) In some cases, a cassette comprises the nucleotide sequence: tgcacacctcctggcgggcagctctgCTGCTGCTGAAGATACTGCTGtgttctggcaatacctgCAGCAGAAACAACAGCA GCAGCAgggaggcctgccctgactgcccacTTTTTG (SEQ ID NO:626; “CUG_2341” in Table 7; FIG. 23). In some cases, the cassette does not include the 3′ TTTTTG sequence;
49) In some cases, a cassette comprises the nucleotide sequence: tgcacacctcctggcgggcagctctgCTGCTGCTGAAGCAACTGCTGtgttctggcaatacctgCAGCAGAAGCAACAGCA GCAGCAgggaggcctgccctgactgcccacTTTTTG (SEQ ID NO:627; “CUG_2398” in Table 7; FIG. 23). In some cases, the cassette does not include the 3′ TTTTTG sequence;
50) In some cases, a cassette comprises the nucleotide sequence: tgcacacctcctggcgggcagctctgCTGCTGCTAAAACTGCTGCTGtgttctggcaatacctgCAGCAGCAGAAAAAGCA GCAGCAgggaggcctgccctgactgcccacTTTTTG (SEQ ID NO:628; “CUG_4789” in Table 7; FIG. 23). In some cases, the cassette does not include the 3′ TTTTTG sequence;
51) In some cases, a cassette comprises the nucleotide sequence: tgcacacctcctggcgggcagctctgCTGCTGCTAAAGCAGCTGCTGtgttctggcaatacctgCAGCAGCAGCAAAAGCA GCAGCAgggaggcctgccctgactgcccacTTTTTG (SEQ ID NO:629; “CUG_4951” in Table 7; FIG. 23). In some cases, the cassette does not include the 3′ TTTTTG sequence;
52) In some cases, a cassette comprises the nucleotide sequence: tgcacacctcctggcgggcagctctgCTGCTGCTAAAGCTACTGCTGtgttctggcaatacctgCAGCAGAAGCAAAAGCA GCAGCAgggaggcctgccctgactgcccacTTTTTG (SEQ ID NO:630; “CUG_5032” in Table 7; FIG. 23). In some cases, the cassette does not include the 3′ TTTTTG sequence;
53) In some cases, a cassette comprises the nucleotide sequence: tgcacacctcctggcgggcagctctgCTGCTGCTAAAGCTGATGCTGtgttctggcaatacctgCAGCAACAGCAAAAGCA GCAGCAgggaggcctgccctgactgcccacTTTTTG (SEQ ID NO:631; “CUG_5113” in Table 7; FIG. 23). In some cases, the cassette does not include the 3′ TTTTTG sequence;
54) In some cases, a cassette comprises the nucleotide sequence: tgcacacctcctggcgggcagctctgCTGCTGCTAATAATGCTGCTGtgttctggcaatacctgCAGCAGCAAAAAAAGCA GCAGCAgggaggcctgccctgactgcccacTTTTTG (SEQ ID NO:632; “CUG_5599” in Table 7; FIG. 23). In some cases, the cassette does not include the 3′ TTTTTG sequence;
55) In some cases, a cassette comprises the nucleotide sequence: tgcacacctcctggcgggcagctctgCTGCTGCTAATACAGCTGCTGtgttctggcaatacctgCAGCAGCAGAAAAAGCA GCAGCAgggaggcctgccctgactgcccacTTTTTG (SEQ ID NO:633; “CUG_5680” in Table 7; FIG. 23). In some cases, the cassette does not include the 3′ TTTTTG sequence;
56) In some cases, a cassette comprises the nucleotide sequence: tgcacacctcctggcgggcagctctgCTGCTGCTAATACTACTGCTGtgttctggcaatacctgCAGCAGAAGAAAAAGCA GCAGCAgggaggcctgccctgactgcccacTTTTTG (SEQ ID NO:634 “CUG_5761” in Table 7; FIG. 23). In some cases, the cassette does not include the 3′ TTTTTG sequence;
57) In some cases, a cassette comprises the nucleotide sequence: tgcacacctcctggcgggcagctctgCTGCTGCTAATACTGATGCTGtgttctggcaatacctgCAGCAACAGAAAAAGCA GCAGCAgggaggcctgccctgactgcccacTTTTTG (SEQ ID NO:635; “CUG_5842” in Table 7; FIG. 23). In some cases, the cassette does not include the 3′ TTTTTG sequence;
58) In some cases, a cassette comprises the nucleotide sequence: tgcacacctcctggcgggcagctctgCTGCTGCTAATGAAGCTGCTGtgttctggcaatacctgCAGCAGCAACAAAAGCA GCAGCAgggaggcctgccctgactgcccacTTTTTG (SEQ ID NO:636; “CUG_6328” in Table 7; FIG. 23). In some cases, the cassette does not include the 3′ TTTTTG sequence;
59) In some cases, a cassette comprises the nucleotide sequence: tgcacacctcctggcgggcagctctgCTGCTGCTAATGATACTGCTGtgttctggcaatacctgCAGCAGAAACAAAAGCA GCAGCAgggaggcctgccctgactgcccacTTTTTG (SEQ ID NO:637; “CUG_6409” in Table 7; FIG. 23). In some cases, the cassette does not include the 3′ TTTTTG sequence;
60) In some cases, a cassette comprises the nucleotide sequence: tgcacacctcctggcgggcagctctgCTGCTGCTAATGATGATGCTGtgttctggcaatacctgCAGCAACAACAAAAGCA GCAGCAgggaggcctgccctgactgcccacTTTTTG (SEQ ID NO:638; “CUG_6490” in Table 7; FIG. 23). In some cases, the cassette does not include the 3′ TTTTTG sequence;
61) In some cases, a cassette comprises the nucleotide sequence: tgcacacctcctggcgggcagctctgCTGCTGCTAATGCAACTGCTGtgttctggcaatacctgCAGCAGAAGCAAAAGCA GCAGCAgggaggcctgccctgactgcccacTTTTTG (SEQ ID NO:639; “CUG_6976” in Table 7; FIG. 23). In some cases, the cassette does not include the 3′ TTTTTG sequence;
62) In some cases, a cassette comprises the nucleotide sequence: tgcacacctcctggcgggcagctctgCTGCTGCTAATGCAGATGCTGtgttctggcaatacctgCAGCAACAGCAAAAGCA GCAGCAgggaggcctgccctgactgcccacTTTTTG (SEQ ID NO:640; “CUG_7057” in Table 7; FIG. 23). In some cases, the cassette does not include the 3′ TTTTTG sequence;
63) In some cases, a cassette comprises the nucleotide sequence: tgcacacctcctggcgggcagctctgCTGCTGCTAATGCTAATGCTGtgttctggcaatacctgCAGCAAAAGCAAAAGCA GCAGCAgggaggcctgccctgactgcccacTTTTTG (SEQ ID NO:641; “CUG_7543” in Table 7; FIG. 23). In some cases, the cassette does not include the 3′ TTTTTG sequence;
64) In some cases, a cassette comprises the nucleotide sequence: tgcacacctcctggcgggcagctctgCTGCTGCTACAAATGCTGCTGtgttctggcaatacctgCAGCAGCAAAAGAAGCA GCAGCAgggaggcctgccctgactgcccacTTTTTG (SEQ ID NO:642; “CUG_9244” in Table 7; FIG. 23). In some cases, the cassette does not include the 3′ TTTTTG sequence;
65) In some cases, a cassette comprises the nucleotide sequence: tgcacacctcctggcgggcagctctgCTGCTGCTACAACAGCTGCTGtgttctggcaatacctgCAGCAGCAGAAGAAGCA GCAGCAgggaggcctgccctgactgcccacTTTTTG (SEQ ID NO:643; “CUG_9325” in Table 7; FIG. 23). In some cases, the cassette does not include the 3′ TTTTTG sequence;
66) In some cases, a cassette comprises the nucleotide sequence: tgcacacctcctggcgggcagctctgCTGCTGCTACAACTACTGCTGtgttctggcaatacctgCAGCAGAAGAAGAAGCA GCAGCAgggaggcctgccctgactgcccacTTTTTG (SEQ ID NO:644; “CUG_9406” in Table 7; FIG. 23). In some cases, the cassette does not include the 3′ TTTTTG sequence;
67) In some cases, a cassette comprises the nucleotide sequence: tgcacacctcctggcgggcagctctgCTGCTGCTACAACTGATGCTGtgttctggcaatacctgCAGCAACAGAAGAAGCA GCAGCAgggaggcctgccctgactgcccacTTTTTG (SEQ ID NO:645; “CUG_9487” in Table 7; FIG. 23). In some cases, the cassette does not include the 3′ TTTTTG sequence;
68) In some cases, a cassette comprises the nucleotide sequence: tgcacacctcctggcgggcagctctgCTGCTGCTACAGATACTGCTGtgttctggcaatacctgCAGCAGAAACAGAAGCA GCAGCAgggaggcctgccctgactgcccacTTTTTG (SEQ ID NO:646; “CUG_10054” in Table 7; FIG. 23). In some cases, the cassette does not include the 3′ TTTTTG sequence;
69) In some cases, a cassette comprises the nucleotide sequence: tgcacacctcctggcgggcagctctgCTGCTGCTACAGCAACTGCTGtgttctggcaatacctgCAGCAGAAGCAGAAGCA GCAGCAgggaggcctgccctgactgcccacTTTTTG (SEQ ID NO:647; “CUG_10621” in Table 7; FIG. 23). In some cases, the cassette does not include the 3′ TTTTTG sequence;
70) In some cases, a cassette comprises the nucleotide sequence: tgcacacctcctggcgggcagctctgCTGCTGCTACAGCTAATGCTGtgttctggcaatacctgCAGCAAAAGCAGAAGCA GCAGCAgggaggcctgccctgactgcccacTTTTTG (SEQ ID NO:648; “CUG_11188” in Table 7; FIG. 23). In some cases, the cassette does not include the 3′ TTTTTG sequence;
71) In some cases, a cassette comprises the nucleotide sequence: tgcacacctcctggcgggcagctctgCTGCTGCTGAAAATGCTGCTGtgttctggcaatacctgCAGCAGCAAAAACAGCA GCAGCAgggaggcctgccctgactgcccacTTTTTG (SEQ ID NO:649; “CUG_222609” in Table 7; FIG. 23). In some cases, the cassette does not include the 3′ TTTTTG sequence;
72) In some cases, a cassette comprises the nucleotide sequence: tgcacacctcctggcgggcagctctgCTGCTGCTGAAACAGCTGCTGtgttctggcaatacctgCAGCAGCAGAAACAGCA GCAGCAgggaggcctgccctgactgcccacTTTTTG (SEQ ID NO:650; “CUG_22690” in Table 7; FIG. 23). In some cases, the cassette does not include the 3′ TTTTTG sequence;
73) In some cases, a cassette comprises the nucleotide sequence: tgcacacctcctggcgggcagctctgCTGCTGCTGAAACTACTGCTGtgttctggcaatacctgCAGCAGAAGAAACAGCA GCAGCAgggaggcctgccctgactgcccacTTTTTG (SEQ ID NO:651; “CUG_22771” in Table 7; FIG. 23). In some cases, the cassette does not include the 3′ TTTTTG sequence;
74) In some cases, a cassette comprises the nucleotide sequence: tgcacacctcctggcgggcagctctgCTGCTGCTGAAACTGATGCTGtgttctggcaatacctgCAGCAACAGAAACAGCA GCAGCAgggaggcctgccctgactgcccacTTTTTG (SEQ ID NO:652; “CUG_22852” in Table 7; FIG. 23). In some cases, the cassette does not include the 3′ TTTTTG sequence;
75) In some cases, a cassette comprises the nucleotide sequence: tgcacacctcctggcgggcagctctgCTGCTGCTGAAGAAGCTGCTGtgttctggcaatacctgCAGCAGCAACAACAGCA GCAGCAgggaggcctgccctgactgcccacTTTTTG (SEQ ID NO:653; “CUG_233338” in Table 7; FIG. 23). In some cases, the cassette does not include the 3′ TTTTTG sequence;
76) In some cases, a cassette comprises the nucleotide sequence: tgcacacctcctggcgggcagctctgCTGCTGCTGAAGATGATGCTGtgttctggcaatacctgCAGCAACAACAACAGCA GCAGCAgggaggcctgccctgactgcccacTTTTTG (SEQ ID NO:654; “CUG_23500” in Table 7; FIG. 23). In some cases, the cassette does not include the 3′ TTTTTG sequence;
77) In some cases, a cassette comprises the nucleotide sequence: tgcacacctcctggcgggcagctctgCTGCTGCTGAAGCAGATGCTGtgttctggcaatacctgCAGCAACAGCAACAGCA GCAGCAgggaggcctgccctgactgcccacTTTTTG (SEQ ID NO:655; “CUG_24067” in Table 7; FIG. 23). In some cases, the cassette does not include the 3′ TTTTTG sequence;
78) In some cases, a cassette comprises the nucleotide sequence: tgcacacctcctggcgggcagctctgCTGCTGCTGAAGCTAATGCTGtgttctggcaatacctgCAGCAAAAGCAACAGCA GCAGCAgggaggcctgccctgactgcccacTTTTTG (SEQ ID NO:656; “CUG_24553” in Table 7; FIG. 23). In some cases, the cassette does not include the 3′ TTTTTG sequence.

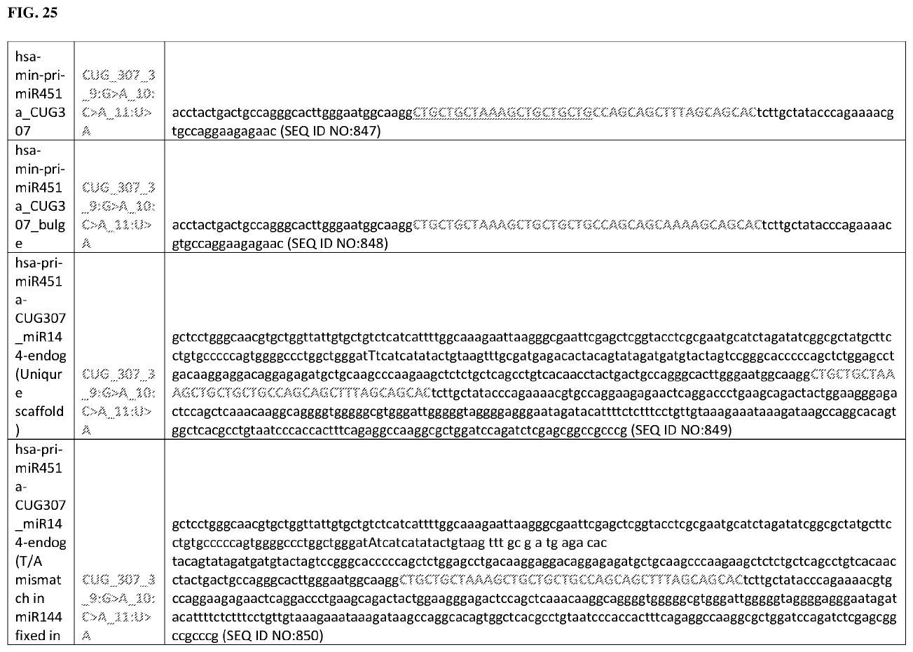

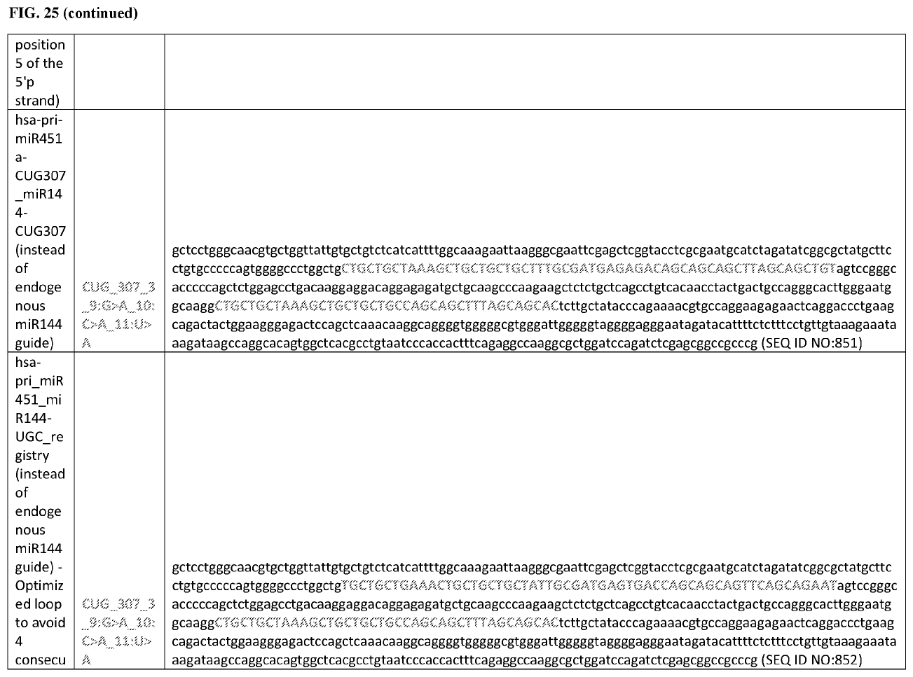

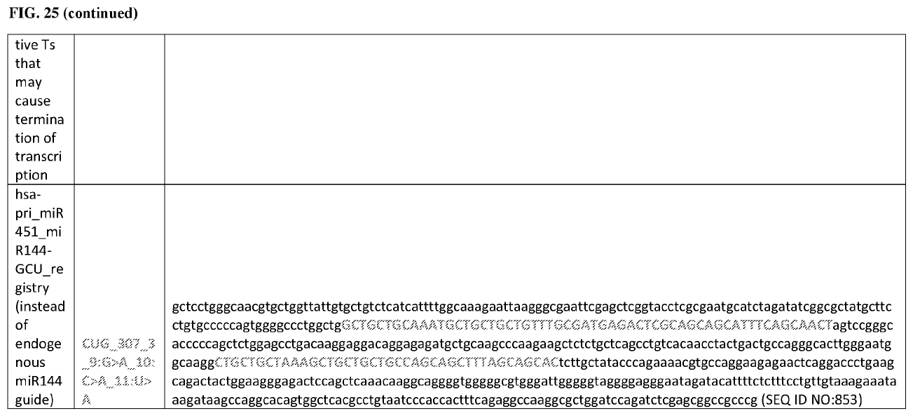

The following are non-limiting examples of cassettes. In the following cassettes: pairs of 5′ flanking and 3′ flanking polynucleotides include: i) SEQ ID NO:854 and SEQ ID NO:855; ii) SEQ ID NO:856 and SEQ ID NO:857; and iii) SEQ ID NO:858 and SEQ ID NO:859. In some cases, the cassette includes a 3′ TTTTTG transcription termination sequence. In some cases, the cassette does not include a 3′ TTTTTG sequence. In some cases, the cassette includes, at the 3′ end, the nucleotide sequence Tₙ, where n is an integer from 5 to 10 (e.g., n is 5, 6, 7, 8, 9, or 10). In some cases, the cassette includes, at the 3′ end, the nucleotide sequence TTTTT. Non-limiting examples include the cassettes shown in FIG. 25.

1) In some cases, a cassette comprises, in order from 5′ to 3′: i) SEQ ID NO:854; ii) SEQ ID NO:379; iii) a sequence that is complementary to SEQ ID NO: 379, where the sequence can comprise from 0 to 10 mismatches (non-complementary nucleotides) to SEQ ID NO: 379; and iv) SEQ ID NO:855. In some cases, a cassette comprises, in order from 5′ to 3′: i) SEQ ID NO:856; ii) SEQ ID NO: 379; iii) a sequence that is complementary to SEQ ID NO: 379, where the sequence can comprise from 0 to 10 mismatches (non-complementary nucleotides) to SEQ ID NO: 379; and iv) SEQ ID NO:857. In some cases, a cassette comprises, in order from 5′ to 3′: i) SEQ ID NO:858; ii) SEQ ID NO: 379; iii) a sequence that is complementary to SEQ ID NO: 379, where the sequence can comprise from 0 to 10 mismatches (non-complementary nucleotides) to SEQ ID NO: 379; and iv) SEQ ID NO:859.
2) In some cases, a cassette comprises, in order from 5′ to 3′: i) SEQ ID NO:854; ii) SEQ ID NO:380; iii) a sequence that is complementary to SEQ ID NO: 380, where the sequence can comprise from 0 to 10 mismatches (non-complementary nucleotides) to SEQ ID NO: 380; and iv) SEQ ID NO:855. In some cases, a cassette comprises, in order from 5′ to 3′: i) SEQ ID NO:856; ii) SEQ ID NO: 380; iii) a sequence that is complementary to SEQ ID NO: 380, where the sequence can comprise from 0 to 10 mismatches (non-complementary nucleotides) to SEQ ID NO: 380; and iv) SEQ ID NO:857. In some cases, a cassette comprises, in order from 5′ to 3′: i) SEQ ID NO:858; ii) SEQ ID NO: 380; iii) a sequence that is complementary to SEQ ID NO: 380, where the sequence can comprise from 0 to 10 mismatches (non-complementary nucleotides) to SEQ ID NO: 380; and iv) SEQ ID NO:859.
3) In some cases, a cassette comprises, in order from 5′ to 3′: i) SEQ ID NO:854; ii) SEQ ID NO:381; iii) a sequence that is complementary to SEQ ID NO: 381, where the sequence can comprise from 0 to 10 mismatches (non-complementary nucleotides) to SEQ ID NO: 381; and iv) SEQ ID NO:855. In some cases, a cassette comprises, in order from 5′ to 3′: i) SEQ ID NO:856; ii) SEQ ID NO: 381; iii) a sequence that is complementary to SEQ ID NO: 381, where the sequence can comprise from 0 to 10 mismatches (non-complementary nucleotides) to SEQ ID NO: 381; and iv) SEQ ID NO:857. In some cases, a cassette comprises, in order from 5′ to 3′: i) SEQ ID NO:858; ii) SEQ ID NO: 381; iii) a sequence that is complementary to SEQ ID NO: 381, where the sequence can comprise from 0 to 10 mismatches (non-complementary nucleotides) to SEQ ID NO: 381; and iv) SEQ ID NO:859.
4) In some cases, a cassette comprises, in order from 5′ to 3′: i) SEQ ID NO:854; ii) SEQ ID NO:382; iii) a sequence that is complementary to SEQ ID NO: 382, where the sequence can comprise from 0 to 10 mismatches (non-complementary nucleotides) to SEQ ID NO: 382; and iv) SEQ ID NO:855. In some cases, a cassette comprises, in order from 5′ to 3′: i) SEQ ID NO:856; ii) SEQ ID NO: 382; iii) a sequence that is complementary to SEQ ID NO: 382, where the sequence can comprise from 0 to 10 mismatches (non-complementary nucleotides) to SEQ ID NO: 382; and iv) SEQ ID NO:857. In some cases, a cassette comprises, in order from 5′ to 3′: i) SEQ ID NO:858; ii) SEQ ID NO: 382; iii) a sequence that is complementary to SEQ ID NO: 382, where the sequence can comprise from 0 to 10 mismatches (non-complementary nucleotides) to SEQ ID NO: 382; and iv) SEQ ID NO:859.
5) In some cases, a cassette comprises, in order from 5′ to 3′: i) SEQ ID NO:854; ii) SEQ ID NO:383; iii) a sequence that is complementary to SEQ ID NO: 383, where the sequence can comprise from 0 to 10 mismatches (non-complementary nucleotides) to SEQ ID NO: 383; and iv) SEQ ID NO:855. In some cases, a cassette comprises, in order from 5′ to 3′: i) SEQ ID NO:856; ii) SEQ ID NO: 383; iii) a sequence that is complementary to SEQ ID NO: 383, where the sequence can comprise from 0 to 10 mismatches (non-complementary nucleotides) to SEQ ID NO: 383; and iv) SEQ ID NO:857. In some cases, a cassette comprises, in order from 5′ to 3′: i) SEQ ID NO:858; ii) SEQ ID NO: 383; iii) a sequence that is complementary to SEQ ID NO: 383, where the sequence can comprise from 0 to 10 mismatches (non-complementary nucleotides) to SEQ ID NO: 383; and iv) SEQ ID NO:859.
6) In some cases, a cassette comprises, in order from 5′ to 3′: i) SEQ ID NO:854; ii) SEQ ID NO:384; iii) a sequence that is complementary to SEQ ID NO: 384, where the sequence can comprise from 0 to 10 mismatches (non-complementary nucleotides) to SEQ ID NO: 384; and iv) SEQ ID NO:855. In some cases, a cassette comprises, in order from 5′ to 3′: i) SEQ ID NO:856; ii) SEQ ID NO: 384; iii) a sequence that is complementary to SEQ ID NO: 384, where the sequence can comprise from 0 to 10 mismatches (non-complementary nucleotides) to SEQ ID NO: 384; and iv) SEQ ID NO:857. In some cases, a cassette comprises, in order from 5′ to 3′: i) SEQ ID NO:858; ii) SEQ ID NO: 384; iii) a sequence that is complementary to SEQ ID NO: 384, where the sequence can comprise from 0 to 10 mismatches (non-complementary nucleotides) to SEQ ID NO: 384; and iv) SEQ ID NO:859.
7) In some cases, a cassette comprises, in order from 5′ to 3′: i) SEQ ID NO:854; ii) SEQ ID NO:385; iii) a sequence that is complementary to SEQ ID NO: 385, where the sequence can comprise from 0 to 10 mismatches (non-complementary nucleotides) to SEQ ID NO: 385; and iv) SEQ ID NO:855. In some cases, a cassette comprises, in order from 5′ to 3′: i) SEQ ID NO:856; ii) SEQ ID NO: 385; iii) a sequence that is complementary to SEQ ID NO: 385, where the sequence can comprise from 0 to 10 mismatches (non-complementary nucleotides) to SEQ ID NO: 385; and iv) SEQ ID NO:857. In some cases, a cassette comprises, in order from 5′ to 3′: i) SEQ ID NO:858; ii) SEQ ID NO: 385; iii) a sequence that is complementary to SEQ ID NO: 385, where the sequence can comprise from 0 to 10 mismatches (non-complementary nucleotides) to SEQ ID NO: 385; and iv) SEQ ID NO:859.
8) In some cases, a cassette comprises, in order from 5′ to 3′: i) SEQ ID NO:854; ii) SEQ ID NO:386; iii) a sequence that is complementary to SEQ ID NO: 386, where the sequence can comprise from 0 to 10 mismatches (non-complementary nucleotides) to SEQ ID NO: 386; and iv) SEQ ID NO:855. In some cases, a cassette comprises, in order from 5′ to 3′: i) SEQ ID NO:856; ii) SEQ ID NO: 386; iii) a sequence that is complementary to SEQ ID NO: 386, where the sequence can comprise from 0 to 10 mismatches (non-complementary nucleotides) to SEQ ID NO: 386; and iv) SEQ ID NO:857. In some cases, a cassette comprises, in order from 5′ to 3′: i) SEQ ID NO:858; ii) SEQ ID NO: 386; iii) a sequence that is complementary to SEQ ID NO: 386, where the sequence can comprise from 0 to 10 mismatches (non-complementary nucleotides) to SEQ ID NO: 386; and iv) SEQ ID NO:859.
9) In some cases, a cassette comprises, in order from 5′ to 3′: i) SEQ ID NO:854; ii) SEQ ID NO:387; iii) a sequence that is complementary to SEQ ID NO: 387, where the sequence can comprise from 0 to 10 mismatches (non-complementary nucleotides) to SEQ ID NO: 387; and iv) SEQ ID NO:855. In some cases, a cassette comprises, in order from 5′ to 3′: i) SEQ ID NO:856; ii) SEQ ID NO: 387; iii) a sequence that is complementary to SEQ ID NO: 387, where the sequence can comprise from 0 to 10 mismatches (non-complementary nucleotides) to SEQ ID NO: 387; and iv) SEQ ID NO:857. In some cases, a cassette comprises, in order from 5′ to 3′: i) SEQ ID NO:858; ii) SEQ ID NO: 387; iii) a sequence that is complementary to SEQ ID NO: 387, where the sequence can comprise from 0 to 10 mismatches (non-complementary nucleotides) to SEQ ID NO: 387; and iv) SEQ ID NO:859.
10) In some cases, a cassette comprises, in order from 5′ to 3′: i) SEQ ID NO:854; ii) SEQ ID NO:388; iii) a sequence that is complementary to SEQ ID NO: 388, where the sequence can comprise from 0 to 10 mismatches (non-complementary nucleotides) to SEQ ID NO: 388; and iv) SEQ ID NO:855. In some cases, a cassette comprises, in order from 5′ to 3′: i) SEQ ID NO:856; ii) SEQ ID NO: 388; iii) a sequence that is complementary to SEQ ID NO: 388, where the sequence can comprise from 0 to 10 mismatches (non-complementary nucleotides) to SEQ ID NO: 388; and iv) SEQ ID NO:857. In some cases, a cassette comprises, in order from 5′ to 3′: i) SEQ ID NO:858; ii) SEQ ID NO: 388; iii) a sequence that is complementary to SEQ ID NO: 388, where the sequence can comprise from 0 to 10 mismatches (non-complementary nucleotides) to SEQ ID NO: 388; and iv) SEQ ID NO:859.
11) In some cases, a cassette comprises, in order from 5′ to 3′: i) SEQ ID NO:854; ii) SEQ ID NO:389; iii) a sequence that is complementary to SEQ ID NO: 389, where the sequence can comprise from 0 to 10 mismatches (non-complementary nucleotides) to SEQ ID NO: 389; and iv) SEQ ID NO:855. In some cases, a cassette comprises, in order from 5′ to 3′: i) SEQ ID NO:856; ii) SEQ ID NO: 389; iii) a sequence that is complementary to SEQ ID NO: 389, where the sequence can comprise from 0 to 10 mismatches (non-complementary nucleotides) to SEQ ID NO: 389; and iv) SEQ ID NO:857. In some cases, a cassette comprises, in order from 5′ to 3′: i) SEQ ID NO:858; ii) SEQ ID NO: 389; iii) a sequence that is complementary to SEQ ID NO: 389, where the sequence can comprise from 0 to 10 mismatches (non-complementary nucleotides) to SEQ ID NO: 389; and iv) SEQ ID NO:859.
12) In some cases, a cassette comprises, in order from 5′ to 3′: i) SEQ ID NO:854; ii) SEQ ID NO:390; iii) a sequence that is complementary to SEQ ID NO: 390, where the sequence can comprise from 0 to 10 mismatches (non-complementary nucleotides) to SEQ ID NO: 390; and iv) SEQ ID NO:855. In some cases, a cassette comprises, in order from 5′ to 3′: i) SEQ ID NO:856; ii) SEQ ID NO: 390; iii) a sequence that is complementary to SEQ ID NO: 390, where the sequence can comprise from 0 to 10 mismatches (non-complementary nucleotides) to SEQ ID NO: 390; and iv) SEQ ID NO:857. In some cases, a cassette comprises, in order from 5′ to 3′: i) SEQ ID NO:858; ii) SEQ ID NO: 390; iii) a sequence that is complementary to SEQ ID NO: 309, where the sequence can comprise from 0 to 10 mismatches (non-complementary nucleotides) to SEQ ID NO: 390; and iv) SEQ ID NO:859.
13) In some cases, a cassette comprises, in order from 5′ to 3′: i) SEQ ID NO:854; ii) SEQ ID NO:391; iii) a sequence that is complementary to SEQ ID NO: 391, where the sequence can comprise from 0 to 10 mismatches (non-complementary nucleotides) to SEQ ID NO: 391; and iv) SEQ ID NO:855. In some cases, a cassette comprises, in order from 5′ to 3′: i) SEQ ID NO:856; ii) SEQ ID NO: 391; iii) a sequence that is complementary to SEQ ID NO: 391, where the sequence can comprise from 0 to 10 mismatches (non-complementary nucleotides) to SEQ ID NO: 391; and iv) SEQ ID NO:857. In some cases, a cassette comprises, in order from 5′ to 3′: i) SEQ ID NO:858; ii) SEQ ID NO: 391; iii) a sequence that is complementary to SEQ ID NO: 391, where the sequence can comprise from 0 to 10 mismatches (non-complementary nucleotides) to SEQ ID NO: 391; and iv) SEQ ID NO:859.
14) In some cases, a cassette comprises, in order from 5′ to 3′: i) SEQ ID NO:854; ii) SEQ ID NO:390; iii) a sequence that is complementary to SEQ ID NO: 390, where the sequence can comprise from 0 to 10 mismatches (non-complementary nucleotides) to SEQ ID NO: 390; and iv) SEQ ID NO:855. In some cases, a cassette comprises, in order from 5′ to 3′: i) SEQ ID NO:856; ii) SEQ ID NO: 390; iii) a sequence that is complementary to SEQ ID NO: 390, where the sequence can comprise from 0 to 10 mismatches (non-complementary nucleotides) to SEQ ID NO: 390; and iv) SEQ ID NO:857. In some cases, a cassette comprises, in order from 5′ to 3′: i) SEQ ID NO:858; ii) SEQ ID NO: 390; iii) a sequence that is complementary to SEQ ID NO: 390, where the sequence can comprise from 0 to 10 mismatches (non-complementary nucleotides) to SEQ ID NO: 390; and iv) SEQ ID NO:859.
15) In some cases, a cassette comprises, in order from 5′ to 3′: i) SEQ ID NO:854; ii) SEQ ID NO: 391; iii) a sequence that is complementary to SEQ ID NO: 391, where the sequence can comprise from 0 to 10 mismatches (non-complementary nucleotides) to SEQ ID NO: 391; and iv) SEQ ID NO:855. In some cases, a cassette comprises, in order from 5′ to 3′: i) SEQ ID NO:856; ii) SEQ ID NO: 391; iii) a sequence that is complementary to SEQ ID NO: 391, where the sequence can comprise from 0 to 10 mismatches (non-complementary nucleotides) to SEQ ID NO: 391; and iv) SEQ ID NO:857. In some cases, a cassette comprises, in order from 5′ to 3′: i) SEQ ID NO:858; ii) SEQ ID NO: 391; iii) a sequence that is complementary to SEQ ID NO: 391, where the sequence can comprise from 0 to 10 mismatches (non-complementary nucleotides) to SEQ ID NO: 391; and iv) SEQ ID NO:859.
16) In some cases, a cassette comprises, in order from 5′ to 3′: i) SEQ ID NO:854; ii) SEQ ID NO:392; iii) a sequence that is complementary to SEQ ID NO: 392, where the sequence can comprise from 0 to 10 mismatches (non-complementary nucleotides) to SEQ ID NO: 392; and iv) SEQ ID NO:855. In some cases, a cassette comprises, in order from 5′ to 3′: i) SEQ ID NO:856; ii) SEQ ID NO: 392; iii) a sequence that is complementary to SEQ ID NO: 392, where the sequence can comprise from 0 to 10 mismatches (non-complementary nucleotides) to SEQ ID NO: 392; and iv) SEQ ID NO:857. In some cases, a cassette comprises, in order from 5′ to 3′: i) SEQ ID NO:858; ii) SEQ ID NO: 392; iii) a sequence that is complementary to SEQ ID NO: 392, where the sequence can comprise from 0 to 10 mismatches (non-complementary nucleotides) to SEQ ID NO: 392; and iv) SEQ ID NO:859.
17) In some cases, a cassette comprises, in order from 5′ to 3′: i) SEQ ID NO:854; ii) SEQ ID NO:393; iii) a sequence that is complementary to SEQ ID NO: 393, where the sequence can comprise from 0 to 10 mismatches (non-complementary nucleotides) to SEQ ID NO: 393; and iv) SEQ ID NO:855. In some cases, a cassette comprises, in order from 5′ to 3′: i) SEQ ID NO:856; ii) SEQ ID NO: 393; iii) a sequence that is complementary to SEQ ID NO: 393, where the sequence can comprise from 0 to 10 mismatches (non-complementary nucleotides) to SEQ ID NO: 393; and iv) SEQ ID NO:857. In some cases, a cassette comprises, in order from 5′ to 3′: i) SEQ ID NO:858; ii) SEQ ID NO: 393; iii) a sequence that is complementary to SEQ ID NO: 393, where the sequence can comprise from 0 to 10 mismatches (non-complementary nucleotides) to SEQ ID NO: 393; and iv) SEQ ID NO:859.
18) In some cases, a cassette comprises, in order from 5′ to 3′: i) SEQ ID NO:854; ii) SEQ ID NO:394; iii) a sequence that is complementary to SEQ ID NO: 394, where the sequence can comprise from 0 to 10 mismatches (non-complementary nucleotides) to SEQ ID NO: 394; and iv) SEQ ID NO:855. In some cases, a cassette comprises, in order from 5′ to 3′: i) SEQ ID NO:856; ii) SEQ ID NO: 394; iii) a sequence that is complementary to SEQ ID NO: 394, where the sequence can comprise from 0 to 10 mismatches (non-complementary nucleotides) to SEQ ID NO: 394; and iv) SEQ ID NO:857. In some cases, a cassette comprises, in order from 5′ to 3′: i) SEQ ID NO:858; ii) SEQ ID NO: 394; iii) a sequence that is complementary to SEQ ID NO: 394, where the sequence can comprise from 0 to 10 mismatches (non-complementary nucleotides) to SEQ ID NO: 394; and iv) SEQ ID NO:859.
19) In some cases, a cassette comprises, in order from 5′ to 3′: i) SEQ ID NO:854; ii) SEQ ID NO:395; iii) a sequence that is complementary to SEQ ID NO: 395, where the sequence can comprise from 0 to 10 mismatches (non-complementary nucleotides) to SEQ ID NO: 395; and iv) SEQ ID NO:855. In some cases, a cassette comprises, in order from 5′ to 3′: i) SEQ ID NO:856; ii) SEQ ID NO: 395; iii) a sequence that is complementary to SEQ ID NO: 395, where the sequence can comprise from 0 to 10 mismatches (non-complementary nucleotides) to SEQ ID NO: 395; and iv) SEQ ID NO:857. In some cases, a cassette comprises, in order from 5′ to 3′: i) SEQ ID NO:858; ii) SEQ ID NO: 395; iii) a sequence that is complementary to SEQ ID NO: 395, where the sequence can comprise from 0 to 10 mismatches (non-complementary nucleotides) to SEQ ID NO: 395; and iv) SEQ ID NO:859.
20) In some cases, a cassette comprises, in order from 5′ to 3′: i) SEQ ID NO:854; ii) SEQ ID NO:396; iii) a sequence that is complementary to SEQ ID NO: 396, where the sequence can comprise from 0 to 10 mismatches (non-complementary nucleotides) to SEQ ID NO: 396; and iv) SEQ ID NO:855. In some cases, a cassette comprises, in order from 5′ to 3′: i) SEQ ID NO:856; ii) SEQ ID NO: 396; iii) a sequence that is complementary to SEQ ID NO: 396, where the sequence can comprise from 0 to 10 mismatches (non-complementary nucleotides) to SEQ ID NO: 396; and iv) SEQ ID NO:857. In some cases, a cassette comprises, in order from 5′ to 3′: i) SEQ ID NO:858; ii) SEQ ID NO: 396; iii) a sequence that is complementary to SEQ ID NO: 396, where the sequence can comprise from 0 to 10 mismatches (non-complementary nucleotides) to SEQ ID NO: 396; and iv) SEQ ID NO:859.
21) In some cases, a cassette comprises, in order from 5′ to 3′: i) SEQ ID NO:854; ii) SEQ ID NO:397; iii) a sequence that is complementary to SEQ ID NO: 397, where the sequence can comprise from 0 to 10 mismatches (non-complementary nucleotides) to SEQ ID NO: 397; and iv) SEQ ID NO:855. In some cases, a cassette comprises, in order from 5′ to 3′: i) SEQ ID NO:856; ii) SEQ ID NO: 397; iii) a sequence that is complementary to SEQ ID NO: 397, where the sequence can comprise from 0 to 10 mismatches (non-complementary nucleotides) to SEQ ID NO: 397; and iv) SEQ ID NO:857. In some cases, a cassette comprises, in order from 5′ to 3′: i) SEQ ID NO:858; ii) SEQ ID NO: 397; iii) a sequence that is complementary to SEQ ID NO: 397, where the sequence can comprise from 0 to 10 mismatches (non-complementary nucleotides) to SEQ ID NO: 397; and iv) SEQ ID NO:859.
22) In some cases, a cassette comprises, in order from 5′ to 3′: i) SEQ ID NO:854; ii) SEQ ID NO:398; iii) a sequence that is complementary to SEQ ID NO: 398, where the sequence can comprise from 0 to 10 mismatches (non-complementary nucleotides) to SEQ ID NO: 398; and iv) SEQ ID NO:855. In some cases, a cassette comprises, in order from 5′ to 3′: i) SEQ ID NO:856; ii) SEQ ID NO: 398; iii) a sequence that is complementary to SEQ ID NO: 398, where the sequence can comprise from 0 to 10 mismatches (non-complementary nucleotides) to SEQ ID NO: 398; and iv) SEQ ID NO:857. In some cases, a cassette comprises, in order from 5′ to 3′: i) SEQ ID NO:858; ii) SEQ ID NO: 398; iii) a sequence that is complementary to SEQ ID NO: 398, where the sequence can comprise from 0 to 10 mismatches (non-complementary nucleotides) to SEQ ID NO: 398; and iv) SEQ ID NO:859.
23) In some cases, a cassette comprises, in order from 5′ to 3′: i) SEQ ID NO:854; ii) SEQ ID NO:399; iii) a sequence that is complementary to SEQ ID NO: 399, where the sequence can comprise from 0 to 10 mismatches (non-complementary nucleotides) to SEQ ID NO: 399; and iv) SEQ ID NO:855. In some cases, a cassette comprises, in order from 5′ to 3′: i) SEQ ID NO:856; ii) SEQ ID NO: 399; iii) a sequence that is complementary to SEQ ID NO: 399, where the sequence can comprise from 0 to 10 mismatches (non-complementary nucleotides) to SEQ ID NO: 399; and iv) SEQ ID NO:857. In some cases, a cassette comprises, in order from 5′ to 3′: i) SEQ ID NO:858; ii) SEQ ID NO: 399; iii) a sequence that is complementary to SEQ ID NO: 399, where the sequence can comprise from 0 to 10 mismatches (non-complementary nucleotides) to SEQ ID NO: 399; and iv) SEQ ID NO:859.
24) In some cases, a cassette comprises, in order from 5′ to 3′: i) SEQ ID NO:854; ii) SEQ ID NO:400; iii) a sequence that is complementary to SEQ ID NO: 400, where the sequence can comprise from 0 to 10 mismatches (non-complementary nucleotides) to SEQ ID NO: 400; and iv) SEQ ID NO:855. In some cases, a cassette comprises, in order from 5′ to 3′: i) SEQ ID NO:856; ii) SEQ ID NO: 400; iii) a sequence that is complementary to SEQ ID NO: 400, where the sequence can comprise from 0 to 10 mismatches (non-complementary nucleotides) to SEQ ID NO: 400; and iv) SEQ ID NO:857. In some cases, a cassette comprises, in order from 5′ to 3′: i) SEQ ID NO:858; ii) SEQ ID NO: 400; iii) a sequence that is complementary to SEQ ID NO: 400, where the sequence can comprise from 0 to 10 mismatches (non-complementary nucleotides) to SEQ ID NO: 400; and iv) SEQ ID NO:859.
25) In some cases, a cassette comprises, in order from 5′ to 3′: i) SEQ ID NO:854; ii) SEQ ID NO:401; iii) a sequence that is complementary to SEQ ID NO: 401, where the sequence can comprise from 0 to 10 mismatches (non-complementary nucleotides) to SEQ ID NO: 401; and iv) SEQ ID NO:855. In some cases, a cassette comprises, in order from 5′ to 3′: i) SEQ ID NO:856; ii) SEQ ID NO: 401; iii) a sequence that is complementary to SEQ ID NO: 401, where the sequence can comprise from 0 to 10 mismatches (non-complementary nucleotides) to SEQ ID NO: 401; and iv) SEQ ID NO:857. In some cases, a cassette comprises, in order from 5′ to 3′: i) SEQ ID NO:858; ii) SEQ ID NO: 401; iii) a sequence that is complementary to SEQ ID NO: 401, where the sequence can comprise from 0 to 10 mismatches (non-complementary nucleotides) to SEQ ID NO: 401; and iv) SEQ ID NO:859.
26) In some cases, a cassette comprises, in order from 5′ to 3′: i) SEQ ID NO:854; ii) SEQ ID NO:402; iii) a sequence that is complementary to SEQ ID NO: 402, where the sequence can comprise from 0 to 10 mismatches (non-complementary nucleotides) to SEQ ID NO: 402; and iv) SEQ ID NO:855. In some cases, a cassette comprises, in order from 5′ to 3′: i) SEQ ID NO:856; ii) SEQ ID NO: 402; iii) a sequence that is complementary to SEQ ID NO: 402, where the sequence can comprise from 0 to 10 mismatches (non-complementary nucleotides) to SEQ ID NO: 402; and iv) SEQ ID NO:857. In some cases, a cassette comprises, in order from 5′ to 3′: i) SEQ ID NO:858; ii) SEQ ID NO: 402; iii) a sequence that is complementary to SEQ ID NO: 402, where the sequence can comprise from 0 to 10 mismatches (non-complementary nucleotides) to SEQ ID NO: 402; and iv) SEQ ID NO:859.
27) In some cases, a cassette comprises, in order from 5′ to 3′: i) SEQ ID NO:854; ii) SEQ ID NO:403; iii) a sequence that is complementary to SEQ ID NO: 403, where the sequence can comprise from 0 to 10 mismatches (non-complementary nucleotides) to SEQ ID NO: 403; and iv) SEQ ID NO:855. In some cases, a cassette comprises, in order from 5′ to 3′: i) SEQ ID NO:856; ii) SEQ ID NO: 403; iii) a sequence that is complementary to SEQ ID NO: 403, where the sequence can comprise from 0 to 10 mismatches (non-complementary nucleotides) to SEQ ID NO: 403; and iv) SEQ ID NO:857. In some cases, a cassette comprises, in order from 5′ to 3′: i) SEQ ID NO:858; ii) SEQ ID NO: 403; iii) a sequence that is complementary to SEQ ID NO: 403, where the sequence can comprise from 0 to 10 mismatches (non-complementary nucleotides) to SEQ ID NO: 403; and iv) SEQ ID NO:859.
28) In some cases, a cassette comprises, in order from 5′ to 3′: i) SEQ ID NO:854; ii) SEQ ID NO:404; iii) a sequence that is complementary to SEQ ID NO: 404, where the sequence can comprise from 0 to 10 mismatches (non-complementary nucleotides) to SEQ ID NO: 404; and iv) SEQ ID NO:855. In some cases, a cassette comprises, in order from 5′ to 3′: i) SEQ ID NO:856; ii) SEQ ID NO: 404; iii) a sequence that is complementary to SEQ ID NO: 404, where the sequence can comprise from 0 to 10 mismatches (non-complementary nucleotides) to SEQ ID NO: 404; and iv) SEQ ID NO:857. In some cases, a cassette comprises, in order from 5′ to 3′: i) SEQ ID NO:858; ii) SEQ ID NO: 404; iii) a sequence that is complementary to SEQ ID NO: 404, where the sequence can comprise from 0 to 10 mismatches (non-complementary nucleotides) to SEQ ID NO: 404; and iv) SEQ ID NO:859.
29) In some cases, a cassette comprises, in order from 5′ to 3′: i) SEQ ID NO:854; ii) SEQ ID NO:405; iii) a sequence that is complementary to SEQ ID NO: 405, where the sequence can comprise from 0 to 10 mismatches (non-complementary nucleotides) to SEQ ID NO: 405; and iv) SEQ ID NO:855. In some cases, a cassette comprises, in order from 5′ to 3′: i) SEQ ID NO:856; ii) SEQ ID NO: 405; iii) a sequence that is complementary to SEQ ID NO: 405, where the sequence can comprise from 0 to 10 mismatches (non-complementary nucleotides) to SEQ ID NO: 405; and iv) SEQ ID NO:857. In some cases, a cassette comprises, in order from 5′ to 3′: i) SEQ ID NO:858; ii) SEQ ID NO: 405; iii) a sequence that is complementary to SEQ ID NO: 405, where the sequence can comprise from 0 to 10 mismatches (non-complementary nucleotides) to SEQ ID NO: 405; and iv) SEQ ID NO:859.
30) In some cases, a cassette comprises, in order from 5′ to 3′: i) SEQ ID NO:854; ii) SEQ ID NO:406; iii) a sequence that is complementary to SEQ ID NO: 406, where the sequence can comprise from 0 to 10 mismatches (non-complementary nucleotides) to SEQ ID NO: 406; and iv) SEQ ID NO:855. In some cases, a cassette comprises, in order from 5′ to 3′: i) SEQ ID NO:856; ii) SEQ ID NO: 406; iii) a sequence that is complementary to SEQ ID NO: 406, where the sequence can comprise from 0 to 10 mismatches (non-complementary nucleotides) to SEQ ID NO: 406; and iv) SEQ ID NO:857. In some cases, a cassette comprises, in order from 5′ to 3′: i) SEQ ID NO:858; ii) SEQ ID NO: 406; iii) a sequence that is complementary to SEQ ID NO: 406, where the sequence can comprise from 0 to 10 mismatches (non-complementary nucleotides) to SEQ ID NO: 406; and iv) SEQ ID NO:859.
31) In some cases, a cassette comprises, in order from 5′ to 3′: i) SEQ ID NO:854; ii) SEQ ID NO:407; iii) a sequence that is complementary to SEQ ID NO: 407, where the sequence can comprise from 0 to 10 mismatches (non-complementary nucleotides) to SEQ ID NO: 407; and iv) SEQ ID NO:855. In some cases, a cassette comprises, in order from 5′ to 3′: i) SEQ ID NO:856; ii) SEQ ID NO: 407; iii) a sequence that is complementary to SEQ ID NO: 407, where the sequence can comprise from 0 to 10 mismatches (non-complementary nucleotides) to SEQ ID NO: 407; and iv) SEQ ID NO:857. In some cases, a cassette comprises, in order from 5′ to 3′: i) SEQ ID NO:858; ii) SEQ ID NO: 407; iii) a sequence that is complementary to SEQ ID NO: 407, where the sequence can comprise from 0 to 10 mismatches (non-complementary nucleotides) to SEQ ID NO: 407; and iv) SEQ ID NO:859.
32) In some cases, a cassette comprises, in order from 5′ to 3′: i) SEQ ID NO:854; ii) SEQ ID NO:408; iii) a sequence that is complementary to SEQ ID NO: 408, where the sequence can comprise from 0 to 10 mismatches (non-complementary nucleotides) to SEQ ID NO: 408; and iv) SEQ ID NO:855. In some cases, a cassette comprises, in order from 5′ to 3′: i) SEQ ID NO:856; ii) SEQ ID NO: 408; iii) a sequence that is complementary to SEQ ID NO: 408, where the sequence can comprise from 0 to 10 mismatches (non-complementary nucleotides) to SEQ ID NO: 408; and iv) SEQ ID NO:857. In some cases, a cassette comprises, in order from 5′ to 3′: i) SEQ ID NO:858; ii) SEQ ID NO: 408; iii) a sequence that is complementary to SEQ ID NO: 408, where the sequence can comprise from 0 to 10 mismatches (non-complementary nucleotides) to SEQ ID NO: 408; and iv) SEQ ID NO:859.
33) In some cases, a cassette comprises, in order from 5′ to 3′: i) SEQ ID NO:854; ii) SEQ ID NO:409; iii) a sequence that is complementary to SEQ ID NO: 409, where the sequence can comprise from 0 to 10 mismatches (non-complementary nucleotides) to SEQ ID NO: 409; and iv) SEQ ID NO:855. In some cases, a cassette comprises, in order from 5′ to 3′: i) SEQ ID NO:856; ii) SEQ ID NO: 409; iii) a sequence that is complementary to SEQ ID NO: 409, where the sequence can comprise from 0 to 10 mismatches (non-complementary nucleotides) to SEQ ID NO: 409; and iv) SEQ ID NO:857. In some cases, a cassette comprises, in order from 5′ to 3′: i) SEQ ID NO:858; ii) SEQ ID NO: 409; iii) a sequence that is complementary to SEQ ID NO: 409, where the sequence can comprise from 0 to 10 mismatches (non-complementary nucleotides) to SEQ ID NO: 409; and iv) SEQ ID NO:859.
34) In some cases, a cassette comprises, in order from 5′ to 3′: i) SEQ ID NO:854; ii) SEQ ID NO:410; iii) a sequence that is complementary to SEQ ID NO: 410, where the sequence can comprise from 0 to 10 mismatches (non-complementary nucleotides) to SEQ ID NO: 410; and iv) SEQ ID NO:855. In some cases, a cassette comprises, in order from 5′ to 3′: i) SEQ ID NO:856; ii) SEQ ID NO: 410; iii) a sequence that is complementary to SEQ ID NO: 410, where the sequence can comprise from 0 to 10 mismatches (non-complementary nucleotides) to SEQ ID NO: 410; and iv) SEQ ID NO:857. In some cases, a cassette comprises, in order from 5′ to 3′: i) SEQ ID NO:858; ii) SEQ ID NO: 410; iii) a sequence that is complementary to SEQ ID NO: 410, where the sequence can comprise from 0 to 10 mismatches (non-complementary nucleotides) to SEQ ID NO: 410; and iv) SEQ ID NO:859.
35) In some cases, a cassette comprises, in order from 5′ to 3′: i) SEQ ID NO:854; ii) SEQ ID NO:411; iii) a sequence that is complementary to SEQ ID NO: 411, where the sequence can comprise from 0 to 10 mismatches (non-complementary nucleotides) to SEQ ID NO: 411; and iv) SEQ ID NO:855. In some cases, a cassette comprises, in order from 5′ to 3′: i) SEQ ID NO:856; ii) SEQ ID NO: 411; iii) a sequence that is complementary to SEQ ID NO: 411, where the sequence can comprise from 0 to 10 mismatches (non-complementary nucleotides) to SEQ ID NO: 411; and iv) SEQ ID NO:857. In some cases, a cassette comprises, in order from 5′ to 3′: i) SEQ ID NO:858; ii) SEQ ID NO: 411; iii) a sequence that is complementary to SEQ ID NO: 411, where the sequence can comprise from 0 to 10 mismatches (non-complementary nucleotides) to SEQ ID NO: 411; and iv) SEQ ID NO:859.
36) In some cases, a cassette comprises, in order from 5′ to 3′: i) SEQ ID NO:854; ii) SEQ ID NO:412; iii) a sequence that is complementary to SEQ ID NO: 412, where the sequence can comprise from 0 to 10 mismatches (non-complementary nucleotides) to SEQ ID NO: 412; and iv) SEQ ID NO:855. In some cases, a cassette comprises, in order from 5′ to 3′: i) SEQ ID NO:856; ii) SEQ ID NO: 412; iii) a sequence that is complementary to SEQ ID NO: 412, where the sequence can comprise from 0 to 10 mismatches (non-complementary nucleotides) to SEQ ID NO: 412; and iv) SEQ ID NO:857. In some cases, a cassette comprises, in order from 5′ to 3′: i) SEQ ID NO:858; ii) SEQ ID NO: 412; iii) a sequence that is complementary to SEQ ID NO: 412, where the sequence can comprise from 0 to 10 mismatches (non-complementary nucleotides) to SEQ ID NO: 412; and iv) SEQ ID NO:859.
37) In some cases, a cassette comprises, in order from 5′ to 3′: i) SEQ ID NO:854; ii) SEQ ID NO:413; iii) a sequence that is complementary to SEQ ID NO: 413, where the sequence can comprise from 0 to 10 mismatches (non-complementary nucleotides) to SEQ ID NO: 413; and iv) SEQ ID NO:855. In some cases, a cassette comprises, in order from 5′ to 3′: i) SEQ ID NO:856; ii) SEQ ID NO: 413; iii) a sequence that is complementary to SEQ ID NO: 413, where the sequence can comprise from 0 to 10 mismatches (non-complementary nucleotides) to SEQ ID NO: 413; and iv) SEQ ID NO:857. In some cases, a cassette comprises, in order from 5′ to 3′: i) SEQ ID NO:858; ii) SEQ ID NO: 413; iii) a sequence that is complementary to SEQ ID NO: 413, where the sequence can comprise from 0 to 10 mismatches (non-complementary nucleotides) to SEQ ID NO: 413; and iv) SEQ ID NO:859.
38) In some cases, a cassette comprises, in order from 5′ to 3′: i) SEQ ID NO:854; ii) SEQ ID NO:414; iii) a sequence that is complementary to SEQ ID NO: 414, where the sequence can comprise from 0 to 10 mismatches (non-complementary nucleotides) to SEQ ID NO: 414; and iv) SEQ ID NO:855. In some cases, a cassette comprises, in order from 5′ to 3′: i) SEQ ID NO:856; ii) SEQ ID NO: 414; iii) a sequence that is complementary to SEQ ID NO: 414, where the sequence can comprise from 0 to 10 mismatches (non-complementary nucleotides) to SEQ ID NO: 414; and iv) SEQ ID NO:857. In some cases, a cassette comprises, in order from 5′ to 3′: i) SEQ ID NO:858; ii) SEQ ID NO: 414; iii) a sequence that is complementary to SEQ ID NO: 414, where the sequence can comprise from 0 to 10 mismatches (non-complementary nucleotides) to SEQ ID NO: 414; and iv) SEQ ID NO:859.
39) In some cases, a cassette comprises, in order from 5′ to 3′: i) SEQ ID NO:854; ii) SEQ ID NO:415; iii) a sequence that is complementary to SEQ ID NO: 415, where the sequence can comprise from 0 to 10 mismatches (non-complementary nucleotides) to SEQ ID NO: 415; and iv) SEQ ID NO:855. In some cases, a cassette comprises, in order from 5′ to 3′: i) SEQ ID NO:856; ii) SEQ ID NO: 415; iii) a sequence that is complementary to SEQ ID NO: 415, where the sequence can comprise from 0 to 10 mismatches (non-complementary nucleotides) to SEQ ID NO: 415; and iv) SEQ ID NO:857. In some cases, a cassette comprises, in order from 5′ to 3′: i) SEQ ID NO:858; ii) SEQ ID NO: 415; iii) a sequence that is complementary to SEQ ID NO: 415, where the sequence can comprise from 0 to 10 mismatches (non-complementary nucleotides) to SEQ ID NO: 415; and iv) SEQ ID NO:859.
40) In some cases, a cassette comprises, in order from 5′ to 3′: i) SEQ ID NO:854; ii) SEQ ID NO:416; iii) a sequence that is complementary to SEQ ID NO: 416, where the sequence can comprise from 0 to 10 mismatches (non-complementary nucleotides) to SEQ ID NO: 416; and iv) SEQ ID NO:855. In some cases, a cassette comprises, in order from 5′ to 3′: i) SEQ ID NO:856; ii) SEQ ID NO: 416; iii) a sequence that is complementary to SEQ ID NO: 416, where the sequence can comprise from 0 to 10 mismatches (non-complementary nucleotides) to SEQ ID NO: 416; and iv) SEQ ID NO:857. In some cases, a cassette comprises, in order from 5′ to 3′: i) SEQ ID NO:858; ii) SEQ ID NO: 416; iii) a sequence that is complementary to SEQ ID NO: 416, where the sequence can comprise from 0 to 10 mismatches (non-complementary nucleotides) to SEQ ID NO: 416; and iv) SEQ ID NO:859.
41) In some cases, a cassette comprises, in order from 5′ to 3′: i) SEQ ID NO:854; ii) SEQ ID NO:417; iii) a sequence that is complementary to SEQ ID NO: 417, where the sequence can comprise from 0 to 10 mismatches (non-complementary nucleotides) to SEQ ID NO: 417; and iv) SEQ ID NO:855. In some cases, a cassette comprises, in order from 5′ to 3′: i) SEQ ID NO:856; ii) SEQ ID NO: 417; iii) a sequence that is complementary to SEQ ID NO: 417, where the sequence can comprise from 0 to 10 mismatches (non-complementary nucleotides) to SEQ ID NO: 417; and iv) SEQ ID NO:857. In some cases, a cassette comprises, in order from 5′ to 3′: i) SEQ ID NO:858; ii) SEQ ID NO: 417; iii) a sequence that is complementary to SEQ ID NO: 417, where the sequence can comprise from 0 to 10 mismatches (non-complementary nucleotides) to SEQ ID NO: 417; and iv) SEQ ID NO:859.
42) In some cases, a cassette comprises, in order from 5′ to 3′: i) SEQ ID NO:854; ii) SEQ ID NO:418; iii) a sequence that is complementary to SEQ ID NO: 418, where the sequence can comprise from 0 to 10 mismatches (non-complementary nucleotides) to SEQ ID NO: 418; and iv) SEQ ID NO:855. In some cases, a cassette comprises, in order from 5′ to 3′: i) SEQ ID NO:856; ii) SEQ ID NO: 418; iii) a sequence that is complementary to SEQ ID NO: 418, where the sequence can comprise from 0 to 10 mismatches (non-complementary nucleotides) to SEQ ID NO: 418; and iv) SEQ ID NO:857. In some cases, a cassette comprises, in order from 5′ to 3′: i) SEQ ID NO:858; ii) SEQ ID NO: 418; iii) a sequence that is complementary to SEQ ID NO: 418, where the sequence can comprise from 0 to 10 mismatches (non-complementary nucleotides) to SEQ ID NO: 418; and iv) SEQ ID NO:859.
43) In some cases, a cassette comprises, in order from 5′ to 3′: i) SEQ ID NO:854; ii) SEQ ID NO:419; iii) a sequence that is complementary to SEQ ID NO: 419, where the sequence can comprise from 0 to 10 mismatches (non-complementary nucleotides) to SEQ ID NO: 419; and iv) SEQ ID NO:855. In some cases, a cassette comprises, in order from 5′ to 3′: i) SEQ ID NO:856; ii) SEQ ID NO: 419; iii) a sequence that is complementary to SEQ ID NO: 419, where the sequence can comprise from 0 to 10 mismatches (non-complementary nucleotides) to SEQ ID NO: 419; and iv) SEQ ID NO:857. In some cases, a cassette comprises, in order from 5′ to 3′: i) SEQ ID NO:858; ii) SEQ ID NO: 419; iii) a sequence that is complementary to SEQ ID NO: 419, where the sequence can comprise from 0 to 10 mismatches (non-complementary nucleotides) to SEQ ID NO: 419; and iv) SEQ ID NO:859.
44) In some cases, a cassette comprises, in order from 5′ to 3′: i) SEQ ID NO:854; ii) SEQ ID NO:420; iii) a sequence that is complementary to SEQ ID NO: 420, where the sequence can comprise from 0 to 10 mismatches (non-complementary nucleotides) to SEQ ID NO: 420; and iv) SEQ ID NO:855. In some cases, a cassette comprises, in order from 5′ to 3′: i) SEQ ID NO:856; ii) SEQ ID NO: 420; iii) a sequence that is complementary to SEQ ID NO: 420, where the sequence can comprise from 0 to 10 mismatches (non-complementary nucleotides) to SEQ ID NO: 420; and iv) SEQ ID NO:857. In some cases, a cassette comprises, in order from 5′ to 3′: i) SEQ ID NO:858; ii) SEQ ID NO: 420; iii) a sequence that is complementary to SEQ ID NO: 420, where the sequence can comprise from 0 to 10 mismatches (non-complementary nucleotides) to SEQ ID NO: 420; and iv) SEQ ID NO:859.
45) In some cases, a cassette comprises, in order from 5′ to 3′: i) SEQ ID NO:854; ii) SEQ ID NO:421; iii) a sequence that is complementary to SEQ ID NO: 421, where the sequence can comprise from 0 to 10 mismatches (non-complementary nucleotides) to SEQ ID NO: 421; and iv) SEQ ID NO:855. In some cases, a cassette comprises, in order from 5′ to 3′: i) SEQ ID NO:856; ii) SEQ ID NO: 421; iii) a sequence that is complementary to SEQ ID NO: 421, where the sequence can comprise from 0 to 10 mismatches (non-complementary nucleotides) to SEQ ID NO: 421; and iv) SEQ ID NO:857. In some cases, a cassette comprises, in order from 5′ to 3′: i) SEQ ID NO:858; ii) SEQ ID NO: 421; iii) a sequence that is complementary to SEQ ID NO: 421, where the sequence can comprise from 0 to 10 mismatches (non-complementary nucleotides) to SEQ ID NO: 421; and iv) SEQ ID NO:859.
46) In some cases, a cassette comprises, in order from 5′ to 3′: i) SEQ ID NO:854; ii) SEQ ID NO:422; iii) a sequence that is complementary to SEQ ID NO: 422, where the sequence can comprise from 0 to 10 mismatches (non-complementary nucleotides) to SEQ ID NO: 422; and iv) SEQ ID NO:855. In some cases, a cassette comprises, in order from 5′ to 3′: i) SEQ ID NO:856; ii) SEQ ID NO: 422; iii) a sequence that is complementary to SEQ ID NO: 422, where the sequence can comprise from 0 to 10 mismatches (non-complementary nucleotides) to SEQ ID NO: 422; and iv) SEQ ID NO:857. In some cases, a cassette comprises, in order from 5′ to 3′: i) SEQ ID NO:858; ii) SEQ ID NO: 422; iii) a sequence that is complementary to SEQ ID NO: 422, where the sequence can comprise from 0 to 10 mismatches (non-complementary nucleotides) to SEQ ID NO: 422; and iv) SEQ ID NO:859.
47) In some cases, a cassette comprises, in order from 5′ to 3′: i) SEQ ID NO:854; ii) SEQ ID NO:423; iii) a sequence that is complementary to SEQ ID NO: 423, where the sequence can comprise from 0 to 10 mismatches (non-complementary nucleotides) to SEQ ID NO: 423; and iv) SEQ ID NO:855. In some cases, a cassette comprises, in order from 5′ to 3′: i) SEQ ID NO:856; ii) SEQ ID NO: 423; iii) a sequence that is complementary to SEQ ID NO: 423, where the sequence can comprise from 0 to 10 mismatches (non-complementary nucleotides) to SEQ ID NO: 423; and iv) SEQ ID NO:857. In some cases, a cassette comprises, in order from 5′ to 3′: i) SEQ ID NO:858; ii) SEQ ID NO: 423; iii) a sequence that is complementary to SEQ ID NO: 423, where the sequence can comprise from 0 to 10 mismatches (non-complementary nucleotides) to SEQ ID NO: 423; and iv) SEQ ID NO:859.
48) In some cases, a cassette comprises, in order from 5′ to 3′: i) SEQ ID NO:854; ii) SEQ ID NO:424; iii) a sequence that is complementary to SEQ ID NO: 424, where the sequence can comprise from 0 to 10 mismatches (non-complementary nucleotides) to SEQ ID NO: 424; and iv) SEQ ID NO:855. In some cases, a cassette comprises, in order from 5′ to 3′: i) SEQ ID NO:856; ii) SEQ ID NO: 424; iii) a sequence that is complementary to SEQ ID NO: 424, where the sequence can comprise from 0 to 10 mismatches (non-complementary nucleotides) to SEQ ID NO: 424; and iv) SEQ ID NO:857. In some cases, a cassette comprises, in order from 5′ to 3′: i) SEQ ID NO:858; ii) SEQ ID NO: 424; iii) a sequence that is complementary to SEQ ID NO: 424, where the sequence can comprise from 0 to 10 mismatches (non-complementary nucleotides) to SEQ ID NO: 424; and iv) SEQ ID NO:859.
49) In some cases, a cassette comprises, in order from 5′ to 3′: i) SEQ ID NO:854; ii) SEQ ID NO:425; iii) a sequence that is complementary to SEQ ID NO: 425, where the sequence can comprise from 0 to 10 mismatches (non-complementary nucleotides) to SEQ ID NO: 425; and iv) SEQ ID NO:855. In some cases, a cassette comprises, in order from 5′ to 3′: i) SEQ ID NO:856; ii) SEQ ID NO: 425; iii) a sequence that is complementary to SEQ ID NO: 425, where the sequence can comprise from 0 to 10 mismatches (non-complementary nucleotides) to SEQ ID NO: 425; and iv) SEQ ID NO:857. In some cases, a cassette comprises, in order from 5′ to 3′: i) SEQ ID NO:858; ii) SEQ ID NO: 425; iii) a sequence that is complementary to SEQ ID NO: 425, where the sequence can comprise from 0 to 10 mismatches (non-complementary nucleotides) to SEQ ID NO: 425; and iv) SEQ ID NO:859.
50) In some cases, a cassette comprises, in order from 5′ to 3′: i) SEQ ID NO:854; ii) SEQ ID NO:426; iii) a sequence that is complementary to SEQ ID NO: 426, where the sequence can comprise from 0 to 10 mismatches (non-complementary nucleotides) to SEQ ID NO: 426; and iv) SEQ ID NO:855. In some cases, a cassette comprises, in order from 5′ to 3′: i) SEQ ID NO:856; ii) SEQ ID NO: 426; iii) a sequence that is complementary to SEQ ID NO: 426, where the sequence can comprise from 0 to 10 mismatches (non-complementary nucleotides) to SEQ ID NO: 426; and iv) SEQ ID NO:857. In some cases, a cassette comprises, in order from 5′ to 3′: i) SEQ ID NO:858; ii) SEQ ID NO: 426; iii) a sequence that is complementary to SEQ ID NO: 426, where the sequence can comprise from 0 to 10 mismatches (non-complementary nucleotides) to SEQ ID NO: 426; and iv) SEQ ID NO:859.
51) In some cases, a cassette comprises, in order from 5′ to 3′: i) SEQ ID NO:854; ii) SEQ ID NO:427; iii) a sequence that is complementary to SEQ ID NO: 427, where the sequence can comprise from 0 to 10 mismatches (non-complementary nucleotides) to SEQ ID NO: 427; and iv) SEQ ID NO:855. In some cases, a cassette comprises, in order from 5′ to 3′: i) SEQ ID NO:856; ii) SEQ ID NO: 427; iii) a sequence that is complementary to SEQ ID NO: 427, where the sequence can comprise from 0 to 10 mismatches (non-complementary nucleotides) to SEQ ID NO: 427; and iv) SEQ ID NO:857. In some cases, a cassette comprises, in order from 5′ to 3′: i) SEQ ID NO:858; ii) SEQ ID NO: 427; iii) a sequence that is complementary to SEQ ID NO: 427, where the sequence can comprise from 0 to 10 mismatches (non-complementary nucleotides) to SEQ ID NO: 427; and iv) SEQ ID NO:859.
52) In some cases, a cassette comprises, in order from 5′ to 3′: i) SEQ ID NO:854; ii) SEQ ID NO:428; iii) a sequence that is complementary to SEQ ID NO: 428, where the sequence can comprise from 0 to 10 mismatches (non-complementary nucleotides) to SEQ ID NO: 428; and iv) SEQ ID NO:855. In some cases, a cassette comprises, in order from 5′ to 3′: i) SEQ ID NO:856; ii) SEQ ID NO: 428; iii) a sequence that is complementary to SEQ ID NO: 428, where the sequence can comprise from 0 to 10 mismatches (non-complementary nucleotides) to SEQ ID NO: 428; and iv) SEQ ID NO:857. In some cases, a cassette comprises, in order from 5′ to 3′: i) SEQ ID NO:858; ii) SEQ ID NO: 428; iii) a sequence that is complementary to SEQ ID NO: 428, where the sequence can comprise from 0 to 10 mismatches (non-complementary nucleotides) to SEQ ID NO: 428; and iv) SEQ ID NO:859.
53) In some cases, a cassette comprises, in order from 5′ to 3′: i) SEQ ID NO:854; ii) SEQ ID NO:429; iii) a sequence that is complementary to SEQ ID NO: 429, where the sequence can comprise from 0 to 10 mismatches (non-complementary nucleotides) to SEQ ID NO: 429; and iv) SEQ ID NO:855. In some cases, a cassette comprises, in order from 5′ to 3′: i) SEQ ID NO:856; ii) SEQ ID NO: 429; iii) a sequence that is complementary to SEQ ID NO: 429, where the sequence can comprise from 0 to 10 mismatches (non-complementary nucleotides) to SEQ ID NO: 429; and iv) SEQ ID NO:857. In some cases, a cassette comprises, in order from 5′ to 3′: i) SEQ ID NO:858; ii) SEQ ID NO: 429; iii) a sequence that is complementary to SEQ ID NO: 429, where the sequence can comprise from 0 to 10 mismatches (non-complementary nucleotides) to SEQ ID NO: 429; and iv) SEQ ID NO:859.
54) In some cases, a cassette comprises, in order from 5′ to 3′: i) SEQ ID NO:854; ii) SEQ ID NO:430; iii) a sequence that is complementary to SEQ ID NO: 430, where the sequence can comprise from 0 to 10 mismatches (non-complementary nucleotides) to SEQ ID NO: 430; and iv) SEQ ID NO:855. In some cases, a cassette comprises, in order from 5′ to 3′: i) SEQ ID NO:856; ii) SEQ ID NO: 430; iii) a sequence that is complementary to SEQ ID NO: 430, where the sequence can comprise from 0 to 10 mismatches (non-complementary nucleotides) to SEQ ID NO: 430; and iv) SEQ ID NO:857. In some cases, a cassette comprises, in order from 5′ to 3′: i) SEQ ID NO:858; ii) SEQ ID NO: 430; iii) a sequence that is complementary to SEQ ID NO: 430, where the sequence can comprise from 0 to 10 mismatches (non-complementary nucleotides) to SEQ ID NO: 430; and iv) SEQ ID NO:859.
55) In some cases, a cassette comprises, in order from 5′ to 3′: i) SEQ ID NO:854; ii) SEQ ID NO:431; iii) a sequence that is complementary to SEQ ID NO: 431, where the sequence can comprise from 0 to 10 mismatches (non-complementary nucleotides) to SEQ ID NO: 431; and iv) SEQ ID NO:855. In some cases, a cassette comprises, in order from 5′ to 3′: i) SEQ ID NO:856; ii) SEQ ID NO: 431; iii) a sequence that is complementary to SEQ ID NO: 431, where the sequence can comprise from 0 to 10 mismatches (non-complementary nucleotides) to SEQ ID NO: 431; and iv) SEQ ID NO:857. In some cases, a cassette comprises, in order from 5′ to 3′: i) SEQ ID NO:858; ii) SEQ ID NO: 431; iii) a sequence that is complementary to SEQ ID NO: 431, where the sequence can comprise from 0 to 10 mismatches (non-complementary nucleotides) to SEQ ID NO: 431; and iv) SEQ ID NO:859.
56) In some cases, a cassette comprises, in order from 5′ to 3′: i) SEQ ID NO:854; ii) SEQ ID NO:432; iii) a sequence that is complementary to SEQ ID NO: 432, where the sequence can comprise from 0 to 10 mismatches (non-complementary nucleotides) to SEQ ID NO: 432; and iv) SEQ ID NO:855. In some cases, a cassette comprises, in order from 5′ to 3′: i) SEQ ID NO:856; ii) SEQ ID NO: 432; iii) a sequence that is complementary to SEQ ID NO: 432, where the sequence can comprise from 0 to 10 mismatches (non-complementary nucleotides) to SEQ ID NO: 432; and iv) SEQ ID NO:857. In some cases, a cassette comprises, in order from 5′ to 3′: i) SEQ ID NO:858; ii) SEQ ID NO: 432; iii) a sequence that is complementary to SEQ ID NO: 432, where the sequence can comprise from 0 to 10 mismatches (non-complementary nucleotides) to SEQ ID NO: 432; and iv) SEQ ID NO:859.
57) In some cases, a cassette comprises, in order from 5′ to 3′: i) SEQ ID NO:854; ii) SEQ ID NO:433; iii) a sequence that is complementary to SEQ ID NO: 433, where the sequence can comprise from 0 to 10 mismatches (non-complementary nucleotides) to SEQ ID NO: 433; and iv) SEQ ID NO:855. In some cases, a cassette comprises, in order from 5′ to 3′: i) SEQ ID NO:856; ii) SEQ ID NO: 433; iii) a sequence that is complementary to SEQ ID NO: 433, where the sequence can comprise from 0 to 10 mismatches (non-complementary nucleotides) to SEQ ID NO: 433; and iv) SEQ ID NO:857. In some cases, a cassette comprises, in order from 5′ to 3′: i) SEQ ID NO:858; ii) SEQ ID NO: 433; iii) a sequence that is complementary to SEQ ID NO: 433, where the sequence can comprise from 0 to 10 mismatches (non-complementary nucleotides) to SEQ ID NO: 433; and iv) SEQ ID NO:859.
58) In some cases, a cassette comprises, in order from 5′ to 3′: i) SEQ ID NO:854; ii) SEQ ID NO:434; iii) a sequence that is complementary to SEQ ID NO: 434, where the sequence can comprise from 0 to 10 mismatches (non-complementary nucleotides) to SEQ ID NO: 434; and iv) SEQ ID NO:855. In some cases, a cassette comprises, in order from 5′ to 3′: i) SEQ ID NO:856; ii) SEQ ID NO: 434; iii) a sequence that is complementary to SEQ ID NO: 434, where the sequence can comprise from 0 to 10 mismatches (non-complementary nucleotides) to SEQ ID NO: 434; and iv) SEQ ID NO:857. In some cases, a cassette comprises, in order from 5′ to 3′: i) SEQ ID NO:858; ii) SEQ ID NO: 434; iii) a sequence that is complementary to SEQ ID NO: 434, where the sequence can comprise from 0 to 10 mismatches (non-complementary nucleotides) to SEQ ID NO: 434; and iv) SEQ ID NO:859.
59) In some cases, a cassette comprises, in order from 5′ to 3′: i) SEQ ID NO:854; ii) SEQ ID NO:435; iii) a sequence that is complementary to SEQ ID NO: 435, where the sequence can comprise from 0 to 10 mismatches (non-complementary nucleotides) to SEQ ID NO: 435; and iv) SEQ ID NO:855. In some cases, a cassette comprises, in order from 5′ to 3′: i) SEQ ID NO:856; ii) SEQ ID NO: 435; iii) a sequence that is complementary to SEQ ID NO: 435, where the sequence can comprise from 0 to 10 mismatches (non-complementary nucleotides) to SEQ ID NO: 435; and iv) SEQ ID NO:857. In some cases, a cassette comprises, in order from 5′ to 3′: i) SEQ ID NO:858; ii) SEQ ID NO: 435; iii) a sequence that is complementary to SEQ ID NO: 435, where the sequence can comprise from 0 to 10 mismatches (non-complementary nucleotides) to SEQ ID NO: 435; and iv) SEQ ID NO:859.
60) In some cases, a cassette comprises, in order from 5′ to 3′: i) SEQ ID NO:854; ii) SEQ ID NO:436; iii) a sequence that is complementary to SEQ ID NO: 436, where the sequence can comprise from 0 to 10 mismatches (non-complementary nucleotides) to SEQ ID NO: 436; and iv) SEQ ID NO:855. In some cases, a cassette comprises, in order from 5′ to 3′: i) SEQ ID NO:856; ii) SEQ ID NO: 436; iii) a sequence that is complementary to SEQ ID NO: 436, where the sequence can comprise from 0 to 10 mismatches (non-complementary nucleotides) to SEQ ID NO: 436; and iv) SEQ ID NO:857. In some cases, a cassette comprises, in order from 5′ to 3′: i) SEQ ID NO:858; ii) SEQ ID NO: 436; iii) a sequence that is complementary to SEQ ID NO: 436, where the sequence can comprise from 0 to 10 mismatches (non-complementary nucleotides) to SEQ ID NO: 436; and iv) SEQ ID NO:859.
61) In some cases, a cassette comprises, in order from 5′ to 3′: i) SEQ ID NO:854; ii) SEQ ID NO:437; iii) a sequence that is complementary to SEQ ID NO: 437, where the sequence can comprise from 0 to 10 mismatches (non-complementary nucleotides) to SEQ ID NO: 437; and iv) SEQ ID NO:855. In some cases, a cassette comprises, in order from 5′ to 3′: i) SEQ ID NO:856; ii) SEQ ID NO: 437; iii) a sequence that is complementary to SEQ ID NO: 437, where the sequence can comprise from 0 to 10 mismatches (non-complementary nucleotides) to SEQ ID NO: 437; and iv) SEQ ID NO:857. In some cases, a cassette comprises, in order from 5′ to 3′: i) SEQ ID NO:858; ii) SEQ ID NO: 437; iii) a sequence that is complementary to SEQ ID NO: 437, where the sequence can comprise from 0 to 10 mismatches (non-complementary nucleotides) to SEQ ID NO: 437; and iv) SEQ ID NO:859.
62) In some cases, a cassette comprises, in order from 5′ to 3′: i) SEQ ID NO:854; ii) SEQ ID NO:438; iii) a sequence that is complementary to SEQ ID NO: 438, where the sequence can comprise from 0 to 10 mismatches (non-complementary nucleotides) to SEQ ID NO: 438; and iv) SEQ ID NO:855. In some cases, a cassette comprises, in order from 5′ to 3′: i) SEQ ID NO:856; ii) SEQ ID NO: 438; iii) a sequence that is complementary to SEQ ID NO: 438, where the sequence can comprise from 0 to 10 mismatches (non-complementary nucleotides) to SEQ ID NO: 438; and iv) SEQ ID NO:857. In some cases, a cassette comprises, in order from 5′ to 3′: i) SEQ ID NO:858; ii) SEQ ID NO: 438; iii) a sequence that is complementary to SEQ ID NO: 438, where the sequence can comprise from 0 to 10 mismatches (non-complementary nucleotides) to SEQ ID NO: 438; and iv) SEQ ID NO:859.
63) In some cases, a cassette comprises, in order from 5′ to 3′: i) SEQ ID NO:854; ii) SEQ ID NO:439; iii) a sequence that is complementary to SEQ ID NO: 439, where the sequence can comprise from 0 to 10 mismatches (non-complementary nucleotides) to SEQ ID NO: 439; and iv) SEQ ID NO:855. In some cases, a cassette comprises, in order from 5′ to 3′: i) SEQ ID NO:856; ii) SEQ ID NO: 439; iii) a sequence that is complementary to SEQ ID NO: 439, where the sequence can comprise from 0 to 10 mismatches (non-complementary nucleotides) to SEQ ID NO: 439; and iv) SEQ ID NO:857. In some cases, a cassette comprises, in order from 5′ to 3′: i) SEQ ID NO:858; ii) SEQ ID NO: 439; iii) a sequence that is complementary to SEQ ID NO: 439, where the sequence can comprise from 0 to 10 mismatches (non-complementary nucleotides) to SEQ ID NO: 439; and iv) SEQ ID NO:859.
64) In some cases, a cassette comprises, in order from 5′ to 3′: i) SEQ ID NO:854; ii) SEQ ID NO:440; iii) a sequence that is complementary to SEQ ID NO: 440, where the sequence can comprise from 0 to 10 mismatches (non-complementary nucleotides) to SEQ ID NO: 440; and iv) SEQ ID NO:855. In some cases, a cassette comprises, in order from 5′ to 3′: i) SEQ ID NO:856; ii) SEQ ID NO: 440; iii) a sequence that is complementary to SEQ ID NO: 440, where the sequence can comprise from 0 to 10 mismatches (non-complementary nucleotides) to SEQ ID NO: 440; and iv) SEQ ID NO:857. In some cases, a cassette comprises, in order from 5′ to 3′: i) SEQ ID NO:858; ii) SEQ ID NO: 440; iii) a sequence that is complementary to SEQ ID NO: 440, where the sequence can comprise from 0 to 10 mismatches (non-complementary nucleotides) to SEQ ID NO: 440; and iv) SEQ ID NO:859.
65) In some cases, a cassette comprises, in order from 5′ to 3′: i) SEQ ID NO:854; ii) SEQ ID NO:441; iii) a sequence that is complementary to SEQ ID NO: 441, where the sequence can comprise from 0 to 10 mismatches (non-complementary nucleotides) to SEQ ID NO: 441; and iv) SEQ ID NO:855. In some cases, a cassette comprises, in order from 5′ to 3′: i) SEQ ID NO:856; ii) SEQ ID NO: 441; iii) a sequence that is complementary to SEQ ID NO: 441, where the sequence can comprise from 0 to 10 mismatches (non-complementary nucleotides) to SEQ ID NO: 441; and iv) SEQ ID NO:857. In some cases, a cassette comprises, in order from 5′ to 3′: i) SEQ ID NO:858; ii) SEQ ID NO: 441; iii) a sequence that is complementary to SEQ ID NO: 441, where the sequence can comprise from 0 to 10 mismatches (non-complementary nucleotides) to SEQ ID NO: 441; and iv) SEQ ID NO:859.
66) In some cases, a cassette comprises, in order from 5′ to 3′: i) SEQ ID NO:854; ii) SEQ ID NO:442; iii) a sequence that is complementary to SEQ ID NO: 442, where the sequence can comprise from 0 to 10 mismatches (non-complementary nucleotides) to SEQ ID NO: 442; and iv) SEQ ID NO:855. In some cases, a cassette comprises, in order from 5′ to 3′: i) SEQ ID NO:856; ii) SEQ ID NO: 442; iii) a sequence that is complementary to SEQ ID NO: 442, where the sequence can comprise from 0 to 10 mismatches (non-complementary nucleotides) to SEQ ID NO: 442; and iv) SEQ ID NO:857. In some cases, a cassette comprises, in order from 5′ to 3′: i) SEQ ID NO:858; ii) SEQ ID NO: 442; iii) a sequence that is complementary to SEQ ID NO: 442, where the sequence can comprise from 0 to 10 mismatches (non-complementary nucleotides) to SEQ ID NO: 442; and iv) SEQ ID NO:859.
67) In some cases, a cassette comprises, in order from 5′ to 3′: i) SEQ ID NO:854; ii) SEQ ID NO:443; iii) a sequence that is complementary to SEQ ID NO: 443, where the sequence can comprise from 0 to 10 mismatches (non-complementary nucleotides) to SEQ ID NO: 443; and iv) SEQ ID NO:855. In some cases, a cassette comprises, in order from 5′ to 3′: i) SEQ ID NO:856; ii) SEQ ID NO: 443; iii) a sequence that is complementary to SEQ ID NO: 443, where the sequence can comprise from 0 to 10 mismatches (non-complementary nucleotides) to SEQ ID NO: 443; and iv) SEQ ID NO:857. In some cases, a cassette comprises, in order from 5′ to 3′: i) SEQ ID NO:858; ii) SEQ ID NO: 443; iii) a sequence that is complementary to SEQ ID NO: 443, where the sequence can comprise from 0 to 10 mismatches (non-complementary nucleotides) to SEQ ID NO: 443; and iv) SEQ ID NO:859.
68) In some cases, a cassette comprises, in order from 5′ to 3′: i) SEQ ID NO:854; ii) SEQ ID NO:444; iii) a sequence that is complementary to SEQ ID NO: 444, where the sequence can comprise from 0 to 10 mismatches (non-complementary nucleotides) to SEQ ID NO: 444; and iv) SEQ ID NO:855. In some cases, a cassette comprises, in order from 5′ to 3′: i) SEQ ID NO:856; ii) SEQ ID NO: 444; iii) a sequence that is complementary to SEQ ID NO: 444, where the sequence can comprise from 0 to 10 mismatches (non-complementary nucleotides) to SEQ ID NO: 444; and iv) SEQ ID NO:857. In some cases, a cassette comprises, in order from 5′ to 3′: i) SEQ ID NO:858; ii) SEQ ID NO: 444; iii) a sequence that is complementary to SEQ ID NO: 444, where the sequence can comprise from 0 to 10 mismatches (non-complementary nucleotides) to SEQ ID NO: 444; and iv) SEQ ID NO:859.
69) In some cases, a cassette comprises, in order from 5′ to 3′: i) SEQ ID NO:854; ii) SEQ ID NO:445; iii) a sequence that is complementary to SEQ ID NO: 445, where the sequence can comprise from 0 to 10 mismatches (non-complementary nucleotides) to SEQ ID NO: 445; and iv) SEQ ID NO:855. In some cases, a cassette comprises, in order from 5′ to 3′: i) SEQ ID NO:856; ii) SEQ ID NO: 445; iii) a sequence that is complementary to SEQ ID NO: 445, where the sequence can comprise from 0 to 10 mismatches (non-complementary nucleotides) to SEQ ID NO: 445; and iv) SEQ ID NO:857. In some cases, a cassette comprises, in order from 5′ to 3′: i) SEQ ID NO:858; ii) SEQ ID NO: 445; iii) a sequence that is complementary to SEQ ID NO: 445, where the sequence can comprise from 0 to 10 mismatches (non-complementary nucleotides) to SEQ ID NO: 445; and iv) SEQ ID NO:859.
70) In some cases, a cassette comprises, in order from 5′ to 3′: i) SEQ ID NO:854; ii) SEQ ID NO:446; iii) a sequence that is complementary to SEQ ID NO: 446, where the sequence can comprise from 0 to 10 mismatches (non-complementary nucleotides) to SEQ ID NO: 446; and iv) SEQ ID NO:855. In some cases, a cassette comprises, in order from 5′ to 3′: i) SEQ ID NO:856; ii) SEQ ID NO: 446; iii) a sequence that is complementary to SEQ ID NO: 446, where the sequence can comprise from 0 to 10 mismatches (non-complementary nucleotides) to SEQ ID NO: 446; and iv) SEQ ID NO:857. In some cases, a cassette comprises, in order from 5′ to 3′: i) SEQ ID NO:858; ii) SEQ ID NO: 446; iii) a sequence that is complementary to SEQ ID NO: 446, where the sequence can comprise from 0 to 10 mismatches (non-complementary nucleotides) to SEQ ID NO: 446; and iv) SEQ ID NO:859.
71) In some cases, a cassette comprises, in order from 5′ to 3′: i) SEQ ID NO:854; ii) SEQ ID NO:447; iii) a sequence that is complementary to SEQ ID NO: 447, where the sequence can comprise from 0 to 10 mismatches (non-complementary nucleotides) to SEQ ID NO: 447; and iv) SEQ ID NO:855. In some cases, a cassette comprises, in order from 5′ to 3′: i) SEQ ID NO:856; ii) SEQ ID NO: 447; iii) a sequence that is complementary to SEQ ID NO: 447, where the sequence can comprise from 0 to 10 mismatches (non-complementary nucleotides) to SEQ ID NO: 447; and iv) SEQ ID NO:857. In some cases, a cassette comprises, in order from 5′ to 3′: i) SEQ ID NO:858; ii) SEQ ID NO: 447; iii) a sequence that is complementary to SEQ ID NO: 447, where the sequence can comprise from 0 to 10 mismatches (non-complementary nucleotides) to SEQ ID NO: 447; and iv) SEQ ID NO:859.
72) In some cases, a cassette comprises, in order from 5′ to 3′: i) SEQ ID NO:854; ii) SEQ ID NO:448; iii) a sequence that is complementary to SEQ ID NO: 448, where the sequence can comprise from 0 to 10 mismatches (non-complementary nucleotides) to SEQ ID NO: 448; and iv) SEQ ID NO:855. In some cases, a cassette comprises, in order from 5′ to 3′: i) SEQ ID NO:856; ii) SEQ ID NO: 448; iii) a sequence that is complementary to SEQ ID NO: 448, where the sequence can comprise from 0 to 10 mismatches (non-complementary nucleotides) to SEQ ID NO: 448; and iv) SEQ ID NO:857. In some cases, a cassette comprises, in order from 5′ to 3′: i) SEQ ID NO:858; ii) SEQ ID NO: 448; iii) a sequence that is complementary to SEQ ID NO: 448, where the sequence can comprise from 0 to 10 mismatches (non-complementary nucleotides) to SEQ ID NO: 448; and iv) SEQ ID NO:859.
73) In some cases, a cassette comprises, in order from 5′ to 3′: i) SEQ ID NO:854; ii) SEQ ID NO:449; iii) a sequence that is complementary to SEQ ID NO: 449, where the sequence can comprise from 0 to 10 mismatches (non-complementary nucleotides) to SEQ ID NO: 449; and iv) SEQ ID NO:855. In some cases, a cassette comprises, in order from 5′ to 3′: i) SEQ ID NO:856; ii) SEQ ID NO: 449; iii) a sequence that is complementary to SEQ ID NO: 449, where the sequence can comprise from 0 to 10 mismatches (non-complementary nucleotides) to SEQ ID NO: 449; and iv) SEQ ID NO:857. In some cases, a cassette comprises, in order from 5′ to 3′: i) SEQ ID NO:858; ii) SEQ ID NO: 449; iii) a sequence that is complementary to SEQ ID NO: 449, where the sequence can comprise from 0 to 10 mismatches (non-complementary nucleotides) to SEQ ID NO: 449; and iv) SEQ ID NO:859.
74) In some cases, a cassette comprises, in order from 5′ to 3′: i) SEQ ID NO:854; ii) SEQ ID NO:450; iii) a sequence that is complementary to SEQ ID NO: 450, where the sequence can comprise from 0 to 10 mismatches (non-complementary nucleotides) to SEQ ID NO: 450; and iv) SEQ ID NO:855. In some cases, a cassette comprises, in order from 5′ to 3′: i) SEQ ID NO:856; ii) SEQ ID NO: 450; iii) a sequence that is complementary to SEQ ID NO: 450, where the sequence can comprise from 0 to 10 mismatches (non-complementary nucleotides) to SEQ ID NO: 450; and iv) SEQ ID NO:857. In some cases, a cassette comprises, in order from 5′ to 3′: i) SEQ ID NO:858; ii) SEQ ID NO: 450; iii) a sequence that is complementary to SEQ ID NO: 450, where the sequence can comprise from 0 to 10 mismatches (non-complementary nucleotides) to SEQ ID NO: 450; and iv) SEQ ID NO:859.
75) In some cases, a cassette comprises, in order from 5′ to 3′: i) SEQ ID NO:854; ii) SEQ ID NO:451; iii) a sequence that is complementary to SEQ ID NO: 451, where the sequence can comprise from 0 to 10 mismatches (non-complementary nucleotides) to SEQ ID NO: 451; and iv) SEQ ID NO:855. In some cases, a cassette comprises, in order from 5′ to 3′: i) SEQ ID NO:856; ii) SEQ ID NO: 451; iii) a sequence that is complementary to SEQ ID NO: 451, where the sequence can comprise from 0 to 10 mismatches (non-complementary nucleotides) to SEQ ID NO: 451; and iv) SEQ ID NO:857. In some cases, a cassette comprises, in order from 5′ to 3′: i) SEQ ID NO:858; ii) SEQ ID NO: 451; iii) a sequence that is complementary to SEQ ID NO: 451, where the sequence can comprise from 0 to 10 mismatches (non-complementary nucleotides) to SEQ ID NO: 451; and iv) SEQ ID NO:859.
76) In some cases, a cassette comprises, in order from 5′ to 3′: i) SEQ ID NO:854; ii) SEQ ID NO:452; iii) a sequence that is complementary to SEQ ID NO: 452, where the sequence can comprise from 0 to 10 mismatches (non-complementary nucleotides) to SEQ ID NO: 452; and iv) SEQ ID NO:855. In some cases, a cassette comprises, in order from 5′ to 3′: i) SEQ ID NO:856; ii) SEQ ID NO: 452; iii) a sequence that is complementary to SEQ ID NO: 452, where the sequence can comprise from 0 to 10 mismatches (non-complementary nucleotides) to SEQ ID NO: 452; and iv) SEQ ID NO:857. In some cases, a cassette comprises, in order from 5′ to 3′: i) SEQ ID NO:858; ii) SEQ ID NO: 452; iii) a sequence that is complementary to SEQ ID NO: 452, where the sequence can comprise from 0 to 10 mismatches (non-complementary nucleotides) to SEQ ID NO: 452; and iv) SEQ ID NO:859.
77) In some cases, a cassette comprises, in order from 5′ to 3′: i) SEQ ID NO:854; ii) SEQ ID NO:453; iii) a sequence that is complementary to SEQ ID NO: 453, where the sequence can comprise from 0 to 10 mismatches (non-complementary nucleotides) to SEQ ID NO: 453; and iv) SEQ ID NO:855. In some cases, a cassette comprises, in order from 5′ to 3′: i) SEQ ID NO:856; ii) SEQ ID NO: 453; iii) a sequence that is complementary to SEQ ID NO: 453, where the sequence can comprise from 0 to 10 mismatches (non-complementary nucleotides) to SEQ ID NO: 453; and iv) SEQ ID NO:857. In some cases, a cassette comprises, in order from 5′ to 3′: i) SEQ ID NO:858; ii) SEQ ID NO: 453; iii) a sequence that is complementary to SEQ ID NO: 453, where the sequence can comprise from 0 to 10 mismatches (non-complementary nucleotides) to SEQ ID NO: 453; and iv) SEQ ID NO:859.
78) In some cases, a cassette comprises, in order from 5′ to 3′: i) SEQ ID NO:854; ii) SEQ ID NO:454; iii) a sequence that is complementary to SEQ ID NO: 454, where the sequence can comprise from 0 to 10 mismatches (non-complementary nucleotides) to SEQ ID NO: 454; and iv) SEQ ID NO:855. In some cases, a cassette comprises, in order from 5′ to 3′: i) SEQ ID NO:856; ii) SEQ ID NO: 454; iii) a sequence that is complementary to SEQ ID NO: 454, where the sequence can comprise from 0 to 10 mismatches (non-complementary nucleotides) to SEQ ID NO: 454; and iv) SEQ ID NO:857. In some cases, a cassette comprises, in order from 5′ to 3′: i) SEQ ID NO:858; ii) SEQ ID NO: 454; iii) a sequence that is complementary to SEQ ID NO: 454, where the sequence can comprise from 0 to 10 mismatches (non-complementary nucleotides) to SEQ ID NO: 454; and iv) SEQ ID NO:859.
79) In some cases, a cassette comprises, in order from 5′ to 3′: i) SEQ ID NO:854; ii) SEQ ID NO:455; iii) a sequence that is complementary to SEQ ID NO: 455, where the sequence can comprise from 0 to 10 mismatches (non-complementary nucleotides) to SEQ ID NO: 455; and iv) SEQ ID NO:855. In some cases, a cassette comprises, in order from 5′ to 3′: i) SEQ ID NO:856; ii) SEQ ID NO: 455; iii) a sequence that is complementary to SEQ ID NO: 455, where the sequence can comprise from 0 to 10 mismatches (non-complementary nucleotides) to SEQ ID NO: 455; and iv) SEQ ID NO:857. In some cases, a cassette comprises, in order from 5′ to 3′: i) SEQ ID NO:858; ii) SEQ ID NO: 455; iii) a sequence that is complementary to SEQ ID NO: 455, where the sequence can comprise from 0 to 10 mismatches (non-complementary nucleotides) to SEQ ID NO: 455; and iv) SEQ ID NO:859.
80) In some cases, a cassette comprises, in order from 5′ to 3′: i) SEQ ID NO:854; ii) SEQ ID NO:456; iii) a sequence that is complementary to SEQ ID NO: 456, where the sequence can comprise from 0 to 10 mismatches (non-complementary nucleotides) to SEQ ID NO: 456; and iv) SEQ ID NO:855. In some cases, a cassette comprises, in order from 5′ to 3′: i) SEQ ID NO:856; ii) SEQ ID NO: 456; iii) a sequence that is complementary to SEQ ID NO: 456, where the sequence can comprise from 0 to 10 mismatches (non-complementary nucleotides) to SEQ ID NO: 456; and iv) SEQ ID NO:857. In some cases, a cassette comprises, in order from 5′ to 3′: i) SEQ ID NO:858; ii) SEQ ID NO: 456; iii) a sequence that is complementary to SEQ ID NO: 456, where the sequence can comprise from 0 to 10 mismatches (non-complementary nucleotides) to SEQ ID NO: 456; and iv) SEQ ID NO:859.
Recombinant Expression Vector Encoding sbRNA

The present disclosure provides a recombinant expression vector comprising a nucleotide sequence encoding a recombinant RNA molecule of the present disclosure (where the recombinant RNA molecule may be referred to as an “artificial microRNA” or an “sbRNA”). In some cases, the nucleotide sequence encoding the recombinant RNA molecule is operably linked to a promoter that is functional in a eukaryotic cell. In some cases, the nucleotide sequence encoding the recombinant RNA molecule is operably linked to an RNA polymerase II promoter. In some cases, the nucleotide sequence encoding the recombinant RNA molecule is operably linked to an RNA polymerase III promoter. In some cases, the nucleotide sequence encoding the recombinant RNA molecule is operably linked to a CMV promoter. In some cases, the nucleotide sequence encoding the recombinant RNA molecule is operably linked to a CAG promoter. In some cases, the nucleotide sequence encoding the recombinant RNA molecule is operably linked to a CBA promoter. In some cases, the nucleotide sequence encoding the recombinant RNA molecule is operably linked to a U6 promoter. In some cases, the nucleotide sequence encoding the recombinant RNA molecule is operably linked to an EF1α promoter. In some cases, the nucleotide sequence encoding the recombinant RNA molecule is operably linked to an H1 promoter. In some cases, the recombinant expression vector comprises a 5′ adeno-associated virus (AAV) inverted terminal repeat (ITR) sequence and a 3′ AAV ITR sequence.

The present disclosure provides a recombinant expression vector comprising a nucleotide sequence encoding a recombinant RNA molecule of the present disclosure, where the recombinant RNA molecule comprises: a) a double-stranded RNA of the present disclosure; and b) a microRNA scaffold comprising a 5′ flanking polynucleotide (also referred to herein as a “5′ leader”), a loop polynucleotide, and a 3′ flanking polynucleotide (also referred to herein as a “3′ trailer”), wherein the recombinant nucleic acid comprises: i) the 5′ flanking polynucleotide; ii) the first strand of the double-stranded RNA; iii) the loop polynucleotide; (iv) the second strand of the double-stranded RNA; and iii) the 3′ trailer polynucleotide; and wherein at least one of the 5′ flanking polynucleotide, the loop polynucleotide, and the 3′ flanking polynucleotide is heterologous to the first and/or the second strand of the double-stranded RNA. The present disclosure provides a recombinant expression vector comprising a nucleotide sequence encoding a recombinant RNA molecule of the present disclosure, where the recombinant RNA molecule comprises: a) a double-stranded RNA of the present disclosure; and b) a microRNA scaffold comprising a 5′ flanking polynucleotide, a loop polynucleotide, and a 3′ flanking polynucleotide, wherein the recombinant nucleic acid comprises: i) the 5′ flanking polynucleotide; ii) the second strand of the double-stranded RNA; iii) the loop polynucleotide; (iv) the first strand of the double-stranded RNA; and iii) the 3′ flanking polynucleotide; and wherein at least one of the 5′ flanking polynucleotide, the loop polynucleotide, and the 3′ flanking polynucleotide is heterologous to the first and/or the second strand of the double-stranded RNA. In some cases, the 5′ flanking polynucleotide, the loop polynucleotide, and the 3′ flanking polynucleotide are derived from miR33.

The present disclosure provides a recombinant expression vector comprising a nucleotide sequence encoding a recombinant RNA molecule of the present disclosure, where the recombinant RNA molecule comprises: a) a double-stranded RNA of the present disclosure; and b) a microRNA scaffold comprising a 5′ flanking polynucleotide (also referred to herein as a “5′ leader”) and a 3′ flanking polynucleotide (also referred to herein as a “3′ trailer”), wherein the recombinant nucleic acid comprises: i) the 5′ flanking polynucleotide; ii) the first strand of the double-stranded RNA; iii) the second strand of the double-stranded RNA; and iv) the 3′ trailer polynucleotide; and wherein one or both of the 5′ flanking polynucleotide and the 3′ flanking polynucleotide is heterologous to the first and/or the second strand of the double-stranded RNA. The present disclosure provides a recombinant expression vector comprising a nucleotide sequence encoding a recombinant RNA molecule of the present disclosure, where the recombinant RNA molecule comprises: a) a double-stranded RNA of the present disclosure; and b) a microRNA scaffold comprising a 5′ flanking polynucleotide and a 3′ flanking polynucleotide, wherein the recombinant nucleic acid comprises: i) the 5′ flanking polynucleotide; ii) the second strand of the double-stranded RNA; iii) the first strand of the double-stranded RNA; and iv) the 3′ flanking polynucleotide; and wherein one or both of 5′ flanking polynucleotide and the 3′ flanking polynucleotide is heterologous to the first and/or the second strand of the double-stranded RNA. In some cases, the 5′ flanking polynucleotide and the 3′ flanking polynucleotide are derived from miR451.

### Recombinant Expression Vector Comprising a Cassette

The present disclosure provides a recombinant expression vector comprising cassette (a “DNA molecule”) of the present disclosure, where the cassette comprises a nucleotide sequence encoding a recombinant RNA molecule of the present disclosure (where the recombinant RNA molecule may be referred to as an “artificial microRNA” or an “sbRNA”). In some cases, the nucleotide sequence encoding the recombinant RNA molecule is operably linked to a promoter that is functional in a eukaryotic cell. In some cases, the nucleotide sequence encoding the recombinant RNA molecule is operably linked to an RNA polymerase II promoter. In some cases, the nucleotide sequence encoding the recombinant RNA molecule is operably linked to an RNA polymerase III promoter. In some cases, the nucleotide sequence encoding the recombinant RNA molecule is operably linked to a CMV promoter. In some cases, the nucleotide sequence encoding the recombinant RNA molecule is operably linked to a U6 promoter. In some cases, the nucleotide sequence encoding the recombinant RNA molecule is operably linked to an EF1α promoter. In some cases, the nucleotide sequence encoding the recombinant RNA molecule is operably linked to an H1 promoter. In some cases, the recombinant expression vector comprises a 5′ adeno-associated virus (AAV) inverted terminal repeat (ITR) sequence and a 3′ AAV ITR sequence.

The following are non-limiting examples of recombinant expression vectors comprising a cassette of the present disclosure.

1) In some cases, a recombinant expression vector comprises a cassette that comprises the nucleotide sequence: tgcacacctcctggcgggcagctctgCTGCTGCTAATGCTGCTGCTGtgttctggcaatacctgCAGCAGCAGCA AAAGCAGCAGCAgggaggcctgccctgactgcccacTTTTTG (SEQ ID NO:579; “CUG-10” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
2) In some cases, a recombinant expression vector comprises a cassette that comprises the nucleotide sequence: tgcacacctcctggcgggcagctctgCTGCTGCTACAGCTGCTGCTGtgttctggcaatacctgCAGCAGCAGCA GAAGCAGCAGCAgggaggcctgccctgactgcccacTTTTTG (SEQ ID NO:580; “CUG_19” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.;
3) In some cases, a recombinant expression vector comprises a cassette that comprises the nucleotide sequence: tgcacacctcctggcgggcagctctgCTGCTGCTACTACTGCTGCTGtgttctggcaatacctgCAGCAGCAGAA GAAGCAGCAGCAgggaggcctgccctgactgcccacTTTTTG (SEQ ID NO:581; “CUG_28” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
4) In some cases, a recombinant expression vector comprises a cassette that comprises the nucleotide sequence: tgcacacctcctggcgggcagctctgCTGCTGCTACTGATGCTGCTGtgttctggcaatacctgCAGCAGCAACA GAAGCAGCAGCAgggaggcctgccctgactgcccacTTTTTG (SEQ ID NO:582; “CUG_37” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
5) In some cases, a recombinant expression vector comprises a cassette that comprises the nucleotide sequence: tgcacacctcctggcgggcagctctgCTGCTGCTACTGCAGCTGCTGtgttctggcaatacctgCAGCAGCAGCA GAAGCAGCAGCAgggaggcctgccctgactgcccacTTTTTG (SEQ ID NO:583; “CUG_46” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
6) In some cases, a recombinant expression vector comprises a cassette that comprises the nucleotide sequence: tgcacacctcctggcgggcagctctgCTGCTGCTACTGCTACTGCTGtgttctggcaatacctgCAGCAGAAGCA GAAGCAGCAGCAgggaggcctgccctgactgcccacTTTTTG (SEQ ID NO:584; CUG_55” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
7) In some cases, a recombinant expression vector comprises a cassette that comprises the nucleotide sequence: tgcacacctcctggcgggcagctctgCTGCTGCTACTGCTGATGCTGtgttctggcaatacctgCAGCAACAGCA GAAGCAGCAGCAgggaggcctgccctgactgcccacTTTTTG (SEQ ID NO:585; “CUG_64” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
8) In some cases, a recombinant expression vector comprises a cassette that comprises the nucleotide sequence: tgcacacctcctggcgggcagctctgCTGCTGCTGAAGCTGCTGCTGtgttctggcaatacctgCAGCAGCAGCA ACAGCAGCAGCAgggaggcctgccctgactgcccacTTTTTG (SEQ ID NO:586; “CUG_118” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
9) In some cases, a recombinant expression vector comprises a cassette that comprises the nucleotide sequence: tgcacacctcctggcgggcagctctgCTGCTGCTGATACTGCTGCTGtgttctggcaatacctgCAGCAGCAGAA ACAGCAGCAGCAgggaggcctgccctgactgcccacTTTTTG (SEQ ID NO:587; “CUG_127” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
10) In some cases, a recombinant expression vector comprises a cassette that comprises the nucleotide sequence: tgcacacctcctggcgggcagctctgCTGCTGCTGATGATGCTGCTGtgttctggcaatacctgCAGCAGCAACA ACAGCAGCAGCAgggaggcctgccctgactgcccacTTTTTG (SEQ ID NO:588; “CUG_136” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
11) In some cases, a recombinant expression vector comprises a cassette that comprises the nucleotide sequence: tgcacacctcctggcgggcagctctgCTGCTGCTGATGCAGCTGCTGtgttctggcaatacctgCAGCAGCAGCA ACAGCAGCAGCAgggaggcctgccctgactgcccacTTTTTG (SEQ ID NO:589; “CUG_145” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
12) In some cases, a recombinant expression vector comprises a cassette that comprises the nucleotide sequence: tgcacacctcctggcgggcagctctgCTGCTGCTGATGCTACTGCTGtgttctggcaatacctgCAGCAGAAGCA ACAGCAGCAGCAgggaggcctgccctgactgcccacTTTTTG (SEQ ID NO:590; “CUG_154” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
13) In some cases, a recombinant expression vector comprises a cassette that comprises the nucleotide sequence: tgcacacctcctggcgggcagctctgCTGCTGCTGATGCTGATGCTGtgttctggcaatacctgCAGCAACAGCA ACAGCAGCAGCAgggaggcctgccctgactgcccacTTTTTG (SEQ ID NO:591; “CUG_163” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
14) In some cases, a recombinant expression vector comprises a cassette that comprises the nucleotide sequence: tgcacacctcctggcgggcagctctgCTGCTGCTGCAACTGCTGCTGtgttctggcaatacctgCAGCAGCAGAA GCAGCAGCAGCAgggaggcctgccctgactgcccacTTTTTG (SEQ ID NO:592; “CUG_217” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
15) In some cases, a recombinant expression vector comprises a cassette that comprises the nucleotide sequence: tgcacacctcctggcgggcagctctgCTGCTGCTGCAGATGCTGCTGtgttctggcaatacctgCAGCAGCAACA GCAGCAGCAGCAgggaggcctgccctgactgcccacTTTTTG (SEQ ID NO:593; “CUG_226” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
16) In some cases, a recombinant expression vector comprises a cassette that comprises the nucleotide sequence: tgcacacctcctggcgggcagctctgCTGCTGCTGCAGCAGCTGCTGtgttctggcaatacctgCAGCAGCAGCA GCAGCAGCAGCAgggaggcctgccctgactgcccacTTTTTG (SEQ ID NO:594; “CUG_235” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
17) In some cases, a recombinant expression vector comprises a cassette that comprises the nucleotide sequence: tgcacacctcctggcgggcagctctgCTGCTGCTGCAGCTACTGCTGtgttctggcaatacctgCAGCAGAAGCA GCAGCAGCAGCAgggaggcctgccctgactgcccacTTTTTG (SEQ ID NO:595; “CUG_244” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
18) In some cases, a recombinant expression vector comprises a cassette that comprises the nucleotide sequence: tgcacacctcctggcgggcagctctgCTGCTGCTGCAGCTGATGCTGtgttctggcaatacctgCAGCAACAGCA GCAGCAGCAGCAgggaggcctgccctgactgcccacTTTTTG (SEQ ID NO:596; “CUG_253” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
19) In some cases, a recombinant expression vector comprises a cassette that comprises the nucleotide sequence: tgcacacctcctggcgggcagctctgCTGCTGCAGCTGCTGCTGCTGtgttctggcaatacctgCAGCAGCAGCA GCAGCAGCAGCAgggaggcctgccctgactgcccacTTTTTG (SEQ ID NO:597; “CUG_NA-A” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
20) In some cases, a recombinant expression vector comprises a cassette that comprises the nucleotide sequence: tgcacacctcctggcgggcagctctgCTGCTGCAACTGCTGCTGCTGtgttctggcaatacctgCAGCAGCAGCA GAAGCAGCAGCAgggaggcctgccctgactgcccacTTTTTG (SEQ ID NO:598; “CUG_NA_B” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
21) In some cases, a recombinant expression vector comprises a cassette that comprises the nucleotide sequence: tgcacacctcctggcgggcagctctgCTGCTGCAGATGCTGCTGCTGtgttctggcaatacctgCAGCAGCAGCA ACAGCAGCAGCAgggaggcctgccctgactgcccacTTTTTG (SEQ ID NO:599; “CUG_NA_C” in Table 7; FIG. 23). In some cases, the cassette does not include the 3′ TTTTTG sequence. In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
22) In some cases, a recombinant expression vector comprises a cassette that comprises the nucleotide sequence: tgcacacctcctggcgggcagctctgCTGCTGCAGCAGCTGCTGCTGtgttctggcaatacctgCAGCAGCAGCA GCAGCAGCAGCAgggaggcctgccctgactgcccacTTTTTG (SEQ ID NO:600; “CUG_NA_D” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
23) In some cases, a recombinant expression vector comprises a cassette that comprises the nucleotide sequence: tgcacacctcctggcgggcagctctgCTGCTGCAGCTACTGCTGCTGtgttctggcaatacctgCAGCAGCAGAA GCAGCAGCAGCAgggaggcctgccctgactgcccacTTTTTG (SEQ ID NO:601; “CUG_NA_E” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
24) In some cases, a recombinant expression vector comprises a cassette that comprises the nucleotide sequence: tgcacacctcctggcgggcagctctgCTGCTGCAGCTGATGCTGCTGtgttctggcaatacctgCAGCAGCAACA GCAGCAGCAGCAgggaggcctgccctgactgcccacTTTTTG (SEQ ID NO:602; “CUG_NA_F” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
25) In some cases, a recombinant expression vector comprises a cassette that comprises the nucleotide sequence: tgcacacctcctggcgggcagctctgCTGCTGCAGCTGCAGCTGCTGtgttctggcaatacctgCAGCAGCAGCA GCAGCAGCAGCAgggaggcctgccctgactgcccacTTTTTG (SEQ ID NO:603; “CUG_NA_G” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
26) In some cases, a recombinant expression vector comprises a cassette that comprises the nucleotide sequence: tgcacacctcctggcgggcagctctgCTGCTGCAGCTGCTACTGCTGtgttctggcaatacctgCAGCAGAAGCA GCAGCAGCAGCAgggaggcctgccctgactgcccacTTTTTG (SEQ ID NO:604; “CUG_NA_H” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
27) In some cases, a recombinant expression vector comprises a cassette that comprises the nucleotide sequence: tgcacacctcctggcgggcagctctgCTGCTGCAGCTGCTGATGCTGtgttctggcaatacctgCAGCAACAGCA GCAGCAGCAGCAgggaggcctgccctgactgcccacTTTTTG (SEQ ID NO:605; “CUG_NA_I” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
28) In some cases, a recombinant expression vector comprises a cassette that comprises the nucleotide sequence: tgcacacctcctggcgggcagctctgCTGCTGCTAAAGCTGCTGCTGtgttctggcaatacctgCAGCAGCAGCA AAAGCAGCAGCAgggaggcctgccctgactgcccacTTTTTG (SEQ ID NO:606; “CUG_307” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
29) In some cases, a recombinant expression vector comprises a cassette that comprises the nucleotide sequence: tgcacacctcctggcgggcagctctgCTGCTGCTAATACTGCTGCTGtgttctggcaatacctgCAGCAGCAGAA AAAGCAGCAGCAgggaggcctgccctgactgcccacTTTTTG (SEQ ID NO:607; “CUG_334” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
30) In some cases, a recombinant expression vector comprises a cassette that comprises the nucleotide sequence: tgcacacctcctggcgggcagctctgCTGCTGCTAATGATGCTGCTGtgttctggcaatacctgCAGCAGCAACA AAAGCAGCAGCAgggaggcctgccctgactgcccacTTTTTG (SEQ ID NO:608; “CUG_361” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
31) In some cases, a recombinant expression vector comprises a cassette that comprises the nucleotide sequence: tgcacacctcctggcgggcagctctgCTGCTGCTAATGCAGCTGCTGtgttctggcaatacctgCAGCAGCAGCA AAAGCAGCAGCAgggaggcctgccctgactgcccacTTTTTG (SEQ ID NO:609; “CUG_388” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
32) In some cases, a recombinant expression vector comprises a cassette that comprises the nucleotide sequence: tgcacacctcctggcgggcagctctgCTGCTGCTAATGCTACTGCTGtgttctggcaatacctgCAGCAGAAGCA AAAGCAGCAGCAgggaggcctgccctgactgcccacTTTTTG (SEQ ID NO:610; “CUG_415” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
33) In some cases, a recombinant expression vector comprises a cassette that comprises the nucleotide sequence: tgcacacctcctggcgggcagctctgCTGCTGCTACAGATGCTGCTGtgttctggcaatacctgCAGCAGCAACA GAAGCAGCAGCAgggaggcctgccctgactgcccacTTTTTG (SEQ ID NO:611; “CUG_631” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
34) In some cases, a recombinant expression vector comprises a cassette that comprises the nucleotide sequence: tgcacacctcctggcgggcagctctgCTGCTGCTACAGCAGCTGCTGtgttctggcaatacctgCAGCAGCAGCA GAAGCAGCAGCAgggaggcctgccctgactgcccacTTTTTG (SEQ ID NO:612; “CUG_658” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
35) In some cases, a recombinant expression vector comprises a cassette that comprises the nucleotide sequence: tgcacacctcctggcgggcagctctgCTGCTGCTACAGCTGATGCTGtgttctggcaatacctgCAGCAACAGCA GAAGCAGCAGCAgggaggcctgccctgactgcccacTTTTTG (SEQ ID NO:613; “CUG_712” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
36) In some cases, a recombinant expression vector comprises a cassette that comprises the nucleotide sequence: tgcacacctcctggcgggcagctctgCTGCTGCTGAAGATGCTGCTGtgttctggcaatacctgCAGCAGCAACA ACAGCAGCAGCAgggaggcctgccctgactgcccacTTTTTG (SEQ ID NO:614; “CUG_2116” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
37) In some cases, a recombinant expression vector comprises a cassette that comprises the nucleotide sequence: tgcacacctcctggcgggcagctctgCTGCTGCTGAAGCAGCTGCTGtgttctggcaatacctgCAGCAGCAGCA ACAGCAGCAGCAgggaggcctgccctgactgcccacTTTTTG (SEQ ID NO:615; “CUG_2143” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
38) In some cases, a recombinant expression vector comprises a cassette that comprises the nucleotide sequence: tgcacacctcctggcgggcagctctgCTGCTGCTGAAGCTACTGCTGtgttctggcaatacctgCAGCAGAAGCA ACAGCAGCAGCAgggaggcctgccctgactgcccacTTTTTG (SEQ ID NO:616; “CUG_2170” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
39) In some cases, a recombinant expression vector comprises a cassette that comprises the nucleotide sequence: tgcacacctcctggcgggcagctctgCTGCTGCTAATGCTGATGCTGtgttctggcaatacctgCAGCAACAGCA AAAGCAGCAGCAgggaggcctgccctgactgcccacTTTTTG (SEQ ID NO:617; “CUG_442” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
40) In some cases, a recombinant expression vector comprises a cassette that comprises the nucleotide sequence: tgcacacctcctggcgggcagctctgCTGCTGCTACAACTGCTGCTGtgttctggcaatacctgCAGCAGCAGAA GAAGCAGCAGCAgggaggcctgccctgactgcccacTTTTTG (SEQ ID NO:618; “CUG_604” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
41) In some cases, a recombinant expression vector comprises a cassette that comprises the nucleotide sequence: tgcacacctcctggcgggcagctctgCTGCTGCTACAGCTACTGCTGtgttctggcaatacctgCAGCAGAAGCA GAAGCAGCAGCAgggaggcctgccctgactgcccacTTTTTG (SEQ ID NO:619; “CUG_685” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
42) In some cases, a recombinant expression vector comprises a cassette that comprises the nucleotide sequence: tgcacacctcctggcgggcagctctgCTGCTGCTGAAACTGCTGCTGtgttctggcaatacctgCAGCAGCAGAA ACAGCAGCAGCAgggaggcctgccctgactgcccacTTTTTG (SEQ ID NO:620; “CUG_2089” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
43) In some cases, a recombinant expression vector comprises a cassette that comprises the nucleotide sequence: tgcacacctcctggcgggcagctctgCTGCTGCTGAAGCTGATGCTGtgttctggcaatacctgCAGCAACAGCA ACAGCAGCAGCAgggaggcctgccctgactgcccacTTTTTG (SEQ ID NO:621; “CUG_2197” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
44) In some cases, a recombinant expression vector comprises a cassette that comprises the nucleotide sequence: tgcacacctcctggcgggcagctctgCTGCTGCTAAAGATGCTGCTGtgttctggcaatacctgCAGCAGCAACA AAAGCAGCAGCAgggaggcctgccctgactgcccacTTTTTG (SEQ ID NO:622; “CUG_4870” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
45) In some cases, a recombinant expression vector comprises a cassette that comprises the nucleotide sequence: tgcacacctcctggcgggcagctctgCTGCTGCTACAGAAGCTGCTGtgttctggcaatacctgCAGCAGCAACA GAAGCAGCAGCAgggaggcctgccctgactgcccacTTTTTG (SEQ ID NO:623; “CUG_9973” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
46) In some cases, a recombinant expression vector comprises a cassette that comprises the nucleotide sequence: tgcacacctcctggcgggcagctctgCTGCTGCTACAGATGATGCTGtgttctggcaatacctgCAGCAACAACA GAAGCAGCAGCAgggaggcctgccctgactgcccacTTTTTG (SEQ ID NO:624; “CUG_1013” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
47) In some cases, a recombinant expression vector comprises a cassette that comprises the nucleotide sequence: tgcacacctcctggcgggcagctctgCTGCTGCTACAGCAGATGCTGtgttctggcaatacctgCAGCAACAGCA GAAGCAGCAGCAgggaggcctgccctgactgcccacTTTTTG (SEQ ID NO:625; “CUG_1070” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
48) In some cases, a recombinant expression vector comprises a cassette that comprises the nucleotide sequence: tgcacacctcctggcgggcagctctgCTGCTGCTGAAGATACTGCTGtgttctggcaatacctgCAGCAGAAACA ACAGCAGCAGCAgggaggcctgccctgactgcccacTTTTTG (SEQ ID NO:626; “CUG_2341” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
49) In some cases, a recombinant expression vector comprises a cassette that comprises the nucleotide sequence: tgcacacctcctggcgggcagctctgCTGCTGCTGAAGCAACTGCTGtgttctggcaatacctgCAGCAGAAGCA ACAGCAGCAGCAgggaggcctgccctgactgcccacTTTTTG (SEQ ID NO:627; “CUG_2398” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
50) In some cases, a recombinant expression vector comprises a cassette that comprises the nucleotide sequence: tgcacacctcctggcgggcagctctgCTGCTGCTAAAACTGCTGCTGtgttctggcaatacctgCAGCAGCAGAA AAAGCAGCAGCAgggaggcctgccctgactgcccacTTTTTG (SEQ ID NO:628; “CUG_4789” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
51) In some cases, a recombinant expression vector comprises a cassette that comprises the nucleotide sequence: tgcacacctcctggcgggcagctctgCTGCTGCTAAAGCAGCTGCTGtgttctggcaatacctgCAGCAGCAGCA AAAGCAGCAGCAgggaggcctgccctgactgcccacTTTTTG (SEQ ID NO:629; “CUG_4951” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
52) In some cases, a recombinant expression vector comprises a cassette that comprises the nucleotide sequence: tgcacacctcctggcgggcagctctgCTGCTGCTAAAGCTACTGCTGtgttctggcaatacctgCAGCAGAAGCA AAAGCAGCAGCAgggaggcctgccctgactgcccacTTTTTG (SEQ ID NO:630; “CUG_5032” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
53) In some cases, a recombinant expression vector comprises a cassette that comprises the nucleotide sequence: tgcacacctcctggcgggcagctctgCTGCTGCTAAAGCTGATGCTGtgttctggcaatacctgCAGCAACAGCA AAAGCAGCAGCAgggaggcctgccctgactgcccacTTTTTG (SEQ ID NO:631; “CUG_5113” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
54) In some cases, a recombinant expression vector comprises a cassette that comprises the nucleotide sequence: tgcacacctcctggcgggcagctctgCTGCTGCTAATAATGCTGCTGtgttctggcaatacctgCAGCAGCAAAA AAAGCAGCAGCAgggaggcctgccctgactgcccacTTTTTG (SEQ ID NO:632; “CUG_5599” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
55) In some cases, a recombinant expression vector comprises a cassette that comprises the nucleotide sequence: tgcacacctcctggcgggcagctctgCTGCTGCTAATACAGCTGCTGtgttctggcaatacctgCAGCAGCAGAA AAAGCAGCAGCAgggaggcctgccctgactgcccacTTTTTG (SEQ ID NO:633; “CUG_5680” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
56) In some cases, a recombinant expression vector comprises a cassette that comprises the nucleotide sequence: tgcacacctcctggcgggcagctctgCTGCTGCTAATACTACTGCTGtgttctggcaatacctgCAGCAGAAGAA AAAGCAGCAGCAgggaggcctgccctgactgcccacTTTTTG (SEQ ID NO:634 “CUG_5761” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
57) In some cases, a recombinant expression vector comprises a cassette that comprises the nucleotide sequence: tgcacacctcctggcgggcagctctgCTGCTGCTAATACTGATGCTGtgttctggcaatacctgCAGCAACAGAA AAAGCAGCAGCAgggaggcctgccctgactgcccacTTTTTG (SEQ ID NO:635; “CUG_5842” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
58) In some cases, a recombinant expression vector comprises a cassette that comprises the nucleotide sequence: tgcacacctcctggcgggcagctctgCTGCTGCTAATGAAGCTGCTGtgttctggcaatacctgCAGCAGCAACA AAAGCAGCAGCAgggaggcctgccctgactgcccacTTTTTG (SEQ ID NO:636; “CUG_6328” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
59) In some cases, a recombinant expression vector comprises a cassette that comprises the nucleotide sequence: tgcacacctcctggcgggcagctctgCTGCTGCTAATGATACTGCTGtgttctggcaatacctgCAGCAGAAACA AAAGCAGCAGCAgggaggcctgccctgactgcccacTTTTTG (SEQ ID NO:637; “CUG_6409” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
60) In some cases, a recombinant expression vector comprises a cassette that comprises the nucleotide sequence: tgcacacctcctggcgggcagctctgCTGCTGCTAATGATGATGCTGtgttctggcaatacctgCAGCAACAACA AAAGCAGCAGCAgggaggcctgccctgactgcccacTTTTTG (SEQ ID NO:638; “CUG_6490” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
61) In some cases, a recombinant expression vector comprises a cassette that comprises the nucleotide sequence: tgcacacctcctggcgggcagctctgCTGCTGCTAATGCAACTGCTGtgttctggcaatacctgCAGCAGAAGCA AAAGCAGCAGCAgggaggcctgccctgactgcccacTTTTTG (SEQ ID NO:639; “CUG_6976” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
62) In some cases, a recombinant expression vector comprises a cassette that comprises the nucleotide sequence: tgcacacctcctggcgggcagctctgCTGCTGCTAATGCAGATGCTGtgttctggcaatacctgCAGCAACAGCA AAAGCAGCAGCAgggaggcctgccctgactgcccacTTTTTG (SEQ ID NO:640; “CUG_7057” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
63) In some cases, a recombinant expression vector comprises a cassette that comprises the nucleotide sequence: tgcacacctcctggcgggcagctctgCTGCTGCTAATGCTAATGCTGtgttctggcaatacctgCAGCAAAAGCA AAAGCAGCAGCAgggaggcctgccctgactgcccacTTTTTG (SEQ ID NO:641; “CUG_7543” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
64) In some cases, a recombinant expression vector comprises a cassette that comprises the nucleotide sequence: tgcacacctcctggcgggcagctctgCTGCTGCTACAAATGCTGCTGtgttctggcaatacctgCAGCAGCAAAA GAAGCAGCAGCAgggaggcctgccctgactgcccacTTTTTG (SEQ ID NO:642; “CUG_9244” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
65) In some cases, a recombinant expression vector comprises a cassette that comprises the nucleotide sequence: tgcacacctcctggcgggcagctctgCTGCTGCTACAACAGCTGCTGtgttctggcaatacctgCAGCAGCAGAA GAAGCAGCAGCAgggaggcctgccctgactgcccacTTTTTG (SEQ ID NO:643; “CUG_9325” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
66) In some cases, a recombinant expression vector comprises a cassette that comprises the nucleotide sequence: tgcacacctcctggcgggcagctctgCTGCTGCTACAACTACTGCTGtgttctggcaatacctgCAGCAGAAGAA GAAGCAGCAGCAgggaggcctgccctgactgcccacTTTTTG (SEQ ID NO:644; “CUG_9406” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
67) In some cases, a recombinant expression vector comprises a cassette that comprises the nucleotide sequence: tgcacacctcctggcgggcagctctgCTGCTGCTACAACTGATGCTGtgttctggcaatacctgCAGCAACAGAA GAAGCAGCAGCAgggaggcctgccctgactgcccacTTTTTG (SEQ ID NO:645; “CUG_9487” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
68) In some cases, a recombinant expression vector comprises a cassette that comprises the nucleotide sequence: tgcacacctcctggcgggcagctctgCTGCTGCTACAGATACTGCTGtgttctggcaatacctgCAGCAGAAACA GAAGCAGCAGCAgggaggcctgccctgactgcccacTTTTTG (SEQ ID NO:646; “CUG_10054” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
69) In some cases, a recombinant expression vector comprises a cassette that comprises the nucleotide sequence: tgcacacctcctggcgggcagctctgCTGCTGCTACAGCAACTGCTGtgttctggcaatacctgCAGCAGAAGCA GAAGCAGCAGCAgggaggcctgccctgactgcccacTTTTTG (SEQ ID NO:647; “CUG_10621” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
70) In some cases, a recombinant expression vector comprises a cassette that comprises the nucleotide sequence: tgcacacctcctggcgggcagctctgCTGCTGCTACAGCTAATGCTGtgttctggcaatacctgCAGCAAAAGCA GAAGCAGCAGCAgggaggcctgccctgactgcccacTTTTTG (SEQ ID NO:648; “CUG_11188” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
71) In some cases, a recombinant expression vector comprises a cassette that comprises the nucleotide sequence: tgcacacctcctggcgggcagctctgCTGCTGCTGAAAATGCTGCTGtgttctggcaatacctgCAGCAGCAAAA ACAGCAGCAGCAgggaggcctgccctgactgcccacTTTTTG (SEQ ID NO:649; “CUG_222609” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
72) In some cases, a recombinant expression vector comprises a cassette that comprises the nucleotide sequence: tgcacacctcctggcgggcagctctgCTGCTGCTGAAACAGCTGCTGtgttctggcaatacctgCAGCAGCAGAA ACAGCAGCAGCAgggaggcctgccctgactgcccacTTTTTG (SEQ ID NO:650; “CUG_22690” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
73) In some cases, a recombinant expression vector comprises a cassette that comprises the nucleotide sequence: tgcacacctcctggcgggcagctctgCTGCTGCTGAAACTACTGCTGtgttctggcaatacctgCAGCAGAAGAA ACAGCAGCAGCAgggaggcctgccctgactgcccacTTTTTG (SEQ ID NO:651; “CUG_22771” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
74) In some cases, a recombinant expression vector comprises a cassette that comprises the nucleotide sequence: tgcacacctcctggcgggcagctctgCTGCTGCTGAAACTGATGCTGtgttctggcaatacctgCAGCAACAGAA ACAGCAGCAGCAgggaggcctgccctgactgcccacTTTTTG (SEQ ID NO:652; “CUG_22852” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
75) In some cases, a recombinant expression vector comprises a cassette that comprises the nucleotide sequence: tgcacacctcctggcgggcagctctgCTGCTGCTGAAGAAGCTGCTGtgttctggcaatacctgCAGCAGCAACA ACAGCAGCAGCAgggaggcctgccctgactgcccacTTTTTG (SEQ ID NO:653; “CUG_233338” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
76) In some cases, a recombinant expression vector comprises a cassette that comprises the nucleotide sequence: tgcacacctcctggcgggcagctctgCTGCTGCTGAAGATGATGCTGtgttctggcaatacctgCAGCAACAACA ACAGCAGCAGCAgggaggcctgccctgactgcccacTTTTTG (SEQ ID NO:654; “CUG_23500” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
77) In some cases, a recombinant expression vector comprises a cassette that comprises the nucleotide sequence: tgcacacctcctggcgggcagctctgCTGCTGCTGAAGCAGATGCTGtgttctggcaatacctgCAGCAACAGCA ACAGCAGCAGCAgggaggcctgccctgactgcccacTTTTTG (SEQ ID NO:655; “CUG_24067” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
78) In some cases, a recombinant expression vector comprises a cassette that comprises the nucleotide sequence: tgcacacctcctggcgggcagctctgCTGCTGCTGAAGCTAATGCTGtgttctggcaatacctgCAGCAAAAGCA ACAGCAGCAGCAgggaggcctgccctgactgcccacTTTTTG (SEQ ID NO:656; “CUG_24553” in Table 7; FIG. 23). In some cases, the cassette does not include the 3′ TTTTTG sequence. In some cases, the cassette includes the nucleotide sequence Tₙ, where n is an integer from 5 to 10 (e.g., n is 5, 6, 7, 8, 9, or 10), in place of the 3′ TTTTTG sequence. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.

The following are further non-limiting examples of recombinant expression vectors comprising a cassette of the present disclosure.

1) In some cases, a recombinant expression vector comprises one of the following cassettes. In some cases, a cassette comprises, in order from 5′ to 3′: i) SEQ ID NO:854; ii) SEQ ID NO:379; iii) a sequence that is complementary to SEQ ID NO: 379, where the sequence can comprise from 0 to 10 mismatches (non-complementary nucleotides) to SEQ ID NO: 379; and iv) SEQ ID NO:855. In some cases, a cassette comprises, in order from 5′ to 3′: i) SEQ ID NO:856; ii) SEQ ID NO: 379; iii) a sequence that is complementary to SEQ ID NO: 379, where the sequence can comprise from 0 to 10 mismatches (non-complementary nucleotides) to SEQ ID NO: 379; and iv) SEQ ID NO:857. In some cases, a cassette comprises, in order from 5′ to 3′: i) SEQ ID NO:858; ii) SEQ ID NO: 379; iii) a sequence that is complementary to SEQ ID NO: 379, where the sequence can comprise from 0 to 10 mismatches (non-complementary nucleotides) to SEQ ID NO: 379; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
2) In some cases, a recombinant expression vector comprises one of the following cassettes. In some cases, a cassette comprises, in order from 5′ to 3′: i) SEQ ID NO:854; ii) SEQ ID NO:380; iii) a sequence that is complementary to SEQ ID NO: 380, where the sequence can comprise from 0 to 10 mismatches (non-complementary nucleotides) to SEQ ID NO: 380; and iv) SEQ ID NO:855. In some cases, a cassette comprises, in order from 5′ to 3′: i) SEQ ID NO:856; ii) SEQ ID NO: 380; iii) a sequence that is complementary to SEQ ID NO: 380, where the sequence can comprise from 0 to 10 mismatches (non-complementary nucleotides) to SEQ ID NO: 380; and iv) SEQ ID NO:857. In some cases, a cassette comprises, in order from 5′ to 3′: i) SEQ ID NO:858; ii) SEQ ID NO: 380; iii) a sequence that is complementary to SEQ ID NO: 380, where the sequence can comprise from 0 to 10 mismatches (non-complementary nucleotides) to SEQ ID NO: 380; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
3) In some cases, a recombinant expression vector comprises one of the following cassettes. In some cases, a cassette comprises, in order from 5′ to 3′: i) SEQ ID NO:854; ii) SEQ ID NO:381; iii) a sequence that is complementary to SEQ ID NO: 381, where the sequence can comprise from 0 to 10 mismatches (non-complementary nucleotides) to SEQ ID NO: 381; and iv) SEQ ID NO:855. In some cases, a cassette comprises, in order from 5′ to 3′: i) SEQ ID NO:856; ii) SEQ ID NO: 381; iii) a sequence that is complementary to SEQ ID NO: 381, where the sequence can comprise from 0 to 10 mismatches (non-complementary nucleotides) to SEQ ID NO: 381; and iv) SEQ ID NO:857. In some cases, a cassette comprises, in order from 5′ to 3′: i) SEQ ID NO:858; ii) SEQ ID NO: 381; iii) a sequence that is complementary to SEQ ID NO: 381, where the sequence can comprise from 0 to 10 mismatches (non-complementary nucleotides) to SEQ ID NO: 381; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
4) In some cases, a recombinant expression vector comprises one of the following cassettes. In some cases, a cassette comprises, in order from 5′ to 3′: i) SEQ ID NO:854; ii) SEQ ID NO:382; iii) a sequence that is complementary to SEQ ID NO: 382, where the sequence can comprise from 0 to 10 mismatches (non-complementary nucleotides) to SEQ ID NO: 382; and iv) SEQ ID NO:855. In some cases, a cassette comprises, in order from 5′ to 3′: i) SEQ ID NO:856; ii) SEQ ID NO: 382; iii) a sequence that is complementary to SEQ ID NO: 382, where the sequence can comprise from 0 to 10 mismatches (non-complementary nucleotides) to SEQ ID NO: 382; and iv) SEQ ID NO:857. In some cases, a cassette comprises, in order from 5′ to 3′: i) SEQ ID NO:858; ii) SEQ ID NO: 382; iii) a sequence that is complementary to SEQ ID NO: 382, where the sequence can comprise from 0 to 10 mismatches (non-complementary nucleotides) to SEQ ID NO: 382; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1a promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
5) In some cases, a recombinant expression vector comprises one of the following cassettes. In some cases, a cassette comprises, in order from 5′ to 3′: i) SEQ ID NO:854; ii) SEQ ID NO:383; iii) a sequence that is complementary to SEQ ID NO: 383, where the sequence can comprise from 0 to 10 mismatches (non-complementary nucleotides) to SEQ ID NO: 383; and iv) SEQ ID NO:855. In some cases, a cassette comprises, in order from 5′ to 3′: i) SEQ ID NO:856; ii) SEQ ID NO: 383; iii) a sequence that is complementary to SEQ ID NO: 383, where the sequence can comprise from 0 to 10 mismatches (non-complementary nucleotides) to SEQ ID NO: 383; and iv) SEQ ID NO: 857. In some cases, a cassette comprises, in order from 5′ to 3′: i) SEQ ID NO:858; ii) SEQ ID NO: 383; iii) a sequence that is complementary to SEQ ID NO: 383, where the sequence can comprise from 0 to 10 mismatches (non-complementary nucleotides) to SEQ ID NO: 383; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
6) In some cases, a recombinant expression vector comprises one of the following cassettes. In some cases, a cassette comprises, in order from 5′ to 3′: i) SEQ ID NO:854; ii) SEQ ID NO:384; iii) a sequence that is complementary to SEQ ID NO: 384, where the sequence can comprise from 0 to 10 mismatches (non-complementary nucleotides) to SEQ ID NO: 384; and iv) SEQ ID NO:855. In some cases, a cassette comprises, in order from 5′ to 3′: i) SEQ ID NO:856; ii) SEQ ID NO: 384; iii) a sequence that is complementary to SEQ ID NO: 384, where the sequence can comprise from 0 to 10 mismatches (non-complementary nucleotides) to SEQ ID NO: 384; and iv) SEQ ID NO:857. In some cases, a cassette comprises, in order from 5′ to 3′: i) SEQ ID NO:858; ii) SEQ ID NO: 384; iii) a sequence that is complementary to SEQ ID NO: 384, where the sequence can comprise from 0 to 10 mismatches (non-complementary nucleotides) to SEQ ID NO: 384; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
7) In some cases, a recombinant expression vector comprises one of the following cassettes. In some cases, a cassette comprises, in order from 5′ to 3′: i) SEQ ID NO:854; ii) SEQ ID NO:385; iii) a sequence that is complementary to SEQ ID NO: 385, where the sequence can comprise from 0 to 10 mismatches (non-complementary nucleotides) to SEQ ID NO: 385; and iv) SEQ ID NO:855. In some cases, a cassette comprises, in order from 5′ to 3′: i) SEQ ID NO:856; ii) SEQ ID NO: 385; iii) a sequence that is complementary to SEQ ID NO: 385, where the sequence can comprise from 0 to 10 mismatches (non-complementary nucleotides) to SEQ ID NO: 385; and iv) SEQ ID NO:857. In some cases, a cassette comprises, in order from 5′ to 3′: i) SEQ ID NO:858; ii) SEQ ID NO: 385; iii) a sequence that is complementary to SEQ ID NO: 385, where the sequence can comprise from 0 to 10 mismatches (non-complementary nucleotides) to SEQ ID NO: 385; and iv) recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
8) In some cases, a recombinant expression vector comprises one of the following cassettes. In some cases, a cassette comprises, in order from 5′ to 3′: i) SEQ ID NO:854; ii) SEQ ID NO:386; iii) a sequence that is complementary to SEQ ID NO: 386, where the sequence can comprise from 0 to 10 mismatches (non-complementary nucleotides) to SEQ ID NO: 386; and iv) SEQ ID NO:855. In some cases, a cassette comprises, in order from 5′ to 3′: i) SEQ ID NO:856; ii) SEQ ID NO: 386; iii) a sequence that is complementary to SEQ ID NO: 386, where the sequence can comprise from 0 to 10 mismatches (non-complementary nucleotides) to SEQ ID NO: 386; and iv) SEQ ID NO:857. In some cases, a cassette comprises, in order from 5′ to 3′: i) SEQ ID NO:858; ii) SEQ ID NO: 386; iii) a sequence that is complementary to SEQ ID NO: 386, where the sequence can comprise from 0 to 10 mismatches (non-complementary nucleotides) to SEQ ID NO: 386; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
9) In some cases, a recombinant expression vector comprises one of the following cassettes. In some cases, a cassette comprises, in order from 5′ to 3′: i) SEQ ID NO:854; ii) SEQ ID NO:387; iii) a sequence that is complementary to SEQ ID NO: 387, where the sequence can comprise from 0 to 10 mismatches (non-complementary nucleotides) to SEQ ID NO: 387; and iv) SEQ ID NO:855. In some cases, a cassette comprises, in order from 5′ to 3′: i) SEQ ID NO:856; ii) SEQ ID NO: 387; iii) a sequence that is complementary to SEQ ID NO: 387, where the sequence can comprise from 0 to 10 mismatches (non-complementary nucleotides) to SEQ ID NO: 387; and iv) SEQ ID NO:857. In some cases, a cassette comprises, in order from 5′ to 3′: i) SEQ ID NO:858; ii) SEQ ID NO: 387; iii) a sequence that is complementary to SEQ ID NO: 387, where the sequence can comprise from 0 to 10 mismatches (non-complementary nucleotides) to SEQ ID NO: 387; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
10) In some cases, a recombinant expression vector comprises one of the following cassettes. In some cases, a cassette comprises, in order from 5′ to 3′: i) SEQ ID NO:854; ii) SEQ ID NO:388; iii) a sequence that is complementary to SEQ ID NO: 388, where the sequence can comprise from 0 to 10 mismatches (non-complementary nucleotides) to SEQ ID NO: 388; and iv) SEQ ID NO:855. In some cases, a cassette comprises, in order from 5′ to 3′: i) SEQ ID NO:856; ii) SEQ ID NO: 388; iii) a sequence that is complementary to SEQ ID NO: 388, where the sequence can comprise from 0 to 10 mismatches (non-complementary nucleotides) to SEQ ID NO: 388; and iv) SEQ ID NO:857. In some cases, a cassette comprises, in order from 5′ to 3′: i) SEQ ID NO:858; ii) SEQ ID NO: 388; iii) a sequence that is complementary to SEQ ID NO: 388, where the sequence can comprise from 0 to 10 mismatches (non-complementary nucleotides) to SEQ ID NO: 388; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
11) In some cases, a recombinant expression vector comprises one of the following cassettes. In some cases, a cassette comprises, in order from 5′ to 3′: i) SEQ ID NO:854; ii) SEQ ID NO:389; iii) a sequence that is complementary to SEQ ID NO: 389, where the sequence can comprise from 0 to 10 mismatches (non-complementary nucleotides) to SEQ ID NO: 389; and iv) SEQ ID NO:855. In some cases, a cassette comprises, in order from 5′ to 3′: i) SEQ ID NO:856; ii) SEQ ID NO: 389; iii) a sequence that is complementary to SEQ ID NO: 389, where the sequence can comprise from 0 to 10 mismatches (non-complementary nucleotides) to SEQ ID NO: 389; and iv) SEQ ID NO: 857. In some cases, a cassette comprises, in order from 5′ to 3′: i) SEQ ID NO:858; ii) SEQ ID NO: 389; iii) a sequence that is complementary to SEQ ID NO: 389, where the sequence can comprise from 0 to 10 mismatches (non-complementary nucleotides) to SEQ ID NO: 389; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
12) In some cases, a recombinant expression vector comprises one of the following cassettes. In some cases, a cassette comprises, in order from 5′ to 3′: i) SEQ ID NO:854; ii) SEQ ID NO:390; iii) a sequence that is complementary to SEQ ID NO: 390, where the sequence can comprise from 0 to 10 mismatches (non-complementary nucleotides) to SEQ ID NO: 390; and iv) SEQ ID NO:855. In some cases, a cassette comprises, in order from 5′ to 3′: i) SEQ ID NO:856; ii) SEQ ID NO: 390; iii) a sequence that is complementary to SEQ ID NO: 390, where the sequence can comprise from 0 to 10 mismatches (non-complementary nucleotides) to SEQ ID NO: 390; and iv) SEQ ID NO:857. In some cases, a cassette comprises, in order from 5′ to 3′: i) SEQ ID NO:858; ii) SEQ ID NO: 390; iii) a sequence that is complementary to SEQ ID NO: 309, where the sequence can comprise from 0 to 10 mismatches (non-complementary nucleotides) to SEQ ID NO: 390; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
13) In some cases, a recombinant expression vector comprises one of the following cassettes. In some cases, a cassette comprises, in order from 5′ to 3′: i) SEQ ID NO:854; ii) SEQ ID NO:391; iii) a sequence that is complementary to SEQ ID NO: 391, where the sequence can comprise from 0 to 10 mismatches (non-complementary nucleotides) to SEQ ID NO: 391; and iv) SEQ ID NO:855. In some cases, a cassette comprises, in order from 5′ to 3′: i) SEQ ID NO:856; ii) SEQ ID NO: 391; iii) a sequence that is complementary to SEQ ID NO: 391, where the sequence can comprise from 0 to 10 mismatches (non-complementary nucleotides) to SEQ ID NO: 391; and iv) SEQ ID NO:857. In some cases, a cassette comprises, in order from 5′ to 3′: i) SEQ ID NO:858; ii) SEQ ID NO: 391; iii) a sequence that is complementary to SEQ ID NO: 391, where the sequence can comprise from 0 to 10 mismatches (non-complementary nucleotides) to SEQ ID NO: 391; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
14) In some cases, a recombinant expression vector comprises one of the following cassettes. In some cases, a cassette comprises, in order from 5′ to 3′: i) SEQ ID NO:854; ii) SEQ ID NO:390; iii) a sequence that is complementary to SEQ ID NO: 390, where the sequence can comprise from 0 to 10 mismatches (non-complementary nucleotides) to SEQ ID NO: 390; and iv) SEQ ID NO:855. In some cases, a cassette comprises, in order from 5′ to 3′: i) SEQ ID NO:856; ii) SEQ ID NO: 390; iii) a sequence that is complementary to SEQ ID NO: 390, where the sequence can comprise from 0 to 10 mismatches (non-complementary nucleotides) to SEQ ID NO: 390; and iv) SEQ ID NO: 857. In some cases, a cassette comprises, in order from 5′ to 3′: i) SEQ ID NO:858; ii) SEQ ID NO: 390; iii) a sequence that is complementary to SEQ ID NO: 390, where the sequence can comprise from 0 to 10 mismatches (non-complementary nucleotides) to SEQ ID NO: 390; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
15) In some cases, a recombinant expression vector comprises one of the following cassettes. In some cases, a cassette comprises, in order from 5′ to 3′: i) SEQ ID NO:854; ii) SEQ ID NO: 391; iii) a sequence that is complementary to SEQ ID NO: 391, where the sequence can comprise from 0 to 10 mismatches (non-complementary nucleotides) to SEQ ID NO: 391; and iv) SEQ ID NO:855. In some cases, a cassette comprises, in order from 5′ to 3′: i) SEQ ID NO:856; ii) SEQ ID NO: 391; iii) a sequence that is complementary to SEQ ID NO: 391, where the sequence can comprise from 0 to 10 mismatches (non-complementary nucleotides) to SEQ ID NO: 391; and iv) SEQ ID NO: 857. In some cases, a cassette comprises, in order from 5′ to 3′: i) SEQ ID NO:858; ii) SEQ ID NO: 391; iii) a sequence that is complementary to SEQ ID NO: 391, where the sequence can comprise from 0 to 10 mismatches (non-complementary nucleotides) to SEQ ID NO: 391; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
16) In some cases, a recombinant expression vector comprises one of the following cassettes. In some cases, a cassette comprises, in order from 5′ to 3′: i) SEQ ID NO:854; ii) SEQ ID NO:392; iii) a sequence that is complementary to SEQ ID NO: 392, where the sequence can comprise from 0 to 10 mismatches (non-complementary nucleotides) to SEQ ID NO: 392; and iv) SEQ ID NO:855. In some cases, a cassette comprises, in order from 5′ to 3′: i) SEQ ID NO:856; ii) SEQ ID NO: 392; iii) a sequence that is complementary to SEQ ID NO: 392, where the sequence can comprise from 0 to 10 mismatches (non-complementary nucleotides) to SEQ ID NO: 392; and iv) SEQ ID NO: 857. In some cases, a cassette comprises, in order from 5′ to 3′: i) SEQ ID NO:858; ii) SEQ ID NO: 392; iii) a sequence that is complementary to SEQ ID NO: 392, where the sequence can comprise from 0 to 10 mismatches (non-complementary nucleotides) to SEQ ID NO: 392; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
17) In some cases, a recombinant expression vector comprises one of the following cassettes. In some cases, a cassette comprises, in order from 5′ to 3′: i) SEQ ID NO:854; ii) SEQ ID NO:393; iii) a sequence that is complementary to SEQ ID NO: 393, where the sequence can comprise from 0 to 10 mismatches (non-complementary nucleotides) to SEQ ID NO: 393; and iv) SEQ ID NO:855. In some cases, a cassette comprises, in order from 5′ to 3′: i) SEQ ID NO:856; ii) SEQ ID NO: 393; iii) a sequence that is complementary to SEQ ID NO: 393, where the sequence can comprise from 0 to 10 mismatches (non-complementary nucleotides) to SEQ ID NO: 393; and iv) SEQ ID NO: 857. In some cases, a cassette comprises, in order from 5′ to 3′: i) SEQ ID NO:858; ii) SEQ ID NO: 393; iii) a sequence that is complementary to SEQ ID NO: 393, where the sequence can comprise from 0 to 10 mismatches (non-complementary nucleotides) to SEQ ID NO: 393; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
18) In some cases, a recombinant expression vector comprises one of the following cassettes. In some cases, a cassette comprises, in order from 5′ to 3′: i) SEQ ID NO:854; ii) SEQ ID NO:394; iii) a sequence that is complementary to SEQ ID NO: 394, where the sequence can comprise from 0 to 10 mismatches (non-complementary nucleotides) to SEQ ID NO: 394; and iv) SEQ ID NO:855. In some cases, a cassette comprises, in order from 5′ to 3′: i) SEQ ID NO:856; ii) SEQ ID NO: 394; iii) a sequence that is complementary to SEQ ID NO: 394, where the sequence can comprise from 0 to 10 mismatches (non-complementary nucleotides) to SEQ ID NO: 394; and iv) SEQ ID NO: 857. In some cases, a cassette comprises, in order from 5′ to 3′: i) SEQ ID NO:858; ii) SEQ ID NO: 394; iii) a sequence that is complementary to SEQ ID NO: 394, where the sequence can comprise from 0 to 10 mismatches (non-complementary nucleotides) to SEQ ID NO: 394; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
19) In some cases, a recombinant expression vector comprises one of the following cassettes. In some cases, a cassette comprises, in order from 5′ to 3′: i) SEQ ID NO:854; ii) SEQ ID NO:395; iii) a sequence that is complementary to SEQ ID NO: 395, where the sequence can comprise from 0 to 10 mismatches (non-complementary nucleotides) to SEQ ID NO: 395; and iv) SEQ ID NO:855. In some cases, a cassette comprises, in order from 5′ to 3′: i) SEQ ID NO:856; ii) SEQ ID NO: 395; iii) a sequence that is complementary to SEQ ID NO: 395, where the sequence can comprise from 0 to 10 mismatches (non-complementary nucleotides) to SEQ ID NO: 395; and iv) SEQ ID NO: 857. In some cases, a cassette comprises, in order from 5′ to 3′: i) SEQ ID NO:858; ii) SEQ ID NO: 395; iii) a sequence that is complementary to SEQ ID NO: 395, where the sequence can comprise from 0 to 10 mismatches (non-complementary nucleotides) to SEQ ID NO: 395; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
20) In some cases, a recombinant expression vector comprises one of the following cassettes. In some cases, a cassette comprises, in order from 5′ to 3′: i) SEQ ID NO:854; ii) SEQ ID NO:396; iii) a sequence that is complementary to SEQ ID NO: 396, where the sequence can comprise from 0 to 10 mismatches (non-complementary nucleotides) to SEQ ID NO: 396; and iv) SEQ ID NO:855. In some cases, a cassette comprises, in order from 5′ to 3′: i) SEQ ID NO:856; ii) SEQ ID NO: 396; iii) a sequence that is complementary to SEQ ID NO: 396, where the sequence can comprise from 0 to 10 mismatches (non-complementary nucleotides) to SEQ ID NO: 396; and iv) SEQ ID NO: 857. In some cases, a cassette comprises, in order from 5′ to 3′: i) SEQ ID NO:858; ii) SEQ ID NO: 396; iii) a sequence that is complementary to SEQ ID NO: 396, where the sequence can comprise from 0 to 10 mismatches (non-complementary nucleotides) to SEQ ID NO: 396; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs. 21) In some cases, a recombinant expression vector comprises one of the following cassettes. In some cases, a cassette comprises, in order from 5′ to 3′: i) SEQ ID NO:854; ii) SEQ ID NO:397; iii) a sequence that is complementary to SEQ ID NO: 397, where the sequence can comprise from 0 to 10 mismatches (non-complementary nucleotides) to SEQ ID NO: 397; and iv) SEQ ID NO:855. In some cases, a cassette comprises, in order from 5′ to 3′: i) SEQ ID NO:856; ii) SEQ ID NO: 397; iii) a sequence that is complementary to SEQ ID NO: 397, where the sequence can comprise from 0 to 10 mismatches (non-complementary nucleotides) to SEQ ID NO: 397; and iv) SEQ ID NO: 857. In some cases, a cassette comprises, in order from 5′ to 3′: i) SEQ ID NO:858; ii) SEQ ID NO: 397; iii) a sequence that is complementary to SEQ ID NO: 397, where the sequence can comprise from 0 to 10 mismatches (non-complementary nucleotides) to SEQ ID NO: 397; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
22) In some cases, a recombinant expression vector comprises one of the following cassettes. In some cases, a cassette comprises, in order from 5′ to 3′: i) SEQ ID NO:854; ii) SEQ ID NO:398; iii) a sequence that is complementary to SEQ ID NO: 398, where the sequence can comprise from 0 to 10 mismatches (non-complementary nucleotides) to SEQ ID NO: 398; and iv) SEQ ID NO:855. In some cases, a cassette comprises, in order from 5′ to 3′: i) SEQ ID NO:856; ii) SEQ ID NO: 398; iii) a sequence that is complementary to SEQ ID NO: 398, where the sequence can comprise from 0 to 10 mismatches (non-complementary nucleotides) to SEQ ID NO: 398; and iv) SEQ ID NO: 857. In some cases, a cassette comprises, in order from 5′ to 3′: i) SEQ ID NO:858; ii) SEQ ID NO: 398; iii) a sequence that is complementary to SEQ ID NO: 398, where the sequence can comprise from 0 to 10 mismatches (non-complementary nucleotides) to SEQ ID NO: 398; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
23) In some cases, a recombinant expression vector comprises one of the following cassettes. In some cases, a cassette comprises, in order from 5′ to 3′: i) SEQ ID NO:854; ii) SEQ ID NO:399; iii) a sequence that is complementary to SEQ ID NO: 399, where the sequence can comprise from 0 to 10 mismatches (non-complementary nucleotides) to SEQ ID NO: 399; and iv) SEQ ID NO:855. In some cases, a cassette comprises, in order from 5′ to 3′: i) SEQ ID NO:856; ii) SEQ ID NO: 399; iii) a sequence that is complementary to SEQ ID NO: 399, where the sequence can comprise from 0 to 10 mismatches (non-complementary nucleotides) to SEQ ID NO: 399; and iv) SEQ ID NO: 857. In some cases, a cassette comprises, in order from 5′ to 3′: i) SEQ ID NO:858; ii) SEQ ID NO: 399; iii) a sequence that is complementary to SEQ ID NO: 399, where the sequence can comprise from 0 to 10 mismatches (non-complementary nucleotides) to SEQ ID NO: 399; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
24) In some cases, a recombinant expression vector comprises one of the following cassettes. In some cases, a cassette comprises, in order from 5′ to 3′: i) SEQ ID NO:854; ii) SEQ ID NO:400; iii) a sequence that is complementary to SEQ ID NO: 400, where the sequence can comprise from 0 to 10 mismatches (non-complementary nucleotides) to SEQ ID NO: 400; and iv) SEQ ID NO:855. In some cases, a cassette comprises, in order from 5′ to 3′: i) SEQ ID NO:856; ii) SEQ ID NO: 400; iii) a sequence that is complementary to SEQ ID NO: 400, where the sequence can comprise from 0 to 10 mismatches (non-complementary nucleotides) to SEQ ID NO: 400; and iv) SEQ ID NO: 857. In some cases, a cassette comprises, in order from 5′ to 3′: i) SEQ ID NO:858; ii) SEQ ID NO: 400; iii) a sequence that is complementary to SEQ ID NO: 400, where the sequence can comprise from 0 to 10 mismatches (non-complementary nucleotides) to SEQ ID NO: 400; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
25) In some cases, a recombinant expression vector comprises one of the following cassettes. In some cases, a cassette comprises, in order from 5′ to 3′: i) SEQ ID NO:854; ii) SEQ ID NO:401; iii) a sequence that is complementary to SEQ ID NO: 401, where the sequence can comprise from 0 to 10 mismatches (non-complementary nucleotides) to SEQ ID NO: 401; and iv) SEQ ID NO:855. In some cases, a cassette comprises, in order from 5′ to 3′: i) SEQ ID NO:856; ii) SEQ ID NO: 401; iii) a sequence that is complementary to SEQ ID NO: 401, where the sequence can comprise from 0 to 10 mismatches (non-complementary nucleotides) to SEQ ID NO: 401; and iv) SEQ ID NO: 857. In some cases, a cassette comprises, in order from 5′ to 3′: i) SEQ ID NO:858; ii) SEQ ID NO: 401; iii) a sequence that is complementary to SEQ ID NO: 401, where the sequence can comprise from 0 to 10 mismatches (non-complementary nucleotides) to SEQ ID NO: 401; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
26) In some cases, a recombinant expression vector comprises one of the following cassettes. In some cases, a cassette comprises, in order from 5′ to 3′: i) SEQ ID NO:854; ii) SEQ ID NO:402; iii) a sequence that is complementary to SEQ ID NO: 402, where the sequence can comprise from 0 to 10 mismatches (non-complementary nucleotides) to SEQ ID NO: 402; and iv) SEQ ID NO:855. In some cases, a cassette comprises, in order from 5′ to 3′: i) SEQ ID NO:856; ii) SEQ ID NO: 402; iii) a sequence that is complementary to SEQ ID NO: 402, where the sequence can comprise from 0 to 10 mismatches (non-complementary nucleotides) to SEQ ID NO: 402; and iv) SEQ ID NO: 857. In some cases, a cassette comprises, in order from 5′ to 3′: i) SEQ ID NO:858; ii) SEQ ID NO: 402; iii) a sequence that is complementary to SEQ ID NO: 402, where the sequence can comprise from 0 to 10 mismatches (non-complementary nucleotides) to SEQ ID NO: 402; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs. 27) In some cases, a recombinant expression vector comprises one of the following cassettes. In some cases, a cassette comprises, in order from 5′ to 3′: i) SEQ ID NO:854; ii) SEQ ID NO:403; iii) a sequence that is complementary to SEQ ID NO: 403, where the sequence can comprise from 0 to 10 mismatches (non-complementary nucleotides) to SEQ ID NO: 403; and iv) SEQ ID NO:855. In some cases, a cassette comprises, in order from 5′ to 3′: i) SEQ ID NO:856; ii) SEQ ID NO: 403; iii) a sequence that is complementary to SEQ ID NO: 403, where the sequence can comprise from 0 to 10 mismatches (non-complementary nucleotides) to SEQ ID NO: 403; and iv) SEQ ID NO: 857. In some cases, a cassette comprises, in order from 5′ to 3′: i) SEQ ID NO:858; ii) SEQ ID NO: 403; iii) a sequence that is complementary to SEQ ID NO: 403, where the sequence can comprise from 0 to 10 mismatches (non-complementary nucleotides) to SEQ ID NO: 403; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
28) In some cases, a recombinant expression vector comprises one of the following cassettes. In some cases, a cassette comprises, in order from 5′ to 3′: i) SEQ ID NO:854; ii) SEQ ID NO:404; iii) a sequence that is complementary to SEQ ID NO: 404, where the sequence can comprise from 0 to 10 mismatches (non-complementary nucleotides) to SEQ ID NO: 404; and iv) SEQ ID NO:855. In some cases, a cassette comprises, in order from 5′ to 3′: i) SEQ ID NO:856; ii) SEQ ID NO: 404; iii) a sequence that is complementary to SEQ ID NO: 404, where the sequence can comprise from 0 to 10 mismatches (non-complementary nucleotides) to SEQ ID NO: 404; and iv) SEQ ID NO: 857. In some cases, a cassette comprises, in order from 5′ to 3′: i) SEQ ID NO:858; ii) SEQ ID NO: 404; iii) a sequence that is complementary to SEQ ID NO: 404, where the sequence can comprise from 0 to 10 mismatches (non-complementary nucleotides) to SEQ ID NO: 404; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
29) In some cases, a recombinant expression vector comprises one of the following cassettes. In some cases, a cassette comprises, in order from 5′ to 3′: i) SEQ ID NO:854; ii) SEQ ID NO:405; iii) a sequence that is complementary to SEQ ID NO: 405, where the sequence can comprise from 0 to 10 mismatches (non-complementary nucleotides) to SEQ ID NO: 405; and iv) SEQ ID NO:855. In some cases, a cassette comprises, in order from 5′ to 3′: i) SEQ ID NO:856; ii) SEQ ID NO: 405; iii) a sequence that is complementary to SEQ ID NO: 405, where the sequence can comprise from 0 to 10 mismatches (non-complementary nucleotides) to SEQ ID NO: 405; and iv) SEQ ID NO: 857. In some cases, a cassette comprises, in order from 5′ to 3′: i) SEQ ID NO:858; ii) SEQ ID NO: 405; iii) a sequence that is complementary to SEQ ID NO: 405, where the sequence can comprise from 0 to 10 mismatches (non-complementary nucleotides) to SEQ ID NO: 405; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
30) In some cases, a recombinant expression vector comprises one of the following cassettes. In some cases, a cassette comprises, in order from 5′ to 3′: i) SEQ ID NO:854; ii) SEQ ID NO:406; iii) a sequence that is complementary to SEQ ID NO: 406, where the sequence can comprise from 0 to 10 mismatches (non-complementary nucleotides) to SEQ ID NO: 406; and iv) SEQ ID NO:855. In some cases, a cassette comprises, in order from 5′ to 3′: i) SEQ ID NO:856; ii) SEQ ID NO: 406; iii) a sequence that is complementary to SEQ ID NO: 406, where the sequence can comprise from 0 to 10 mismatches (non-complementary nucleotides) to SEQ ID NO: 406; and iv) SEQ ID NO: 857. In some cases, a cassette comprises, in order from 5′ to 3′: i) SEQ ID NO:858; ii) SEQ ID NO: 406; iii) a sequence that is complementary to SEQ ID NO: 406, where the sequence can comprise from 0 to 10 mismatches (non-complementary nucleotides) to SEQ ID NO: 406; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
31) In some cases, a recombinant expression vector comprises one of the following cassettes. In some cases, a cassette comprises, in order from 5′ to 3′: i) SEQ ID NO:854; ii) SEQ ID NO:407; iii) a sequence that is complementary to SEQ ID NO: 407, where the sequence can comprise from 0 to 10 mismatches (non-complementary nucleotides) to SEQ ID NO: 407; and iv) SEQ ID NO:855. In some cases, a cassette comprises, in order from 5′ to 3′: i) SEQ ID NO:856; ii) SEQ ID NO: 407; iii) a sequence that is complementary to SEQ ID NO: 407, where the sequence can comprise from 0 to 10 mismatches (non-complementary nucleotides) to SEQ ID NO: 407; and iv) SEQ ID NO: 857. In some cases, a cassette comprises, in order from 5′ to 3′: i) SEQ ID NO:858; ii) SEQ ID NO: 407; iii) a sequence that is complementary to SEQ ID NO: 407, where the sequence can comprise from 0 to 10 mismatches (non-complementary nucleotides) to SEQ ID NO: 407; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
32) In some cases, a recombinant expression vector comprises one of the following cassettes. In some cases, a cassette comprises, in order from 5′ to 3′: i) SEQ ID NO:854; ii) SEQ ID NO:408; iii) a sequence that is complementary to SEQ ID NO: 408, where the sequence can comprise from 0 to 10 mismatches (non-complementary nucleotides) to SEQ ID NO: 408; and iv) SEQ ID NO:855. In some cases, a cassette comprises, in order from 5′ to 3′: i) SEQ ID NO:856; ii) SEQ ID NO: 408; iii) a sequence that is complementary to SEQ ID NO: 408, where the sequence can comprise from 0 to 10 mismatches (non-complementary nucleotides) to SEQ ID NO: 408; and iv) SEQ ID NO: 857. In some cases, a cassette comprises, in order from 5′ to 3′: i) SEQ ID NO:858; ii) SEQ ID NO: 408; iii) a sequence that is complementary to SEQ ID NO: 408, where the sequence can comprise from 0 to 10 mismatches (non-complementary nucleotides) to SEQ ID NO: 408; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
33) In some cases, a recombinant expression vector comprises one of the following cassettes. In some cases, a cassette comprises, in order from 5′ to 3′: i) SEQ ID NO:854; ii) SEQ ID NO:409; iii) a sequence that is complementary to SEQ ID NO: 409, where the sequence can comprise from 0 to 10 mismatches (non-complementary nucleotides) to SEQ ID NO: 409; and iv) SEQ ID NO:855. In some cases, a cassette comprises, in order from 5′ to 3′: i) SEQ ID NO:856; ii) SEQ ID NO: 409; iii) a sequence that is complementary to SEQ ID NO: 409, where the sequence can comprise from 0 to 10 mismatches (non-complementary nucleotides) to SEQ ID NO: 409; and iv) SEQ ID NO: 857. In some cases, a cassette comprises, in order from 5′ to 3′: i) SEQ ID NO:858; ii) SEQ ID NO: 409; iii) a sequence that is complementary to SEQ ID NO: 409, where the sequence can comprise from 0 to 10 mismatches (non-complementary nucleotides) to SEQ ID NO: 409; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
34) In some cases, a recombinant expression vector comprises one of the following cassettes. In some cases, a cassette comprises, in order from 5′ to 3′: i) SEQ ID NO:854; ii) SEQ ID NO:410; iii) a sequence that is complementary to SEQ ID NO: 410, where the sequence can comprise from 0 to 10 mismatches (non-complementary nucleotides) to SEQ ID NO: 410; and iv) SEQ ID NO:855. In some cases, a cassette comprises, in order from 5′ to 3′: i) SEQ ID NO:856; ii) SEQ ID NO: 410; iii) a sequence that is complementary to SEQ ID NO: 410, where the sequence can comprise from 0 to 10 mismatches (non-complementary nucleotides) to SEQ ID NO: 410; and iv) SEQ ID NO: 857. In some cases, a cassette comprises, in order from 5′ to 3′: i) SEQ ID NO:858; ii) SEQ ID NO: 410; iii) a sequence that is complementary to SEQ ID NO: 410, where the sequence can comprise from 0 to 10 mismatches (non-complementary nucleotides) to SEQ ID NO: 410; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
35) In some cases, a recombinant expression vector comprises one of the following cassettes. In some cases, a cassette comprises, in order from 5′ to 3′: i) SEQ ID NO:854; ii) SEQ ID NO:411; iii) a sequence that is complementary to SEQ ID NO: 411, where the sequence can comprise from 0 to 10 mismatches (non-complementary nucleotides) to SEQ ID NO: 411; and iv) SEQ ID NO:855. In some cases, a cassette comprises, in order from 5′ to 3′: i) SEQ ID NO:856; ii) SEQ ID NO: 411; iii) a sequence that is complementary to SEQ ID NO: 411, where the sequence can comprise from 0 to 10 mismatches (non-complementary nucleotides) to SEQ ID NO: 411; and iv) SEQ ID NO: 857. In some cases, a cassette comprises, in order from 5′ to 3′: i) SEQ ID NO:858; ii) SEQ ID NO: 411; iii) a sequence that is complementary to SEQ ID NO: 411, where the sequence can comprise from 0 to 10 mismatches (non-complementary nucleotides) to SEQ ID NO: 411; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
36) In some cases, a recombinant expression vector comprises one of the following cassettes. In some cases, a cassette comprises, in order from 5′ to 3′: i) SEQ ID NO:854; ii) SEQ ID NO:412; iii) a sequence that is complementary to SEQ ID NO: 412, where the sequence can comprise from 0 to 10 mismatches (non-complementary nucleotides) to SEQ ID NO: 412; and iv) SEQ ID NO:855. In some cases, a cassette comprises, in order from 5′ to 3′: i) SEQ ID NO:856; ii) SEQ ID NO: 412; iii) a sequence that is complementary to SEQ ID NO: 412, where the sequence can comprise from 0 to 10 mismatches (non-complementary nucleotides) to SEQ ID NO: 412; and iv) SEQ ID NO: 857. In some cases, a cassette comprises, in order from 5′ to 3′: i) SEQ ID NO:858; ii) SEQ ID NO: 412; iii) a sequence that is complementary to SEQ ID NO: 412, where the sequence can comprise from 0 to 10 mismatches (non-complementary nucleotides) to SEQ ID NO: 412; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
37) In some cases, a recombinant expression vector comprises one of the following cassettes. In some cases, a cassette comprises, in order from 5′ to 3′: i) SEQ ID NO:854; ii) SEQ ID NO:413; iii) a sequence that is complementary to SEQ ID NO: 413, where the sequence can comprise from 0 to 10 mismatches (non-complementary nucleotides) to SEQ ID NO: 413; and iv) SEQ ID NO:855. In some cases, a cassette comprises, in order from 5′ to 3′: i) SEQ ID NO:856; ii) SEQ ID NO: 413; iii) a sequence that is complementary to SEQ ID NO: 413, where the sequence can comprise from 0 to 10 mismatches (non-complementary nucleotides) to SEQ ID NO: 413; and iv) SEQ ID NO: 857. In some cases, a cassette comprises, in order from 5′ to 3′: i) SEQ ID NO:858; ii) SEQ ID NO: 413; iii) a sequence that is complementary to SEQ ID NO: 413, where the sequence can comprise from 0 to 10 mismatches (non-complementary nucleotides) to SEQ ID NO: 413; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
38) In some cases, a recombinant expression vector comprises one of the following cassettes. In some cases, a cassette comprises, in order from 5′ to 3′: i) SEQ ID NO:854; ii) SEQ ID NO:414; iii) a sequence that is complementary to SEQ ID NO: 414, where the sequence can comprise from 0 to 10 mismatches (non-complementary nucleotides) to SEQ ID NO: 414; and iv) SEQ ID NO:855. In some cases, a cassette comprises, in order from 5′ to 3′: i) SEQ ID NO:856; ii) SEQ ID NO: 414; iii) a sequence that is complementary to SEQ ID NO: 414, where the sequence can comprise from 0 to 10 mismatches (non-complementary nucleotides) to SEQ ID NO: 414; and iv) SEQ ID NO: 857. In some cases, a cassette comprises, in order from 5′ to 3′: i) SEQ ID NO:858; ii) SEQ ID NO: 414; iii) a sequence that is complementary to SEQ ID NO: 414, where the sequence can comprise from 0 to 10 mismatches (non-complementary nucleotides) to SEQ ID NO: 414; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
39) In some cases, a recombinant expression vector comprises one of the following cassettes. In some cases, a cassette comprises, in order from 5′ to 3′: i) SEQ ID NO:854; ii) SEQ ID NO:415; iii) a sequence that is complementary to SEQ ID NO: 415, where the sequence can comprise from 0 to 10 mismatches (non-complementary nucleotides) to SEQ ID NO: 415; and iv) SEQ ID NO:855. In some cases, a cassette comprises, in order from 5′ to 3′: i) SEQ ID NO:856; ii) SEQ ID NO: 415; iii) a sequence that is complementary to SEQ ID NO: 415, where the sequence can comprise from 0 to 10 mismatches (non-complementary nucleotides) to SEQ ID NO: 415; and iv) SEQ ID NO: 857. In some cases, a cassette comprises, in order from 5′ to 3′: i) SEQ ID NO:858; ii) SEQ ID NO: 415; iii) a sequence that is complementary to SEQ ID NO: 415, where the sequence can comprise from 0 to 10 mismatches (non-complementary nucleotides) to SEQ ID NO: 415; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
40) In some cases, a recombinant expression vector comprises one of the following cassettes. In some cases, a cassette comprises, in order from 5′ to 3′: i) SEQ ID NO:854; ii) SEQ ID NO:416; iii) a sequence that is complementary to SEQ ID NO: 416, where the sequence can comprise from 0 to 10 mismatches (non-complementary nucleotides) to SEQ ID NO: 416; and iv) SEQ ID NO:855. In some cases, a cassette comprises, in order from 5′ to 3′: i) SEQ ID NO:856; ii) SEQ ID NO: 416; iii) a sequence that is complementary to SEQ ID NO: 416, where the sequence can comprise from 0 to 10 mismatches (non-complementary nucleotides) to SEQ ID NO: 416; and iv) SEQ ID NO: 857. In some cases, a cassette comprises, in order from 5′ to 3′: i) SEQ ID NO:858; ii) SEQ ID NO: 416; iii) a sequence that is complementary to SEQ ID NO: 416, where the sequence can comprise from 0 to 10 mismatches (non-complementary nucleotides) to SEQ ID NO: 416; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
41) In some cases, a recombinant expression vector comprises one of the following cassettes. In some cases, a cassette comprises, in order from 5′ to 3′: i) SEQ ID NO:854; ii) SEQ ID NO:417; iii) a sequence that is complementary to SEQ ID NO: 417, where the sequence can comprise from 0 to 10 mismatches (non-complementary nucleotides) to SEQ ID NO: 417; and iv) SEQ ID NO:855. In some cases, a cassette comprises, in order from 5′ to 3′: i) SEQ ID NO:856; ii) SEQ ID NO: 417; iii) a sequence that is complementary to SEQ ID NO: 417, where the sequence can comprise from 0 to 10 mismatches (non-complementary nucleotides) to SEQ ID NO: 417; and iv) SEQ ID NO: 857. In some cases, a cassette comprises, in order from 5′ to 3′: i) SEQ ID NO:858; ii) SEQ ID NO: 417; iii) a sequence that is complementary to SEQ ID NO: 417, where the sequence can comprise from 0 to 10 mismatches (non-complementary nucleotides) to SEQ ID NO: 417; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs. 42) In some cases, a recombinant expression vector comprises one of the following cassettes. In some cases, a cassette comprises, in order from 5′ to 3′: i) SEQ ID NO:854; ii) SEQ ID NO:418; iii) a sequence that is complementary to SEQ ID NO: 418, where the sequence can comprise from 0 to 10 mismatches (non-complementary nucleotides) to SEQ ID NO: 418; and iv) SEQ ID NO:855. In some cases, a cassette comprises, in order from 5′ to 3′: i) SEQ ID NO:856; ii) SEQ ID NO: 418; iii) a sequence that is complementary to SEQ ID NO: 418, where the sequence can comprise from 0 to 10 mismatches (non-complementary nucleotides) to SEQ ID NO: 418; and iv) SEQ ID NO: 857. In some cases, a cassette comprises, in order from 5′ to 3′: i) SEQ ID NO:858; ii) SEQ ID NO: 418; iii) a sequence that is complementary to SEQ ID NO: 418, where the sequence can comprise from 0 to 10 mismatches (non-complementary nucleotides) to SEQ ID NO: 418; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
43) In some cases, a recombinant expression vector comprises one of the following cassettes. In some cases, a cassette comprises, in order from 5′ to 3′: i) SEQ ID NO:854; ii) SEQ ID NO:419; iii) a sequence that is complementary to SEQ ID NO: 419, where the sequence can comprise from 0 to 10 mismatches (non-complementary nucleotides) to SEQ ID NO: 419; and iv) SEQ ID NO:855. In some cases, a cassette comprises, in order from 5′ to 3′: i) SEQ ID NO:856; ii) SEQ ID NO: 419; iii) a sequence that is complementary to SEQ ID NO: 419, where the sequence can comprise from 0 to 10 mismatches (non-complementary nucleotides) to SEQ ID NO: 419; and iv) SEQ ID NO: 857. In some cases, a cassette comprises, in order from 5′ to 3′: i) SEQ ID NO:858; ii) SEQ ID NO: 419; iii) a sequence that is complementary to SEQ ID NO: 419, where the sequence can comprise from 0 to 10 mismatches (non-complementary nucleotides) to SEQ ID NO: 419; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
44) In some cases, a recombinant expression vector comprises one of the following cassettes. In some cases, a cassette comprises, in order from 5′ to 3′: i) SEQ ID NO:854; ii) SEQ ID NO:420; iii) a sequence that is complementary to SEQ ID NO: 420, where the sequence can comprise from 0 to 10 mismatches (non-complementary nucleotides) to SEQ ID NO: 420; and iv) SEQ ID NO:855. In some cases, a cassette comprises, in order from 5′ to 3′: i) SEQ ID NO:856; ii) SEQ ID NO: 420; iii) a sequence that is complementary to SEQ ID NO: 420, where the sequence can comprise from 0 to 10 mismatches (non-complementary nucleotides) to SEQ ID NO: 420; and iv) SEQ ID NO: 857. In some cases, a cassette comprises, in order from 5′ to 3′: i) SEQ ID NO:858; ii) SEQ ID NO: 420; iii) a sequence that is complementary to SEQ ID NO: 420, where the sequence can comprise from 0 to 10 mismatches (non-complementary nucleotides) to SEQ ID NO: 420; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
45) In some cases, a recombinant expression vector comprises one of the following cassettes. In some cases, a cassette comprises, in order from 5′ to 3′: i) SEQ ID NO:854; ii) SEQ ID NO:421; iii) a sequence that is complementary to SEQ ID NO: 421, where the sequence can comprise from 0 to 10 mismatches (non-complementary nucleotides) to SEQ ID NO: 421; and iv) SEQ ID NO:855. In some cases, a cassette comprises, in order from 5′ to 3′: i) SEQ ID NO:856; ii) SEQ ID NO: 421; iii) a sequence that is complementary to SEQ ID NO: 421, where the sequence can comprise from 0 to 10 mismatches (non-complementary nucleotides) to SEQ ID NO: 421; and iv) SEQ ID NO: 857. In some cases, a cassette comprises, in order from 5′ to 3′: i) SEQ ID NO:858; ii) SEQ ID NO: 421; iii) a sequence that is complementary to SEQ ID NO: 421, where the sequence can comprise from 0 to 10 mismatches (non-complementary nucleotides) to SEQ ID NO: 421; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
46) In some cases, a recombinant expression vector comprises one of the following cassettes. In some cases, a cassette comprises, in order from 5′ to 3′: i) SEQ ID NO:854; ii) SEQ ID NO:422; iii) a sequence that is complementary to SEQ ID NO: 422, where the sequence can comprise from 0 to 10 mismatches (non-complementary nucleotides) to SEQ ID NO: 422; and iv) SEQ ID NO:855. In some cases, a cassette comprises, in order from 5′ to 3′: i) SEQ ID NO:856; ii) SEQ ID NO: 422; iii) a sequence that is complementary to SEQ ID NO: 422, where the sequence can comprise from 0 to 10 mismatches (non-complementary nucleotides) to SEQ ID NO: 422; and iv) SEQ ID NO: 857. In some cases, a cassette comprises, in order from 5′ to 3′: i) SEQ ID NO:858; ii) SEQ ID NO: 422; iii) a sequence that is complementary to SEQ ID NO: 422, where the sequence can comprise from 0 to 10 mismatches (non-complementary nucleotides) to SEQ ID NO: 422; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
47) In some cases, a recombinant expression vector comprises one of the following cassettes. In some cases, a cassette comprises, in order from 5′ to 3′: i) SEQ ID NO:854; ii) SEQ ID NO:423; iii) a sequence that is complementary to SEQ ID NO: 423, where the sequence can comprise from 0 to 10 mismatches (non-complementary nucleotides) to SEQ ID NO: 423; and iv) SEQ ID NO:855. In some cases, a cassette comprises, in order from 5′ to 3′: i) SEQ ID NO:856; ii) SEQ ID NO: 423; iii) a sequence that is complementary to SEQ ID NO: 423, where the sequence can comprise from 0 to 10 mismatches (non-complementary nucleotides) to SEQ ID NO: 423; and iv) SEQ ID NO: 857. In some cases, a cassette comprises, in order from 5′ to 3′: i) SEQ ID NO:858; ii) SEQ ID NO: 423; iii) a sequence that is complementary to SEQ ID NO: 423, where the sequence can comprise from 0 to 10 mismatches (non-complementary nucleotides) to SEQ ID NO: 423; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
48) In some cases, a recombinant expression vector comprises one of the following cassettes. In some cases, a cassette comprises, in order from 5′ to 3′: i) SEQ ID NO:854; ii) SEQ ID NO:424; iii) a sequence that is complementary to SEQ ID NO: 424, where the sequence can comprise from 0 to 10 mismatches (non-complementary nucleotides) to SEQ ID NO: 424; and iv) SEQ ID NO:855. In some cases, a cassette comprises, in order from 5′ to 3′: i) SEQ ID NO:856; ii) SEQ ID NO: 424; iii) a sequence that is complementary to SEQ ID NO: 424, where the sequence can comprise from 0 to 10 mismatches (non-complementary nucleotides) to SEQ ID NO: 424; and iv) SEQ ID NO: 857. In some cases, a cassette comprises, in order from 5′ to 3′: i) SEQ ID NO:858; ii) SEQ ID NO: 424; iii) a sequence that is complementary to SEQ ID NO: 424, where the sequence can comprise from 0 to 10 mismatches (non-complementary nucleotides) to SEQ ID NO: 424; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
49) In some cases, a recombinant expression vector comprises one of the following cassettes. In some cases, a cassette comprises, in order from 5′ to 3′: i) SEQ ID NO:854; ii) SEQ ID NO:425; iii) a sequence that is complementary to SEQ ID NO: 425, where the sequence can comprise from 0 to 10 mismatches (non-complementary nucleotides) to SEQ ID NO: 425; and iv) SEQ ID NO:855. In some cases, a cassette comprises, in order from 5′ to 3′: i) SEQ ID NO:856; ii) SEQ ID NO: 425; iii) a sequence that is complementary to SEQ ID NO: 425, where the sequence can comprise from 0 to 10 mismatches (non-complementary nucleotides) to SEQ ID NO: 425; and iv) SEQ ID NO: 857. In some cases, a cassette comprises, in order from 5′ to 3′: i) SEQ ID NO:858; ii) SEQ ID NO: 425; iii) a sequence that is complementary to SEQ ID NO: 425, where the sequence can comprise from 0 to 10 mismatches (non-complementary nucleotides) to SEQ ID NO: 425; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
50) In some cases, a recombinant expression vector comprises one of the following cassettes. In some cases, a cassette comprises, in order from 5′ to 3′: i) SEQ ID NO:854; ii) SEQ ID NO:426; iii) a sequence that is complementary to SEQ ID NO: 426, where the sequence can comprise from 0 to 10 mismatches (non-complementary nucleotides) to SEQ ID NO: 426; and iv) SEQ ID NO:855. In some cases, a cassette comprises, in order from 5′ to 3′: i) SEQ ID NO:856; ii) SEQ ID NO: 426; iii) a sequence that is complementary to SEQ ID NO: 426, where the sequence can comprise from 0 to 10 mismatches (non-complementary nucleotides) to SEQ ID NO: 426; and iv) SEQ ID NO: 857. In some cases, a cassette comprises, in order from 5′ to 3′: i) SEQ ID NO:858; ii) SEQ ID NO: 426; iii) a sequence that is complementary to SEQ ID NO: 426, where the sequence can comprise from 0 to 10 mismatches (non-complementary nucleotides) to SEQ ID NO: 426; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs. 51) In some cases, a recombinant expression vector comprises one of the following cassettes. In some cases, a cassette comprises, in order from 5′ to 3′: i) SEQ ID NO:854; ii) SEQ ID NO:427; iii) a sequence that is complementary to SEQ ID NO: 427, where the sequence can comprise from 0 to 10 mismatches (non-complementary nucleotides) to SEQ ID NO: 427; and iv) SEQ ID NO:855. In some cases, a cassette comprises, in order from 5′ to 3′: i) SEQ ID NO:856; ii) SEQ ID NO: 427; iii) a sequence that is complementary to SEQ ID NO: 427, where the sequence can comprise from 0 to 10 mismatches (non-complementary nucleotides) to SEQ ID NO: 427; and iv) SEQ ID NO: 857. In some cases, a cassette comprises, in order from 5′ to 3′: i) SEQ ID NO:858; ii) SEQ ID NO: 427; iii) a sequence that is complementary to SEQ ID NO: 427, where the sequence can comprise from 0 to 10 mismatches (non-complementary nucleotides) to SEQ ID NO: 427; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
52) In some cases, a recombinant expression vector comprises one of the following cassettes. In some cases, a cassette comprises, in order from 5′ to 3′: i) SEQ ID NO:854; ii) SEQ ID NO:428; iii) a sequence that is complementary to SEQ ID NO: 428, where the sequence can comprise from 0 to 10 mismatches (non-complementary nucleotides) to SEQ ID NO: 428; and iv) SEQ ID NO:855. In some cases, a cassette comprises, in order from 5′ to 3′: i) SEQ ID NO:856; ii) SEQ ID NO: 428; iii) a sequence that is complementary to SEQ ID NO: 428, where the sequence can comprise from 0 to 10 mismatches (non-complementary nucleotides) to SEQ ID NO: 428; and iv) SEQ ID NO: 857. In some cases, a cassette comprises, in order from 5′ to 3′: i) SEQ ID NO:858; ii) SEQ ID NO: 428; iii) a sequence that is complementary to SEQ ID NO: 428, where the sequence can comprise from 0 to 10 mismatches (non-complementary nucleotides) to SEQ ID NO: 428; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
53) In some cases, a recombinant expression vector comprises one of the following cassettes. In some cases, a cassette comprises, in order from 5′ to 3′: i) SEQ ID NO:854; ii) SEQ ID NO:429; iii) a sequence that is complementary to SEQ ID NO: 429, where the sequence can comprise from 0 to 10 mismatches (non-complementary nucleotides) to SEQ ID NO: 429; and iv) SEQ ID NO:855. In some cases, a cassette comprises, in order from 5′ to 3′: i) SEQ ID NO:856; ii) SEQ ID NO: 429; iii) a sequence that is complementary to SEQ ID NO: 429, where the sequence can comprise from 0 to 10 mismatches (non-complementary nucleotides) to SEQ ID NO: 429; and iv) SEQ ID NO: 857. In some cases, a cassette comprises, in order from 5′ to 3′: i) SEQ ID NO:858; ii) SEQ ID NO: 429; iii) a sequence that is complementary to SEQ ID NO: 429, where the sequence can comprise from 0 to 10 mismatches (non-complementary nucleotides) to SEQ ID NO: 429; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
54) In some cases, a recombinant expression vector comprises one of the following cassettes. In some cases, a cassette comprises, in order from 5′ to 3′: i) SEQ ID NO:854; ii) SEQ ID NO:430; iii) a sequence that is complementary to SEQ ID NO: 430, where the sequence can comprise from 0 to 10 mismatches (non-complementary nucleotides) to SEQ ID NO: 430; and iv) SEQ ID NO:855. In some cases, a cassette comprises, in order from 5′ to 3′: i) SEQ ID NO:856; ii) SEQ ID NO: 430; iii) a sequence that is complementary to SEQ ID NO: 430, where the sequence can comprise from 0 to 10 mismatches (non-complementary nucleotides) to SEQ ID NO: 430; and iv) SEQ ID NO: 857. In some cases, a cassette comprises, in order from 5′ to 3′: i) SEQ ID NO:858; ii) SEQ ID NO: 430; iii) a sequence that is complementary to SEQ ID NO: 430, where the sequence can comprise from 0 to 10 mismatches (non-complementary nucleotides) to SEQ ID NO: 430; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
55) In some cases, a recombinant expression vector comprises one of the following cassettes. In some cases, a cassette comprises, in order from 5′ to 3′: i) SEQ ID NO:854; ii) SEQ ID NO:431; iii) a sequence that is complementary to SEQ ID NO: 431, where the sequence can comprise from 0 to 10 mismatches (non-complementary nucleotides) to SEQ ID NO: 431; and iv) SEQ ID NO:855. In some cases, a cassette comprises, in order from 5′ to 3′: i) SEQ ID NO:856; ii) SEQ ID NO: 431; iii) a sequence that is complementary to SEQ ID NO: 431, where the sequence can comprise from 0 to 10 mismatches (non-complementary nucleotides) to SEQ ID NO: 431; and iv) SEQ ID NO: 857. In some cases, a cassette comprises, in order from 5′ to 3′: i) SEQ ID NO:858; ii) SEQ ID NO: 431; iii) a sequence that is complementary to SEQ ID NO: 431, where the sequence can comprise from 0 to 10 mismatches (non-complementary nucleotides) to SEQ ID NO: 431; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
56) In some cases, a recombinant expression vector comprises one of the following cassettes. In some cases, a cassette comprises, in order from 5′ to 3′: i) SEQ ID NO:854; ii) SEQ ID NO:432; iii) a sequence that is complementary to SEQ ID NO: 432, where the sequence can comprise from 0 to 10 mismatches (non-complementary nucleotides) to SEQ ID NO: 432; and iv) SEQ ID NO:855. In some cases, a cassette comprises, in order from 5′ to 3′: i) SEQ ID NO:856; ii) SEQ ID NO: 432; iii) a sequence that is complementary to SEQ ID NO: 432, where the sequence can comprise from 0 to 10 mismatches (non-complementary nucleotides) to SEQ ID NO: 432; and iv) SEQ ID NO: 857. In some cases, a cassette comprises, in order from 5′ to 3′: i) SEQ ID NO:858; ii) SEQ ID NO: 432; iii) a sequence that is complementary to SEQ ID NO: 432, where the sequence can comprise from 0 to 10 mismatches (non-complementary nucleotides) to SEQ ID NO: 432; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
57) In some cases, a recombinant expression vector comprises one of the following cassettes. In some cases, a cassette comprises, in order from 5′ to 3′: i) SEQ ID NO:854; ii) SEQ ID NO:433; iii) a sequence that is complementary to SEQ ID NO: 433, where the sequence can comprise from 0 to 10 mismatches (non-complementary nucleotides) to SEQ ID NO: 433; and iv) SEQ ID NO:855. In some cases, a cassette comprises, in order from 5′ to 3′: i) SEQ ID NO:856; ii) SEQ ID NO: 433; iii) a sequence that is complementary to SEQ ID NO: 433, where the sequence can comprise from 0 to 10 mismatches (non-complementary nucleotides) to SEQ ID NO: 433; and iv) SEQ ID NO: 857. In some cases, a cassette comprises, in order from 5′ to 3′: i) SEQ ID NO:858; ii) SEQ ID NO: 433; iii) a sequence that is complementary to SEQ ID NO: 433, where the sequence can comprise from 0 to 10 mismatches (non-complementary nucleotides) to SEQ ID NO: 433; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
58) In some cases, a recombinant expression vector comprises one of the following cassettes. In some cases, a cassette comprises, in order from 5′ to 3′: i) SEQ ID NO:854; ii) SEQ ID NO:434; iii) a sequence that is complementary to SEQ ID NO: 434, where the sequence can comprise from 0 to 10 mismatches (non-complementary nucleotides) to SEQ ID NO: 434; and iv) SEQ ID NO:855. In some cases, a cassette comprises, in order from 5′ to 3′: i) SEQ ID NO:856; ii) SEQ ID NO: 434; iii) a sequence that is complementary to SEQ ID NO: 434, where the sequence can comprise from 0 to 10 mismatches (non-complementary nucleotides) to SEQ ID NO: 434; and iv) SEQ ID NO: 857. In some cases, a cassette comprises, in order from 5′ to 3′: i) SEQ ID NO:858; ii) SEQ ID NO: 434; iii) a sequence that is complementary to SEQ ID NO: 434, where the sequence can comprise from 0 to 10 mismatches (non-complementary nucleotides) to SEQ ID NO: 434; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
59) In some cases, a recombinant expression vector comprises one of the following cassettes. In some cases, a cassette comprises, in order from 5′ to 3′: i) SEQ ID NO:854; ii) SEQ ID NO:435; iii) a sequence that is complementary to SEQ ID NO: 435, where the sequence can comprise from 0 to 10 mismatches (non-complementary nucleotides) to SEQ ID NO: 435; and iv) SEQ ID NO:855. In some cases, a cassette comprises, in order from 5′ to 3′: i) SEQ ID NO:856; ii) SEQ ID NO: 435; iii) a sequence that is complementary to SEQ ID NO: 435, where the sequence can comprise from 0 to 10 mismatches (non-complementary nucleotides) to SEQ ID NO: 435; and iv) SEQ ID NO: 857. In some cases, a cassette comprises, in order from 5′ to 3′: i) SEQ ID NO:858; ii) SEQ ID NO: 435; iii) a sequence that is complementary to SEQ ID NO: 435, where the sequence can comprise from 0 to 10 mismatches (non-complementary nucleotides) to SEQ ID NO: 435; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
60) In some cases, a recombinant expression vector comprises one of the following cassettes. In some cases, a cassette comprises, in order from 5′ to 3′: i) SEQ ID NO:854; ii) SEQ ID NO:436; iii) a sequence that is complementary to SEQ ID NO: 436, where the sequence can comprise from 0 to 10 mismatches (non-complementary nucleotides) to SEQ ID NO: 436; and iv) SEQ ID NO:855. In some cases, a cassette comprises, in order from 5′ to 3′: i) SEQ ID NO:856; ii) SEQ ID NO: 436; iii) a sequence that is complementary to SEQ ID NO: 436, where the sequence can comprise from 0 to 10 mismatches (non-complementary nucleotides) to SEQ ID NO: 436; and iv) SEQ ID NO: 857. In some cases, a cassette comprises, in order from 5′ to 3′: i) SEQ ID NO:858; ii) SEQ ID NO: 436; iii) a sequence that is complementary to SEQ ID NO: 436, where the sequence can comprise from 0 to 10 mismatches (non-complementary nucleotides) to SEQ ID NO: 436; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
61) In some cases, a recombinant expression vector comprises one of the following cassettes. In some cases, a cassette comprises, in order from 5′ to 3′: i) SEQ ID NO:854; ii) SEQ ID NO:437; iii) a sequence that is complementary to SEQ ID NO: 437, where the sequence can comprise from 0 to 10 mismatches (non-complementary nucleotides) to SEQ ID NO: 437; and iv) SEQ ID NO:855. In some cases, a cassette comprises, in order from 5′ to 3′: i) SEQ ID NO:856; ii) SEQ ID NO: 437; iii) a sequence that is complementary to SEQ ID NO: 437, where the sequence can comprise from 0 to 10 mismatches (non-complementary nucleotides) to SEQ ID NO: 437; and iv) SEQ ID NO: 857. In some cases, a cassette comprises, in order from 5′ to 3′: i) SEQ ID NO:858; ii) SEQ ID NO: 437; iii) a sequence that is complementary to SEQ ID NO: 437, where the sequence can comprise from 0 to 10 mismatches (non-complementary nucleotides) to SEQ ID NO: 437; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
62) In some cases, a recombinant expression vector comprises one of the following cassettes. In some cases, a cassette comprises, in order from 5′ to 3′: i) SEQ ID NO:854; ii) SEQ ID NO:438; iii) a sequence that is complementary to SEQ ID NO: 438, where the sequence can comprise from 0 to 10 mismatches (non-complementary nucleotides) to SEQ ID NO: 438; and iv) SEQ ID NO:855. In some cases, a cassette comprises, in order from 5′ to 3′: i) SEQ ID NO:856; ii) SEQ ID NO: 438; iii) a sequence that is complementary to SEQ ID NO: 438, where the sequence can comprise from 0 to 10 mismatches (non-complementary nucleotides) to SEQ ID NO: 438; and iv) SEQ ID NO: 857. In some cases, a cassette comprises, in order from 5′ to 3′: i) SEQ ID NO:858; ii) SEQ ID NO: 438; iii) a sequence that is complementary to SEQ ID NO: 438, where the sequence can comprise from 0 to 10 mismatches (non-complementary nucleotides) to SEQ ID NO: 438; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
63) In some cases, a recombinant expression vector comprises one of the following cassettes. In some cases, a cassette comprises, in order from 5′ to 3′: i) SEQ ID NO:854; ii) SEQ ID NO:439; iii) a sequence that is complementary to SEQ ID NO: 439, where the sequence can comprise from 0 to 10 mismatches (non-complementary nucleotides) to SEQ ID NO: 439; and iv) SEQ ID NO:855. In some cases, a cassette comprises, in order from 5′ to 3′: i) SEQ ID NO:856; ii) SEQ ID NO: 439; iii) a sequence that is complementary to SEQ ID NO: 439, where the sequence can comprise from 0 to 10 mismatches (non-complementary nucleotides) to SEQ ID NO: 439; and iv) SEQ ID NO: 857. In some cases, a cassette comprises, in order from 5′ to 3′: i) SEQ ID NO:858; ii) SEQ ID NO: 439; iii) a sequence that is complementary to SEQ ID NO: 439, where the sequence can comprise from 0 to 10 mismatches (non-complementary nucleotides) to SEQ ID NO: 439; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
64) In some cases, a recombinant expression vector comprises one of the following cassettes. In some cases, a cassette comprises, in order from 5′ to 3′: i) SEQ ID NO:854; ii) SEQ ID NO:440; iii) a sequence that is complementary to SEQ ID NO: 440, where the sequence can comprise from 0 to 10 mismatches (non-complementary nucleotides) to SEQ ID NO: 440; and iv) SEQ ID NO:855. In some cases, a cassette comprises, in order from 5′ to 3′: i) SEQ ID NO:856; ii) SEQ ID NO: 440; iii) a sequence that is complementary to SEQ ID NO: 440, where the sequence can comprise from 0 to 10 mismatches (non-complementary nucleotides) to SEQ ID NO: 440; and iv) SEQ ID NO: 857. In some cases, a cassette comprises, in order from 5′ to 3′: i) SEQ ID NO:858; ii) SEQ ID NO: 440; iii) a sequence that is complementary to SEQ ID NO: 440, where the sequence can comprise from 0 to 10 mismatches (non-complementary nucleotides) to SEQ ID NO: 440; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
65) In some cases, a recombinant expression vector comprises one of the following cassettes. In some cases, a cassette comprises, in order from 5′ to 3′: i) SEQ ID NO:854; ii) SEQ ID NO:441; iii) a sequence that is complementary to SEQ ID NO: 441, where the sequence can comprise from 0 to 10 mismatches (non-complementary nucleotides) to SEQ ID NO: 441; and iv) SEQ ID NO:855. In some cases, a cassette comprises, in order from 5′ to 3′: i) SEQ ID NO:856; ii) SEQ ID NO: 441; iii) a sequence that is complementary to SEQ ID NO: 441, where the sequence can comprise from 0 to 10 mismatches (non-complementary nucleotides) to SEQ ID NO: 441; and iv) SEQ ID NO: 857. In some cases, a cassette comprises, in order from 5′ to 3′: i) SEQ ID NO:858; ii) SEQ ID NO: 441; iii) a sequence that is complementary to SEQ ID NO: 441, where the sequence can comprise from 0 to 10 mismatches (non-complementary nucleotides) to SEQ ID NO: 441; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs. 66) In some cases, a recombinant expression vector comprises one of the following cassettes. In some cases, a cassette comprises, in order from 5′ to 3′: i) SEQ ID NO:854; ii) SEQ ID NO:442; iii) a sequence that is complementary to SEQ ID NO: 442, where the sequence can comprise from 0 to 10 mismatches (non-complementary nucleotides) to SEQ ID NO: 442; and iv) SEQ ID NO:855. In some cases, a cassette comprises, in order from 5′ to 3′: i) SEQ ID NO:856; ii) SEQ ID NO: 442; iii) a sequence that is complementary to SEQ ID NO: 442, where the sequence can comprise from 0 to 10 mismatches (non-complementary nucleotides) to SEQ ID NO: 442; and iv) SEQ ID NO: 857. In some cases, a cassette comprises, in order from 5′ to 3′: i) SEQ ID NO:858; ii) SEQ ID NO: 442; iii) a sequence that is complementary to SEQ ID NO: 442, where the sequence can comprise from 0 to 10 mismatches (non-complementary nucleotides) to SEQ ID NO: 442; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
67) In some cases, a recombinant expression vector comprises one of the following cassettes. In some cases, a cassette comprises, in order from 5′ to 3′: i) SEQ ID NO:854; ii) SEQ ID NO:443; iii) a sequence that is complementary to SEQ ID NO: 443, where the sequence can comprise from 0 to 10 mismatches (non-complementary nucleotides) to SEQ ID NO: 443; and iv) SEQ ID NO:855. In some cases, a cassette comprises, in order from 5′ to 3′: i) SEQ ID NO:856; ii) SEQ ID NO: 443; iii) a sequence that is complementary to SEQ ID NO: 443, where the sequence can comprise from 0 to 10 mismatches (non-complementary nucleotides) to SEQ ID NO: 443; and iv) SEQ ID NO: 857. In some cases, a cassette comprises, in order from 5′ to 3′: i) SEQ ID NO:858; ii) SEQ ID NO: 443; iii) a sequence that is complementary to SEQ ID NO: 443, where the sequence can comprise from 0 to 10 mismatches (non-complementary nucleotides) to SEQ ID NO: 443; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
68) In some cases, a recombinant expression vector comprises one of the following cassettes. In some cases, a cassette comprises, in order from 5′ to 3′: i) SEQ ID NO:854; ii) SEQ ID NO:444; iii) a sequence that is complementary to SEQ ID NO: 444, where the sequence can comprise from 0 to 10 mismatches (non-complementary nucleotides) to SEQ ID NO: 444; and iv) SEQ ID NO:855. In some cases, a cassette comprises, in order from 5′ to 3′: i) SEQ ID NO:856; ii) SEQ ID NO: 444; iii) a sequence that is complementary to SEQ ID NO: 444, where the sequence can comprise from 0 to 10 mismatches (non-complementary nucleotides) to SEQ ID NO: 444; and iv) SEQ ID NO: 857. In some cases, a cassette comprises, in order from 5′ to 3′: i) SEQ ID NO:858; ii) SEQ ID NO: 444; iii) a sequence that is complementary to SEQ ID NO: 444, where the sequence can comprise from 0 to 10 mismatches (non-complementary nucleotides) to SEQ ID NO: 444; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
69) In some cases, a recombinant expression vector comprises one of the following cassettes. In some cases, a cassette comprises, in order from 5′ to 3′: i) SEQ ID NO:854; ii) SEQ ID NO:445; iii) a sequence that is complementary to SEQ ID NO: 445, where the sequence can comprise from 0 to 10 mismatches (non-complementary nucleotides) to SEQ ID NO: 445; and iv) SEQ ID NO:855. In some cases, a cassette comprises, in order from 5′ to 3′: i) SEQ ID NO:856; ii) SEQ ID NO: 445; iii) a sequence that is complementary to SEQ ID NO: 445, where the sequence can comprise from 0 to 10 mismatches (non-complementary nucleotides) to SEQ ID NO: 445; and iv) SEQ ID NO: 857. In some cases, a cassette comprises, in order from 5′ to 3′: i) SEQ ID NO:858; ii) SEQ ID NO: 445; iii) a sequence that is complementary to SEQ ID NO: 445, where the sequence can comprise from 0 to 10 mismatches (non-complementary nucleotides) to SEQ ID NO: 445; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
70) In some cases, a recombinant expression vector comprises one of the following cassettes. In some cases, a cassette comprises, in order from 5′ to 3′: i) SEQ ID NO:854; ii) SEQ ID NO:446; iii) a sequence that is complementary to SEQ ID NO: 446, where the sequence can comprise from 0 to 10 mismatches (non-complementary nucleotides) to SEQ ID NO: 446; and iv) SEQ ID NO:855. In some cases, a cassette comprises, in order from 5′ to 3′: i) SEQ ID NO:856; ii) SEQ ID NO: 446; iii) a sequence that is complementary to SEQ ID NO: 446, where the sequence can comprise from 0 to 10 mismatches (non-complementary nucleotides) to SEQ ID NO: 446; and iv) SEQ ID NO: 857. In some cases, a cassette comprises, in order from 5′ to 3′: i) SEQ ID NO:858; ii) SEQ ID NO: 446; iii) a sequence that is complementary to SEQ ID NO: 446, where the sequence can comprise from 0 to 10 mismatches (non-complementary nucleotides) to SEQ ID NO: 446; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
71) In some cases, a recombinant expression vector comprises one of the following cassettes. In some cases, a cassette comprises, in order from 5′ to 3′: i) SEQ ID NO:854; ii) SEQ ID NO:447; iii) a sequence that is complementary to SEQ ID NO: 447, where the sequence can comprise from 0 to 10 mismatches (non-complementary nucleotides) to SEQ ID NO: 447; and iv) SEQ ID NO:855. In some cases, a cassette comprises, in order from 5′ to 3′: i) SEQ ID NO:856; ii) SEQ ID NO: 447; iii) a sequence that is complementary to SEQ ID NO: 447, where the sequence can comprise from 0 to 10 mismatches (non-complementary nucleotides) to SEQ ID NO: 447; and iv) SEQ ID NO: 857. In some cases, a cassette comprises, in order from 5′ to 3′: i) SEQ ID NO:858; ii) SEQ ID NO: 447; iii) a sequence that is complementary to SEQ ID NO: 447, where the sequence can comprise from 0 to 10 mismatches (non-complementary nucleotides) to SEQ ID NO: 447; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
72) In some cases, a recombinant expression vector comprises one of the following cassettes. In some cases, a cassette comprises, in order from 5′ to 3′: i) SEQ ID NO:854; ii) SEQ ID NO:448; iii) a sequence that is complementary to SEQ ID NO: 448, where the sequence can comprise from 0 to 10 mismatches (non-complementary nucleotides) to SEQ ID NO: 448; and iv) SEQ ID NO:855. In some cases, a cassette comprises, in order from 5′ to 3′: i) SEQ ID NO:856; ii) SEQ ID NO: 448; iii) a sequence that is complementary to SEQ ID NO: 448, where the sequence can comprise from 0 to 10 mismatches (non-complementary nucleotides) to SEQ ID NO: 448; and iv) SEQ ID NO: 857. In some cases, a cassette comprises, in order from 5′ to 3′: i) SEQ ID NO:858; ii) SEQ ID NO: 448; iii) a sequence that is complementary to SEQ ID NO: 448, where the sequence can comprise from 0 to 10 mismatches (non-complementary nucleotides) to SEQ ID NO: 448; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
73) In some cases, a recombinant expression vector comprises one of the following cassettes. In some cases, a cassette comprises, in order from 5′ to 3′: i) SEQ ID NO:854; ii) SEQ ID NO:449; iii) a sequence that is complementary to SEQ ID NO: 449, where the sequence can comprise from 0 to 10 mismatches (non-complementary nucleotides) to SEQ ID NO: 449; and iv) SEQ ID NO:855. In some cases, a cassette comprises, in order from 5′ to 3′: i) SEQ ID NO:856; ii) SEQ ID NO: 449; iii) a sequence that is complementary to SEQ ID NO: 449, where the sequence can comprise from 0 to 10 mismatches (non-complementary nucleotides) to SEQ ID NO: 449; and iv) SEQ ID NO: 857. In some cases, a cassette comprises, in order from 5′ to 3′: i) SEQ ID NO:858; ii) SEQ ID NO: 449; iii) a sequence that is complementary to SEQ ID NO: 449, where the sequence can comprise from 0 to 10 mismatches (non-complementary nucleotides) to SEQ ID NO: 449; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
74) In some cases, a recombinant expression vector comprises one of the following cassettes. In some cases, a cassette comprises, in order from 5′ to 3′: i) SEQ ID NO:854; ii) SEQ ID NO:450; iii) a sequence that is complementary to SEQ ID NO: 450, where the sequence can comprise from 0 to 10 mismatches (non-complementary nucleotides) to SEQ ID NO: 450; and iv) SEQ ID NO:855. In some cases, a cassette comprises, in order from 5′ to 3′: i) SEQ ID NO:856; ii) SEQ ID NO: 450; iii) a sequence that is complementary to SEQ ID NO: 450, where the sequence can comprise from 0 to 10 mismatches (non-complementary nucleotides) to SEQ ID NO: 450; and iv) SEQ ID NO: 857. In some cases, a cassette comprises, in order from 5′ to 3′: i) SEQ ID NO:858; ii) SEQ ID NO: 450; iii) a sequence that is complementary to SEQ ID NO: 450, where the sequence can comprise from 0 to 10 mismatches (non-complementary nucleotides) to SEQ ID NO: 450; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
75) In some cases, a recombinant expression vector comprises one of the following cassettes. In some cases, a cassette comprises, in order from 5′ to 3′: i) SEQ ID NO:854; ii) SEQ ID NO:451; iii) a sequence that is complementary to SEQ ID NO: 451, where the sequence can comprise from 0 to 10 mismatches (non-complementary nucleotides) to SEQ ID NO: 451; and iv) SEQ ID NO:855. In some cases, a cassette comprises, in order from 5′ to 3′: i) SEQ ID NO:856; ii) SEQ ID NO: 451; iii) a sequence that is complementary to SEQ ID NO: 451, where the sequence can comprise from 0 to 10 mismatches (non-complementary nucleotides) to SEQ ID NO: 451; and iv) SEQ ID NO: 857. In some cases, a cassette comprises, in order from 5′ to 3′: i) SEQ ID NO:858; ii) SEQ ID NO: 451; iii) a sequence that is complementary to SEQ ID NO: 451, where the sequence can comprise from 0 to 10 mismatches (non-complementary nucleotides) to SEQ ID NO: 451; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
76) In some cases, a recombinant expression vector comprises one of the following cassettes. In some cases, a cassette comprises, in order from 5′ to 3′: i) SEQ ID NO:854; ii) SEQ ID NO:452; iii) a sequence that is complementary to SEQ ID NO: 452, where the sequence can comprise from 0 to 10 mismatches (non-complementary nucleotides) to SEQ ID NO: 452; and iv) SEQ ID NO:855. In some cases, a cassette comprises, in order from 5′ to 3′: i) SEQ ID NO:856; ii) SEQ ID NO: 452; iii) a sequence that is complementary to SEQ ID NO: 452, where the sequence can comprise from 0 to 10 mismatches (non-complementary nucleotides) to SEQ ID NO: 452; and iv) SEQ ID NO: 857. In some cases, a cassette comprises, in order from 5′ to 3′: i) SEQ ID NO:858; ii) SEQ ID NO: 452; iii) a sequence that is complementary to SEQ ID NO: 452, where the sequence can comprise from 0 to 10 mismatches (non-complementary nucleotides) to SEQ ID NO: 452; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
77) In some cases, a recombinant expression vector comprises one of the following cassettes. In some cases, a cassette comprises, in order from 5′ to 3′: i) SEQ ID NO:854; ii) SEQ ID NO:453; iii) a sequence that is complementary to SEQ ID NO: 453, where the sequence can comprise from 0 to 10 mismatches (non-complementary nucleotides) to SEQ ID NO: 453; and iv) SEQ ID NO:855. In some cases, a cassette comprises, in order from 5′ to 3′: i) SEQ ID NO:856; ii) SEQ ID NO: 453; iii) a sequence that is complementary to SEQ ID NO: 453, where the sequence can comprise from 0 to 10 mismatches (non-complementary nucleotides) to SEQ ID NO: 453; and iv) SEQ ID NO: 857. In some cases, a cassette comprises, in order from 5′ to 3′: i) SEQ ID NO:858; ii) SEQ ID NO: 453; iii) a sequence that is complementary to SEQ ID NO: 453, where the sequence can comprise from 0 to 10 mismatches (non-complementary nucleotides) to SEQ ID NO: 453; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
78) In some cases, a recombinant expression vector comprises one of the following cassettes. In some cases, a cassette comprises, in order from 5′ to 3′: i) SEQ ID NO:854; ii) SEQ ID NO:454; iii) a sequence that is complementary to SEQ ID NO: 454, where the sequence can comprise from 0 to 10 mismatches (non-complementary nucleotides) to SEQ ID NO: 454; and iv) SEQ ID NO:855. In some cases, a cassette comprises, in order from 5′ to 3′: i) SEQ ID NO:856; ii) SEQ ID NO: 454; iii) a sequence that is complementary to SEQ ID NO: 454, where the sequence can comprise from 0 to 10 mismatches (non-complementary nucleotides) to SEQ ID NO: 454; and iv) SEQ ID NO: 857. In some cases, a cassette comprises, in order from 5′ to 3′: i) SEQ ID NO:858; ii) SEQ ID NO: 454; iii) a sequence that is complementary to SEQ ID NO: 454, where the sequence can comprise from 0 to 10 mismatches (non-complementary nucleotides) to SEQ ID NO: 454; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
79) In some cases, a recombinant expression vector comprises one of the following cassettes. In some cases, a cassette comprises, in order from 5′ to 3′: i) SEQ ID NO:854; ii) SEQ ID NO:455; iii) a sequence that is complementary to SEQ ID NO: 455, where the sequence can comprise from 0 to 10 mismatches (non-complementary nucleotides) to SEQ ID NO: 455; and iv) SEQ ID NO:855. In some cases, a cassette comprises, in order from 5′ to 3′: i) SEQ ID NO:856; ii) SEQ ID NO: 455; iii) a sequence that is complementary to SEQ ID NO: 455, where the sequence can comprise from 0 to 10 mismatches (non-complementary nucleotides) to SEQ ID NO: 455; and iv) SEQ ID NO: 857. In some cases, a cassette comprises, in order from 5′ to 3′: i) SEQ ID NO:858; ii) SEQ ID NO: 455; iii) a sequence that is complementary to SEQ ID NO: 455, where the sequence can comprise from 0 to 10 mismatches (non-complementary nucleotides) to SEQ ID NO: 455; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.
80) In some cases, a recombinant expression vector comprises one of the following cassettes. In some cases, a cassette comprises, in order from 5′ to 3′: i) SEQ ID NO:854; ii) SEQ ID NO:456; iii) a sequence that is complementary to SEQ ID NO: 456, where the sequence can comprise from 0 to 10 mismatches (non-complementary nucleotides) to SEQ ID NO: 456; and iv) SEQ ID NO:855. In some cases, a cassette comprises, in order from 5′ to 3′: i) SEQ ID NO:856; ii) SEQ ID NO: 456; iii) a sequence that is complementary to SEQ ID NO: 456, where the sequence can comprise from 0 to 10 mismatches (non-complementary nucleotides) to SEQ ID NO: 456; and iv) SEQ ID NO: 857. In some cases, a cassette comprises, in order from 5′ to 3′: i) SEQ ID NO:858; ii) SEQ ID NO: 456; iii) a sequence that is complementary to SEQ ID NO: 456, where the sequence can comprise from 0 to 10 mismatches (non-complementary nucleotides) to SEQ ID NO: 456; and iv) SEQ ID NO:859. In some cases, the cassette is operably linked to a promoter that is functional in a eukaryotic cell. In some cases, the cassette is operably linked to an RNA polymerase II promoter. In some cases, the cassette is operably linked to an RNA polymerase III promoter. In some cases, the cassette is operably linked to a CAG promoter. In some cases, the cassette is operably linked to a CBA promoter. In some cases, the cassette is operably linked to a CMV promoter. In some cases, the cassette is operably linked to a U6 promoter. In some cases, the cassette is operably linked to an EF1α promoter. In some cases, the cassette is operably linked to an H1 promoter. In some cases, the recombinant expression vector comprises a 5′ AAV ITR sequence and a 3′ AAV ITR sequence. In some cases, the AAV ITRs are AAV9 ITRs. In some cases, the AAV ITRs are AAV2 ITRs.

### Delivery Vehicles

A recombinant expression vector of the present disclosure can be present in a delivery vehicle. Thus, the present disclosure provides a delivery vehicle comprising a recombinant expression vector of the present disclosure. In some cases, the delivery vehicle is a non-viral delivery vehicle. In some cases, the delivery vehicle is a lipid nanoparticle. In some cases, the delivery vehicle is a viral delivery vehicle. In some cases, the viral delivery vehicle is a recombinant AAV virion. Suitable AAV virions include those with AAV2 capsid, an AAV9 capsid, and the like.

Suitable lipid nanoparticles can include, e.g., one or more cationic lipids, lipids modified with poly (ethylene glycol) (“PEGylated lipids”), and the like. Suitable cationic lipids include, but are not limited to, XTC (2,2-dilinoleyl-4-dimethylaminoethyl-[1,3]-dioxolane), MC3 (((6Z,9Z,28Z,31Z)-heptatriaconta-6,9,28,31-tetraen-19-yl 4-(dimethylamino) butanoate), ALNY-100 ((3aR,5s,6aS)-N,N-dimethyl-2,2-di((92,12Z)-octadeca-9,12-dienyl)tetrahydr-o-3aH-cyclopenta[d][1,3]dioxol-5-amine)), NC98-5 (4,7,13-tris(3-oxo-3-(undecylamino)propyl)-N1,N16-diundecyl-4,7,10,13-tet-raazahexadecane-1,16-diamide), DODAP (1,2-dioleyl-3-dimethylammonium propane), HGT4003, ICE, HGT5000, cis or trans HGT5001, DOTAP (1,2-dioleyl-3-trimethylammonium propane), DOTMA (1,2-di-O-octadecenyl-3-trimethylammonium propane), DLinDMA, DLin-KC2-DMA, and C12-200. Other suitable lipids that can be included in a lipid nanoparticle include, but are not limited to, DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DPPE (1,2-dipalmitoyl-sn-glycero-3-phosphoethanolamine), DMPE (1,2-dimyristoyl-sn-glycero-3-phosphoethanolamine), DOPG (1,2-dioleoyl-sn-glycero-3-phospho-(1′-rac-glycerol)), and cholesterol. Suitable PEGylated lipids include, e.g., PEG-DSG (1,2-Distearoyl-rac-glycero-3-methoxypolyethylene glycol conjugated to, e.g., PEG-1000, PEG-2000, PEG-5000, and the like), PEG-DMG (1,2-Dimyristoyl-rac-glycerol conjugated to PEG), and PEG-ceramides.

### D. Pharmaceutical Compositions

The disclosure provides pharmaceutical compositions comprising nucleic acids (e.g., DNA), expression cassettes, or vectors encoding double stranded RNAs described herein and a pharmaceutically acceptable carrier. As used herein, the term “pharmaceutically acceptable” refers to those compounds, materials, compositions, and/or dosage forms which are, within the scope of sound medical judgment, suitable for use in contact with cells and/or tissues without excessive toxicity, irritation, allergic response, or other problem or complication, commensurate with a reasonable benefit/risk ratio.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Each carrier must be “acceptable” in the sense of being compatible with the other ingredients of the formulation and not injurious to the cell or tissue being contacted. Additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As is well known in the medical arts, the dosage for any one patient depends upon many factors, including the patient's size, weight, body surface area, age, the level of expression of inhibitory RNA expression required to achieve a therapeutic effect, stability of the inhibitory nucleic acid, specific disease being treated, stage of disease, sex, time and route of administration, general health, and other drugs being administered concurrently.

In some embodiments, rAAVs as described herein are administered to a subject in an amount of about 1×10⁶ VG (viral genomes) to about 1×10¹⁶ VG per subject, or about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹¹, 1.1×10¹², 1.2×10¹², 1.3×10¹², 1.4×10¹², 1.5×10¹², 1.6×10¹², 1.7×10¹², 1.8×10¹², 1.9×10¹², 2×10¹², 3×10¹², 4×10¹², 4.1×10², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10², 8.2×10¹², 8.3×10¹², 8.4×10¹², 8.5×10¹², 8.6×10¹², 8.7×10¹², 8.8×10¹², 8.9×10¹², 9×10¹², 1×10¹¹, 2×10¹¹, 3×10¹¹, 4×10¹¹, 5×10¹¹, 6×10¹¹, 6.7×10¹¹, 7×10¹¹, 8×10¹¹, 9×10¹¹, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject. In some embodiments, rAAV particles as described herein are administered to a subject in an amount of about 1×10⁶ VG/kg to about 1×10¹⁶ VG/kg, or about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰ , 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 3×10¹², 4×10¹², 4.1×10², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10², 8.2×10¹², 8.3×10¹², 8.4×10¹², 8.5×10¹², 8.6×10¹², 8.7×10¹², 8.8×10¹², 8.9×10¹², 9×10¹², 1×10¹¹, 2×10¹¹, 3×10¹¹, 4×10¹¹, 5×10¹¹, 6×10¹¹, 6.7×10¹¹, 7×10, 8×10, 9×10, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/kg.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's disease, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions (e.g., pharmaceutical compositions) may be administered by any route, including enteral (e.g., oral), parenteral, intravenous, intramuscular, intra-arterial, intramedullary, intrathecal, subpial, intraparenchymal, intrastriatal, intracranial, intracisternal, intra-cerebral, intracerebral ventricular, intraocular, intraventricular, intralumbar, subcutaneous, transdermal, intradermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. In general, the most appropriate route of administration will depend upon a variety of factors including the nature of the agent (e.g., its stability in the environment of the gastrointestinal tract), and/or the condition of the subject. In some embodiments, compositions are directly injected into the CNS of the subject. In some embodiments, direct injection into the CNS is intracerebral injection, intracerebral ventricular injection, intraparenchymal injection, intrathecal injection, intrastriatal injection, subpial injection, or any combination thereof. In some embodiments, direct injection into the CNS is direct injection into the cerebrospinal fluid (CSF) of the subject, optionally wherein the direct injection is intracisternal injection, intraventricular injection, and/or intralumbar injection. In some embodiments, compositions are administered by a combination of direct injection into the CNS and by a route that is not directly injected into the CNS (e.g., intravenously).

In some embodiments, pharmaceutical compositions comprising rAAV particles are formulated to reduce aggregation of rAAV particles, particularly where high rAAV particle concentrations are present (e.g., ˜10¹³ VG/ml or more). Methods for reducing aggregation of rAAV particles are well known in the art and, include, for example, addition of surfactants, pH adjustment, salt concentration adjustment, etc. (See, e.g., Wright F R, et al., Molecular Therapy (2005) 12:171-178, incorporated herein by reference in its entirety).

### D. Kits

In some embodiments, the compositions provided herein may be assembled into pharmaceutical or research kits to facilitate their use in therapeutic or research use. A kit may include one or more containers comprising: (a) expression cassette or vector encoding a double stranded RNA as described herein; (b) instructions for use; and optionally (c) reagents for transducing the kit component (a) into a host cell. In some embodiments, the kit component (a) may be in a pharmaceutical formulation and dosage suitable for a particular use and mode of administration. For example, the kit component (a) may be presented in unit-dose or multi-dose containers, such as sealed ampoules or vials. The components of the kit may require mixing one or more components prior to use or may be prepared in a premixed state. The components of the kit may be in liquid or solid form, and may require addition of a solvent or further dilution. The components of the kit may be sterile. The instructions may be in written or electronic form and may be associated with the kit (e.g., written insert, CD, DVD) or provided via internet or web-based communication. The kit may be shipped and stored at a refrigerated or frozen temperature.

### E. Methods of Use

The present disclosure provides nucleic acids (e.g., DNA), expression cassettes, recombinant expression vectors comprising a cassette, recombinant expression vectors encoding double stranded RNAs, or pharmaceutical compositions described herein for use in a method of therapy.

The present disclosure provides method for selectively reducing translation of a disease-associated CAG repeat-containing RNA in an individual having a CAG repeat expansion disorder, the method comprising administering to the individual an effective amount of a recombinant expression vector of the present disclosure, a delivery vehicle of the present disclosure, a viral particle of the present disclosure, or a pharmaceutical composition of the present disclosure. In some cases, the repeat expansion disorder is Huntington's disease, spinocerebellar ataxia type 1, spinocerebellar ataxia type 2, spinocerebellar ataxia type 3, spinocerebellar ataxia type 6, spinocerebellar ataxia type 7, spinocerebellar ataxia type 12, spinocerebellar ataxia type 17, spinal and bulbar muscular atrophy, dentatorubral pallidoluysian atrophy, or cleidocranial dysplasia. In some cases, said administering comprises direct injection to the central nervous system of the individual. In some cases, the direct injection is intracerebral injection, intracerebral ventricular injection intraparenchymal injection, intrathecal injection, intrastriatal injection, intrathalamic injection, intracisternal magna injection, subpial injection, or any combination thereof. In some cases, said administering provides for a ratio of a polypeptide encoded by the non-disease-associated CAG repeat-containing RNA to a polypeptide encoded by disease-associated CAG repeat-containing RNA of greater than 1.0. In some cases, said administering provides for a ratio of a polypeptide encoded by the non-disease-associated CAG repeat-containing RNA to a polypeptide encoded by disease-associated CAG repeat-containing RNA of from 1.1 to 1.8 (e.g., from 1.1 to 1.4, from 1.4 to 1.6, or from 1.6 to 1.8). In some cases, said administering provides for a ratio of a polypeptide encoded by the non-disease-associated CAG repeat-containing RNA to a polypeptide encoded by disease-associated CAG repeat-containing RNA of greater than 1.8 (e.g., 2.0, from 2.0 to 2.2, from 2.2 to 2.4, from 2.4 to 2.6, from 2.6 to 2.8, from 2.8 to 3.0).

The present disclosure provides methods for reducing or inhibiting expression of a CAG repeat containing RNA (e.g., mRNA or pre-mRNA) in a mammalian cell, comprising introducing into the mammalian cell a double stranded RNA comprising from 5′ to 3′:
(a) a 5′ leader sequence;
(b) a 5′ stem comprising or substantially comprising a passenger sequence;
(c) a 5′ linker;
(d) a terminal loop;
(e) a 3′ linker;
(f) a 3′ stem comprising or substantially comprising a guide sequence; and
(g) a 3′ trailer sequence;
wherein the guide sequence targets a CAG repeat region of a CAG repeat containing RNA (e.g., mRNA or pre-mRNA); and wherein the double stranded RNA is cleaved by the mammalian cell to produce a siRNA comprising the guide sequence and the passenger sequence, wherein the guide sequence of the siRNA comprises the 1-5 base mismatches relative to the CAG repeat region located at positions 8-16 of the guide sequence of the siRNA.

In some embodiments, inhibiting expression of a CAG repeat containing mRNA comprises inhibiting expression of the polyglutamine containing protein encoded by the CAG repeat containing mRNA, such as for example by binding to the CAG repeat region of a CAG repeat containing mRNA and blocking translation of the CAG repeat containing protein rather than by degradation of the CAG repeat containing mRNA.

In some embodiments, the method reduces or inhibits expression of a pathologic or pathogenic allele of a CAG repeat containing RNA. In some embodiments, the method selectively reduces or inhibits expression of a pathologic or pathogenic allele of a CAG repeat containing RNA compared to expression of a normal allele of a CAG repeat containing RNA.

In some embodiments, the double stranded RNA is contained within a pre-miRNA scaffold, pri-miRNA scaffold, or shRNA. In some embodiments, the double stranded RNA is cleaved by DROSHA and/or DICER in the mammalian cell to yield the siRNA.

In some embodiments, at least one mismatch relative to the CAG repeat region is located at positions 8-12 of the guide sequence of the siRNA. In some embodiments, at least one mismatch relative to the CAG repeat region is located at positions 9-11 of the guide sequence of the siRNA.

In some embodiments, two or more of the mismatches relative to the CAG repeat region located at positions 8-16 of the guide sequence of the siRNA are contiguous. In some embodiments, at least one mismatch relative to the CAG repeat region located at positions 8-16 of the guide sequence of the siRNA is not adjacent to another mismatch located at positions 8-16 of the guide sequence of the siRNA.

In some embodiments, the 5′ leader sequence is not complementary to the 3′ trailer sequence. In some embodiments, the 5′ leader sequence is partially complementary to the 3′ trailer sequence. In some embodiments, the 3′ trailer sequence comprises a polyU tail. In some embodiments, the polyU tail has 3 to 6 uridines.

In some embodiments, the 5′ leader sequence and/or 3′ trailer sequence is about 1 nucleotide to about 500 nucleotides in length. In some embodiments, the 5′ leader sequence and/or 3′ trailer sequence is about 1 nucleotide to about 400 nucleotides, about 1 nucleotide to about 300 nucleotides, about 1 nucleotide to about 200 nucleotides, about 1 nucleotide to about 100 nucleotides, about 1 nucleotide to about 75 nucleotides about 1 nucleotide to about 50 nucleotides, about 1 nucleotide to about 25 nucleotides, about 1 nucleotide to about 20 nucleotides, about 1 nucleotide to about 15 nucleotides, or about 1 nucleotide to about 10 nucleotides in length.

In some embodiments, the guide sequence has about 15 to about 30 nucleotides. In some embodiments, the guide sequence has about 19 to about 24 nucleotides. In some embodiments, the guide sequence has a nucleotide sequence set forth in Tables B1-B2. In some embodiments, the passenger sequence is 100% complementary to the guide sequence. In some embodiments, the passenger sequence is partially complementary to the guide sequence. In some embodiments, the passenger sequence comprises one to ten or one to five base mismatches or bulges relative to the guide sequence.

In some embodiments, the 5′ linker is 100% complementary to the 3′ linker.

In some embodiments, the terminal loop has about 4 nucleotides to about 1,000 nucleotides, about 4 nucleotides to about 900 nucleotides, about 4 nucleotides to about 800 nucleotides, about 4 nucleotides to about 700 nucleotides, about 4 nucleotides to about 600 nucleotides, about 4 nucleotides to about 500 nucleotides, about 4 nucleotides to about 400 nucleotides, about 4 nucleotides to about 300 nucleotides, about 4 nucleotides to about 200 nucleotides, about 4 nucleotides to about 100 nucleotides, about 4 nucleotides to about 90 nucleotides about 4 nucleotides to about 80 nucleotides, about 4 nucleotides to about 70 nucleotides, about 4 nucleotides to about 50 nucleotides, about 4 nucleotides to about 40 nucleotides, about 4 nucleotides to about 30 nucleotides, about 4 nucleotides to about 20 nucleotides, about 4 nucleotides to about 15 nucleotides, or about 4 nucleotides to about 10 nucleotides. Additional features of the double stranded RNA are described herein.

In some embodiments, the method comprises introducing a vector comprising a nucleic acid encoding the double stranded RNA into the mammalian cell. In some embodiments, the nucleic acid encoding the double stranded RNA is operably linked to a promoter. In some embodiments, the promoter is an RNA polymerase II promoter or an RNA polymerase III promoter.

In some embodiments, the siRNAs produced from the double stranded RNA in the mammalian cell and having 1-5 base mismatches relative to the CAG repeat region located at their predicted positions within positions 8-16 of the guide sequence are at an abundance of at least 10%, 15%, 20%, 25%, 30%, 35%, 40%, 45%, 50%, 55%, 60%, 65%, 70%, 75%, 80%, 85%, 90%, 95% or more of the siRNAs produced from the double stranded RNA by the mammalian cell. % abundance of a siRNA correctly processed from a double stranded RNA may be measured by obtaining the number of RNA sequence reads that have the correctly processed 5′ end and align to the reference CAG repeat region containing reference transcript, and dividing by the total number of RNA sequence reads of siRNAs aligned to the reference CAG repeat region containing reference transcript.

In some embodiments, the CAG repeat containing mRNA is HTT, Ataxin1, Ataxin2, Ataxin3, CACNA1A, Ataxin7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, or MAB21L1. In some embodiments, the CAG repeat containing mRNA contains an expanded number of repeats.

In some embodiments, the mammalian cell is a CNS cell. In some embodiments, the mammalian cell is a non-neuronal cell or neuronal cell of the CNS. In some embodiments, the non-neuronal cell of the CNS is a glial cell, astrocyte, or microglial cell. In some embodiments, the mammalian cell is in vitro. In some embodiments, the mammalian cell is a non-CNS cell. In some embodiments, the mammalian cell is a fibroblast. In some embodiments, the mammalian cell is from a subject having one or more symptoms of a polyglutamine disease or suspected of having a polyglutamine disease or having a predisposition for a polyglutamine disease.

The present disclosure provides methods for reducing or inhibiting expression of a CAG repeat containing mRNA in a subject in need thereof, comprising administering to the subject a nucleic acid molecule (e.g., DNA) encoding a double stranded RNA of the disclosure, an expression cassette comprising the nucleic acid molecule encoding the double stranded RNA of the disclosure; a vector comprising the nucleic acid molecule encoding the double stranded RNA of the disclosure, or a pharmaceutical composition thereof.

In some embodiments, the inhibiting expression of a CAG repeat containing mRNA comprises inhibiting expression of the polyglutamine containing protein encoded by the CAG repeat containing mRNA, such as for example by binding to the CAG repeat region of a CAG repeat containing mRNA and blocking or repressing translation of the CAG repeat containing protein rather than by degradation of the CAG repeat containing mRNA.

In some embodiments, the CAG repeat containing mRNA or pre-mRNA is HTT, Ataxin1, Ataxin2, Ataxin3, CACNA1A, Ataxin7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, or MAB21L1.

In some embodiments, the CAG repeat containing mRNA or pre-mRNA contains an expanded number of repeats. In some embodiments, the subject has a polyglutamine disease, meaning exhibiting symptoms of the polyglutamine disease. In some embodiments, the subject is at risk of developing a polyglutamine disease and does not yet show signs of the polyglutamine disease. In some embodiments, the polyglutamine disease is associated with HTT, Ataxin1, Ataxin2, Ataxin3, CACNA1A, Ataxin7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, or MAB21L1. In some embodiments, the polyglutamine disease is a neurodegenerative disease. In some embodiments, the subject has Huntington's disease, spinocerebellar ataxia type 1, spinocerebellar ataxia type 2, spinocerebellar ataxia type 3, spinocerebellar ataxia type 6, spinocerebellar ataxia type 7, spinocerebellar ataxia type 12, spinocerebellar ataxia type 17, amyotrophic lateral sclerosis, spinal bulbar muscular atrophy, dentatorubral pallidoluysian atrophy, cleidocranial dysplasia, retinitis pigmentosa, myotonic dystrophy 1, or branchiootorenal syndrome 2, or is at risk of developing Huntington's disease, spinocerebellar ataxia type 1, spinocerebellar ataxia type 2, spinocerebellar ataxia type 3, spinocerebellar ataxia type 6, spinocerebellar ataxia type 7, spinocerebellar ataxia type 12, spinocerebellar ataxia type 17, amyotrophic lateral sclerosis, spinal bulbar muscular atrophy, dentatorubral pallidoluysian atrophy, cleidocranial dysplasia, retinitis pigmentosa, myotonic dystrophy 1, or branchiootorenal syndrome 2 and does not yet show signs of the disease.

The present disclosure also provides methods for treating a subject having a polyglutamine disease or at risk of developing a polyglutamine disease, comprising administering to the subject a nucleic acid molecule (e.g., DNA) encoding a double stranded RNA of the disclosure, an expression cassette comprising the nucleic acid molecule encoding the double stranded RNA of the disclosure; a vector comprising the nucleic acid molecule encoding the double stranded RNA of the disclosure, or a pharmaceutical composition thereof.

As used herein, the term “treat” refers to preventing or delaying onset of a polyglutamine disease; reducing severity of polyglutamine disease; reducing or preventing development of symptoms characteristic of polyglutamine disease; preventing worsening of symptoms characteristic of polyglutamine disease, or any combination thereof. In some embodiments, a treatment of a subject involves subjects having a polyglutamine disease or at risk of developing a polyglutamine disease and does not yet show signs of the polyglutamine disease.

In some embodiments, the methods for treatment of the present disclosure comprise administration as a monotherapy or in combination with one or more additional therapies for the treatment of the polyglutamine disease. Combination therapy may mean administration of the compositions of the present disclosure (e.g., a nucleic acid molecule (e.g., DNA) encoding a double stranded RNA of the disclosure, an expression cassette comprising the nucleic acid molecule encoding the double stranded RNA of the disclosure; a vector comprising the nucleic acid molecule encoding the double stranded RNA of the disclosure, or a pharmaceutical composition thereof) to the subject concurrently, prior to, subsequent to one or more additional therapies. Concurrent administration of combination therapy may mean that the compositions of the present disclosure (e.g., a nucleic acid molecule (e.g., DNA) encoding a double stranded RNA of the disclosure, an expression cassette comprising the nucleic acid molecule encoding the double stranded RNA of the disclosure; a vector comprising the nucleic acid molecule encoding the double stranded RNA of the disclosure, or a pharmaceutical composition thereof) and additional therapy are formulated for administration in the same dosage form or administered in separate dosage forms.

The present disclosure also provides methods of increasing abundance of a processed artificial miRNA that targets a CAG repeat containing RNA in a cell, wherein the artificial miRNA is encoded by a viral vector, comprising administering to the cell:

(a) a viral vector encoding a double stranded RNA according to any one of the embodiments described herein; or (b) a viral vector encoding an artificial miRNA according to any one of the embodiments described herein, wherein the artificial miRNA comprises a guide sequence, a passenger sequence, and optionally a terminal loop, wherein the guide sequence targets a CAG repeat region of a CAG repeat containing RNA and comprises 1-5 base mismatches relative to the CAG repeat region, wherein the base mismatches are located at positions 8-16 of the guide sequence.

In some embodiments, at least one mismatch relative to the CAG repeat region is located at positions 8-12 of the guide sequence. In some embodiments, at least one mismatch relative to the CAG repeat region is located at positions 9-11 of the guide sequence. In some embodiments, two or more of the mismatches relative to the CAG repeat region located at positions 8-16 of the guide sequence are contiguous.

In some embodiments, the guide sequence comprises or consists of a sequence selected from Tables B1-B2.

In some embodiments, the artificial miRNA further comprises a 5′ leader sequence and a 3′ trailer sequence. In some embodiments, the artificial miRNA comprises a 5′ leader sequence, a 3′ trailer sequence, and loop sequence selected from Table E.

In some embodiments, the CAG repeat containing RNA is an mRNA or pre-mRNA transcript. In some embodiments, the CAG repeat containing RNA is HTT, Ataxin1, Ataxin2, Ataxin3, CACNA1A, Ataxin7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, or MAB21L1.

In some embodiments, the abundance of the processed artificial miRNA is at least about 10%, 15%, 20%, 25%, 30%, 35%, 40%, 45%, 50%, 55%, 60%, 65%, 70%, 75%, 80%, 85%, 90% or more. % abundance of a correctly processed artificial miRNA may be measured by obtaining the number of RNA sequence reads of artificial miRNAs that have the correctly processed 5′ end and align to the reference CAG repeat region containing reference transcript, and dividing by the total number of RNA sequence reads of artificial miRNAs aligned to the reference CAG repeat region containing reference transcript.

In some embodiments, the processed artificial miRNA targets a pathogenic or pathologic allele of a CAG repeat containing RNA. In some embodiments, the processed artificial miRNA selectively targets a pathogenic or pathologic allele of a CAG repeat containing RNA compared to a normal allele of a CAG repeat containing RNA. In some embodiments, the pathogenic or pathologic allele of the CAG repeat containing RNA contains at least 30 consecutive CAG repeats.

In some embodiments, the cell is in a subject. In some embodiments, the subject is human. In some embodiments, the subject has or is suspected of having a CAG repeat expansion disorder.

The present disclosure also provides methods of inhibiting expression of a CAG repeat containing RNA in a cell comprising administering to the cell: (a) a viral vector encoding a double stranded RNA according to any of the embodiments described herein; or (b) a viral vector encoding an artificial miRNA, wherein the artificial miRNA comprises a guide sequence, a passenger sequence, and optionally a terminal loop, wherein the guide sequence targets a CAG repeat region of a CAG repeat containing RNA and comprises 1-5 base mismatches relative to the CAG repeat region, wherein the base mismatches are located at positions 8-16 of the guide sequence.

In some embodiments, at least one mismatch relative to the CAG repeat region is located at positions 8-12 of the guide sequence. In some embodiments, at least one mismatch relative to the CAG repeat region is located at positions 9-11 of the guide sequence. In some embodiments, two or more of the mismatches relative to the CAG repeat region located at positions 8-16 of the guide sequence are contiguous. In some embodiments, the guide sequence comprises 3, 4, or 5 base mismatches relative to the CAG repeat region.

In some embodiments, the guide sequence comprises of consists of a sequence selected from Tables B1-B2.

In some embodiments, the artificial miRNA of C1(b) further comprises a 5′ leader sequence and a 3′ trailer sequence. In some embodiments, the artificial miRNA comprises a 5′ leader sequence, a 3′ trailer sequence, and loop sequence selected from Table E.

In some embodiments, the processed artificial miRNA targets a pathogenic or pathologic allele of a CAG repeat containing RNA. In some embodiments, the processed artificial miRNA selectively targets a pathogenic or pathologic allele of a CAG repeat containing RNA compared to a normal allele of a CAG repeat containing RNA. In some embodiments, the pathogenic or pathologic allele of the CAG repeat containing RNA contains at least 30 consecutive CAG repeats.

In some embodiments, the CAG repeat containing RNA is a mRNA or pre-mRNA transcript. In some embodiments, the CAG repeat containing RNA is HTT, Ataxin1, Ataxin2, Ataxin3, CACNA1A, Ataxin7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, or MAB21L1.

In some embodiments, the guide sequence of the artificial miRNA has 0 predicted perfectly matching off-target transcripts. In some embodiments, the guide sequence of the artificial miRNA has 0-1 predicted off-target transcripts having perfect 17mer match within positions 1-21 of the guide sequence. In some embodiments, the guide sequence of the artificial miRNA has 0 predicted perfectly matching off-target transcripts. In some embodiments, the guide sequence of the artificial miRNA has 0-4 predicted off-target transcripts having perfect 17mer match within positions 1-21 of the guide sequence. In some embodiments, the guide sequence of the artificial miRNA with one substitution has 0-2 predicted off-target transcripts. In some embodiments, the guide sequence of the artificial miRNA has a predicted off-target transcript profile as set forth in Table B4.

In some embodiments, the cell is in a subject. In some embodiments, the subject is human. In some embodiments, the subject has or is suspected of having a CAG repeat expansion disorder.

In some embodiments, a subject treated in any of the methods described herein is a mammal (e.g., mouse, rat), preferably a primate (e.g., monkey, chimpanzee), or human.

In any of the methods of treatment described herein, a composition of the present disclosure (e.g., nucleic acid or expression cassette encoding a double stranded RNA, vector, or pharmaceutical composition according to the disclosure) may be administered to the subject by intrathecal, subpial, intraparenchymal, intrastriatal, intracranial, intracisternal, intra-cerebral, intracerebral ventricular, intraocular, intraventricular, intralumbar, intraocular, parenteral, intravenous, intramuscular, intra-arterial, subcutaneous, transdermal, interdermal, rectal, intravaginal, intraperitoneal, mucosal administration or any combination thereof.

In some embodiments, a composition of the present disclosure (e.g., inhibitory nucleic acid, isolated nucleic acid comprising an expression cassette encoding an inhibitory nucleic acid, vector, rAAV particle, pharmaceutical composition) is directly injected into the CNS of the subject. In some embodiments, direct injection into the CNS is intracerebral injection, intraparenchymal injection, intrathecal injection, subpial injection, or any combination thereof. In some embodiments, direct injection into the CNS is intracerebral ventricular injection. In some embodiments, direct injection into the CNS is direct injection into the cerebrospinal fluid (CSF) of the subject, optionally wherein the direct injection is intracisternal injection, intraventricular injection, intralumbar injection, or any combination thereof. In some embodiments, administration to the subject is accomplished by a combination of direct injection to the CNS and by a route that is not directly injected into the CNS (e.g., intravenously).

In some embodiments, the methods of the present disclosure reduce expression or activity of the CAG repeat containing RNA in a cell by at least 10%, at least 15%, at least 20%, at least 25%, at least 30%, at least 35%, at least 40%, at least 45%, at least 50%, at least 60%, at least 70%, at least 80%, at least 90% at least 95% or more in a cell compared to the expression level of the CAG repeat containing mRNA in a cell that has not been contacted with the double stranded RNA. In some embodiments, the methods of the present disclosure reduces the CAG repeat containing mRNA expression or activity in a cell by 10-20%, 10-30%, 10-40%, 10-50%, 10-60%, 10-70%, 10-80%, 10-90%, 10-95%, 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% compared to the expression level of the CAG repeat containing mRNA in a cell that has not been contacted with the double stranded RNA. In some embodiments, the methods of the present disclosure reduce expression or activity of the pathogenic or pathologic allele of the CAG repeat containing RNA. In some embodiments, the pathogenic or pathologic allele of the CAG repeat containing RNA contains at least 30 consecutive CAG repeats.

In some embodiments, the methods of the present disclosure reduce CAG repeat containing RNA expression or activity in the CNS of a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more in the CNS compared to the expression level of the CAG repeat containing mRNA in the CNS of an untreated subject. In some embodiments, the methods of the present disclosure reduces CAG repeat containing mRNA expression or activity in the CNS of a subject by 10-20%, 10-30%, 10-40%, 10-50%, 10-60%, 10-70%, 10-80%, 10-90%, 10-95%, 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% compared to the expression level of the CAG repeat containing mRNA in the CNS of an untreated subject. In some embodiments, the methods of the present disclosure reduce expression or activity of the pathogenic or pathologic allele of the CAG repeat containing RNA in the CNS of a subject. In some embodiments, the pathogenic or pathologic allele of the CAG repeat containing RNA contains at least 30 consecutive CAG repeats.

In some embodiments, the methods of the present disclosure are used for selective reduction of expression or activity of the pathogenic or pathologic allele of a CAG repeat containing RNA (having an expanded CAG repeat in a cell). In some embodiments, the pathogenic or pathologic allele of the CAG repeat containing RNA contains at least 30 consecutive CAG repeats. In some embodiments, a selective reduction of the pathogenic or pathologic allele of the CAG repeat containing RNA is at least 1.1×, 1.2×, 1.3×, 1.4×, 1.5×, 1.6×, 1.7×, 1.8×, 1.9×, 2×, 3×, 4×, 5×, 6×, 7×, 8×, 9×, 10×, 20×, 30×, 40×, 50×, 60×, 70×, 80×, 90×, 100×, 200× or more compared to the reduction of expression of the normal (wildtype) allele of the CAG repeat containing RNA. In some embodiments, the CAG repeat containing RNA is selected from HTT, Ataxin 1, Ataxin 2, Ataxin 3, CACNA1A, Ataxin 7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, and MAB21L1.

In some embodiments, the methods of the present disclosure are used for selective reduction of expression or activity of the pathogenic or pathologic allele of a CAG repeat containing RNA in the CNS of a subject. In some embodiments, the pathogenic or pathologic allele of the CAG repeat containing RNA contains at least 30 consecutive CAG repeats. In some embodiments, a selective reduction of the CAG repeat containing RNA having an expanded CAG repeat is at least 1.1×, 1.2×, 1.3×, 1.4×, 1.5×, 1.6×, 1.7×, 1.8×, 1.9×, 2× or more compared to the normal (wildtype) allele for the CAG repeat containing RNA. In some embodiments, the subject has or is suspected of having a CAG repeat expansion disease or disorder. In some embodiments, the CAG repeat containing RNA is selected from HTT, Ataxin 1, Ataxin 2, Ataxin 3, CACNA1A, Ataxin 7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, and MAB21L1.

### Examples of Non-Limiting Aspects of the Disclosure

### Aspects Set A

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A double stranded RNA comprising from 5′ to 3′:
(a) a 5′ leader sequence;
(b) a 5′ stem comprising a passenger sequence or a guide sequence;
(c) a 5′ linker of 1-6 bases;
(d) a terminal loop;
(e) a 3′ linker of 1-6 bases;
(f) a 3′ stem comprising:
   (i) a guide sequence if the 5′ stem comprises the passenger sequence; or
   (ii) a passenger sequence if the 5′ stem comprises the guide sequence; and
(g) a 3′ trailer sequence;
wherein the guide sequence targets a CAG repeat region of a CAG repeat containing RNA and comprises 1-5 base mismatches relative to the CAG repeat region, wherein the base mismatches are located at positions 8-16 of the guide sequence.
   Aspect 2. The double stranded RNA of aspect 1, wherein the double stranded RNA is contained within a pre-miRNA scaffold, pri-miRNA scaffold, or shRNA, optionally wherein the pre-miRNA scaffold or pri-miRNA scaffold is a mir-155 scaffold, a mir-33 scaffold, a mir-E scaffold, or any of the scaffolds set forth in Table E.
   Aspect 3. The double stranded RNA of aspect 1 or 2, wherein the 5′ leader sequence is not complementary to the 3′ trailer sequence.
   Aspect 4. The double stranded RNA of aspect 1 or 2, wherein the 5′ leader sequence is partially complementary to the 3′ trailer sequence.
   Aspect 5. The double stranded RNA of any one of aspects 1-4, wherein the 3′ trailer sequence comprises a polyU tail.
   Aspect 6. The double stranded RNA of aspect 5, wherein the polyU tail has 3 to 6 Us.
   Aspect 7. The double stranded RNA of any one of aspects 1-6, wherein the 5′ leader sequence and/or 3′ trailer sequence is about 1 nucleotide to about 500 nucleotides in length.
   Aspect 8. The double stranded RNA of aspect 7, wherein the 5′ leader sequence and/or 3′ trailer sequence is about 1 nucleotide to about 400 nucleotides, about 1 nucleotide to about 300 nucleotides, about 1 nucleotide to about 200 nucleotides, about 1 nucleotide to about 100 nucleotides, about 1 nucleotide to about 75 nucleotides about 1 nucleotide to about 50 nucleotides, about 1 nucleotide to about 25 nucleotides, about 1 nucleotide to about 20 nucleotides, about 1 nucleotide to about 15 nucleotides, or about 1 nucleotide to about 10 nucleotides in length.
   Aspect 9. The double stranded RNA of any one of aspects 1-8, wherein:
(i) the 5′ stem comprises the passenger sequence and the 5′ leader sequence comprises a portion of the 5′ end of the passenger sequence and/or the 3′ stem comprises the guide sequence 3′ trailer sequence comprises a portion of the 3′ end of the guide sequence; or
(ii) the 5′ stem comprises the guide sequence and the 5′ leader sequence comprises a portion of the 5′ end of the guide sequence and/or the 3′ stem comprises the passenger sequence 3′ trailer sequence comprises a portion of the 3′ end of the passenger sequence.
   Aspect 10. The double stranded RNA of any one of aspects 1-9, wherein the guide sequence has about 15 to about 30 nucleotides.
   Aspect 11. The double stranded RNA of aspect 10, wherein the guide sequence has about 19 to about 24 nucleotides.
   Aspect 12. The double stranded RNA of any one of aspects 1-11, wherein the guide sequence has a nucleotide sequence selected from Tables B1-B2.
   Aspect 13. The double stranded RNA of any one of aspects 1-12, wherein the passenger sequence is 100% complementary to the guide sequence.
   Aspect 14. The double stranded RNA of any one of aspects 1-12, wherein the passenger sequence is partially complementary to the guide sequence.
   Aspect 15. The double stranded RNA of aspect 14, wherein the passenger sequence comprises one to five base mismatches or bulges relative to the guide sequence.
   Aspect 16. The double stranded RNA of any one of aspects 1-15, wherein the 5′ linker is 100% complementary to the 3′ linker.
   Aspect 17. The double stranded RNA of any one of aspects 1-16, wherein the terminal loop has about 4 nucleotides to about 1,000 nucleotides, about 4 nucleotides to about 900 nucleotides, about 4 nucleotides to about 800 nucleotides, about 4 nucleotides to about 700 nucleotides, about 4 nucleotides to about 600 nucleotides, about 4 nucleotides to about 500 nucleotides, about 4 nucleotides to about 400 nucleotides, about 4 nucleotides to about 300 nucleotides, about 4 nucleotides to about 200 nucleotides, about 4 nucleotides to about 100 nucleotides, about 4 nucleotides to about 90 nucleotides about 4 nucleotides to about 80 nucleotides, about 4 nucleotides to about 70 nucleotides, about 4 nucleotides to about 50 nucleotides, about 4 nucleotides to about 40 nucleotides, about 4 nucleotides to about 30 nucleotides, about 4 nucleotides to about 20 nucleotides, about 4 nucleotides to about 15 nucleotides, or about 4 nucleotides to about 10 nucleotides.
   Aspect 18. The double stranded RNA of any one of aspects 1-17, wherein at least one mismatch relative to the CAG repeat region is located at positions 8-12 of the guide sequence.
   Aspect 19. The double stranded RNA of aspect 17, wherein at least one mismatch relative to the CAG repeat region is located at positions 9-11 of the guide sequence.
   Aspect 20. The double stranded RNA of any one of aspects 1-19, wherein two or more of the mismatches relative to the CAG repeat region located at positions 8-16 of the guide sequence are contiguous.
   Aspect 21. The double stranded RNA of any one of aspects 1-19, wherein at least one mismatch relative to the CAG repeat region located at positions 8-16 is not adjacent to another mismatch located at positions 8-16.
   Aspect 22. The double stranded RNA of any one of aspects 1-21, wherein the 1-5 base mismatches in the guide sequence do not base pair with the corresponding nucleotide(s) of the targeted CAG repeat region of the CAG repeat containing mRNA by Watson-Crick base pairing or by wobble base pairing.
   Aspect 23. The double stranded RNA of any one of aspects 1-22, wherein the double stranded RNA is cleaved to produce a shRNA comprising the guide sequence and the passenger sequence.
   Aspect 24. The double stranded RNA of aspect 23, wherein the double stranded RNA is cleaved by DROSHA to produce the shRNA.
   Aspect 25. The double stranded RNA of any one of aspects 1-24, wherein the double stranded RNA is cleaved to produce a small interfering RNA (siRNA) comprising the guide sequence and the passenger sequence.
   Aspect 26. The double stranded RNA of aspect 25, wherein the double stranded RNA is cleaved by DROSHA and DICER to produce the siRNA.
   Aspect 27. The double stranded RNA of aspect 25 or 26, wherein the guide sequence of the siRNA comprises 1-5 base mismatches relative to the CAG repeat region located at positions 8-16 of the guide sequence.
   Aspect 28. The double stranded RNA of aspect 23 or 24, wherein shRNA has an overhang at its 5′ end or at its 3′ end.
   Aspect 29. The double stranded RNA of aspect 23 or 24, wherein the 5′ overhang or 3′ overhang is about 1-6 nucleotides.
   Aspect 30. The double stranded RNA of aspect 23 or 24, wherein the shRNA does not have a 5′ overhang or 3′ overhang.
   Aspect 31. The double stranded RNA of aspect 23 or 24, wherein the shRNA has about 38 to about 50 nucleotides.
   Aspect 32. The double stranded RNA of any one of aspects 1-31, wherein the CAG repeat containing RNA is a mRNA or pre-mRNA transcript.
   Aspect 33. The double stranded RNA of any one of aspects 1-32, wherein the CAG repeat containing RNA is HTT, Ataxin1, Ataxin2, Ataxin3, CACNA1A, Ataxin7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, or MAB21L1.
   Aspect 34. A double stranded RNA comprising:
A) from 5′ to 3′:
   (a) a 5′ leader sequence;
   (b) a 5′ stem comprising a passenger sequence or a guide sequence;
   (c) a 5′ linker;
   (d) a terminal loop;
   (e) a 3′ linker;
   (f) a 3′ stem comprising:
   (i) a guide sequence if the 5′ stem comprises the passenger sequence; or
   (ii) a passenger sequence if the 5′ stem comprises the guide sequence; and
(g) a 3′ trailer sequence;
wherein:
   (i) the guide sequence targets a CAG repeat region of a CAG repeat containing RNA and comprises 1-5 base mismatches relative to the CAG repeat region, and
   (ii) the double stranded RNA is cleaved to produce a small interfering RNA (siRNA) comprising the guide sequence and the passenger sequence, wherein the guide sequence of the siRNA comprises 1-5 base mismatches relative to the CAG repeat region located at positions 8-16 of the guide sequence of the siRNA; or
B) comprising from 5′ to 3′:
   (i) a guide sequence that targets a CAG repeat region of a CAG repeat containing RNA and comprises 1-5 base mismatches relative to the CAG repeat region; and
   (ii) a passenger sequence that is substantially complementary to the guide sequence, wherein
   the double stranded RNA is cleaved to produce a siRNA comprising the guide sequence and the passenger sequence, wherein the guide sequence of the siRNA comprises 1-5 base mismatches relative to the CAG repeat region located at positions 8-16 of the guide sequence of the siRNA.
      Aspect 35. The double stranded RNA of aspect 34, wherein the double stranded RNA is comprised in a pre-miRNA scaffold, pri-miRNA scaffold, or shRNA, optionally wherein the pre-miRNA scaffold or pri-miRNA scaffold is a mir-155 scaffold, a mir-33 scaffold, a mir-E scaffold, or any of the scaffolds set forth in Table E.
      Aspect 36. The double stranded RNA of aspect 34, wherein the double stranded RNA is cleaved by DROSHA and DICER to produce the siRNA.
      Aspect 37. The double stranded RNA of aspect 34 or 35, wherein the 5′ leader sequence is not complementary to the 3′ trailer sequence.
      Aspect 38. The double stranded RNA of aspect 34 or 35, wherein the 5′ leader sequence is partially complementary to the 3′ trailer sequence.
      Aspect 39. The double stranded RNA of any one of aspects 34-38, wherein the 3′ trailer sequence comprises a polyT tail.
      Aspect 40. The double stranded RNA of aspect 39, wherein the polyT tail has 3 to 6 Ts.
      Aspect 41. The double stranded RNA of any one of aspects 34-40, wherein the 5′ leader sequence and/or 3′ trailer sequence is about 1 nucleotide to about 500 nucleotides in length.
      Aspect 42. The double stranded RNA of aspect 41, wherein the 5′ leader sequence and/or 3′ trailer sequence is about 1 nucleotide to about 400 nucleotides, about 1 nucleotide to about 300 nucleotides, about 1 nucleotide to about 200 nucleotides, about 1 nucleotide to about 100 nucleotides, about 1 nucleotide to about 75 nucleotides about 1 nucleotide to about 50 nucleotides, about 1 nucleotide to about 25 nucleotides, about 1 nucleotide to about 20 nucleotides, about 1 nucleotide to about 15 nucleotides, or about 1 nucleotide to about 10 nucleotides in length.
      Aspect 43. The double stranded RNA of any one of aspects 34-42, wherein:
(i) the 5′ stem comprises the passenger sequence and the 5′ leader sequence comprises a portion of the 5′ end of the passenger sequence and/or the 3′ stem comprises the guide sequence 3′ trailer sequence comprises a portion of the 3′ end of the guide sequence; or
(ii) the 5′ stem comprises the guide sequence and the 5′ leader sequence comprises a portion of the 5′ end of the guide sequence and/or the 3′ stem comprises the passenger sequence 3′ trailer sequence comprises a portion of the 3′ end of the passenger sequence.
   Aspect 44. The double stranded RNA of any one of aspects 34-43, wherein the guide sequence has about 15 to about 30 nucleotides.
   Aspect 45. The double stranded RNA of aspect 44, wherein the guide sequence has about 19 to about 24 nucleotides.
   Aspect 46. The double stranded RNA of any one of aspects 34-45, wherein the guide sequence has a nucleotide sequence selected from Tables B1-B2.
   Aspect 47. The double stranded RNA of any one of aspects 34-46, wherein the passenger sequence is 100% complementary to the guide sequence.
   Aspect 48. The double stranded RNA of any one of aspects 34-46, wherein the passenger sequence is partially complementary to the guide sequence.
   Aspect 49. The double stranded RNA of aspect 48, wherein the passenger sequence comprises one to five base mismatches or bulges relative to the guide sequence.
   Aspect 50. The double stranded RNA of any one of aspects 34-49, wherein the 5′ linker has 1-6 bases and/or the 3′ linker has 1-6 bases.
   Aspect 51. The double stranded RNA of any one of aspects 34-50, wherein the 5′ linker is 100% complementary to the 3′ linker.
   Aspect 52. The double stranded RNA of any one of aspects 34-51, wherein the terminal loop has about 4 nucleotides to about 1,000 nucleotides, about 4 nucleotides to about 900 nucleotides, about 4 nucleotides to about 800 nucleotides, about 4 nucleotides to about 700 nucleotides, about 4 nucleotides to about 600 nucleotides, about 4 nucleotides to about 500 nucleotides, about 4 nucleotides to about 400 nucleotides, about 4 nucleotides to about 300 nucleotides, about 4 nucleotides to about 200 nucleotides, about 4 nucleotides to about 100 nucleotides, about 4 nucleotides to about 90 nucleotides about 4 nucleotides to about 80 nucleotides, about 4 nucleotides to about 70 nucleotides, about 4 nucleotides to about 50 nucleotides, about 4 nucleotides to about 40 nucleotides, about 4 nucleotides to about 30 nucleotides, about 4 nucleotides to about 20 nucleotides, about 4 nucleotides to about 15 nucleotides, or about 4 nucleotides to about 10 nucleotides.
   Aspect 53. The double stranded RNA of any one of aspects 34-52, wherein at least one mismatch relative to the CAG repeat region is located at positions 8-12 of the guide sequence of the siRNA.
   Aspect 54. The double stranded RNA of aspect 53, wherein at least one mismatch relative to the CAG repeat region is located at positions 9-11 of the guide sequence of the siRNA.
   Aspect 55. The double stranded RNA of any one of aspects 34-54, wherein two or more of the mismatches relative to the CAG repeat region located at positions 8-16 of the guide sequence of the siRNA are contiguous.
   Aspect 56. The double stranded RNA of any one of aspects 34-54, wherein at least one mismatch relative to the CAG repeat region located at positions 8-16 of the guide sequence of the siRNA is not adjacent to another mismatch located at positions 8-16 of the guide sequence of the siRNA.
   Aspect 57. The double stranded RNA of any one of aspects 34-56, wherein the 1-5 base mismatches in the guide sequence of the siRNA do not base pair with the corresponding nucleotide(s) of the targeted CAG repeat region of the CAG repeat containing mRNA by Watson-Crick base pairing or by wobble base pairing.
   Aspect 58. The double stranded RNA of any one of aspects 34-57, wherein the CAG repeat containing RNA is a mRNA or pre-mRNA transcript.
   Aspect 59. The double stranded RNA of any one of aspects 34-58, wherein the CAG repeat containing mRNA is HTT, Ataxin1, Ataxin2, Ataxin3, CACNA1A, Ataxin7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, or MAB21L1.
   Aspect 60. A nucleic acid encoding the double stranded RNA of any one of aspects 1-59.
   Aspect 61. An expression cassette comprising the nucleic acid of aspect 60, operably linked to a promoter.
   Aspect 62. The expression cassette of aspect 61, wherein the promoter is an RNA polymerase promoter.
   Aspect 63. The expression cassette of aspect 62, wherein the RNA polymerase promoter is RNA polymerase II promoter or RNA polymerase III promoter.
   Aspect 64. The expression cassette of aspect 63, wherein the RNA polymerase III promoter is a U6, H1, or 7SK promoter.
   Aspect 65. The expression cassette of aspect 63, wherein the RNA polymerase II promoter is a U1, CMV, PGK, CAG, EIF1a, or synaptophysin promoter.
   Aspect 66. The expression cassette of any one of aspects 61-65, wherein the expression cassette is flanked by a 5′ adeno-associated virus (AAV) inverted terminal repeat (ITR) sequence and a 3′ AAV ITR sequence, or variants thereof.
   Aspect 67. The expression cassette of aspect 66, wherein one of the ITR sequences lacks a functional terminal resolution site.
   Aspect 68. The expression cassette of aspect 66 or 67, wherein the 5′ and 3′ ITRs are derived from an AAV serotype selected from the group consisting of: AAV1, AAV2, AAV5, AAV6, AAV6.2, AAV7, AAV8, AAV9, AAVRh10, AAV11, and variants thereof.
   Aspect 69. A vector comprising the nucleic acid molecule of aspect 60 or the expression cassette of any one of aspects 61-68.
   Aspect 70. The vector of aspect 79, wherein the vector is an adeno-associated virus (AAV) vector.
   Aspect 71. The AAV vector of aspect 70, further comprising a capsid protein, optionally wherein the capsid protein is an AAV1, AAV2, AAV5, AAV6, AAV6.2, AAV7, AAV8, AAV9, AAVRh10, or AAV11 capsid protein.
   Aspect 72. The AAV vector of aspect 70 or 71, wherein the AAV vector is a self-complementary AAV vector.
   Aspect 73. A pharmaceutical composition, or unit dosage form comprising the nucleic acid of aspect 60, the expression cassette of any one of aspects 61-68, or vector of any one of aspect 69-72, and a pharmaceutically acceptable carrier.
   Aspect 74. A method for reducing expression of a CAG repeat containing RNA in a subject in need thereof, comprising administering to the subject the nucleic acid of aspect 60, the expression cassette of any one of aspects 61-68, the vector of any one of aspect 69-72, or the pharmaceutical composition of aspect 73.
   Aspect 75. The method of aspect 74, wherein the inhibiting expression of a CAG repeat containing RNA comprises inhibiting expression of the polyglutamine containing protein encoded by the CAG repeat containing RNA.
   Aspect 76. The method of aspect 74 or 75, wherein the CAG repeat containing RNA is a mRNA or pre-mRNA transcript.
   Aspect 77. The method of any one of aspects 74-76, wherein the subject has or is suspected of having a polyglutamine disease, optionally wherein the polyglutamine disease is associated with: HTT, Ataxin1, Ataxin2, Ataxin3, CACNA1A, Ataxin7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, or MAB21L1.
   Aspect 78. The method of aspect 77, wherein the polyglutamine disease is Huntington's disease, spinocerebellar ataxia type 1, spinocerebellar ataxia type 2, spinocerebellar ataxia type 3, spinocerebellar ataxia type 6, spinocerebellar ataxia type 7, spinocerebellar ataxia type 12, spinocerebellar ataxia type 17, amyotrophic lateral sclerosis, spinal bulbar muscular atrophy, dentatorubral pallidoluysian atrophy, cleidocranial dysplasia, retinitis pigmentosa, myotonic dystrophy 1, or branchiootorenal syndrome 2.
   Aspect 79. The method of any one of aspects 74-78, wherein the administration comprises direct injection to the central nervous system (CNS) of the subject.
   Aspect 80. The method of aspect 79, wherein the direct injection is intracerebral injection, intraparenchymal injection, intrathecal injection, intrastriatal injection, subpial injection, or any combination thereof.
   Aspect 81. The method of any one of aspects 74-78, wherein the administration comprises intraocular, parenteral, intravenous, intramuscular, intra-arterial, subcutaneous, transdermal, interdermal, rectal, intravaginal, intraperitoneal, or mucosal administration.
   Aspect 82. A method of inhibiting expression of a CAG repeat containing RNA in a mammalian cell comprising introducing into the mammalian cell a double stranded RNA comprising from 5′ to 3′:
(a) a 5′ leader sequence;
(b) a 5′ stem comprising a passenger sequence;
(c) a 5′ linker;
(d) a terminal loop;
(e) a 3′ linker;
(f) a 3′ stem comprising a guide sequence; and
(g) a 3′ trailer sequence;
wherein the guide sequence targets a CAG repeat region of a CAG repeat containing RNA; and
wherein the double stranded RNA is cleaved by the mammalian cell to produce a siRNA comprising the guide sequence and the passenger sequence, wherein the guide sequence of the siRNA comprises the 1-5 base mismatches relative to the CAG repeat region located at positions 8-16 of the guide sequence of the siRNA.
   Aspect 83. The method of aspect 82, wherein the inhibiting expression of a CAG repeat containing mRNA comprises inhibiting expression of the polyglutamine containing protein encoded by the CAG repeat containing mRNA.
   Aspect 84. The method of aspect 82, wherein the double stranded RNA is comprised in a pre-miRNA scaffold, pri-miRNA scaffold, or shRNA.
   Aspect 85. The method of aspect 82-84, wherein the double stranded RNA is cleaved by DROSHA and DICER in the mammalian cell to yield the siRNA.
   Aspect 86. The method of any one of aspects 82-85, wherein at least one mismatch relative to the CAG repeat region is located at positions 8-12 of the guide sequence of the siRNA.
   Aspect 87. The method of aspect 86, wherein at least one mismatch relative to the CAG repeat region is located at positions 9-11 of the guide sequence of the siRNA.
   Aspect 88. The method of any one of aspects 82-87, wherein two or more of the mismatches relative to the CAG repeat region located at positions 8-16 of the guide sequence of the siRNA are contiguous.
   Aspect 89. The method of any one of aspects 82-88, wherein at least one mismatch relative to the CAG repeat region located at positions 8-16 of the guide sequence of the siRNA is not adjacent to another mismatch located at positions 8-16 of the guide sequence of the siRNA.
   Aspect 90. The method of any one of aspects 82-89, wherein the 5′ leader sequence is not complementary to the 3′ trailer sequence.
   Aspect 91. The method of any one of aspects 82-89, wherein the 5′ leader sequence is partially complementary to the 3′ trailer sequence.
   Aspect 92. The method of any one of aspects 82-91, wherein the 3′ trailer sequence comprises a polyU tail.
   Aspect 93. The method of aspect 92, wherein the polyU tail has 3 to 6 uridines.
   Aspect 94. The method of any one of aspects 82-93, wherein the 5′ leader sequence and/or 3′ trailer sequence is about 1 nucleotide to about 500 nucleotides in length.
   Aspect 95. The method of aspect 94, wherein the 5′ leader sequence and/or 3′ trailer sequence is about 1 nucleotide to about 400 nucleotides, about 1 nucleotide to about 300 nucleotides, about 1 nucleotide to about 200 nucleotides, about 1 nucleotide to about 100 nucleotides, about 1 nucleotide to about 75 nucleotides about 1 nucleotide to about 50 nucleotides, about 1 nucleotide to about 25 nucleotides, about 1 nucleotide to about 20 nucleotides, about 1 nucleotide to about 15 nucleotides, or about 1 nucleotide to about 10 nucleotides in length.
   Aspect 96. The method of any one of aspects 82-95, wherein the guide sequence has about 15 to about 30 nucleotides.
   Aspect 97. The method of any one of aspects 82-95, wherein the guide sequence has about 19 to about 24 nucleotides.
   Aspect 98. The method of any one of aspects 82-97, wherein the guide sequence has a nucleotide sequence set selected from Tables B1-B2.
   Aspect 99. The method of any one of aspects 82-98, wherein the passenger sequence is 100% complementary to the guide sequence.
   Aspect 100. The method of any one of aspects 82-98, wherein the passenger sequence is partially complementary to the guide sequence.
   Aspect 101. The method of aspect 100, wherein the passenger sequence comprises one to five base mismatches or bulges relative to the guide sequence.
   Aspect 102. The method of any one of aspects 82-101, wherein the 5′ linker is 100% complementary to the 3′ linker.
   Aspect 103. The method of any one of aspects 82-102, wherein the terminal loop has about 4 nucleotides to about 1,000 nucleotides, about 4 nucleotides to about 900 nucleotides, about 4 nucleotides to about 800 nucleotides, about 4 nucleotides to about 700 nucleotides, about 4 nucleotides to about 600 nucleotides, about 4 nucleotides to about 500 nucleotides, about 4 nucleotides to about 400 nucleotides, about 4 nucleotides to about 300 nucleotides, about 4 nucleotides to about 200 nucleotides, about 4 nucleotides to about 100 nucleotides, about 4 nucleotides to about 90 nucleotides about 4 nucleotides to about 80 nucleotides, about 4 nucleotides to about 70 nucleotides, about 4 nucleotides to about 50 nucleotides, about 4 nucleotides to about 40 nucleotides, about 4 nucleotides to about 30 nucleotides, about 4 nucleotides to about 20 nucleotides, about 4 nucleotides to about 15 nucleotides, or about 4 nucleotides to about 10 nucleotides.
   Aspect 104. The method of any one of aspects 82-103, comprising introducing a vector comprising a nucleic acid encoding the double stranded RNA into the mammalian cell.
   Aspect 105. The method of aspect 104, wherein the nucleic acid encoding the double stranded RNA is operably linked to a promoter.
   Aspect 106. The method of aspect 105, wherein the promoter is an RNA polymerase II promoter of RNA polymerase III promoter.
   Aspect 107. The method of any one of aspects 82-106, wherein the siRNAs produced from the double stranded RNA in the mammalian cell and having 1-5 base mismatches relative to the CAG repeat region located at their predicted positions within positions 8-16 of the guide sequence are at an abundance of at least 10% of the siRNAs produced from the double stranded RNA by the mammalian cell.
   Aspect 108. The method of any one of aspects 82-107, wherein the CAG repeat containing RNA is a mRNA or pre-mRNA transcript.
   Aspect 109. The method of any one of aspects 82-108, wherein the CAG repeat containing RNA is HTT, Ataxin1, Ataxin2, Ataxin3, CACNA1A, Ataxin7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, or MAB21L1.
   Aspect 110. A method of increasing abundance of a processed artificial miRNA that targets a CAG repeat containing RNA in a cell, wherein the artificial miRNA is encoded by a viral vector, comprising administering to the cell:
(a) a viral vector encoding a double stranded RNA according to any one of aspects 1-59; or
(b) a viral vector encoding the artificial miRNA, wherein the artificial miRNA comprises a guide sequence, a passenger sequence, and optionally a terminal loop, wherein the guide sequence targets a CAG repeat region of a CAG repeat containing RNA and comprises 1-5 base mismatches relative to the CAG repeat region, wherein the base mismatches are located at positions 8-16 of the guide sequence.
   Aspect 111. The method of aspect 110, wherein at least one mismatch relative to the CAG repeat region is located at positions 8-12 of the guide sequence.
   Aspect 112. The method of aspect 110, wherein at least one mismatch relative to the CAG repeat region is located at positions 9-11 of the guide sequence.
   Aspect 113. The method of any one of aspects 110-112, wherein two or more of the mismatches relative to the CAG repeat region located at positions 8-16 of the guide sequence are contiguous.
   Aspect 114. The method of any one of aspects 110-113, wherein the guide sequence comprises or consists of a sequence selected from Tables B1-B2.
   Aspect 115. The method of any one of aspects 110-114, wherein the artificial miRNA of aspect 110(b) further comprises a 5′ leader sequence and a 3′ trailer sequence.
   Aspect 116. The method of any one of aspects 110-115, wherein the artificial miRNA comprises a 5′ leader sequence, a 3′ trailer sequence, and loop sequence selected from Table E.
   Aspect 117. The method of any one of aspects 110-116, wherein the CAG repeat containing RNA is an mRNA or pre-mRNA transcript.
   Aspect 118. The method of any one of aspects 110-117, wherein the CAG repeat containing RNA is a pathologic allele of a CAG repeat containing gene.
   Aspect 119. The method of aspect 118, wherein the pathologic allele of the CAG repeat containing gene contains at least 30 consecutive CAG repeats.
   Aspect 120. The method of any one of aspects 110-119, wherein the CAG repeat containing RNA is HTT, Ataxin1, Ataxin2, Ataxin3, CACNA1A, Ataxin7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, or MAB21L1.
   Aspect 121. The method of any one of aspects 110-120, wherein the abundance of the processed artificial miRNA is at least about 10%, 15%, 20%, 25%, 30%, 35%, 40%, 45%, 50%, 55%, 60%, 65%, 70%, 75%, 80%, 85%, 90% or more.
   Aspect 122. The method of any one of aspects 110-121, wherein the cell is in a subject.
   Aspect 123. The method of aspect 122, wherein the subject is human.
   Aspect 124. The method of aspect 123, wherein the subject has a CAG repeat expansion disorder.
   Aspect 125. A method of inhibiting expression of a CAG repeat containing RNA in a cell comprising administering to the cell:
(a) a viral vector encoding a double stranded RNA according to any one of aspects 1-59; or
(b) a viral vector encoding an artificial miRNA, wherein the artificial miRNA comprises a guide sequence, a passenger sequence, and optionally a terminal loop, wherein the guide sequence targets a CAG repeat region of a CAG repeat containing RNA and comprises 1-5 base mismatches relative to the CAG repeat region, wherein the base mismatches are located at positions 8-16 of the guide sequence.
   Aspect 126. The method of aspect 125, wherein at least one mismatch relative to the CAG repeat region is located at positions 8-12 of the guide sequence.
   Aspect 127. The method of aspect 125, wherein at least one mismatch relative to the CAG repeat region is located at positions 9-11 of the guide sequence.
   Aspect 128. The method of any one of aspects 125-127, wherein two or more of the mismatches relative to the CAG repeat region located at positions 8-16 of the guide sequence are contiguous.
   Aspect 129. The method of any one of aspects 125-128, wherein the guide sequence comprises 3, 4, or 5 base mismatches relative to the CAG repeat region.
   Aspect 130. The method of any one of aspects 125-129, wherein the guide sequence comprises or consists of a sequence selected from Tables B1-B2.
   Aspect 131. The method of any one of aspects 125-130, wherein the artificial miRNA of C1(b) further comprises a 5′ leader sequence and a 3′ trailer sequence.
   Aspect 132. The method of any one of aspects 125-131, wherein the artificial miRNA comprises a 5′ leader sequence, a 3′ trailer sequence, and loop sequence selected from Table E.
   Aspect 133. The method of any one of aspects 125-132, wherein the CAG repeat containing RNA is an mRNA or pre-mRNA transcript.
   Aspect 134. The method of any one of aspects 125-133, wherein the CAG repeat containing RNA is a pathologic allele of a CAG repeat containing gene.
   Aspect 135. The method of aspect 134, wherein the pathologic allele of the CAG repeat containing gene contains at least 30 consecutive CAG repeats.
   Aspect 136. The method of any one of aspects 125-135, wherein the CAG repeat containing RNA is HTT, Ataxin1, Ataxin2, Ataxin3, CACNA1A, Ataxin7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, or MAB21L1.
   Aspect 137. The method of any one of aspects 125-136, wherein the guide sequence of the artificial miRNA has 0 predicted perfectly matching off-target transcripts.
   Aspect 138. The method of any one of aspects 125-137, wherein the guide sequence of the artificial miRNA has 0-1 predicted off-target transcripts having perfect 17mer match within positions 1-21.
   Aspect 139. The method of any one of aspects 125-138, wherein the guide sequence of the artificial miRNA has 0-2 predicted matching off-target transcripts with one mismatch.
   Aspect 140. The method of any one of aspects 125-139, wherein the guide sequence of the artificial miRNA has a predicted off-target transcript profile as set forth in Table B4.
   Aspect 141. The method of any one of aspects 125-140, wherein the cell is in a subject.
   Aspect 142. The method of aspect 141, wherein the subject is human.
   Aspect 143. The method of aspect 142, wherein the subject has a CAG repeat expansion disorder.
   Aspect 144. A method for treating a polyglutamine expansion disease in a subject in need thereof, comprising administering to the subject the nucleic acid of aspect 60, the expression cassette of any one of aspects 61-68, the vector of any one of aspect 69-72, or the pharmaceutical composition of aspect 73.
   Aspect 145. The method of aspect 144, wherein the polyglutamine expansion disease is associated with: HTT, Ataxin1, Ataxin2, Ataxin3, CACNA1A, Ataxin7, PPP2R2B, TBP, Androgen receptor, Atrophin, MLLT3, BMP2K, THAP11, ZFHX3, POU3F2, MAML2, SMARCA2, MAML3, ORC4, RUNX2, MED12, EP400, MAGI1, UMAD1, DM1-AS, AC007161.3, IRF2BPL, or MAB21L1.
   Aspect 146. The method of aspect 144, wherein the polyglutamine expansion disease is Huntington's disease, spinocerebellar ataxia type 1, spinocerebellar ataxia type 2, spinocerebellar ataxia type 3, spinocerebellar ataxia type 6, spinocerebellar ataxia type 7, spinocerebellar ataxia type 12, spinocerebellar ataxia type 17, amyotrophic lateral sclerosis, spinal bulbar muscular atrophy, dentatorubral pallidoluysian atrophy, cleidocranial dysplasia, retinitis pigmentosa, myotonic dystrophy 1, or branchiootorenal syndrome 2.
   Aspect 147. The method of any one of aspects 144-146, wherein the administration comprises direct injection to the central nervous system (CNS) of the subject.
   Aspect 148. The method of aspect 147, wherein the direct injection is intracerebral injection, intraparenchymal injection, intrathecal injection, intrastriatal injection, subpial injection, or any combination thereof.
   Aspect 149. The method of any one of aspects 144-146, wherein the administration comprises intraocular, parenteral, intravenous, intramuscular, intra-arterial, subcutaneous, transdermal, interdermal, rectal, intravaginal, intraperitoneal, or mucosal administration.

### Aspects Set B

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A double-stranded RNA comprising
a) a first strand that hybridizes to a target CAG repeat region of a CAG repeat containing RNA; and
b) a second strand that hybridizes to the first strand,
wherein the first strand comprises:
   i) a first mismatch to the target CAG repeat region; and
   ii) at least a second mismatch to the target CAG repeat region,
wherein:
i) when the first mismatch is at position 8 based on the numbering of SEQ ID NO:743 (CUGCUGCUGCUGCUGCUGCUG (SEQ ID NO:743)), SEQ ID NO:866(GCUGCUGCUGCUGCUGCUGCU (SEQ ID NO:866)), or SEQ ID NO:867 (UGCUGCUGCUGCUGCUGCUGC (SEQ ID NO:867)), the second mismatch is from is from 1 to 8 bases 3′ of the first mismatch;
ii) when the first mismatch is at position 9 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867), the second mismatch is from 1 to 7 bases 3′ of the first mismatch;
iii) when the first mismatch is at position 10 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)), the second mismatch is from 1 to 6 bases 3′ of the first mismatch; and iv) when the first mismatch is at position 11 based on the numbering of SEQ ID NO:743 (CUGCUGCUGCUGCUGCUGCUG (SEQ ID NO:/743)), SEQ ID NO:866 (GCUGCUGCUGCUGCUGCUGCU (SEQ ID NO:866)), or (UGCUGCUGCUGCUGCUGCUGC (SEQ ID NO:867)), the second mismatch is from 1 to 5 bases 3′ of the first mismatch.
   Aspect 2. The double-stranded RNA of aspect 1, wherein each mismatch is generated by a substitution that is independently selected from:
a) a substitution of a G with an A, a U, or a C;
b) a substitution of a U with an A, a G, or a C;
c) a substitution of a C with an A, a U, or a G.
   Aspect 3. The double-stranded RNA of aspect 1 or aspect 2, wherein the first strand comprises:
i) a first mismatch to the target CAG repeat region, wherein the first mismatch is at position 8 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867)); and
ii) a second mismatch to the target CAG repeat region, wherein the second mismatch is from 1 to 8 bases 3′ of the first mismatch.
   Aspect 4. The double-stranded RNA of any one of aspects 1-3, wherein the first strand is a variant comprising at least a first and a second substitution of the nucleotide sequence: CUGCUGCUGCUGCUGCUGCUG (SEQ ID NO:743) wherein the first substitution generates the first mismatch and is at position 8 based on the numbering of CUGCUGCUGCUGCUGCUGCUG (SEQ ID NO:743) and the second substitution generates the second mismatch and is from 1 to 8 bases 3′ of the first mismatch.
   Aspect 5. The double-stranded RNA of any one of aspects 1-3, wherein the first strand is a variant comprising at least a first and a second substitution of the nucleotide sequence: GCUGCUGCUGCUGCUGCUGCU (SEQ ID NO:866), wherein the first substitution generates the first mismatch and is at position 8 based on the numbering of GCUGCUGCUGCUGCUGCUGCU (SEQ ID NO:866), and the second substitution generates the second mismatch and is from 1 to 8 bases 3′ of the first mismatch.
   Aspect 6. The double-stranded RNA of any one of aspects 1-3, wherein the first strand is a variant comprising at least a first and a second substitution of the nucleotide sequence: UGCUGCUGCUGCUGCUGCUGC (SEQ ID NO:867), wherein the first substitution generates the first mismatch and is at position 8 based on the numbering of UGCUGCUGCUGCUGCUGCUGC (SEQ ID NO:867), and the second substitution generates the second mismatch and is from 1 to 8 bases 3′ of the first mismatch.
   Aspect 7. The double-stranded RNA of any one of aspects 3-6, wherein the first strand comprises no more than 2 mismatches with the target CAG repeat region.
   Aspect 8. The double-stranded RNA of any one of aspects 3-6, wherein the first strand comprises no more than 3 mismatches with the target CAG repeat region.
   Aspect 9. The double-stranded RNA of any one of aspects 3-6, wherein the first strand comprises no more than 4 mismatches with the target CAG repeat region.
   Aspect 10. The double-stranded RNA of aspect 3 or aspect 4, wherein the first strand comprises a nucleotide sequence selected from:

i)
(SEQ ID NO: 317)
CUGCUGCAACUGCUGCUGCUG (CUG_NA_B);
ii)
(SEQ ID NO: 318)
CUGCUGCAGAUGCUGCUGCUG (CUG_NA_C);
iii)
(SEQ ID NO: 319)
CUGCUGCAGCAGCUGCUGCUG (CUG_NA_D);
iv)
(SEQ ID NO: 320)
CUGCUGCAGCUACUGCUGCUG(CUG_NA_E);
v)
(SEQ ID NO: 321)
CUGCUGCAGCUGAUGCUGCUG (CUG_NA_F);
vi)
(SEQ ID NO: 322)
CUGCUGCAGCUGCAGCUGCUG (CUG_NA_G);
vii)
(SEQ ID NO: 323)
CUGCUGCAGCUGCUACUGCUG (CUG_NA_H);
and
viii)
(SEQ ID NO: 324)
CUGCUGCAGCUGCUGAUGCUG (CUG_NA_I).

Aspect 11. The double-stranded RNA of aspect 3 or aspect 4, wherein the first strand comprises a nucleotide sequence selected from SEQ ID NOs:804-819.
Aspect 12. The double-stranded RNA of aspect 1 or aspect 2, wherein the first strand comprises a first mismatch to the target CAG repeat region, wherein the first mismatch is at position 8 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867)); wherein the first strand comprises a second mismatch and a third mismatch to the target CAG repeat region, and wherein the second and third mismatches are from 1 to 8 bases 3′ of the first mismatch.
Aspect 13. The double-stranded RNA of any one of aspects 1, 2, and 12, wherein the first strand is a variant comprising at least a first, a second, and a third substitution of the nucleotide sequence CUGCUGCUGCUGCUGCUGCUG (SEQ ID NO:743), wherein the first substitution generates the first mismatch and is at position 8 based on the numbering of CUGCUGCUGCUGCUGCUGCUG (SEQ ID NO:743), wherein the second substitution generates the second mismatch and the third substitution generates the third mismatch, and wherein the second substitution and the third substitution are from 1 to 8 bases 3′ of the first mismatch.
Aspect 14. The double-stranded RNA of any one of aspects 1, 2, and 12, wherein the first strand is a variant comprising at least a first, a second, and a third substitution of the nucleotide sequence GCUGCUGCUGCUGCUGCUGCU (SEQ ID NO:866), and wherein the first substitution generates the first mismatch and is at position 8 based on the numbering of GCUGCUGCUGCUGCUGCUGCU (SEQ ID NO:866), wherein the second substitution generates the second mismatch and the third substitution generates the third mismatch, and wherein the second substitution and the third substitution are from 1 to 8 bases 3′ of the first mismatch.
Aspect 15. The double-stranded RNA of any one of aspects 1, 2, and 12, wherein the first strand is a variant comprising at least a first, a second, and a third substitution of the nucleotide sequence UGCUGCUGCUGCUGCUGCUGC (SEQ ID NO:867), and wherein the first substitution generates the first mismatch and is at position 8 based on the numbering of UGCUGCUGCUGCUGCUGCUGC (SEQ ID NO:867), wherein the second substitution generates the second mismatch and the third substitution generates the third mismatch, and wherein the second substitution and the third substitution are from 1 to 8 bases 3′ of the first mismatch.
Aspect 16. The double-stranded RNA of any one of aspects 12-15, wherein the first strand comprises no more than 3 mismatches with the target CAG repeat region.
Aspect 17. The double-stranded RNA of any one of aspects 12-15, wherein the first strand comprises no more than 4 mismatches with the target CAG repeat region.
Aspect 18. The double-stranded RNA of aspect 1 or aspect 2, wherein the first strand comprises a first mismatch to the target CAG repeat region, wherein the first mismatch is at position 8 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867)); wherein the first strand comprises a second mismatch, a third mismatch, and a fourth mismatch to the target CAG repeat region, and wherein the second, third, and fourth mismatches are from 1 to 8 bases 3′ of the first mismatch.
Aspect 19. The double-stranded RNA of any one of aspects 1, 2, and 18, wherein the first strand is a variant comprising at least a first, a second, a third, and a fourth substitution of the nucleotide sequence CUGCUGCUGCUGCUGCUGCUG (SEQ ID NO:743), wherein the first substitution generates the first mismatch and is at position 8 based on the numbering of CUGCUGCUGCUGCUGCUGCUG (SEQ ID NO:743), wherein the second substitution generates the second mismatch, the third substitution generates the third mismatch, and the fourth substitution generates the fourth mismatch, and wherein the second, third, and fourth substitutions are from 1 to 8 bases 3′ of the first mismatch.
Aspect 20. The double-stranded RNA of any one of aspects 1, 2, and 18, wherein the first strand is a variant comprising at least a first, a second, a third, and a fourth substitution of the nucleotide sequence GCUGCUGCUGCUGCUGCUGCU (SEQ ID NO:866), wherein the first substitution generates the first mismatch and is at position 8 based on the numbering of GCUGCUGCUGCUGCUGCUGCU (SEQ ID NO:866), wherein the second substitution generates the second mismatch, the third substitution generates the third mismatch, and the fourth substitution generates the fourth mismatch, and wherein the second, third, and fourth substitutions are from 1 to 8 bases 3′ of the first mismatch.
Aspect 21. The double-stranded RNA of any one of aspects 1, 2, and 18, wherein the first strand is a variant comprising at least a first, a second, a third, and a fourth substitution of the nucleotide sequence UGCUGCUGCUGCUGCUGCUGC (SEQ ID NO:867), wherein the first substitution generates the first mismatch and is at position 8 based on the numbering of UGCUGCUGCUGCUGCUGCUGC (SEQ ID NO:867), wherein the second substitution generates the second mismatch, the third substitution generates the third mismatch, and the fourth substitution generates the fourth mismatch, and wherein the second, third, and fourth substitutions are from 1 to 8 bases 3′ of the first mismatch.
Aspect 22. The double-stranded RNA of any one of aspects 18-21, wherein the first strand comprises no more than 4 mismatches with the target CAG repeat region.
Aspect 23. The double-stranded RNA of aspect 1 or aspect 2, wherein the first strand comprises:
i) a first mismatch to the target CAG repeat region, wherein the first mismatch is at position 9 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867), and
ii) a second mismatch to the target CAG repeat region, wherein the second mismatch is from 1 to 7 bases 3′ of the first mismatch.
   Aspect 24. The double-stranded RNA of any one of aspects 1, 2, and 23, wherein the first strand is a variant comprising at least a first and a second substitution of the nucleotide sequence CUGCUGCUGCUGCUGCUGCUG (SEQ ID NO:743), wherein the first substitution generates the first mismatch and is at position 9 based on the numbering of CUGCUGCUGCUGCUGCUGCUG (SEQ ID NO:743), and the second substitution generates the second mismatch and is from 1 to 7 bases 3′ of the first mismatch.
   Aspect 25. The double-stranded RNA of any one of aspects 1, 2, and 23, wherein the first strand is a variant comprising at least a first and a second substitution of the nucleotide sequence GCUGCUGCUGCUGCUGCUGCU (SEQ ID NO:866), wherein the first substitution generates the first mismatch and is at position 9 based on the numbering of GCUGCUGCUGCUGCUGCUGCU (SEQ ID NO:866), and the second substitution generates the second mismatch and is from 1 to 7 bases 3′ of the first mismatch.
   Aspect 26. The double-stranded RNA of any one of aspects 1, 2, and 23, wherein the first strand is a variant comprising at least a first and a second substitution of the nucleotide sequence UGCUGCUGCUGCUGCUGCUGC (SEQ ID NO:867), wherein the first substitution generates the first mismatch and is at position 9 based on the numbering of UGCUGCUGCUGCUGCUGCUGC (SEQ ID NO:867), and the second substitution generates the second mismatch and is from 1 to 7 bases 3′ of the first mismatch.
   Aspect 27. The double-stranded RNA of aspect 23, wherein the first strand comprises a nucleotide sequence selected from SEQ ID NOs:298-304.
   Aspect 28. The double-stranded RNA of any one of aspects 23-27, wherein the first strand comprises no more than 2 mismatches with the target CAG repeat region.
   Aspect 29. The double-stranded RNA of any one of aspects 23-27, wherein the first strand comprises no more than 3 mismatches with the target CAG repeat region.
   Aspect 30. The double-stranded RNA of any one of aspects 23-27, wherein the first strand comprises no more than 4 mismatches with the target CAG repeat region.
   Aspect 31. The double-stranded RNA of aspect 1 or aspect 2, wherein the first mismatch is at position 9 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867), wherein the first strand comprises a second mismatch and a third mismatch to the target CAG repeat region, and wherein the second and third mismatches are from 1 to 7 bases 3′ of the first mismatch.
   Aspect 32. The double-stranded RNA of any one of aspects 1, 2, and 31, wherein the first strand is a variant comprising at least a first, a second, and a third substitution of the nucleotide sequence CUGCUGCUGCUGCUGCUGCUG (SEQ ID NO:743), wherein the first substitution generates the first mismatch and is at position 9 based on the numbering of CUGCUGCUGCUGCUGCUGCUG (SEQ ID NO:743), wherein the second substitution generates the second mismatch and the third substitution generates the third mismatch, and wherein the second substitution and the third substitution are from 1 to 7 bases 3′ of the first mismatch.
   Aspect 33. The double-stranded RNA of any one of aspects 1, 2, and 31, wherein the first strand is a variant comprising at least a first, a second, and a third substitution of the nucleotide sequence GCUGCUGCUGCUGCUGCUGCU (SEQ ID NO:866), wherein the first substitution generates the first mismatch and is at position 9 based on the numbering of GCUGCUGCUGCUGCUGCUGCU (SEQ ID NO:866), wherein the second substitution generates the second mismatch and the third substitution generates the third mismatch, and wherein the second substitution and the third substitution are from 1 to 7 bases 3′ of the first mismatch.
   Aspect 34. The double-stranded RNA of any one of aspects 1, 2, and 31, wherein the first strand is a variant comprising at least a first, a second, and a third substitution of the nucleotide sequence UGCUGCUGCUGCUGCUGCUGC (SEQ ID NO:867), wherein the first substitution generates the first mismatch and is at position 9 based on the numbering of UGCUGCUGCUGCUGCUGCUGC (SEQ ID NO:867), wherein the second substitution generates the second mismatch and the third substitution generates the third mismatch, and wherein the second substitution and the third substitution are from 1 to 7 bases 3′ of the first mismatch.
   Aspect 35. The double-stranded RNA of any one of aspects 31-34, wherein the first strand comprises no more than 3 mismatches with the target CAG repeat region.
   Aspect 36. The double-stranded RNA of any one of aspects 31-34, wherein the first strand comprises no more than 4 mismatches with the target CAG repeat region.
   Aspect 37. The double-stranded RNA of aspect 31, wherein the first strand comprises a nucleotide sequence selected from:

i)
(SEQ ID NO: 325)
CUGCUGCUAAAGCUGCUGCUG (CUG_307);
ii)
(SEQ ID NO: 326)
CUGCUGCUAAUACUGCUGCUG (CUG_334);
iii)
(SEQ ID NO: 327)
CUGCUGCUAAUGAUGCUGCUG (CUG_361);
iv)
(SEQ ID NO: 328)
CUGCUGCUAAUGCAGCUGCUG(CUG_388);
v)
(SEQ ID NO: 329)
CUGCUGCUAAUGCUACUGCUG (CUG_415);
vi)
(SEQ ID NO: 330)
CUGCUGCUACAGAUGCUGCUG (CUG_631);
vii)
(SEQ ID NO: 331)
CUGCUGCUACAGCAGCUGCUG (CUG_658);
viii)
(SEQ ID NO: 332)
CUGCUGCUACAGCUGAUGCUG (CUG_712);
ix)
(SEQ ID NO: 336)
CUGCUGCUAAUGCUGAUGCUG (CUG_442);
x)
(SEQ ID NO: 337)
CUGCUGCUACAACUGCUGCUG (CUG_604);
and
xi)
(SEQ ID NO: 338)
CUGCUGCUACAGCUACUGCUG (CUG_685).

Aspect 38. The double-stranded RNA of aspect 1 or aspect 2, wherein the first strand comprises a first mismatch to the target CAG repeat region, wherein the first mismatch is at position 9 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867), wherein the first strand comprises a second mismatch, a third mismatch, and a fourth mismatch to the target CAG repeat region, and wherein the second, third, and fourth mismatches are from 1 to 7 bases 3′ of the first mismatch.
Aspect 39. The double-stranded RNA of any one of aspects 1, 2, and 38, wherein the first strand is a variant comprising at least a first, a second, a third, and a fourth substitution of the nucleotide sequence CUGCUGCUGCUGCUGCUGCUG (SEQ ID NO:743), wherein the first substitution generates the first mismatch and is at position 9 based on the numbering of CUGCUGCUGCUGCUGCUGCUG (SEQ ID NO: 743), wherein the second substitution generates the second mismatch, the third substitution generates the third mismatch, and the fourth substitution generates the fourth mismatch, and wherein the second, third, and fourth substitutions are from 1 to 7 bases 3′ of the first mismatch.
Aspect 40. The double-stranded RNA of any one of aspects 1, 2, and 38, wherein the first strand is a variant comprising at least a first, a second, a third, and a fourth substitution of the nucleotide sequence GCUGCUGCUGCUGCUGCUGCU (SEQ ID NO:866), wherein the first substitution generates the first mismatch and is at position 9 based on the numbering of GCUGCUGCUGCUGCUGCUGCU (SEQ ID NO:866), wherein the second substitution generates the second mismatch, the third substitution generates the third mismatch, and the fourth substitution generates the fourth mismatch, and wherein the second, third, and fourth substitutions are from 1 to 7 bases 3′ of the first mismatch.
Aspect 41. The double-stranded RNA of any one of aspects 1, 2, and 38, wherein the first strand is a variant comprising at least a first, a second, a third, and a fourth substitution of the nucleotide sequence UGCUGCUGCUGCUGCUGCUGC (SEQ ID NO:867), wherein the first substitution generates the first mismatch and is at position 9 based on the numbering of UGCUGCUGCUGCUGCUGCUGC (SEQ ID NO:867), wherein the second substitution generates the second mismatch, the third substitution generates the third mismatch, and the fourth substitution generates the fourth mismatch, and wherein the second, third, and fourth substitutions are from 1 to 7 bases 3′ of the first mismatch.
Aspect 42. The double-stranded RNA of any one of aspects 38-41, wherein the first strand comprises no more than 4 mismatches with the target CAG repeat region.
Aspect 43. The double-stranded RNA of claim 38, wherein the first strand comprises a nucleotide sequence selected from SEQ ID NOs:341-344.
Aspect 44. The double-stranded RNA of claim 38, wherein the first strand comprises a nucleotide sequence selected from SEQ ID NOs:347-367.
Aspect 45. The double-stranded RNA of aspect 1 or aspect 2, wherein the first strand comprises:
i) a first mismatch to the target CAG repeat region, wherein the first mismatch is at position 10 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)); and
ii) a second mismatch to the target CAG repeat region, wherein the second mismatch is from 1 to 6 bases 3′ of the first mismatch.
   Aspect 46. The double-stranded RNA of any one of aspects 1, 2, or 45, wherein the first strand is a variant comprising at least a first and a second substitution of the nucleotide sequence CUGCUGCUGCUGCUGCUGCUG (SEQ ID NO:743), wherein the first substitution generates the first mismatch and is at position 10 based on the numbering of CUGCUGCUGCUGCUGCUGCUG (SEQ ID NO:743), and wherein the second substitution generates the second mismatch and is from 1 to 6 bases 3′ of the first mismatch.
   Aspect 47. The double-stranded RNA of any one of aspects 1, 2, or 45, wherein the first strand is a variant comprising at least a first and a second substitution of the nucleotide sequence GCUGCUGCUGCUGCUGCUGCU (SEQ ID NO:866), wherein the first substitution generates the first mismatch and is at position 10 based on the numbering of GCUGCUGCUGCUGCUGCUGCU (SEQ ID NO:866), and wherein the second substitution generates the second mismatch and is from 1 to 6 bases 3′ of the first mismatch.
   Aspect 48. The double-stranded RNA of any one of aspects 1, 2, or 45, wherein the first strand is a variant comprising at least a first and a second substitution of the nucleotide sequence UGCUGCUGCUGCUGCUGCUGC (SEQ ID NO:867), wherein the first substitution generates the first mismatch and is at position 10 based on the numbering of UGCUGCUGCUGCUGCUGCUGC (SEQ ID NO:867), and wherein the second substitution generates the second mismatch and is from 1 to 6 bases 3′ of the first mismatch.
   Aspect 49. The double-stranded RNA of any one of aspects 45-48, wherein the first strand comprises no more than 2 mismatches with the target CAG repeat region.
   Aspect 50. The double-stranded RNA of any one of aspects 45-48, wherein the first strand comprises no more than 3 mismatches with the target CAG repeat region.
   Aspect 51. The double-stranded RNA of any one of aspects 45-48, wherein the first strand comprises no more than 4 mismatches with the target CAG repeat region.
   Aspect 52. The double-stranded RNA of aspect 45, wherein the first strand comprises a nucleotide sequence selected from SEQ ID NOs:305-310.
   Aspect 53. The double-stranded RNA of aspect 1 or aspect 2, wherein the first mismatch is at position 10 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)), wherein the first strand comprises a second mismatch and a third mismatch to the target CAG repeat region, and wherein the second and third mismatches are from 1 to 6 bases 3′ of the first mismatch.
   Aspect 54. The double-stranded RNA of any one of aspects 1, 2, or 53, wherein the first strand is a variant comprising at least a first, a second, and a third substitution of the nucleotide sequence CUGCUGCUGCUGCUGCUGCUG (SEQ ID NO:743), wherein the first substitution generates the first mismatch and is at position 10 based on the numbering of CUGCUGCUGCUGCUGCUGCUG (SEQ ID NO:743), wherein the second substitution generates the second mismatch and the third substitution generates the third mismatch, and wherein the second substitution and the third substitution are from 1 to 6 bases 3′ of the first mismatch.
   Aspect 55. The double-stranded RNA of any one of aspects 1, 2, or 53, wherein the first strand is a variant comprising at least a first, a second, and a third substitution of the nucleotide sequence GCUGCUGCUGCUGCUGCUGCU (SEQ ID NO:866), wherein the first substitution generates the first mismatch and is at position 10 based on the numbering of GCUGCUGCUGCUGCUGCUGCU (SEQ ID NO:866), wherein the second substitution generates the second mismatch and the third substitution generates the third mismatch, and wherein the second substitution and the third substitution are from 1 to 6 bases 3′ of the first mismatch.
   Aspect 56. The double-stranded RNA of any one of aspects 1, 2, or 53, wherein the first strand is a variant comprising at least a first, a second, and a third substitution of the nucleotide sequence UGCUGCUGCUGCUGCUGCUGC (SEQ ID NO:867), wherein the first substitution generates the first mismatch and is at position 10 based on the numbering UGCUGCUGCUGCUGCUGCUGC (SEQ ID NO:867), wherein the second substitution generates the second mismatch and the third substitution generates the third mismatch, and wherein the second substitution and the third substitution are from 1 to 6 bases 3′ of the first mismatch.
   Aspect 57. The double-stranded RNA of any one of aspects 53-56, wherein the first strand comprises no more than 3 mismatches with the target CAG repeat region.
   Aspect 58. The double-stranded RNA of any one of aspects 53-56, wherein the first strand comprises no more than 4 mismatches with the target CAG repeat region.
   Aspect 59. The double-stranded RNA of aspect 53, wherein the first strand comprises a nucleotide sequence selected from:

i)
(SEQ ID NO: 333)
CUGCUGCUGAAGAUGCUGCUG (CUG_2116);
ii)
(SEQ ID NO: 334)
CUGCUGCUGAAGCAGCUGCUG (CUG_2143);
iii)
(SEQ ID NO: 335)
CUGCUGCUGAAGCUACUGCUG (CUG_2170);
iv)
(SEQ ID NO: 339)
CUGCUGCUGAAACUGCUGCUG (CUG_2089);
and
v)
(SEQ ID NO: 340)
CUGCUGCUGAAGCUGAUGCUG (CUG_2197).

Aspect 60. The double-stranded RNA of aspect 1 or aspect 2, wherein the first strand comprises a first mismatch to the target CAG repeat region, wherein the first mismatch is at position 10 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)), wherein the first strand comprises a second mismatch, a third mismatch, and a fourth mismatch to the target CAG repeat region, and wherein the second, third, and fourth mismatches are from 1 to 6 bases 3′ of the first mismatch.
Aspect 61. The double-stranded RNA of aspect 1, 2, or 60, wherein the first strand is a variant comprising at least a first, a second, a third, and a fourth substitution of the nucleotide sequence CUGCUGCUGCUGCUGCUGCUG (SEQ ID NO:743), wherein the first substitution generates the first mismatch and is at position 10 based on the numbering of CUGCUGCUGCUGCUGCUGCUG (SEQ ID NO:743), wherein the second substitution generates the second mismatch, the third substitution generates the third mismatch, and the fourth substitution generates the fourth mismatch, and wherein the second, third, and fourth substitutions are from 1 to 6 bases 3′ of the first mismatch.
Aspect 62. The double-stranded RNA of aspect 1, 2, or 60, wherein the first strand is a variant comprising at least a first, a second, a third, and a fourth substitution of the nucleotide sequence GCUGCUGCUGCUGCUGCUGCU (SEQ ID NO:866), wherein the first substitution generates the first mismatch and is at position 10 based on the numbering of CUGCUGCUGCUGCUGCUGCUG (SEQ ID NO:866), wherein the second substitution generates the second mismatch, the third substitution generates the third mismatch, and the fourth substitution generates the fourth mismatch, and wherein the second, third, and fourth substitutions are from 1 to 6 bases 3′ of the first mismatch.
Aspect 63. The double-stranded RNA of aspect 1, 2, or 60, wherein the first strand is a variant comprising at least a first, a second, a third, and a fourth substitution of the nucleotide sequence UGCUGCUGCUGCUGCUGCUGC (SEQ ID NO:867), wherein the first substitution generates the first mismatch and is at position 10 based on the numbering of UGCUGCUGCUGCUGCUGCUGC (SEQ ID NO:867), wherein the second substitution generates the second mismatch, the third substitution generates the third mismatch, and the fourth substitution generates the fourth mismatch, and wherein the second, third, and fourth substitutions are from 1 to 6 bases 3′ of the first mismatch.
Aspect 64. The double-stranded RNA of any one of aspects 60-63, wherein the first strand comprises no more than 4 mismatches with the target CAG repeat region.
Aspect 65. The double-stranded RNA of aspect 60, wherein the first strand comprises a nucleotide sequence selected from SEQ ID NOs:345, 346, and 368-375.
Aspect 66. The double-stranded RNA of aspect 1 or aspect 2, wherein the first strand comprises:
i) a first mismatch to the target CAG repeat region, wherein the first mismatch is at position 11 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)); and
ii) a second mismatch to the target CAG repeat region, wherein the second mismatch is from 1 to 5 bases 3′ of the first mismatch.
   Aspect 67. The double-stranded RNA of any one of aspects 1, 2, and 66, wherein the first strand is a variant comprising at least a first and a second substitution of the nucleotide sequence CUGCUGCUGCUGCUGCUGCUG (SEQ ID NO:743), wherein the first substitution generates the first mismatch and is at position 11 based on the numbering of CUGCUGCUGCUGCUGCUGCUG (SEQ ID NO:743), and the second substitution generates the second mismatch and is from 1 to 5 bases 3′ of the first mismatch.
   Aspect 68. The double-stranded RNA of any one of aspects 1, 2, and 66, wherein the first strand is a variant comprising at least a first and a second substitution of the nucleotide sequence GCUGCUGCUGCUGCUGCUGCU (SEQ ID NO:866), wherein the first substitution generates the first mismatch and is at position 11 based on the numbering of GCUGCUGCUGCUGCUGCUGCU (SEQ ID NO:866), and the second substitution generates the second mismatch and is from 1 to 5 bases 3′ of the first mismatch.
   Aspect 69. The double-stranded RNA of any one of aspects 1, 2, and 66, wherein the first strand is a variant comprising at least a first and a second substitution of the nucleotide sequence UGCUGCUGCUGCUGCUGCUGC (SEQ ID NO:867), wherein the first substitution generates the first mismatch and is at position 11 based on the numbering of UGCUGCUGCUGCUGCUGCUGC (SEQ ID NO:867), and the second substitution generates the second mismatch and is from 1 to 5 bases 3′ of the first mismatch.
   Aspect 70. The double-stranded RNA of any one of aspects 66-69, wherein the first strand comprises no more than 2 mismatches with the target CAG repeat region.
   Aspect 71. The double-stranded RNA of any one of aspects 66-69, wherein the first strand comprises no more than 3 mismatches with the target CAG repeat region.
   Aspect 72. The double-stranded RNA of any one of aspects 66-69, wherein the first strand comprises no more than 4 mismatches with the target CAG repeat region.
   Aspect 73. The double-stranded RNA of aspect 66, wherein the first strand comprises a nucleotide sequence selected from:

i)
(SEQ ID NO: 311)
CUGCUGCUGCAACUGCUGCUG (CUG_217);
ii)
(SEQ ID NO: 312)
CUGCUGCUGCAGAUGCUGCUG (CUG_226);
iii)
(SEQ ID NO: 313)
CUGCUGCUGCAGCAGCUGCUG (CUG_235);
iv)
(SEQ ID NO: 314)
CUGCUGCUGCAGCUACUGCUG (CUG_244);
and
v)
(SEQ ID NO: 315)
CUGCUGCUGCAGCUGAUGCUG (CUG_253).

Aspect 74. The double-stranded RNA of aspect 66, wherein the first strand comprises a nucleotide sequence selected from SEQ ID NOs:793-803.
Aspect 75. The double-stranded RNA of aspect 1 or aspect 2, wherein the first strand comprises a first mismatch to the target CAG repeat region, wherein the first mismatch is at position 11 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)); and wherein the first strand comprises a second mismatch and a third mismatch to the target CAG repeat region, and wherein the second and third mismatches are from 1 to 5 bases 3′ of the first mismatch.
Aspect 76. The double-stranded RNA of any one of aspects 1, 2, and 75, wherein the first strand is a variant comprising at least a first, a second, and a third substitution of the nucleotide sequence CUGCUGCUGCUGCUGCUGCUG (SEQ ID NO:743), wherein the first substitution generates the first mismatch and is at position 11 based on the numbering of CUGCUGCUGCUGCUGCUGCUG (SEQ ID NO:743), wherein the second substitution generates the second mismatch and the third substitution generates the third mismatch, and wherein the second substitution and the third substitution are from 1 to 5 bases 3′ of the first mismatch.
Aspect 77. The double-stranded RNA of any one of aspects 1, 2, and 75, wherein the first strand is a variant comprising at least a first, a second, and a third substitution of the nucleotide sequence GCUGCUGCUGCUGCUGCUGCU (SEQ ID NO:866), wherein the first substitution generates the first mismatch and is at position 11 based on the numbering of GCUGCUGCUGCUGCUGCUGCU (SEQ ID NO:866), wherein the second substitution generates the second mismatch and the third substitution generates the third mismatch, and wherein the second substitution and the third substitution are from 1 to 5 bases 3′ of the first mismatch.
Aspect 78. The double-stranded RNA of any one of aspects 1, 2, and 75, wherein the first strand is a variant comprising at least a first, a second, and a third substitution of the nucleotide sequence UGCUGCUGCUGCUGCUGCUGC (SEQ ID NO:867), wherein the first substitution generates the first mismatch and is at position 11 based on the numbering of UGCUGCUGCUGCUGCUGCUGC (SEQ ID NO:867), wherein the second substitution generates the second mismatch and the third substitution generates the third mismatch, and wherein the second substitution and the third substitution are from 1 to 5 bases 3′ of the first mismatch.
Aspect 79. The double-stranded RNA of any one of aspects 75-78, wherein the first strand comprises no more than 3 mismatches with the target CAG repeat region.
Aspect 80. The double-stranded RNA of any one of aspects 75-78, wherein the first strand comprises no more than 4 mismatches with the target CAG repeat region.
Aspect 81. The double-stranded RNA of aspect 1 or aspect 2, wherein the first strand comprises a first mismatch to the target CAG repeat region, wherein the first mismatch is at position 11 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)), wherein the first strand comprises a second mismatch, a third mismatch, and a fourth mismatch to the target CAG repeat region, and wherein the second, third, and fourth mismatches are from 1 to 5 bases 3′ of the first mismatch.
Aspect 82. The double-stranded RNA of any one of aspects 1, 2, and 81, wherein the first strand is a variant comprising at least a first, a second, a third, and a fourth substitution of the nucleotide sequence CUGCUGCUGCUGCUGCUGCUG (SEQ ID NO:743), wherein the first substitution generates the first mismatch and is at position 11 based on the numbering of CUGCUGCUGCUGCUGCUGCUG (SEQ ID NO:743), wherein the second substitution generates the second mismatch, the third substitution generates the third mismatch, and the fourth substitution generates the fourth mismatch, and wherein the second, third, and fourth substitutions are from 1 to 5 bases 3′ of the first mismatch.
Aspect 83. The double-stranded RNA of any one of aspects 1, 2, and 81, wherein the first strand is a variant comprising at least a first, a second, a third, and a fourth substitution of the nucleotide sequence GCUGCUGCUGCUGCUGCUGCU (SEQ ID NO:866), wherein the first substitution generates the first mismatch and is at position 11 based on the numbering of GCUGCUGCUGCUGCUGCUGCU (SEQ ID NO:866), wherein the second substitution generates the second mismatch, the third substitution generates the third mismatch, and the fourth substitution generates the fourth mismatch, and wherein the second, third, and fourth substitutions are from 1 to 5 bases 3′ of the first mismatch.
Aspect 84. The double-stranded RNA of any one of aspects 1, 2, and 81, wherein the first strand is a variant comprising at least a first, a second, a third, and a fourth substitution of the nucleotide sequence UGCUGCUGCUGCUGCUGCUGC (SEQ ID NO:867), wherein the first substitution generates the first mismatch and is at position 11 based on the numbering of UGCUGCUGCUGCUGCUGCUGC (SEQ ID NO:867), wherein the second substitution generates the second mismatch, the third substitution generates the third mismatch, and the fourth substitution generates the fourth mismatch, and wherein the second, third, and fourth substitutions are from 1 to 5 bases 3′ of the first mismatch.
Aspect 85. The double-stranded RNA of any one of aspects 81-84, wherein the first strand comprises no more than 4 mismatches with the target CAG repeat region.
Aspect 86. The double-stranded RNA of any one of aspects 1-85, wherein the second strand is 100% complementary to the first strand.
Aspect 87. The double-stranded RNA of any one of aspects 1-85, wherein the second strand comprises from 1 to 10 mismatches, from 3 to 5 mismatches, from 4 to 7 mismatches, or from 5 to 10 mismatches, to the first strand.
Aspect 88. The double-stranded RNA of any one of aspects 1-85, wherein the double-stranded RNA has a length of from 18 bases to 25 nucleotides, from 19 to 25 nucleotides, from 19 to 23 nucleotides, or from 19 to 21 nucleotides.
Aspect 89. The double-stranded RNA of any one of aspects 1-85, wherein the double-stranded RNA has a length of from 21 nucleotides to 25 nucleotides.
Aspect 90. A DNA molecule comprising a nucleotide sequence encoding the first strand as set forth in any one of aspects 1-89, wherein the nucleotide sequence is operably linked to a promoter that is functional in a eukaryotic cell.
Aspect 91. A recombinant nucleic acid comprising:
a1) the double-stranded RNA of any one of aspects 1-89; and
b1) a microRNA scaffold comprising a 5′ flanking polynucleotide, a loop polynucleotide, and a 3′ flanking polynucleotide,
wherein the recombinant nucleic acid comprises:
   i) the 5′ flanking polynucleotide;
   ii) the first strand of the double-stranded RNA;
   iii) the loop polynucleotide;
   iv) the second strand of the double-stranded RNA; and
   iv) the 3′ flanking polynucleotide;
wherein at least one of the 5′ flanking polynucleotide, the loop polynucleotide, and the 3′ flanking polynucleotide is heterologous to the first and/or the second strand of the double-stranded RNA; or
a2) the double-stranded RNA of any one of aspects 1-89; and
b2) a microRNA scaffold comprising a 5′ flanking polynucleotide, a loop polynucleotide, and a 3′ flanking polynucleotide,
wherein the recombinant nucleic acid comprises:
   i) the 5′ flanking polynucleotide;
   ii) the second strand of the double-stranded RNA;
   iii) the loop polynucleotide;
   iv) the first strand of the double-stranded RNA; and
   v) the 3′ flanking polynucleotide;
wherein at least one of the 5′ flanking polynucleotide, the loop polynucleotide, and the 3′ flanking polynucleotide is heterologous to the first and/or the second strand of the double-stranded RNA; or
a3) the double-stranded RNA of any one of aspects 1-89; and
b3) a microRNA scaffold comprising a 5′ flanking polynucleotide and a 3′ flanking polynucleotide,
wherein the recombinant nucleic acid comprises:
   i) the 5′ flanking polynucleotide;
   ii) the first strand of the double-stranded RNA;
   iii) the second strand of the double-stranded RNA; and
   iv) the 3′ flanking polynucleotide;
wherein one or both of the 5′ flanking polynucleotide and the 3′ flanking polynucleotide is heterologous to the first and/or the second strand of the double-stranded RNA; or
a4) the double-stranded RNA of any one of aspects 1-89; and
b4) a microRNA scaffold comprising a 5′ flanking polynucleotide and a 3′ flanking polynucleotide,
wherein the recombinant nucleic acid comprises:
   i) the 5′ flanking polynucleotide;
   ii) the second strand of the double-stranded RNA;
   iii) the first strand of the double-stranded RNA; and
   iv) the 3′ flanking polynucleotide;
wherein one or both of the 5′ flanking polynucleotide and the 3′ flanking polynucleotide is heterologous to the first and/or the second strand of the double-stranded RNA.
   Aspect 92. The recombinant nucleic acid of aspect 91, wherein the recombinant nucleic acid comprises:
a) the 5′ flanking polynucleotide;
b) the first strand of the double-stranded RNA;
c) the loop polynucleotide;
d) the second strand of the double-stranded RNA; and
e) the 3′ flanking polynucleotide.
   Aspect 93. The recombinant nucleic acid of aspect 91 or aspect 92, wherein the 5′ flanking polynucleotide, the loop polynucleotide, and the 3′ flanking polynucleotide are derived from miR33.
   Aspect 94. The recombinant nucleic acid of aspect 91, wherein the recombinant nucleic acid comprises:
a) the 5′ flanking polynucleotide;
b) the first strand of the double-stranded RNA;
c) the second strand of the double-stranded RNA; and
d) the 3′ flanking polynucleotide.
   Aspect 95. The recombinant nucleic acid of aspect 91 or aspect 94, wherein the 5′ flanking polynucleotide and the 3′ flanking polynucleotide are derived from miR451.
   Aspect 96. A DNA molecule comprising a nucleotide sequence encoding a recombinant nucleic acid according to any one of aspects 91-95.
   Aspect 97. The DNA molecule of aspect 96, wherein the 5′ flanking polynucleotide is encoded by the nucleotide sequence: tgcacacctcctggcgggcagctctg (SEQ ID NO:738).
   Aspect 98. The DNA molecule of aspect 96 or aspect 97, wherein the loop polynucleotide is encoded by the nucleotide sequence: tgttctggcaatacctg (SEQ ID NO:739).
   Aspect 99. The DNA molecule of any one of aspects 96-98, wherein the 3′ flanking polynucleotide is encoded by the nucleotide sequence: gggaggcctgccctgactgcccac (SEQ ID NO:740).
   Aspect 100. The DNA molecule of any one of aspects 96-99, comprising the nucleotide sequence set forth in any one of SEQ ID NOs:579-656.
   Aspect 101. The DNA molecule of aspect 96, wherein the 5′ flanking polynucleotide is encoded by the nucleotide sequence acctactgactgccagggcacttgggaatggcaagg (SEQ ID NO:854).
   Aspect 102. The DNA molecule of aspect 96 or aspect 101, wherein the 3′ flanking polynucleotide is encoded by the nucleotide sequence tcttgctatacccagaaaacgtgccaggaagagaac (SEQ ID NO:855).
   Aspect 103. The DNA molecule of aspect 96, wherein:
i) the 5′ flanking polynucleotide is encoded by the nucleotide sequence acctactgactgccagggcacttgggaatggcaagg (SEQ ID NO:854);
ii) the 3′ flanking polynucleotide is encoded by the nucleotide sequence tcttgctatacccagaaaacgtgccaggaagagaac (SEQ ID NO:855); and
iii) the first strand or the second strand is encoded by the nucleotide sequence set forth in any one of SEQ ID NOs:379-456.
   Aspect 104. The DNA molecule of aspect 96, wherein the 5′ flanking polynucleotide is encoded by the nucleotide sequence set forth in SEQ ID NO:856.
   Aspect 105. The DNA molecule of aspect 96 or aspect 104, wherein the 3′ flanking polynucleotide is encoded by the nucleotide sequence set forth in SEQ ID NO:857.
   Aspect 106. The DNA molecule of aspect 96, wherein:
i) the 5′ flanking polynucleotide is encoded by the nucleotide sequence set forth in SEQ ID NO:856;
ii) the 3′ flanking polynucleotide is encoded by the nucleotide sequence set forth in SEQ ID NO:857; and
iii) the first strand or the second strand is encoded by the nucleotide sequence set forth in any one of SEQ ID NOs:379-456.
   Aspect 107. The DNA molecule of aspect 96, wherein the 5′ flanking polynucleotide is encoded by the nucleotide sequence set forth in SEQ ID NO:858.
   Aspect 108. The DNA molecule of aspect 96 or aspect 107, wherein the 3′ flanking polynucleotide is encoded by the nucleotide sequence set forth in SEQ ID NO:859.
   Aspect 109. The DNA molecule of aspect 96, wherein:
i) the 5′ flanking polynucleotide is encoded by the nucleotide sequence set forth in SEQ ID NO:858;
ii) the 3′ flanking polynucleotide is encoded by the nucleotide sequence set forth in SEQ ID NO:859; and
iii) the first strand or the second strand is encoded by the nucleotide sequence set forth in any one of SEQ ID NOs:379-456.
   Aspect 110. A recombinant expression vector comprising the DNA molecule of any one of aspects 96 to 109.
   Aspect 111. The recombinant expression vector of aspect 110, wherein the nucleotide sequence is operably linked to a promoter that is functional in a eukaryotic cell.
   Aspect 112. The recombinant expression vector of aspect 111, wherein the promoter is an RNA polymerase II promoter or an RNA polymerase III promoter.
   Aspect 113. The recombinant expression vector of aspect 111 or aspect 112, wherein the promoter is a CAG promoter, a CBA promoter, a CMV promoter, a U6 promoter, an EF1α promoter, or an H1 promoter.
   Aspect 114. The recombinant expression vector of any one of aspects 110-113, wherein the recombinant expression vector comprises a 5′ adeno-associated virus (AAV) inverted terminal repeat (ITR) sequence and a 3′ AAV ITR sequence.
   Aspect 115. A recombinant expression vector comprising a nucleotide sequence encoding the recombinant nucleic acid of any one of aspects 91-95.
   Aspect 116. The recombinant expression vector of aspect 115, wherein the nucleotide sequence is operably linked to a promoter that is functional in a eukaryotic cell.
   Aspect 117. The recombinant expression vector of aspect 116, wherein the promoter is an RNA polymerase II promoter or an RNA polymerase III promoter.
   Aspect 118. The recombinant expression vector of aspect 116 or aspect 117, wherein the promoter is a CAG promoter, a CBA promoter a CMV promoter, a U6 promoter, an EF1α promoter, or an H1 promoter.
   Aspect 119. The recombinant expression vector of any one of aspects 115-118, wherein the recombinant expression vector comprises a 5′ adeno-associated virus (AAV) inverted terminal repeat (ITR) sequence and a 3′ AAV ITR sequence.
   Aspect 120. A delivery vehicle comprising the recombinant expression vector of any one of aspects 110-119.
   Aspect 121. The delivery vehicle of aspect 120, wherein the delivery vehicle a non-viral delivery vehicle.
   Aspect 122. The delivery vehicle of aspect 121, wherein the delivery vehicle is a lipid nanoparticle.
   Aspect 123. The delivery vehicle of aspect 120, wherein the delivery vehicle is a viral particle.
   Aspect 124. A viral particle comprising the recombinant expression vector of any one of aspects 110-119.
   Aspect 125. The viral particle of aspect 126, wherein the viral particle is an adeno-associated virus (AAV) particle.
   Aspect 126. The viral particle of aspect 125, wherein the AAV particle comprises an AAV9 capsid.
   Aspect 127. The viral particle of aspect 125, wherein the AAV particle comprises an AAV2 capsid.
   Aspect 128. A composition comprising:
a) the recombinant expression vector of any one of aspects 110-119; and
b) a pharmaceutically acceptable excipient.
   Aspect 129. A composition comprising:
a) the delivery vehicle of any one of aspects 120-123; and
b) a pharmaceutically acceptable excipient.
   Aspect 130. A composition comprising:
a) a viral particle comprising the recombinant expression vector of any one of aspects 124-127; and
b) a pharmaceutically acceptable excipient.
   Aspect 131. A method for selectively reducing translation of a disease-associated CAG repeat-containing RNA in an individual having a CAG repeat expansion disorder, the method comprising administering to the individual an effective amount of the expression vector of any one of aspects 110-119, delivery vehicle of any one of aspects 120-123, the viral particle of any one of aspects 124-127, or the pharmaceutical composition of any one of aspects 128-130.
   Aspect 132. The method of aspect 131, wherein the repeat expansion disorder is Huntington's disease, spinocerebellar ataxia type 1, spinocerebellar ataxia type 2, spinocerebellar ataxia type 3, spinocerebellar ataxia type 6, spinocerebellar ataxia type 7, spinocerebellar ataxia type 12, spinocerebellar ataxia type 17, spinal and bulbar muscular atrophy, dentatorubral pallidoluysian atrophy, or cleidocranial dysplasia.
   Aspect 133. The method of aspect 131 or aspect 132, wherein said administering comprises direct injection to the central nervous system of the individual.
   Aspect 134. The method of aspect 133, wherein the direct injection is intracerebral ventricular injection, intraparenchymal injection, intrathecal injection, intrastriatal injection, intrathalamic injection, intracisternal magna injection, subpial injection, or any combination thereof.
   Aspect 135. The method of any one of aspects 131-134, wherein said administering provides for a ratio of a polypeptide encoded by the non-disease-associated CAG repeat-containing RNA to a polypeptide encoded by disease-associated CAG repeat-containing RNA of greater than 1.0.
   Aspect 136. The method of any one of aspects 131-134, wherein said administering provides for a ratio of a polypeptide encoded by non-disease-associated CAG repeat-containing RNA to a polypeptide encoded by disease-associated CAG repeat-containing RNA of from 1.1 to 1.8.
   Aspect 137. The method of any one of aspects 131-134, wherein said administering provides for a ratio of a polypeptide encoded by non-disease-associated CAG repeat-containing RNA to a polypeptide encoded by disease-associated CAG repeat-containing RNA of greater than 1.8.

### EXAMPLES

### Materials and Methods

Cloning of CAG Repeat-Targeted shRNA Expression Vectors

Lentiviral pLKO.1-TRC shRNA expression vector was obtained from Addgene (10878) and EGFP inserted upstream of the puromycin resistance gene separated by a T2A polypeptide linker. Additionally, an XhoI restriction site downstream of the shRNA cloning site was mutated. This custom vector, pxTRC-EGFP-puro, was subsequently digested with EcoRI and AgeI to release a 1.9 kb stuffer sequence, treated with FastAP (Thermo Fisher) to dephosphorylate, then gel-purified. The linearized and purified vector was then used to add shRNA inserts, which were purchased from Integrated DNA Technologies (IDT) as single strands and annealed into duplexes. Duplexes were designed to possess an additional 16 bps on each end that matched the pxTRC-EGFP-puro vector immediately upstream and downstream of the AgeI and EcoRI restriction enzyme cut sites to enable recombination-based cloning with In-Fusion (Takara Bio).

Ligated plasmids were transformed into NEB Stable chemically competent cells (New England Biolabs) and grown on LB-agar-carbenicillin plates at 30° C. Individual colonies were grown in liquid culture in LB-carbenicillin at 30° C., extracted with standard mini-prep or midi-prep procedures, and positive clones confirmed by Sanger sequencing.

Next-Generation Sequencing of Expressed shRNAs

shRNA expression vectors (500 ng) were electroporated into 30-40,000 cells with a Neon Transfection System (Invitrogen) and grown at 37° C. in 5% CO₂ for 84 h. Human embryonic Kidney (HEK) 293T cells (ATCC) were grown in Eagle's minimum essential medium (EMEM) supplemented with 1× non-essential amino acids (NEAA) (Thermo Fisher), 5% fetal bovine serum (FBS), and 5% cosmic calf serum (CCS) while patient-derived fibroblasts (Coriell Cell Repository, GM04281) were grown in EMEM supplemented with 20% FBS, 1 mM sodium pyruvate, and 1×NEAA. Cells were harvested by trypsinization and processed for small RNA extraction using the mirVana miRNA Isolation Kit (Invitrogen) following the manufacturer's recommended protocol. RNA was quantified by UV spectrophotometry with a Nanodrop instrument and sent for sequencing at a core facility. Small RNAs were sequenced by Illumina after preparation with a TruSeq Small RNA Library kit. Small RNA sequencing results were analyzed by a custom bioinformatic workflow. Briefly, data was first converted to a fastq file and run through Trimmomatic using the following command line:
java -jar trimmomatic-0.39.jar SE -threads 12 -trimlog shC9-2_trimlog.txt /.fastq ILLUMINACLIP:QIAseq.fa:2:30:10 LEADING:3 TRAILING:3 SLIDINGWINDOW:4:15 MINLEN:15

Reference FASTA files were created for the first and second halves of the designed shRNA called A strand (5′ arm) and B strand (3′ arm). These reference sequences for each shRNA arm sequence included up to 10 additional nucleotides of flanking sequence. The reference FASTA files were assigned to their respective strand directory and the trimmed fastq was inserted in each strand folder. The command bowtie-build -r reference_list.fasta reference_list was used in each reference folder to create three reference FASTA files for each strand. The command line bowtie -S -p 12 -v 1 -a --best ref trim_.fastq>sam_trim_fastq.sam was used in each strand directory to create .sam files. Tablet software was used to create tablet files from each strand by opening a new assembly and aligning the .sam file and the reference FASTA file. Tablet .txt files were obtained for both A and B strands for each sample.

A custom excel file was created to analyze each tablet file. Each strand was analyzed in separate tabs of excel. The tablet file was pasted into excel where it read start position, end position, and length of each shRNA reported. A table of read lengths and total reads was created from the tablet file. A bar graph was created to report percentage of reads for each read length listed out of the total number of reads reported. Then a table was created to build potential shRNA reads beginning from start position 1 of the reference sequence and ending with the final start position possible for the reference strand length. A similar table was created that listed potential shRNA reads using the end positions from the tablet file. Then a table was created to analyze specific shRNA reads with exact start and end positions.

### Generation of Luciferase-Based Model Cells for CAG Repeat-Containing Gene Expression

To create a rapid surrogate assay for measuring relative knock-down of huntingtin (HTT) wild-type (wt) and mutant (mut) protein expression, HTT exon 1 fragments of wt and mut mRNA were cloned from the cDNA of patient-derived fibroblasts (GM04281). These contained the exon 1 sequence upstream of the CAG repeat, the CAG repeat itself, followed by a short fragment of the exon 1 sequence downstream of the repeat. These clones were designed to contain different peptide tags (HA and FLAG tags) on the wt and mut variants via PCR and then cloned into a vector upstream of and in-frame with an mCherry reporter protein in a pINC3G vector. The inducible Tet-on promoter was replaced with CMV a promoter and, a firefly luciferase gene was inserted downstream of and in-frame with the mCherry gene via a T2A linker. All vectors were confirmed by Sanger sequencing.

Luciferase Activity Assays for shRNA Activity

Luciferase assays were established and performed by co-transfecting HEK293T cells on 24-well plates with 50.0 ng of HTT wt or mut luciferase, 5.0 ng RSV-Renilla (Promega), and 12.5 ng indicated shRNA-expressing constructs. Forty-eight hr after transfection, dual luciferase reporter assays were performed to determine the knockdown efficiency of the designed shRNAs. Firefly luciferase values were first normalized to Renilla luciferase values and then compared to scramble shRNA controls. Luciferase experiments were performed at least 3 times and statistical analysis was performed using two-way ANOVA followed by Tukey's post hoc test.

### Western Blot Analyses for Knockdown of Mutant and Wildtype Huntingtin in Patient-Derived Cells

Western blot assays were performed essentially as previously reported (Hu et al., 2009; Gagnon et al., 2010). Briefly, cells were harvested by trypsinization, protein extracted with a cell lysis buffer and centrifugation, and protein quantified by BCA or Bradford assay. Protein samples (12-15 ug) were separated by SDS-PAGE on 5% (50:1 acrylamide:bis-acrylamide) tris-acetate gels using Novex Tris-Acetate SDS Running Buffer (Invitrogen). Gels were typically resolved for 6-8 h at 100-120 volts at 4-10° C. HTT proteins were transferred to Hybond-C Extra (Amersham Biosciences) nitrocellulose for 2.5-3 h. HTT protein bands were detected with anti-HTT primary antibody (MAB2166, Chemicon) at 1:5000 dilution and anti-mouse HRP conjugate antibody (Jackson Immunoresearch) at 1:5000 dilution. Tubulin loading controls were performed on a separate gel via standard procedures. Protein was visualized by chemiluminescence on an iBright imager (Thermo Fisher) or by exposure to film. Total HTT protein levels were first normalized to tubulin loading control levels, then normalized to treatment controls before plotting. Quantification was performed with ImageJ and data plotted with Prism software using a Hill plot equation.

Packaging of shRNA Lentiviral and Adeno-Associated Virus (AAV) Particles

To generate lentiviral particles, HEK293T cells on 100 mm plates (˜60% confluent) were transfected with CMV-VSV-G (Addgene #8454), CMV-dR8.2 dvpr (Addgene #8454), and the pLKO1 shRNA lentiviral construct. Forty-eight hours after transfection, lentiviral particle-containing media was removed from cells, filtered to remove cell debris, and centrifuged at 70,000×g for 2 hours to concentrate the lentiviral particles. Lentiviral particles were suspended in PBS and stored at −80° C. To generate AAV-particles, U6 promoter driven shRNAs were sublconed downstream of the bGH poly A signal in pAAV-CAG-Emerald plasmid (plasmid backbone pAAV-MCS, Takara) using PCR. HEK293T cells were transfected with pHelper (Takara), pAAV-DJ (Cell Biolabs Inc.), and indicated shRNA-expressing pAAV CAG-Emerald plasmids. Seventy-two hr after transfection AAV particles were extracted from cells using AAVpro Extraction Solution (Takara). Viral genome content was determined by qPCR using primers that amplify a region with the ITR.

### Example 1: Optimizing Expression and Processing of CAG Repeat-Targeted shRNAs

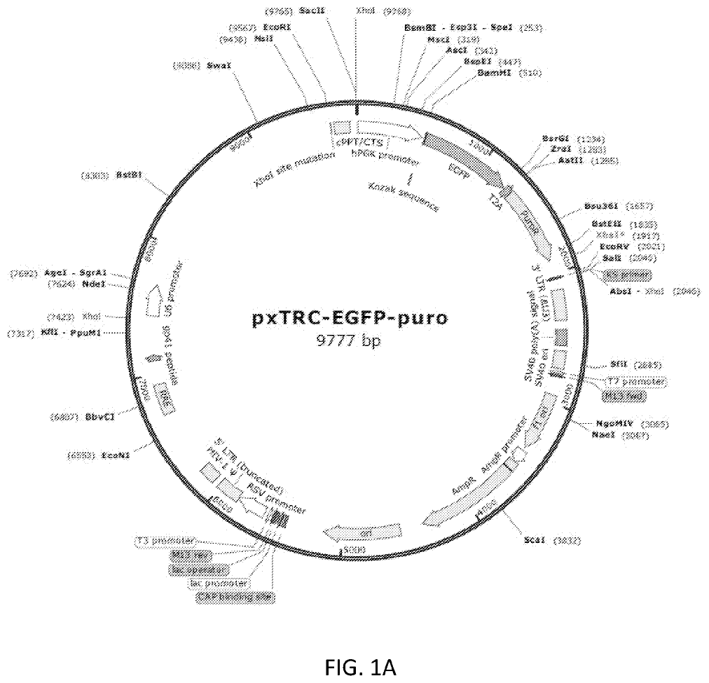

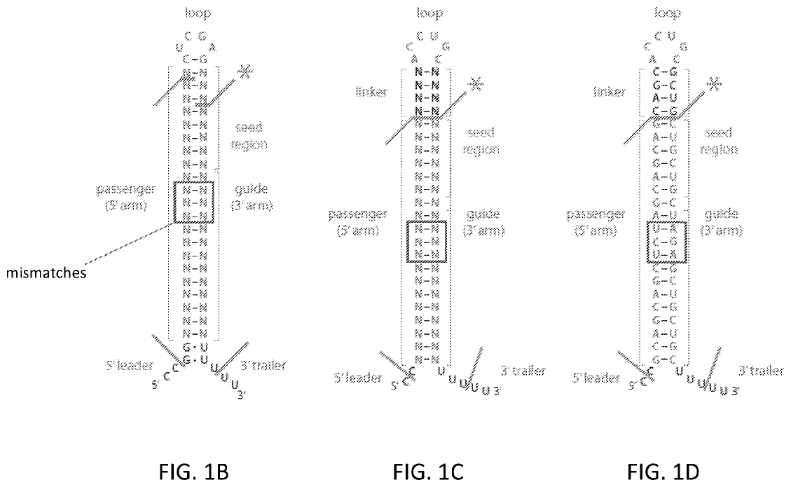

shRNAs were cloned into a U6 promoter-driven expression vector following a modification of a previously reported protocol (Moffat et al., 2006). The expression vector was customized by including an EGFP gene upstream of the puromycin resistance gene to help monitor transfection efficiency by fluorescence, resulting in the vector pxTRC-EGFP-puro (FIG. 1A). Two shRNAs were cloned using the expression and processing scaffold of the original pxTRC-EGFP-puro vector (Moffat et al., 2006). These were shHD1 and shHD1-L, which were designed to either match perfectly or contain three central mismatches (positions 10-12) to the target, respectively. Control shRNAs that target endogenous PARP1 (shPARP), HTT protein outside of the CAG repeat (shHD2.4), and a non-targeting shRNA against mCherry (shMChry) were also cloned (Table 1).

TABLE 1
CAG Repeat-Targeting shRNA Cloning Inserts

| shRNA | | |
|---|---|---|
| name | shRNA cloning insert sequence (5′-3′) | Scaffold |
| shHD1 | GTGGAAAGGACGAAACA ccgg CAGCAGCAGCAGCAGCAGCAG | pTRC Pol III |
| | ctcgag CTGCTGCTGCTGCTGCTGCTG tttttg | |
| | AATTCTCGACCTCGAGACAA [SEQ ID NO: 51] | |
| shHD1L | GTGGAAAGGACGAAACA ccgg CAGCAGCAGTTTCAGCAGCAG | pTRC Pol III |
| | ctcgag CTGCTGCTGAAACTGCTGCTG tttttg | |
| | AATTCTCGACCTCGAGACAA [SEQ ID NO: 52] | |
| shPARP | GTGGAAAGGACGAAACA ccgg CCGAGAAATCTCTTACCTCAA | pTRC Pol III |
| | ctcgag TTGAGGTAAGAGATTTCTCGG tttttg | |
| | AATTCTCGACCTCGAGACAA [SEQ ID NO: 53] | |
| shHD2.4 | GTGGAAAGGACGAAACA ccgg GACCGTGTGAATCATTGTCTA | pTRC Pol III |
| | ctcgag TAGACAATGATTCACACGGTC tttttg | |
| | AATTCTCGACCTGGTGACAA [SEQ ID NO: 54] | |
| shMChry | GTGGAAAGGACGAAACA ccgg GAGGATAACTCGGCCATAATA | pTRC Pol III |
| | ctcgag TATTATGGCCGAGTTATCCTC tttttg | |
| | AATTCTCGACCTGGTGACAA [SEQ ID NO: 55] | |
| shHD1-1 | GTGGAAAGGACGAAACA cc GCAGCAGCAGCAGCAGCAG CAGC | modified pTRC |
| | acctgc GCTG CTGCTGCTGCTGCTGCTGC tttttg | Pol III |
| | AATTCTCGACCTGGTGACAA [SEQ ID NO: 56] | |
| shHD1L-1 | GTGGAAAGGACGAAACA cc GCAGCAGCTCTAGCAGCAG CAGC | modified pTRC |
| | acctgc GCTG CTGCTGCTAGAGCTGCTGC tttttg | Pol III |
| | AATTCTCGACCTGGTGACAA [SEQ ID NO: 57] | |
| shHD1L-1a | GTGGAAAGGACGAAACA cc CAGCAGCACAAGCAGCAG CAGC | modified pTRC |
| | ctcgag GCT GCTGCTGCAAATGCTGCTG tttttg | Pol III |
| | AATTCTCGACCTGGTGACAA [SEQ ID NO: 58] | |
| shHD1L-1b | GTGGAAAGGACGAAACA cc GCAGCAGCCAAAGCAGCAG CAGC | modified pTRC |
| | gtgaagccacagatg GCTG CTGCTGCTAAAGCTGCTGC tttttg | Pol III |
| | AATTCTCGACCTGGTGACAA [SEQ ID NO: 59] | |
| shHD1L-1c | GTGGAAAGGACGAAACA cc GCAGCAGCCAAAGCAGCAG CAGC | modified pTRC |
| | gttctggcaatacct GCTG CTGCTGCTAAAGCTGCTGC tttttg | Pol III |
| | AATTCTCGACCTGGTGACAA [SEQ ID NO: 60] | |
| shHD1L-1d | GTGGAAAGGACGAAACA cc CTGCTGCTAAAGCTGCTG CTGC | modified pTRC |
| | ctcgag GCA GCAGCAGCCAAAGCAGCAG tttttg | Pol III |
| | AATTCTCGACCTGGTGACAA [SEQ ID NO: 61] | |
| shMCHR-1 | GTGGAAAGGACGAAACA ccgg GAGGATAACTCGGCCATAATA CG | modified pTRC |
| | acctgc GCta TTATGGCCGAGTTATCCTC tttttg | Pol III |
| | AATTCTCGACCTGGTGACAA [SEQ ID NO: 62] | |

Bold uppercase sequence matches target cloning vector for recombination-based cloning. Italic sequence represents expected mature guide and passenger strands of mature duplex RNA. Underlined sequence represents mismatched sequence compared to potential targets or deviations from repeat sequence. Lowercase sequence represents internal loop of shRNA hairpin. Only the sense strand of a duplex DNA insert is shown.

Prior to proceeding with experimental target knock-down studies, next-generation small RNA sequencing was performed after transfection of shPARP, shHD1, and shHD1L into patient-derived Huntington disease (HD) fibroblast cells. Sequencing revealed imperfect processing of shRNAs that shifted the expected 5′ end of the guide strand (the 3′ shRNA hairpin arm) by 3 nucleotides into the seed region toward the 3′ end for all three shRNAs (FIG. 1B). While this likely results in functional shRNAs for perfectly matched targets like PARP-1, it placed mismatches at positions 7-9 for shHD1-L instead of 10-12. Since the seed region of small RNAs loaded into Argonaute enzymes ranges from position 1-8, this would partially disrupt seed pairing and efficient binding to the targeted CAG repeat. In addition, the perfectly matched shHD1 revealed very low abundance for the mature guide strand (Table 2). The repetitive nature and high GC-content of an shHD1 hairpin during Drosha processing likely interferes with accurate processing due to ambiguous shifting of the hairpin structure and therefore processing register. These results suggested that introduction of unique, non-repetitive sequence in the hairpin, such as the three tandem adenosines that were added to create mismatches during target binding for shHD1-L, is beneficial by creating a predictable RNA hairpin fold and processing register.

TABLE 2
Processing Accuracy and Efficiency of shRNAs

| shRNA Name | Mismatches | | Guide RNA | |
|---|---|---|---|---|
| (CAG repeat | (passenger, | | 5′ | Relative |
| targeted) | guide) | Design | Processing | Abundance |
| shHD1 | none | XhoI loop | 20% | very low |
| shHD1-1 | none | ACCTGC loop | 50% | low |
| shHD1L-1 | (9-11, 9-11) | no central bulge | 97% | high |
| shHD1L-1a | (9-11, 9-11) | central bulge | 55% | low |
| shHD1L-1b | (9-11, 9-11) | central bulge, | 95% | high |
| | | miR30 loop | | |
| shHD1L-1c | (9-11, 9-11) | central bulge, | 95% | high |
| | | miR33 loop | | |
| shHD1L-1d | (9-11, 9-11) | central bulge, | 95% | very low |
| | | arm swap | | |
| shHD-33part | (9-11, 9-11) | central bulge, miR33 | 25% | very low |
| | | part scaffold | | |
| shHD-33part-3′k | (9-11, 9-11) | 3′ arm kink, | 3% | very low |
| | | miR33 part | | |
| shHD-33full | (9-11, 9-11) | central bulge, miR33 | 90% | high |
| | | full scaffold, arm swap | | |
| shHD-33full- | (9-11, 7-11) | miR33 bulge, | 85% | high |
| mimic | | miR33 full scaffold | | |

To correct for shifted guide RNA processing patterns in CAG repeat-targeted shRNAs, two new shRNAs, shHD1-1 and shHD1L-1 were designed. These designs adjusted flanking sequence to accommodate the implied cleavage and processing patterns of Drosha and Dicer. The first few nucleotides upstream and downstream of the hairpin arms were adjusted such that they did not pair (C-T mismatches), added a short 4 nucleotide linker between each arm and the internal loop, and changed the loop to an asymmetric sequence predicted to generate a well-defined 6-nucleotide loop (FIG. 1C). Also, shRNAs were transfected into HEK 293T cells, which provide higher small RNA yields for sequencing. These changes appeared to largely correct the processing defects and generate relatively abundant, mature shRNAs with the desired cleavage patterns. For the shHD1L-1 shRNA, -97% of guide strands were accurately processed with a perfectly complementary seed region at positions 1-8 and properly placed central mismatches (designed to be at positions 9-11) (FIG. 1D, Table 2). For shHD1-1, however, the abundance of the guide strand was still low despite correctly shifted processing patterns (Table 2). Thus, GC-rich repetitive shRNAs may pose challenges for Drosha or Dicer processing due to their stability and ability to shift pairing register between the two hairpin arms.

To explore new designs based on this modified scaffold, four additional CAG repeat-targeted shRNAs, shHD1L-1a through shHD1L-1d, were cloned (Table 1). These designs included intentional mismatches at the center of the shRNA hairpin itself to generate more natural miRNA-like structures, in addition to changing loops and swapping hairpin arm sequences. The shHD1L-1a design replaced the asymmetric loop with the palindromic loop used in the original design. Sequencing revealed that the original asymmetric loop was a potential source of low yield and improper processing as this design produced heterogeneity in the 5′ end of the guide strand and reduced overall abundance (Table 2). The internal loop was then replaced with native miR30 and miR33 loops for shHD1L-1b and shHD1L-1c, respectively. Both of these designs rescued processing back to high abundance and very high processing accuracy (Table 2). Finally, shHD1L-1d was designed for the guide strand to be processed from the 5′ arm of the hairpin. This arm swap resulted in similar processing defects as seen for shHD1L-1a, such as poor processing predictability, reduced abundance, and a common 1-nucleotide shift toward the 3′ end of the guide strand.

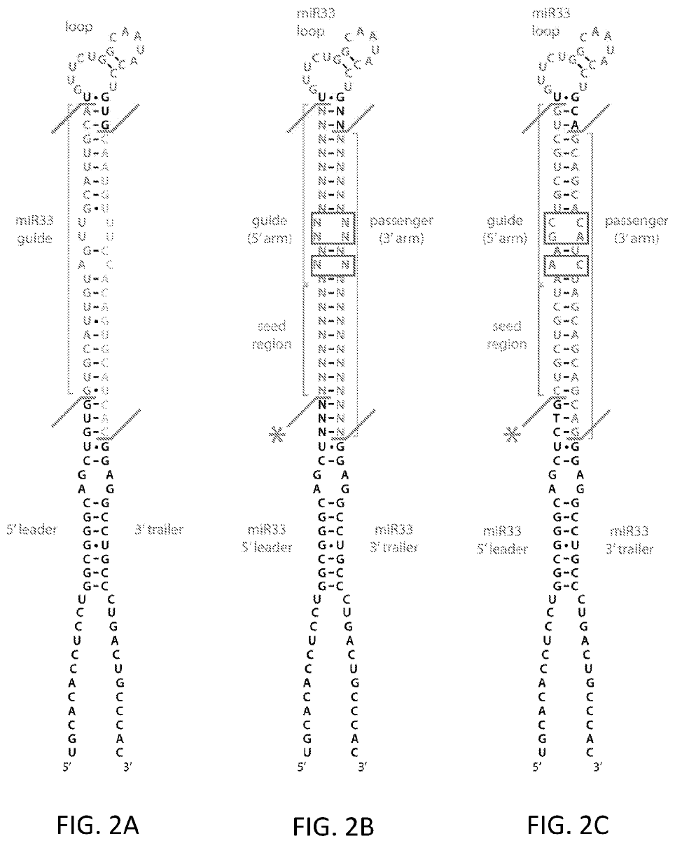

Many therapeutic shRNAs are designed to be embedded within a natural miRNA scaffold. Therefore, a miR33 scaffold was selected for central mismatch CAG repeat-targeted shRNAs (Xie et al., 2020) (FIGS. 2A-2B). Designs included a partial miR33 flanking scaffold (shHD-33part), a partial scaffold with a hairpin kink (shHD-33part-3′k), a full miR33 flanking scaffold (shHD-33full), and a full scaffold with the hairpin structure designed to also mimic that of miR33 (shHD-33full-mimic) (Table 3). All designs were created to generate a bulge or kink in the shRNA hairpin structure and the guide strand is processed from the 5′ hairpin arm rather than the 3′ arm. The shHD-33part and shHD-33part-3′k designs were partial miR33 scaffolds, lacking full miR33 5′ leader and 3′ trailer flanking sequences. Both designs exhibited very poor abundance and low guide strand processing predictability (Table 2). In contrast, shHD-33full and shHD-33full-mimic processed to high accuracy (85-90%) at the 5′ end of the guide strand and reached relatively high abundance (Table 2). These designs mimicked the miR33 miRNA structure, with shHD33-full-mimic creating a central bulge quite similar to miR33 (FIG. 2C). Thus, the miR33 scaffold designs provided robust processing by generating predictable 5′ end processing products, accurate placement of central mismatches, and relatively high abundance of mature guide strands.

TABLE 3
CAG Repeat-Targeted shRNA miR33 Scaffold Cloning Inserts.

| shRNA name | shRNA cloning insert sequence (5′-3′) | Scaffold |
|---|---|---|
| shHD-33part | GTGGAAAGGACGAAACA g ctg | miR33 pTRC Pol III |
| | CTGCTGCTAAAGCTGCTGCTG t | |
| | gttctggcaatacct g ca | |
| | GCAGCAGCCAAAGCAGCAGCA ga tttttg | |
| | AATTCTCGACCTGGTGACAA [SEQ ID NO: 63] | |
| shHD-33part-3′k | GTGGAAAGGACGAAACA g ctg | miR33 pTRC Pol III |
| | CTGCTGCTAAAGCTGCTGCTG t | |
| | gttctggcaatacct g ca | |
| | GCAGCAGCTTAGCAGCAGCA ga tttttg | |
| | AATTCTCGACCTGGTGACAA [SEQ ID NO: 64] | |
| shHD-33full | GTGGAAAGGACGAAACA | miR33 pTRC Pol III |
| | tgcacacctcctggcgggcagct ctg | |
| | CTGCTGCTAAAGCTGCTGCTG t | |
| | gttctggcaatacct g ca | |
| | GCAGCAGCCAAAGCAGCAGCAG gg | |
| | aggcctgccctgactgcccac tttttg | |
| | AATTCTCGACCTGGTGACAA [SEQ ID NO: 65] | |
| shHD-33full- | GTGGAAAGGACGAAACA | miR33 pTRC Pol III |
| mimic | tgcacacctcctggcgggcagct ctg | |
| | CTGCTGCTAAAGCTGCTGCTG t | |
| | gttctggcaatacct g ca | |
| | GCAGCACATCTAGCAGCAGCAG gg | |
| | aggcctgccctgactgcccac tttttg | |
| | AATTCTCGACCTGGTGACAA [SEQ ID NO: 66] | |

Bold uppercase sequence matches target cloning vector for recombination-based cloning. Italic sequence represents expected mature guide and passenger strands of mature duplex RNA. Underlined sequence represents mismatched sequence compared to potential targets or deviations from repeat sequence. Lowercase sequence represents internal loop of shRNA hairpin. Only the sense strand of a duplex DNA insert is shown.

### Example 2: Selective Reduction of CAG Repeat Expansion Gene Expression with CAG Repeat-Targeted shRNAs in a Luciferase-Based Model System

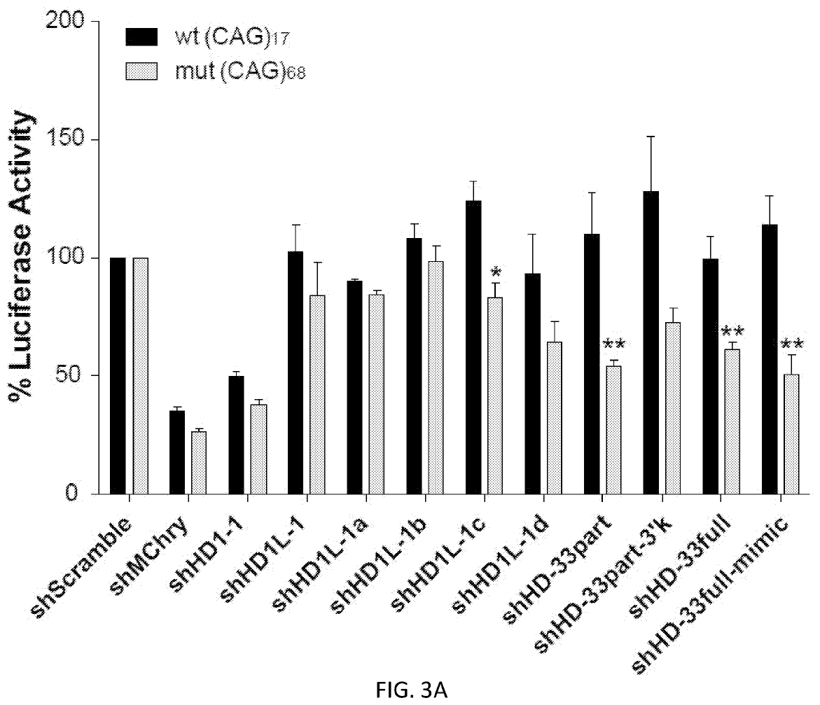

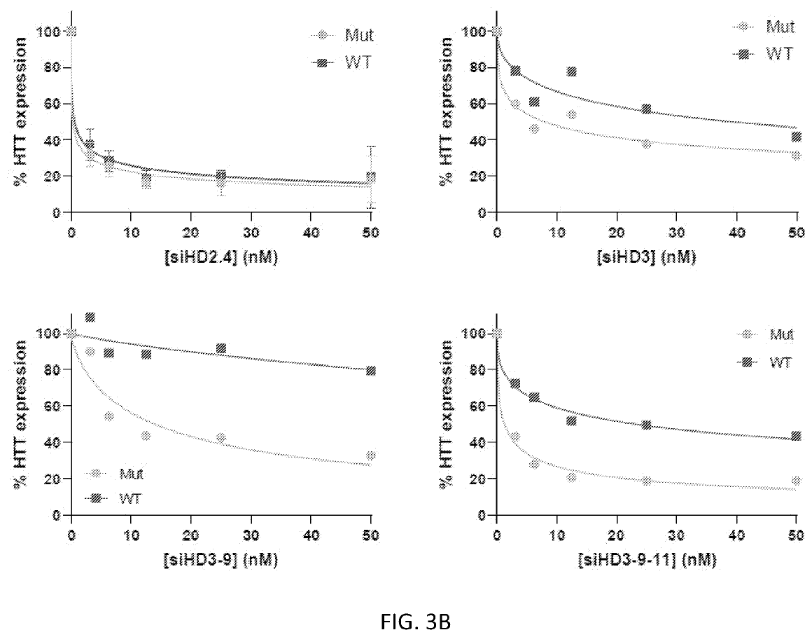

To evaluate the efficiency and allele selectivity of CAG repeat-targeted shRNAs, a luciferase-based reporter assay was utilized in HEK 293T cells. Fragments of HTT exon 1 from wt (containing 17 CAG repeats) or mut (containing 68 CAG repeats) mRNA were cloned in-frame and upstream of firefly luciferase. HEK293Ts were co-transfected with wt or mut luciferase reporter plasmids with control or CAG repeat targeted shRNA (FIG. 3A). Luciferase activity for each shRNA was set relative to control scramble shRNA. The positive control shMchry and shHD1 shRNAs displayed 50-60% reduction of luciferase activity for both wt (CAG17) and mut (CAG68). No significant reduction in luciferase activity was observed for HD1L-1, HD1L-1a, HD1L-1b, HD1L-d, or HD-33part-3′k when comparing wt to mut. Significant decreases in mut activity relative to wt were observed for HD1L-1c, HD-33part, HD-33full and HD-33full-mimic.

### Example 3: Knockdown of Huntingtin Gene Expression with CAG Repeat-Targeted shRNAs

Small interfering RNAs (siRNAs) targeting CAG repeats and possessing central mismatches have previously been demonstrated to selectively knockdown the endogenous mutant HTT allele (Hu et al., 2009). To confirm that our system was responding similarly to siRNAs, four control siRNAs in patient-derived fibroblasts (GM04281) were transfected (Table 4). Control siRNAs targeting outside of the repeat (siHD2.4) and targeting the CAG repeat directly with perfect pairing (siHD-3) both knocked-down protein expression similarly when assayed by Western blot (FIG. 3B). As expected, these siRNAs did not exhibit strong allele selective inhibition, especially siHD2.4. Conversely, both siHD-3-p9 and siHD-3-p9-11, which introduce central mismatches to the target at positions 9 and 9-11, respectively, provided a window of allele selective knockdown. When HTT expression was assayed by Western blot, each produced a greater reduction in the mutant HTT versus the wt HTT allele over a certain concentration range (FIG. 3B). These results correlate with previous reports using siRNAs (Hu et al., 2009).

TABLE 4
Synthetic CAG Repeat-Targeted siRNAs.

| siRNA name | siRNA sequence (5′-3′) |
|---|---|
| siHD2.4_as | rUrArGrArCrArArUrGrArUrUrCrArCrArCrGrGtt (SEQ ID NO: 67) |
| siHD-3_as | rGrCrUrGrCrUrGrCrUrGrCrUrGrCrUrGrCrUrGtt (SEQ ID NO: 68) |
| siHD-3-p9_as | rGrCrUrGrCrUrGrCrArGrCrUrGrCrUrGrCrUrGtt (SEQ ID NO: 69) |
| siHD-3-p9-11_as | rGrCrUrGrCrUrGrCrArArArUrGrCrUrGrCrUrGtt (SEQ ID NO: 70) |
| siHD2.4_s | rCrCrGrUrGrUrGrArArUrCrArUrUrGrUrCrUrAtt (SEQ ID NO: 71) |
| siHD-3_s | rCrArGrCrArGrCrArGrCrArGrCrArGrCrArGrCtt (SEQ ID NO: 72) |
| siHD-3-p9_s | rCrArGrCrArGrCrArGrCrUrGrCrArGrCrArGrCtt (SEQ ID NO: 73) |
| siHD-3-p9-11_s | rCrArGrCrArGrCrArUrUrUrGrCrArGrCrArGrCtt (SEQ ID NO: 74) |

″rN″ indicates an RNA nucleotide

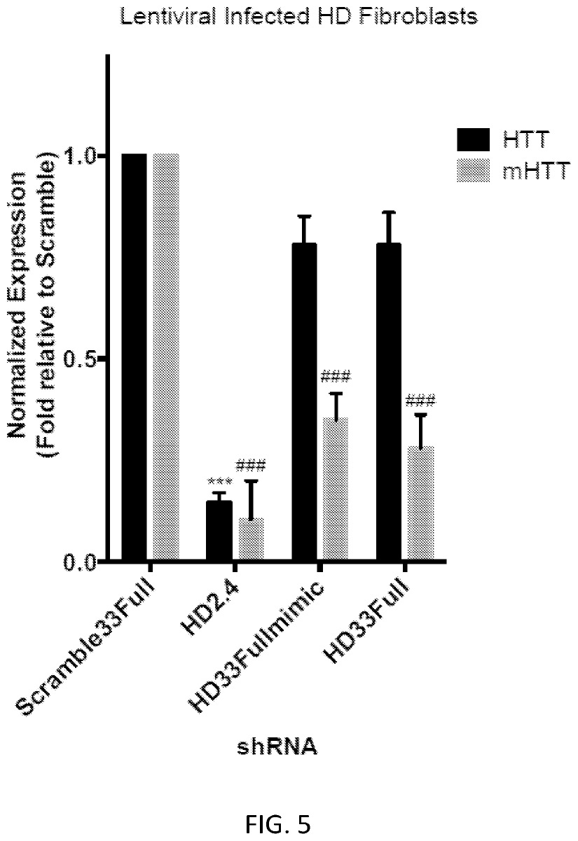

To determine whether CAG repeat-targeted shRNAs could produce allele-selective inhibition of endogenous mut HTT, shRNAs expressed from a miR33 scaffold including shScramble, shHD2.4, shHD-33full and shHD-33full-mimic were packaged into lentiviral particles and transduced patient-derived fibroblasts. Cells were stably infected at greater than 90% efficiency based on microscopy for GFP. After 7 days, cells were harvested and HTT expression assayed by Western blot. Compared to shScramble transduced patient-derived fibroblasts, both wt and mut HTT were significantly decreased by HD2.4. Compared to shScramble transduced patient-derived fibroblasts, allele-selective shRNAs significantly decreased expression of mut-HTT in cells transduced with shHD-33full and shHD33-fullmimic (FIG. 5).

shRNA expression cassettes were electroporated into GM04281 fibroblasts. After 3.5 days, cells were assayed for HTT expression by Western blot. By Western blot analysis, we found that several shRNAs, including shHD1-1, shHD1L-1, and shHD1L-1a through -1d, were not producing a substantial reduction of HTT expression. Microscopy and flow cytometry measuring EGFP expression typically revealed 30-40% efficiency using this transfection protocol. Further testing of shRNAs expressed from miR33 scaffolds, which processed well based on small RNA sequencing, also did not provide robust knockdown. These, results suggested that shRNA expression may not be achieving high levels in patient-derived fibroblasts.

### Example 4: shRNA Abundance of Mismatched Vs. Perfect Matched Guide Sequences

% abundance of a correctly processed artificial miRNA may be measured by obtaining the number of RNA sequence reads of artificial miRNAs that have the correctly processed 5′ end and align to the reference CAG repeat region containing reference transcript, and dividing by the total number of RNA sequence reads of artificial miRNAs aligned to the reference CAG repeat region containing reference transcript

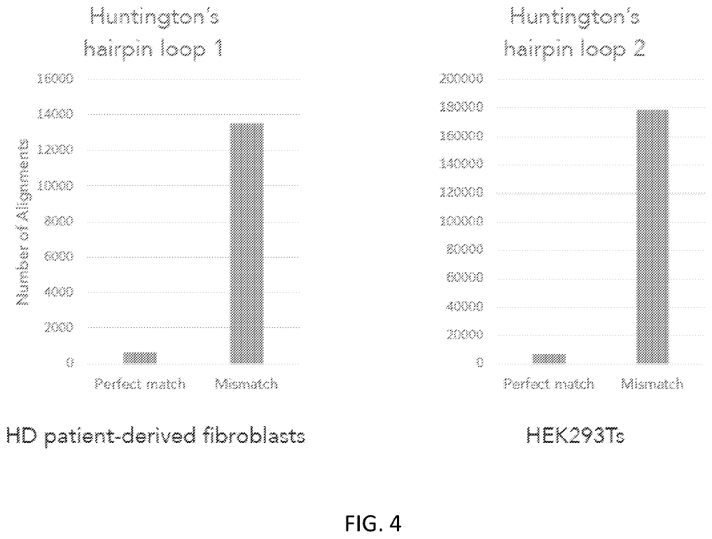

As shown in Table 5 and FIG. 4, the presence of mismatch(es) at positions 8-16 of the guide sequence targeting CAG repeat region of HTT mRNA improves the abundance or virally encoded shRNA compared to perfectly matched guide sequences. The results are consistent across different hairpin loops and cell types.

TABLE 5
Abundance of Mismatched vs. Perfect Matched shRNAs

| | | | | # of | | | | Most | |
|---|---|---|---|---|---|---|---|---|---|
| | Cell | | | align- | | | | abundant | |
| Construct | type | Type | Strand | ments | Loop | Sequence | Round | read | % |
| shHD_1_ | HD | Perfect | A | 434 | ctc | CAGCAGCAGC | 2 | AGCAGCAGCA | 12% |
| A | fibro | | | | gag | AGCAGCAGCA | | GCAGCAGCAG | |
| | blast | | | | | G (SEQ ID | | (SEQ ID | |
| | | | | | | NO: 746) | | NO: 747) | |
| shHD_1_ | HD | Perfect | B | 627 | ctc | CTGCTGCTGC | 2 | CTGCTGCTGC | 11% |
| B | fibro | | | | gag | TGCTGCTGCT | | TGCTGCTGtt | |
| | blast | | | | | G (SEQ ID | | t (SEQ ID | |
| | | | | | | NO: 748) | | NO: 749) | |
| shHD_ | HD | Mismatch | A | 193 | ctc | CAGCAGCAGt | 2 | gCAGCAGCAG | 32% |
| 1L_A | fibro | | | | gag | ttCAGCAGCA | | tttCAGCAG | |
| | blast | | | | | G (SEQ ID | | (SEQ ID | |
| | | | | | | NO: 501) | | NO: 750) | |
| shHD_ | HD | Mismatch | B | 13517 | ctc | CTGCTGCTGa | 2 | CTGCTGaaaC | 44% |
| 1L_B | fibro | | | | gag | aaCTGCTGCT | | TGCTGCTGtt | |
| | blast | | | | | G (SEQ ID | | ttt (SEQ | |
| | | | | | | NO: 420) | | ID NO: 751) | |
| shHD1_1_ | HEK293T | Perfect | A | 4658 | acc | GCAGCAGCAG | 3 | CAGCAGCAGC | 32% |
| A | | | | | tgc | CAGCAGCAGC | | AGCAGCAGCA | |
| | | | | | | AGC (SEQ | | GC (SEQ ID | |
| | | | | | | ID NO: 752) | | NO: 753) | |
| shHD1_1_ | HEK293T | Perfect | B | 7543 | acc | GCTGCTGCTG | 3 | CTGCTGCTGC | 20% |
| B | | | | | tgc | CTGCTGCTGC | | TGCTGCTGCt | |
| | | | | | | TGC (SEQ | | (SEQ ID | |
| | | | | | | ID NO: 754) | | NO: 755) | |
| shHD1L_ | HEK293T | Mismatch | A | 5434 | acc | GCAGCAGCTC | 3 | cGCAGCAGCT | 67% |
| 1_A | | | | | tgc | TAGCAGCAGC | | CTAGCAGCAG | |
| | | | | | | AGC (SEQ | | (SEQ ID | |
| | | | | | | ID NO: 756) | | NO: 757) | |
| shHD1L_ | HEK293T | Mismatch | B | 178998 | acc | GCTGCTGCTG | 3 | CTGCTGCTAG | 71% |
| 1_B | | | | | tgc | CTAGAGCTGC | | AGCTGCTGCt | |
| | | | | | | TGC (SEQ | | tt (SEQ ID | |
| | | | | | | ID NO: 758) | | NO: 759) | |

### Example 5: In Vivo Knockdown

The expression construct U6-shHD33-Full-Mimic was cloned into an AAV9 vector backbone, and AAV9 was produced using triple transfection into mammalian cells and purified by double CsCl density gradient ultra-centrifugation, desalting, and filter sterilization (Signagen Laboratories). AAV vectors were titered via quantitative polymerase chain reaction (qPCR). As a negative control, Scrambled shRNA was also packaged into AAV9 (AAV9-shScr) using the same manufacturing process. All of the expression cassettes contained green fluorescent protein (GFP)-encoding sequences in order to assess transduction efficiency.

The vector preparation was administered via bilateral striatal injection into anesthetized male zQ175 HET mice (B6J.129S1-Htt^{tm1Mfe}/190ChdiJ) using stereotaxic injection at 4 weeks of age. zQ175 mice express the human HTT exon 1 sequence with a ˜190 CAG repeat tract in one allele and a normal mouse HTT in the other allele. Menalled et al. (2012) PLoS ONE 7:e49838. A 2.0 uL volume of test article was pump delivered at a rate of 0.1 uL/minute. Mice received AAV9-shScr (8.0E9 vg/mouse) or AAV9-shHD33-Full-Mimic (8.0E9 vg/mouse).

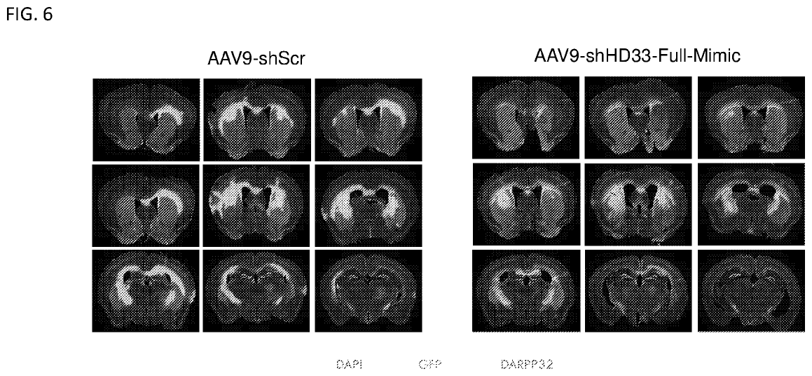

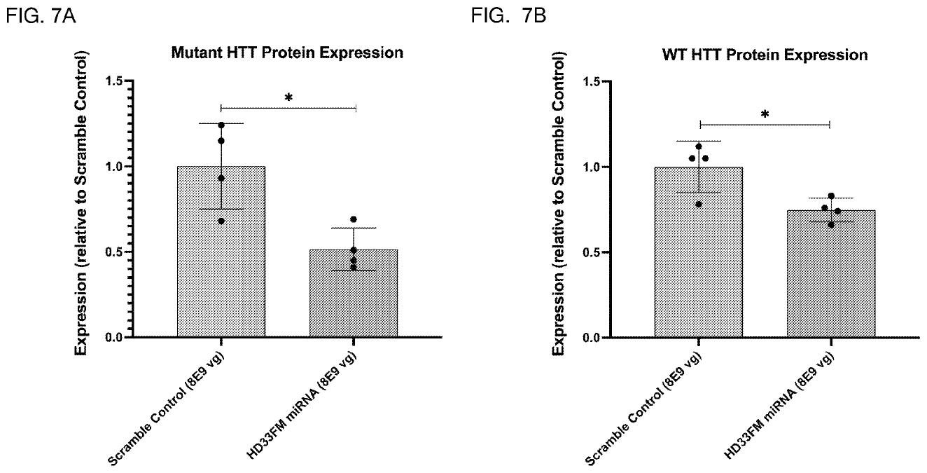

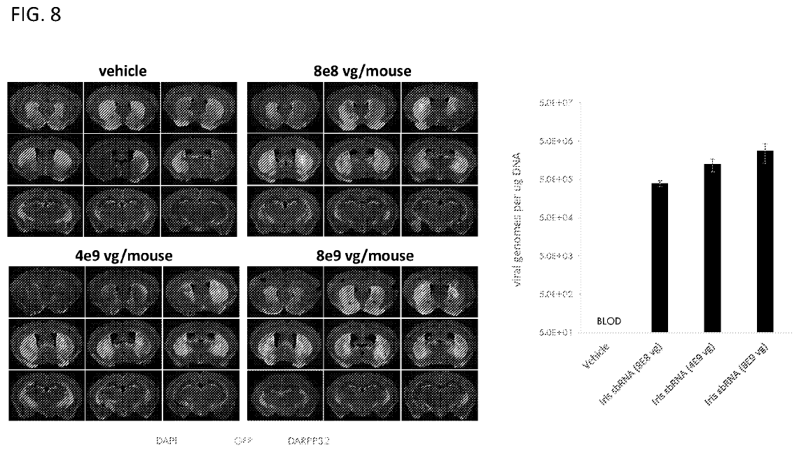

Six weeks after injections, brain tissue was harvested, and GFP distribution was analyzed by histology. Co-localization of DARPP32 and GFP staining demonstrated transduction of zQ175 striatum (FIG. 6). Western blot analysis of wild-type (WT) and mut HTT protein expression showed significant knockdown of mut HTT in the AAV9-shHD33-Full-Mimic treatment group relative to the AAV9-shScr treatment group (FIG. 7). Furthermore, AAV9-shHD33-Full-Mimic showed preferential knockdown of the mut HTT allele relative to the WT HTT allele.

### Example 6: Allele-Selective Knockdown in zQ175 Mouse Model of Huntington's Disease

### Materials & Methods

The expression construct U6-shHD33-Full-Mimic (Iris sbRNA) was cloned into an AAV9 vector backbone, and AAV9 was produced using triple transfection into mammalian cells and purified by double CsCl density gradient ultra-centrifugation, desalting, and filter sterilization (Signagen Laboratories). Virus was formulated in PBS plus 0.005% Pluronic F-68. The expression cassettes contained GFP in order to assess transduction efficiency. AAV vectors were titered via qPCR. As a negative control, formulation vehicle was delivered to one group of transgenic mice and one group of wild-type littermates.

The vector preparation was administered via bilateral striatal injection into anesthetized male zQ175 transgenic mice (B6J.129S1-Htt^{tm1Mfc}/190ChdiJ) and wild-type littermates using stereotaxic injection at 4 weeks of age. A 2.0 μL volume of test article was pump delivered at a rate of 0.1 μL/minute. Mice received test article as outlined in Table 9.

TABLE 9

| | | | | Dose |
|---|---|---|---|---|
| Group | Number | Genotype | Test Article | vg/mouse |
| 1 | 5 | wild-type (littermate) | vehicle | n/a |
| 2 | 5 | zQ175 transgenic | vehicle | n/a |
| 3 | 5 | zQ175 transgenic | Scramble | 8e9 |
| | | | shRNA (control) | |
| 4 | 5 | zQ175 transgenic | Iris sbRNA | 8e8 |
| 5 | 5 | zQ175 transgenic | Iris sbRNA | 4e9 |
| 6 | 5 | zQ175 transgenic | Iris sbRNA | 8e9 |

“Iris sbRNA” refers to a recombinant AAV comprising SEQ ID NO:606, a nucleotide sequence that encodes an sbRNA that, when processed within a cell, produces a double-stranded RNA comprising a first strand that is SEQ ID NO:325 and a second strand that is SEQ ID NO:555 (CUG_307 of Table 6). Iris sbRNA is also referred herein to as “shHD33 full mimic.”

Body weights of animals were taken at baseline in order to ensure even distribution between treatment groups. Weights were then assessed once per week until the time of tissue collection when animals were weighed before being collected.

Six weeks after injections, brain tissue was harvested. One animal per group (6 animals in total) were anesthetized with isoflurane and transcardially perfused with PBS until the liver was clear followed by approximately 50 mL of 4% PFA. The whole brain was harvested and placed into 25 mL vials with 4% PFA and stored at 4° C. for 24 hours. After 24 hours, brains were transferred to 30% sucrose in PBS and stored at 4° C. For the remaining 4 animals in each group (24 animals in total), the animals were euthanized via rapid decapitation, and the brain was excised from the skull and rinsed in chilled 0.9% sterile saline to remove excess blood. Brains were microdissected by region under RNase-free conditions on top of a pre-wetted (0.9% saline) piece of clean (free of DNA and RNA) filter paper resting on a glass petri dish packed in wet ice. The following structures were dissected: striatum, cortex, and cerebellum. Each tissue sample was placed into individual 1.5 mL Eppendorf tubes and snap frozen in liquid nitrogen.

Striatal tissue samples were homogenized in lysis buffer from the miRvana miRNA isolation kit (Invitrogen). DNA was extracted using the All Prep DNA/RNA kit (Qiagen), and separate small RNA and total RNA fractions were extracted using the miRvana miRNA isolation kit. qPCR was performed by loading 100 ng DNA per reaction with TaqMan Universal PCR Master Mix II (Applied Biosystems) and using primer/probe sets targeting GFP (forward: GGAGCGCACCATCTTCTT (SEQ ID NO:860), reverse: ATGTTGCCGTCCTCCTTG (SEQ ID NO:861), probe: AAGGACGACGGCAACTACAAGACC (SEQ ID NO:862)) and mouse GAPDH (forward: TCACCTGGCCTACAGGATAAA (SEQ ID NO:863), reverse: GCAAACGGGAAGGAAATGAATG (SEQ ID NO:864), probe: ACCCAGCCGAGAGGAATGAGGTTA (SEQ ID NO:865)). Reactions were run on the Quantstudio 7 Pro (Applied Biosystems).

Whole brains were mounted onto a sliding microtome and frozen with dry ice. Coronal sections (30 m) were cut, with every 8^{th} section collected for immunofluorescent staining. Brains were stained as floating sections in a 12-well dish. Tissue sections were washed twice for 5 minutes each in PBS, then blocked in a solution containing 5% donkey serum and 0.3% Triton X-100 in PBS for 2 hours at room temperature. After blocking, tissue sections were incubated with rabbit monoclonal anti-DARPP32 (1:100, Abcam, ab40801) primary antibodies overnight at 4° C. Tissue sections were washed again three times for 15 minutes each in 0.3% Triton X-100 in PBS. Tissue sections were incubated with Alexa Fluor 594 goat anti-rabbit secondary antibody (1:500, Life Technologies, A11012) for 2 hours at room temperature. Tissue sections were washed again three times for 15 minutes each in 0.3% Triton X-100 in PBS, followed by 4′,6-diamidino-2-phenylindole (DAPI) nuclear stain (Abcam) for 20 minutes at room temperature. Tissue sections were washed twice in PBS, then mounted onto slides and coverslipped. Images of the sections were taken using an Axioscan Z (Zeiss) microscope.

Striatal tissue samples were homogenized in lysis buffer (150 mM NaCl, 50 mM Tris pH 7.5, 1 mM EDTA, 1% Triton X-100, and 1% protease inhibitor). Total protein was quantified using the Micro BCA Protein Assay kit (ThermoFisher). 35 μg/well of protein was loaded into 5.5% Tris-acetate polyacrylamide gels for Western blot analysis. Gels were run in Tris-Acetate running buffer at 70 V for 15 minutes then at 120 V for 3 hours. After electrophoresis, proteins were transferred to an NC membrane (Bio-Rad). The membrane was blocked in 5% milk in PBST and TBST separately for 1 hour at room temperature. The membrane was incubated with primary antibodies specific for mouse monoclonal anti-HTT (1:1,000, Sigma Aldrich, MAB2166) and rabbit monoclonal anti-vinculin (1:10,000, Abcam, ab129002) overnight at 4° C. The membrane was washed three times for 5 minutes each in PBST/TBST, then incubated with corresponding AffiniPure Peroxidase-conjugated secondary antibodies: donkey anti-mouse IgG (H+L) (1:4000, Jackson ImmunoResearch Laboratories, 715-035-150), donkey anti-rabbit IgG (H+L) (1:5000, Jackson ImmunoResearch Laboratories, 711-035-152) for 1.5 hours at room temperature. The membrane was washed three more times for 10 minutes each in PBST/TBST. The membrane was then incubated with SuperSignal West Pico Chemiluminescent Substrate (Thermo Scientific) for 3 minutes at room temperature. Images were acquired on the iBright 1500 (Invitrogen), and band intensity was quantified using ImageJ.

### Results

All animals had surgery performed at 4 weeks of age and survived until tissue collection at 10 weeks of age, 6 weeks post dose administration. No abnormalities were noted throughout the study and no premature deaths were reported. zQ175 transgenic animals treated with either Scramble shRNA control at 8e9 vg or Iris sbRNA at 8e9 vg weighed significantly less than zQ175 transgenic animals treated with vehicle starting at 8 weeks of age and continuing through the rest of the study.

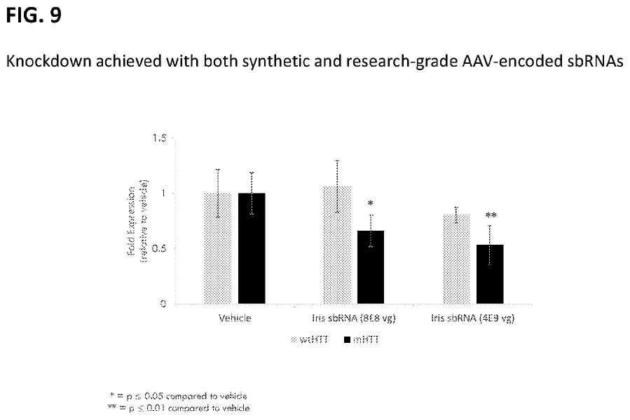

Viral genomes were quantified by qPCR and demonstrated dose-dependent sbRNA genome localization to the striatum (FIG. 8, right panel). GFP distribution was analyzed by immunofluorescence. Co-localization of DARPP32 and GFP staining demonstrated dose-dependent transduction of the zQ175 striatum (FIG. 8, left panel. Western blot analysis of WT and mut HTT protein expression showed significant knockdown of mut HTT in the Iris sbRNA treatment group relative to the vehicle group at doses of 8e8 vg and 4e9 vg (FIG. 9). Furthermore, Iris sbRNA showed preferential knockdown of the mut HTT allele relative to the WT HTT allele.

### Example 7: Allele-Selective Knockdown in ATXN2 Mouse Model of SCA2

### Materials & Methods

The expression construct U6-shHD33-Full-Mimic was cloned into an AAV9 vector backbone, and AAV9 was produced using triple transfection into mammalian cells and purified by double CsCl density gradient ultra-centrifugation, desalting, and filter sterilization (Signagen Laboratories). Virus was formulated in PBS plus 0.005% Pluronic F-68. The expression cassettes contained GFP in order to assess transduction efficiency. AAV vectors were titered via qPCR. As a negative control, formulation vehicle was delivered to one group of transgenic mice and one group of wild-type littermates.

The vector preparation was administered via unilateral (right hemisphere) intracerebral ventricular injection into anesthetized male ATXN2-Q127 transgenic mice (C57BL6/J×DBA/2J hybrid background) and wild-type littermates using stereotaxic injection at 8 weeks of age. ATXN2-Q127 mice contain the complete human ataxin-2 cDNA encoding 127 glutamine repeats (Q127) under the control of the Pcp2 (Purkinje cell-specific) promoter (Hansen et al. (2013) Hum Mol Genet. 22:271). A 10.0 μL volume of test article was pump delivered at a rate of 1 μL/minute. Mice received test article as outlined in Table 10.

TABLE 10

| | | | | Dose |
|---|---|---|---|---|
| Group | Number | Genotype | Test Article | vg/mouse |
| 1 | 5 | wild-type (littermate) | vehicle | n/a |
| 2 | 5 | ATXN2-Q127 transgenic | vehicle | n/a |
| 3 | 5 | ATXN2-Q127 transgenic | Scramble shRNA | 2e11 |
| | | | (control) | |
| 4 | 5 | ATXN2-Q127 transgenic | Iris sbRNA | 5e10 |
| 5 | 5 | ATXN2-Q127 transgenic | Iris sbRNA | 2e11 |

Body weights of animals were taken at baseline in order to ensure even distribution between treatment groups. Weights were then assessed once per week until the time of tissue collection when animals were weighed before being collected.

Six weeks after injections, brain tissue was harvested. One animal per group (6 animals in total) were anesthetized with isoflurane and transcardially perfused with PBS until the liver was clear followed by approximately 50 mL of 4% PFA. The whole brain was harvested and placed into 25 mL vials with 4% PFA and stored at 4° C. for 24 hours. After 24 hours, brains were transferred to 30% sucrose in PBS and stored at 4° C. For the remaining 4 animals (or 3 animals for the wild-type group) in each group (19 animals in total), the animals were euthanized via rapid decapitation, and the brain was excised from the skull and rinsed in chilled 0.9% sterile saline to remove excess blood. Brains were microdissected by region under RNase-free conditions on top of a pre-wetted (0.9% saline) piece of clean (free of DNA and RNA) filter paper resting on a glass petri dish packed in wet ice. The following structures were dissected: striatum, cortex, and cerebellum. Each tissue sample was placed into individual 1.5 mL Eppendorf tubes and snap frozen in liquid nitrogen.

Cerebellum tissue samples were homogenized in lysis buffer from the miRvana miRNA isolation kit (Invitrogen). DNA was extracted using the All Prep DNA/RNA kit (Qiagen), and separate small RNA and total RNA fractions were extracted using the miRvana miRNA isolation kit. qPCR was performed by loading 400 ng DNA per reaction with TaqMan Universal PCR Master Mix II (Applied Biosystems) and using primer/probe sets targeting GFP (forward: GGAGCGCACCATCTTCTT (SEQ ID NO:860), reverse: ATGTTGCCGTCCTCCTTG (SEQ ID NO:861), probe: AAGGACGACGGCAACTACAAGACC (SEQ ID NO:862)) and mouse GAPDH (forward: TCACCTGGCCTACAGGATAAA (SEQ ID NO:863), reverse: GCAAACGGGAAGGAAATGAATG (SEQ ID NO:864), probe: ACCCAGCCGAGAGGAATGAGGTTA (SEQ ID NO:865)). Reactions were run on the Quantstudio 7 Pro (Applied Biosystems).

Whole brains were mounted onto a sliding microtome and frozen with dry ice. Sagittal sections (60 m) were cut, with every 8* section collected for immunofluorescent staining. Brains were stained as floating sections in a 12-well dish. Tissue sections were washed three times for 5 minutes each in PBS, then blocked in a solution containing 5% donkey serum and 0.3% Triton X-100 for 2 hours at room temperature. After blocking, tissue sections were incubated with mouse monoclonal anti-calbindin (1:100, Sigma Aldrich, SAB4200543) primary antibodies overnight at 4° C. Tissue sections were washed again three times for 15 minutes each in 0.3% Triton X-100 in PBS. Tissue sections were incubated with Alexa Fluor 594 goat anti-mouse secondary antibody (1:500, Life Technologies, A11005) for 2 hours at room temperature. Tissue sections were washed again three times for 15 minutes each in 0.3% Triton X-100 in PBS, followed by 4′,6-diamidino-2-phenylindole (DAPI) nuclear stain (Abcam) for 20 minutes at room temperature. Tissue sections were washed twice in PBS, then mounted onto slides and coverslipped. Images of the sections were taken using an Axioscan Z (Zeiss) microscope.

Cerebellum tissue samples were homogenized in extraction buffer (25 mM Tris-HCl pH 7.6, 300 mM NaCl, 0.5% Nonidet P-40, 2 mM EDTA, 2 mM MgCl₂, 0.5 M urea and protease inhibitors). Homogenized tissue was centrifuged, and the supernatant was collected. Equal volumes of protein were loaded into sodium dodecyl-sulfate polyacrylamide gels for Western blot analysis. Gels were run in running buffer (25 mM Tris, 192 mM Glycine, 0.1% SDS, pH 8.3) at 40 mA and maximum voltage for 2 hours. After electrophoresis, proteins were transferred to a Hybond PVDF membrane (Amersham Bioscience) in transfer buffer (25 mM Tris, 192 mM glycine, 20% (v/v) methanol, pH 8.3) at 20 V and maximum amperage for 1.5 hours using a semidry transfer system (Bio-Rad). The membrane was blocked in 4% skim milk in 0.1% Tween 20/PBS for 2 hours at 4° C. The membrane was incubated with the following primary antibodies: mouse anti-Ataxin-2 antibody (1:4,000, BD Biosciences, 611378), mouse monoclonal anti-GFP antibody (1:2,000, Santa Cruz, sc-9996), mouse monoclonal Anti-Calbindin-D-28K antibody (1:7,000, Sigma-Aldrich, C9848), rabbit polyclonal anti-RGS8 antibody (1:5,000, Novus Biologicals, NBP2-20153), mouse monoclonal anti-PCP-2 antibody (1:2,000, Santa Cruz, sc-137064), rabbit monoclonal anti-GAPDH (1:5,000, Cell Signaling Technology, 2118), mouse monoclonal anti-o-Actin-peroxidase antibody (1:30,000, Sigma-Aldrich, A3854), rabbit polyclonal anti-Ataxin-1 Antibody (1:4,000, Cell Signaling Technology, 2177), rabbit polyclonal anti-AAK1 Antibody (1:4,000, Cell Signaling Technology, 79832), rabbit monoclonal anti-Brn2/POU3F2 (1:4,000, Cell Signaling Technology, 12137), rabbit polyclonal anti-TBP Antibody (1:5,000, Cell Signaling Technology, 8515), rabbit polyclonal anti-AF9 Polyclonal antibody (1:4,000), Proteintech, 12825-1-AP) overnight at 4° C. The membrane was washed three times for 10 minutes each in 0.1% Tween 20/PBS, then incubated with corresponding Peroxidase-conjugated secondary antibodies: goat anti-rabbit IgG (H+L) antibody (1:5000, Jackson ImmunoResearch Laboratories, 111-035-144), donkey anti-mouse IgG (H+L) (1:5000, Jackson ImmunoResearch Laboratories, 715-035-150), and horse anti-mouse IgG (H+L) antibody (1:5,000, Vector laboratories, PI-2000) in 4% skim milk in 0.1% Tween 20/PBS for 2 hours at 4° C. The membrane was washed three more times for 10 minutes each in 0.1% Tween 20/PBS. The membrane was then incubated with Immobilon Western Chemiluminescent HRP Substrate (EMD Millipore) according to the manufacturer's protocol. Images were acquired on the ChemiDoc MP imager (Bio-Rad), and band intensity was quantified using ImageJ.

### Results

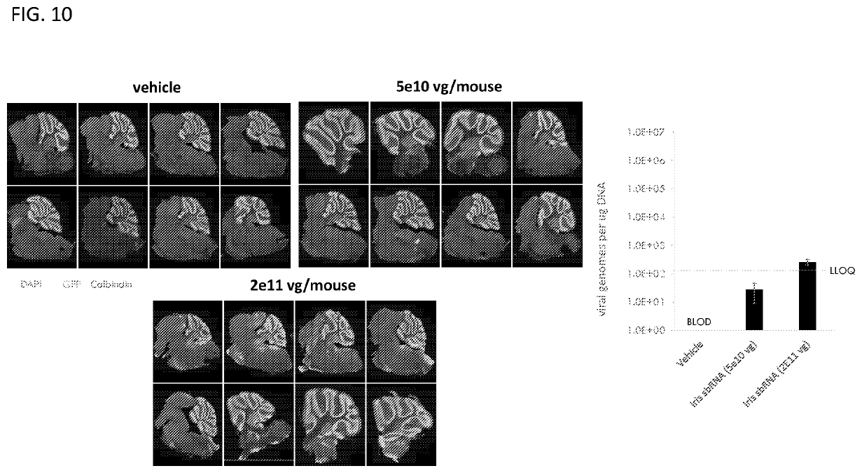

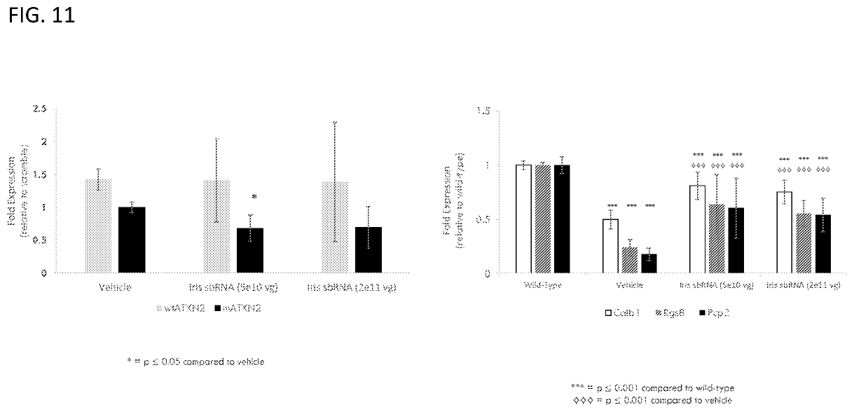

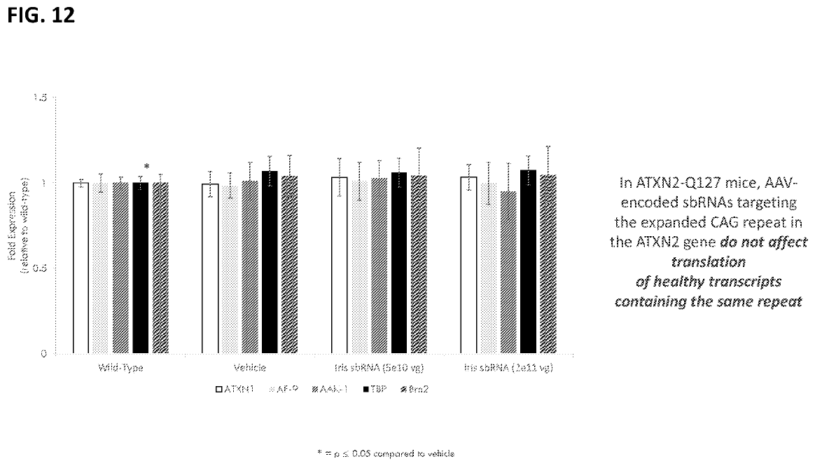

All animals had surgery performed at 8 weeks of age and survived until tissue collection at 14 weeks of age (6 weeks post dose administration), with the exception of one wild-type animal treated with vehicle that was found dead on the day of tissue collection at 14 weeks of age. Viral genomes were quantified by qPCR and demonstrated detectable sbRNA genome localization to the cerebellum (FIG. 10, right panel). GFP distribution was analyzed by immunofluorescence. Co-localization of calbindin and GFP staining demonstrated dose-dependent transduction of the ATXN2-Q127 cerebellum (FIG. 10, left panel). Western blot analysis of WT and mut ATXN2 protein expression showed significant knockdown of mut ATXN2 in the Iris sbRNA treatment group relative to the vehicle group at a dose of 5e10 vg (FIG. 11, left panel). In addition, Iris sbRNA showed preferential knockdown of the mut ATXN2 allele relative to the WT ATXN2 allele. Treatment with Iris sbRNA resulted in partial restoration of the expression of key cerebellar genes Calb1, Rgs8, and Pcp2, molecular demonstration of downstream improvement as a result of mut ATXN2 knock-down (FIG. 11, right panel). Furthermore, Western blot (WB) analysis of the expression of other genes containing endogenous CAG repeats (ATXN1, AF-9, AAK-1, TBP, and Brn2) demonstrated that sbRNAs targeting the expanded CAG repeat in the ATXN2 gene do not affect translation of healthy transcripts containing the same repeat (FIG. 12).

### Example 8: Register does not Impact Knock-Down Efficiency for Cag Repeats

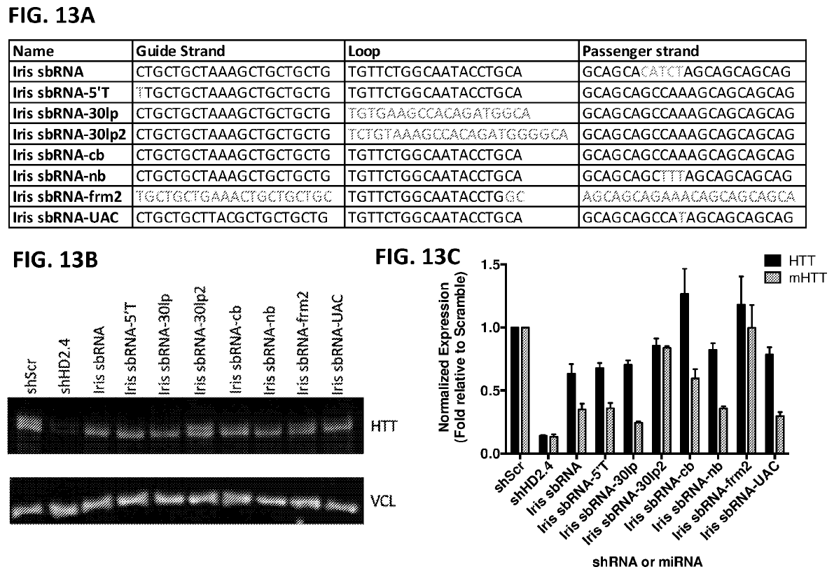

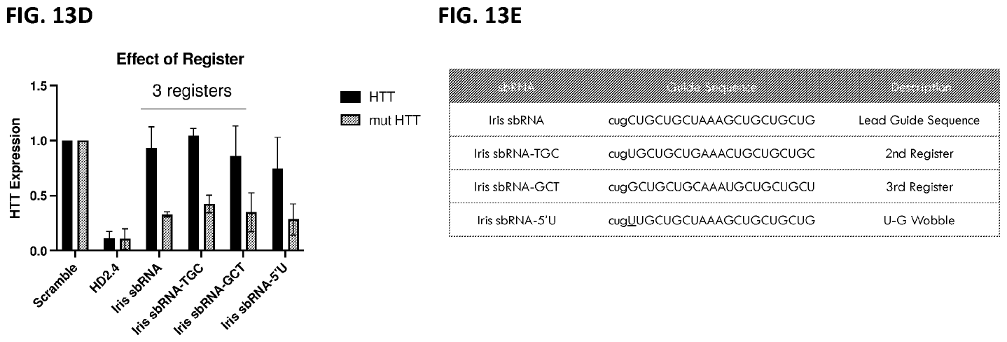

Lentivirus shRNA expression plasmids and lentiviral particles were produced as described in the Materials and Methods section above. HD fibroblasts were infected with lentiviral particles that express the indicated shRNA/miRNA sequences. WB were performed 5 days after infection using anti-HTT and anti-Vinculin, as described in the Materials and Methods section above. Knock-down of mHTT was observed for all sequences evaluated (FIG. 13A-13C). These data demonstrate that 1) different combinations of mismatches between the first and second strand of the double stranded RNA (e.g. 3 mismatches in the “Iris sbRNA” and 0 mismatches in the “Iris sbRNA-nb” lentivirus samples) and 2) different mismatches within the first strand of the double stranded RNA (e.g. TAC mismatches in the “Iris-sbRNA-UAC” and AAA mismatches in the “Iris sbRNA” lentivirus samples) are capable of efficient knock-down of mHTT and allele-selectivity.

FIG. 13A-13C. FIG. 13A. Design of additional guide strand, loop, and passenger strand sequences. 30lp: miR-30 loop; 30lp2: variant of the miR-30 loop; cb: 3 mismatches between the first and second strand of double stranded RNA; nb: no bulge (no mismatches between first and second strand of double stranded RNA); frm2: frame 2 (register 2) with 2 mismatches in the 3′ end of the loop; UAC: central mismatches are TAC (rather than AAA). FIG. 13B. HD patient-derived fibroblasts were transduced with lentivirus encoding the miRNA variants described in 2D. FIG. 13C. Quantification of the western blot data in FIG. 13D.

Lentivirus shRNA expression plasmids and lentiviral particles were produced as described in the Materials and Methods section above. These particles included samples which allowed for comparison of the same miRNA scaffold containing first strand sequences representative of the 1st registry (denoted HD33FM), 2nd registry (denoted “TGC”), or 3rd registry (denoted “GCT”). HD patient-derived fibroblasts were transduced with the indicated sbRNA-expressing lentiviral particles at 10 MOI. HTT expression levels were evaluated by Western blot 7 days after transduction and normalized to Scramble33Full wtHTT or mHTT. Prior to collection, GFP fluorescence was assessed to confirm infection. The level of mHTT knock-down and the level of wtHTT expression were similar between the HD33FM, TGC, and GCT lentivirus samples (FIG. 13D and FIG. 13E). These data demonstrate that sequences initiated at any of the three possible nucleotides within the repeat (e.g. the first strand sequence starting with C (1st registry), U (2nd registry), or G (3rd registry) are capable of efficient knock-down of mHTT and allele-selectivity.

### Example 9: Allele-Selective Reduction of CAG Repeat Expansion Protein Expression with CAG Repeat-Targeted miRNAs in a Luciferase-Based Model System

Guide sequence candidates were generated to systematically evaluate the effect of the number and position of mismatches, as well the starting position of the guide within the repeat (i.e. C, U, or G). Guide sequences containing a single mismatch in positions 8, 9, 10, and 11 were generated. Guide sequences containing double mismatches, where at least one mismatch is located in position 8, 9, 10, or 11, and the second mismatch is located within positions 8-16, were generated. Triple and quadruple mismatches, where at least two mismatches are located in position 8, 9, 10, or 11, and the remaining mismatches are located within positions 8-16, were also included.

To evaluate the starting position of the guide, sequences were generated containing three mismatches, where at least two mismatches are located in position 8, 9, 10, or 11, and the remaining mismatches are located within positions 8-16. These sequences initiated at any of the three possible nucleotides within the repeat (i.e. C, U, or G).

In silico analyses were conducted to remove guide sequences that have the potential for deleterious off-target effects. Sequences were synthesized if the following criteria for off-target genes were met:
off-target genes containing fully complementary sequence to guide sequence positions 1-21
≤2 off-target genes containing fully complementary 17mer within guide sequence positions 1-21
If off-target gene(s) contain fully complementary 17mer:
   Off-target gene(s) cannot be tumor suppressor gene(s)
   Off-target gene(s) cannot have high expression in caudate or putamen relative to whole brain or relative to all other tissues
   If fully complementary 17mer contains fully complementary seed sequence (guide sequence positions 2-8):
      Off-target gene(s) cannot be highly expressed in caudate or putamen relative to other genes

124 guide sequence candidates that passed the in silico analysis were embedded within the mir-33 scaffold (SEQ ID NO:X), synthesized, and cloned into a custom mammalian expression plasmid that contains a U6 promoter driving the miRNA and a human PGK promoter driving GFP-T2A-Puromycin.

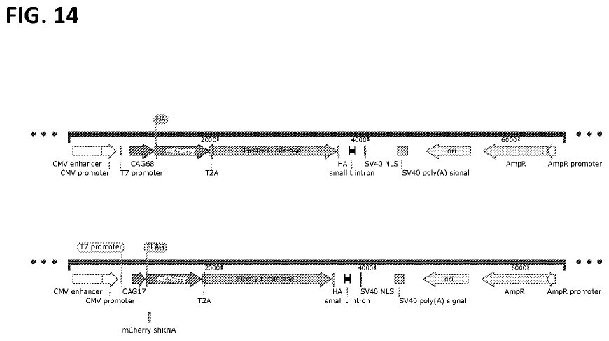

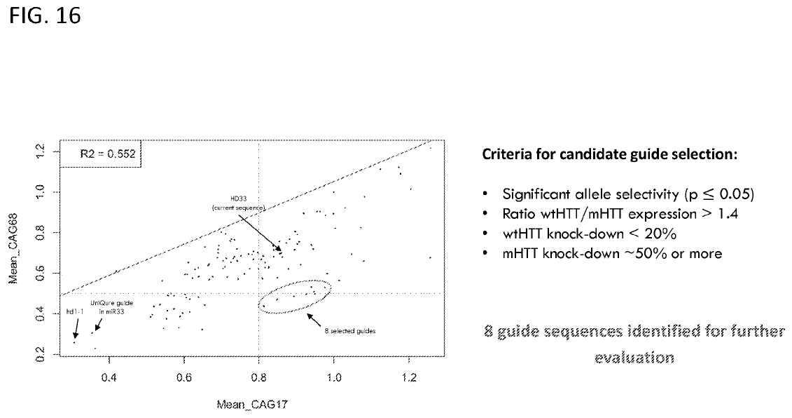

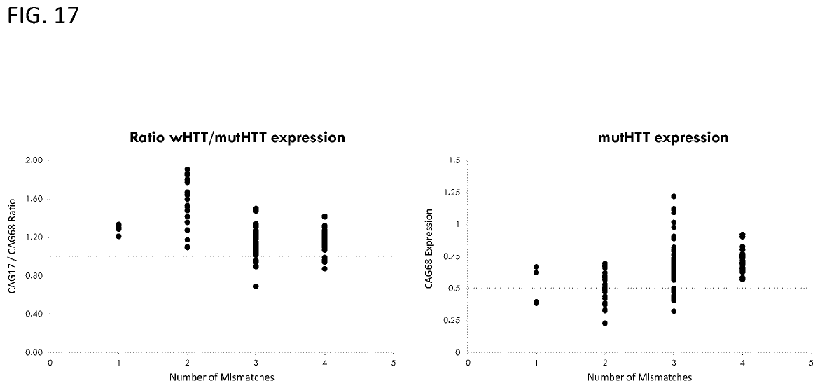

To evaluate the efficiency and allele selectivity of CAG repeat-targeted miRNAs, fragments of HTT exon 1 from wild-type (containing 17 CAG repeats) or mutant (containing 68 CAG repeats) mRNA were cloned in-frame and upstream of firefly luciferase (FIG. 14). Luciferase assays were established and performed by co-transfecting HEK293T cells on 96-well plates (40,000 cells/well) with 6.0 ng of wild-type (wt) or mutant (mut) HTT luciferase plasmid, 0.9 ng CMV-Renilla plasmid (Promega), and 0.75 ng indicated miRNA-expressing plasmid using calcium phosphate transfection method. Forty-eight hours after transfection, cells were lysed in 25 μL of 1× Passive Lysis buffer (Promega) for 40 min, and dual luciferase reporter assays (Hampf and Gossen (2006) Anal. Biochem. 356:94-99) were performed using 10 μL of lysate to determine the knockdown efficiency of the designed miRNAs. Firefly luciferase values were first normalized to Renilla luciferase values and then normalized to scramble miRNA controls. Luciferase experiments were performed at least 3 times and statistical analysis was performed using multiple t-tests comparing wt vs mut normalized luciferase activity for each miRNA. The positive control miRNA HD1-1 construct (targeting the CAG repeat with no mismatches) resulted in ˜70% decrease in luciferase activity for both wt and mut HTT constructs. Reduction in mut HTT expression >50% was observed for 27 of the 124 guide sequences evaluated. A relative preservation of wt HTT expression (<20% knock-down) was observed for 55 of the 124 guide sequences evaluated (FIG. 16). Allele-selectivity for mut HTT (defined as a ratio of wt HTT/mut HTT expression>1.4) was observed for 26 of the 124 guide sequences evaluated. Significant decreases (p<0.05) in mut HTT activity relative to wt HTT were observed for 19 of the 124 tested miRNAs.

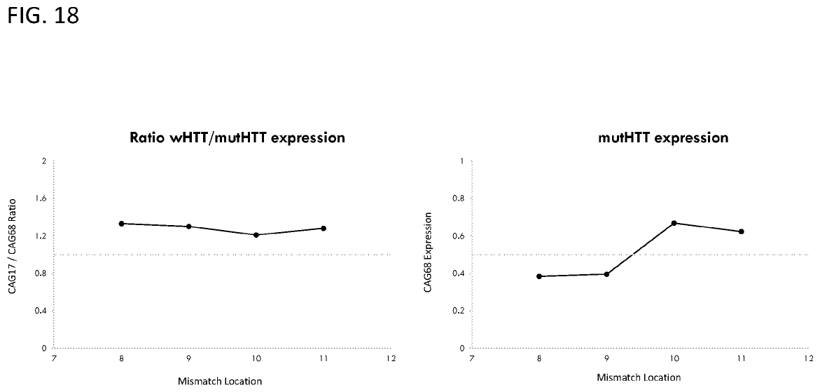

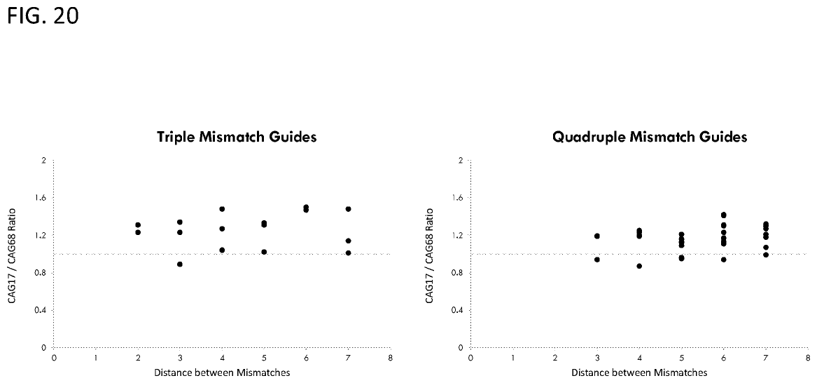

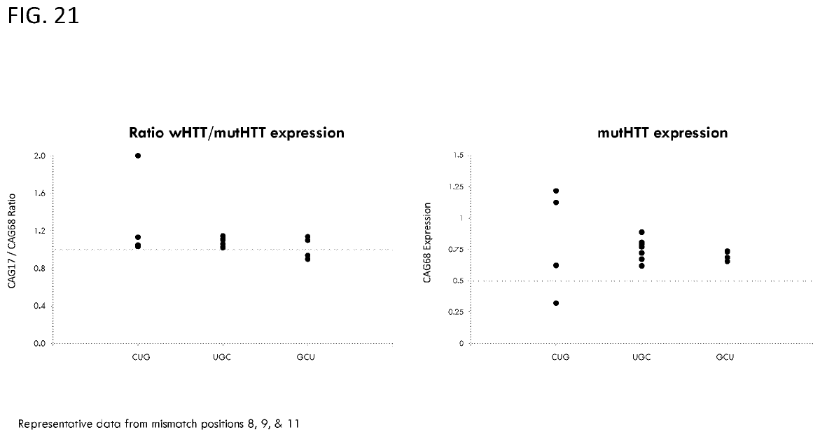

For guide sequences containing a single mismatch, mismatches located in position 8 or 9 showed improved knock-down of mut HTT and allele-selectivity toward mut HTT compared to mismatches located in position 10 or 11 (FIG. 18). There were no strong trends observed related to starting mismatch position or distance between mismatches for guide sequences containing three or four mismatches (FIG. 20). Similarly, there were no strong trends observed related to the starting position of the guide within the repeat (i.e. C, U, or G) for guide sequences containing three mismatches (FIG. 21).

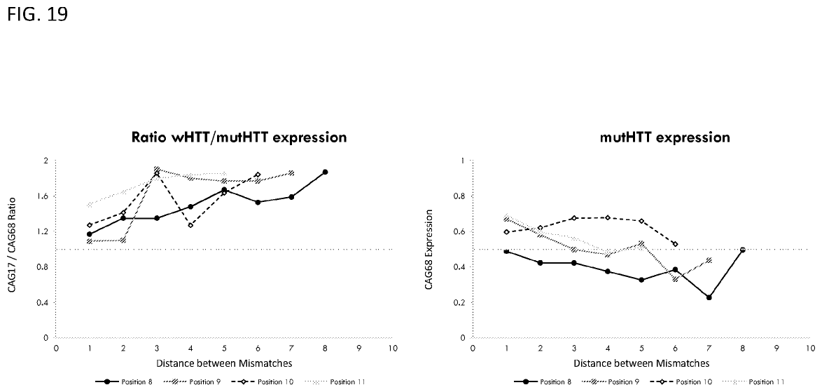

Overall, guide sequences containing two mismatches showed the best combination of knock-down of mut HTT and allele-selectivity toward mut HTT (FIG. 17). Of the guide sequences containing two mismatches, knock-down of mut HTT and allele-selectivity toward mut HTT were demonstrated for all sequences with initial mismatches in position 8, 9, 10, or 11. The level of knock-down of mut HTT and allele-selectivity toward mut HTT increased as the distance between mismatches increased (FIG. 19).

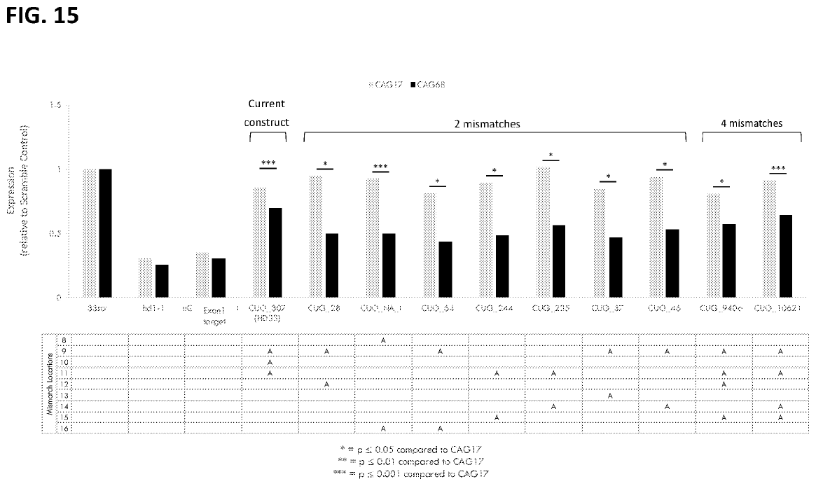

Nine guide sequences were identified as the top candidates, based on the following criteria: 1) significant decreases (p<0.05) in mut HTT activity relative to wt HTT, 2) allele-selectivity for mut HTT (defined as a ratio of wt HTT/mut HTT expression >1.4), 3) reduction in mut HTT expression >50%, and 4) relative preservation of wt HTT expression (<20% knock-down). The mut HTT and wt HTT expression levels following transfection with these 9 guide sequences is shown in (FIG. 15).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A double-stranded RNA comprising
a) a first strand that hybridizes to a target CAG repeat region of a CAG repeat containing RNA; and
b) a second strand that hybridizes to the first strand,
wherein the first strand comprises:
i) a first mismatch to the target CAG repeat region; and
ii) at least a second mismatch to the target CAG repeat region,
wherein:
i) when the first mismatch is at position 8 based on the numbering of SEQ ID NO:743 (CUGCUGCUGCUGCUGCUGCUG (SEQ ID NO:743)), SEQ ID NO:866(GCUGCUGCUGCUGCUGCUGCU (SEQ ID NO:866)), or SEQ ID NO:867 (UGCUGCUGCUGCUGCUGCUGC (SEQ ID NO:867)), the second mismatch is from is from 1 to 8 bases 3′ of the first mismatch;
ii) when the first mismatch is at position 9 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867), the second mismatch is from 1 to 7 bases 3′ of the first mismatch;
iii) when the first mismatch is at position 10 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)), the second mismatch is from 1 to 6 bases 3′ of the first mismatch; and
iv) when the first mismatch is at position 11 based on the numbering of SEQ ID NO:743 (CUGCUGCUGCUGCUGCUGCUG (SEQ ID NO:/743)), SEQ ID NO:866 (GCUGCUGCUGCUGCUGCUGCU (SEQ ID NO:866)), or (UGCUGCUGCUGCUGCUGCUGC (SEQ ID NO:867)), the second mismatch is from 1 to 5 bases 3′ of the first mismatch.

2. The double-stranded RNA of claim 1, wherein each mismatch is generated by a substitution that is independently selected from:
a) a substitution of a G with an A, a U, or a C;
b) a substitution of a U with an A, a G, or a C;
c) a substitution of a C with an A, a U, or a G.

3. The double-stranded RNA of claim 1 or claim 2, wherein the first strand comprises:
i) a first mismatch to the target CAG repeat region, wherein the first mismatch is at position 8 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867)); and
ii) a second mismatch to the target CAG repeat region, wherein the second mismatch is from 1 to 8 bases 3′ of the first mismatch.

4. The double-stranded RNA of any one of claims 1-3, wherein the first strand is a variant comprising at least a first and a second substitution of the nucleotide sequence: CUGCUGCUGCUGCUGCUGCUG (SEQ ID NO:743) wherein the first substitution generates the first mismatch and is at position 8 based on the numbering of CUGCUGCUGCUGCUGCUGCUG (SEQ ID NO:743) and the second substitution generates the second mismatch and is from 1 to 8 bases 3′ of the first mismatch.

5. The double-stranded RNA of any one of claims 1-3, wherein the first strand is a variant comprising at least a first and a second substitution of the nucleotide sequence: GCUGCUGCUGCUGCUGCUGCU (SEQ ID NO:866), wherein the first substitution generates the first mismatch and is at position 8 based on the numbering of GCUGCUGCUGCUGCUGCUGCU (SEQ ID NO:866), and the second substitution generates the second mismatch and is from 1 to 8 bases 3′ of the first mismatch.

6. The double-stranded RNA of any one of claims 1-3, wherein the first strand is a variant comprising at least a first and a second substitution of the nucleotide sequence: UGCUGCUGCUGCUGCUGCUGC (SEQ ID NO:867), wherein the first substitution generates the first mismatch and is at position 8 based on the numbering of UGCUGCUGCUGCUGCUGCUGC (SEQ ID NO:867), and the second substitution generates the second mismatch and is from 1 to 8 bases 3′ of the first mismatch.

7. The double-stranded RNA of any one of claims 3-6, wherein the first strand comprises no more than 2 mismatches with the target CAG repeat region.

8. The double-stranded RNA of any one of claims 3-6, wherein the first strand comprises no more than 3 mismatches with the target CAG repeat region.

9. The double-stranded RNA of any one of claims 3-6, wherein the first strand comprises no more than 4 mismatches with the target CAG repeat region.

10. The double-stranded RNA of claim 3 or claim 4, wherein the first strand comprises a nucleotide sequence selected from:
i)
(SEQ ID NO: 317)
CUGCUGCAACUGCUGCUGCUG (CUG_NA_B);
ii)
(SEQ ID NO: 318)
CUGCUGCAGAUGCUGCUGCUG (CUG_NA_C);
iii)
(SEQ ID NO: 319)
CUGCUGCAGCAGCUGCUGCUG (CUG_NA_D);
iv)
(SEQ ID NO: 320)
CUGCUGCAGCUACUGCUGCUG(CUG_NA_E);
v)
(SEQ ID NO: 321)
CUGCUGCAGCUGAUGCUGCUG (CUG_NA_F);
vi)
(SEQ ID NO: 322)
CUGCUGCAGCUGCAGCUGCUG (CUG_NA_G);
vii)
(SEQ ID NO: 323)
CUGCUGCAGCUGCUACUGCUG (CUG_NA_H);
and
viii)
(SEQ ID NO: 324)
CUGCUGCAGCUGCUGAUGCUG (CUG_NA_I).

11. The double-stranded RNA of claim 3 or claim 4, wherein the first strand comprises a nucleotide sequence selected from SEQ ID NOs:804-819.

12. The double-stranded RNA of claim 1 or claim 2, wherein the first strand comprises a first mismatch to the target CAG repeat region, wherein the first mismatch is at position 8 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867)); wherein the first strand comprises a second mismatch and a third mismatch to the target CAG repeat region, and wherein the second and third mismatches are from 1 to 8 bases 3′ of the first mismatch.

13. The double-stranded RNA of any one of claims 1, 2, and 12, wherein the first strand is a variant comprising at least a first, a second, and a third substitution of the nucleotide sequence CUGCUGCUGCUGCUGCUGCUG (SEQ ID NO:743), wherein the first substitution generates the first mismatch and is at position 8 based on the numbering of CUGCUGCUGCUGCUGCUGCUG (SEQ ID NO:743), wherein the second substitution generates the second mismatch and the third substitution generates the third mismatch, and wherein the second substitution and the third substitution are from 1 to 8 bases 3′ of the first mismatch.

14. The double-stranded RNA of any one of claims 1, 2, and 12, wherein the first strand is a variant comprising at least a first, a second, and a third substitution of the nucleotide sequence GCUGCUGCUGCUGCUGCUGCU (SEQ ID NO:866), and wherein the first substitution generates the first mismatch and is at position 8 based on the numbering of GCUGCUGCUGCUGCUGCUGCU (SEQ ID NO:866), wherein the second substitution generates the second mismatch and the third substitution generates the third mismatch, and wherein the second substitution and the third substitution are from 1 to 8 bases 3′ of the first mismatch.

15. The double-stranded RNA of any one of claims 1, 2, and 12, wherein the first strand is a variant comprising at least a first, a second, and a third substitution of the nucleotide sequence UGCUGCUGCUGCUGCUGCUGC (SEQ ID NO:867), and wherein the first substitution generates the first mismatch and is at position 8 based on the numbering of UGCUGCUGCUGCUGCUGCUGC (SEQ ID NO:867), wherein the second substitution generates the second mismatch and the third substitution generates the third mismatch, and wherein the second substitution and the third substitution are from 1 to 8 bases 3′ of the first mismatch.

16. The double-stranded RNA of any one of claims 12-15, wherein the first strand comprises no more than 3 mismatches with the target CAG repeat region.

17. The double-stranded RNA of any one of claims 12-15, wherein the first strand comprises no more than 4 mismatches with the target CAG repeat region.

18. The double-stranded RNA of claim 1 or claim 2, wherein the first strand comprises a first mismatch to the target CAG repeat region, wherein the first mismatch is at position 8 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867)); wherein the first strand comprises a second mismatch, a third mismatch, and a fourth mismatch to the target CAG repeat region, and wherein the second, third, and fourth mismatches are from 1 to 8 bases 3′ of the first mismatch.

19. The double-stranded RNA of any one of claims 1, 2, and 18, wherein the first strand is a variant comprising at least a first, a second, a third, and a fourth substitution of the nucleotide sequence CUGCUGCUGCUGCUGCUGCUG (SEQ ID NO:743), wherein the first substitution generates the first mismatch and is at position 8 based on the numbering of CUGCUGCUGCUGCUGCUGCUG (SEQ ID NO:743), wherein the second substitution generates the second mismatch, the third substitution generates the third mismatch, and the fourth substitution generates the fourth mismatch, and wherein the second, third, and fourth substitutions are from 1 to 8 bases 3′ of the first mismatch.

20. The double-stranded RNA of any one of claims 1, 2, and 18, wherein the first strand is a variant comprising at least a first, a second, a third, and a fourth substitution of the nucleotide sequence GCUGCUGCUGCUGCUGCUGCU (SEQ ID NO:866), wherein the first substitution generates the first mismatch and is at position 8 based on the numbering of GCUGCUGCUGCUGCUGCUGCU (SEQ ID NO:866), wherein the second substitution generates the second mismatch, the third substitution generates the third mismatch, and the fourth substitution generates the fourth mismatch, and wherein the second, third, and fourth substitutions are from 1 to 8 bases 3′ of the first mismatch.

21. The double-stranded RNA of any one of claims 1, 2, and 18, wherein the first strand is a variant comprising at least a first, a second, a third, and a fourth substitution of the nucleotide sequence UGCUGCUGCUGCUGCUGCUGC (SEQ ID NO:867), wherein the first substitution generates the first mismatch and is at position 8 based on the numbering of UGCUGCUGCUGCUGCUGCUGC (SEQ ID NO:867), wherein the second substitution generates the second mismatch, the third substitution generates the third mismatch, and the fourth substitution generates the fourth mismatch, and wherein the second, third, and fourth substitutions are from 1 to 8 bases 3′ of the first mismatch.

22. The double-stranded RNA of any one of claims 18-21, wherein the first strand comprises no more than 4 mismatches with the target CAG repeat region.

23. The double-stranded RNA of claim 1 or claim 2, wherein the first strand comprises:
i) a first mismatch to the target CAG repeat region, wherein the first mismatch is at position 9 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867), and
ii) a second mismatch to the target CAG repeat region, wherein the second mismatch is from 1 to 7 bases 3′ of the first mismatch.

24. The double-stranded RNA of any one of claims 1, 2, and 23, wherein the first strand is a variant comprising at least a first and a second substitution of the nucleotide sequence CUGCUGCUGCUGCUGCUGCUG (SEQ ID NO:743), wherein the first substitution generates the first mismatch and is at position 9 based on the numbering of CUGCUGCUGCUGCUGCUGCUG (SEQ ID NO:743), and the second substitution generates the second mismatch and is from 1 to 7 bases 3′ of the first mismatch.

25. The double-stranded RNA of any one of claims 1, 2, and 23, wherein the first strand is a variant comprising at least a first and a second substitution of the nucleotide sequence GCUGCUGCUGCUGCUGCUGCU (SEQ ID NO:866), wherein the first substitution generates the first mismatch and is at position 9 based on the numbering of GCUGCUGCUGCUGCUGCUGCU (SEQ ID NO:866), and the second substitution generates the second mismatch and is from 1 to 7 bases 3′ of the first mismatch.

26. The double-stranded RNA of any one of claims 1, 2, and 23, wherein the first strand is a variant comprising at least a first and a second substitution of the nucleotide sequence UGCUGCUGCUGCUGCUGCUGC (SEQ ID NO:867), wherein the first substitution generates the first mismatch and is at position 9 based on the numbering of UGCUGCUGCUGCUGCUGCUGC (SEQ ID NO:867), and the second substitution generates the second mismatch and is from 1 to 7 bases 3′ of the first mismatch.

27. The double-stranded RNA of claim 23, wherein the first strand comprises a nucleotide sequence selected from SEQ ID NOs:298-304.

28. The double-stranded RNA of any one of claims 23-27, wherein the first strand comprises no more than 2 mismatches with the target CAG repeat region.

29. The double-stranded RNA of any one of claims 23-27, wherein the first strand comprises no more than 3 mismatches with the target CAG repeat region.

30. The double-stranded RNA of any one of claims 23-27, wherein the first strand comprises no more than 4 mismatches with the target CAG repeat region.

31. The double-stranded RNA of claim 1 or claim 2, wherein the first mismatch is at position 9 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867), wherein the first strand comprises a second mismatch and a third mismatch to the target CAG repeat region, and wherein the second and third mismatches are from 1 to 7 bases 3′ of the first mismatch.

32. The double-stranded RNA of any one of claims 1, 2, and 31, wherein the first strand is a variant comprising at least a first, a second, and a third substitution of the nucleotide sequence CUGCUGCUGCUGCUGCUGCUG (SEQ ID NO:743), wherein the first substitution generates the first mismatch and is at position 9 based on the numbering of CUGCUGCUGCUGCUGCUGCUG (SEQ ID NO:743), wherein the second substitution generates the second mismatch and the third substitution generates the third mismatch, and wherein the second substitution and the third substitution are from 1 to 7 bases 3′ of the first mismatch.

33. The double-stranded RNA of any one of claims 1, 2, and 31, wherein the first strand is a variant comprising at least a first, a second, and a third substitution of the nucleotide sequence GCUGCUGCUGCUGCUGCUGCU (SEQ ID NO:866), wherein the first substitution generates the first mismatch and is at position 9 based on the numbering of GCUGCUGCUGCUGCUGCUGCU (SEQ ID NO:866), wherein the second substitution generates the second mismatch and the third substitution generates the third mismatch, and wherein the second substitution and the third substitution are from 1 to 7 bases 3′ of the first mismatch.

34. The double-stranded RNA of any one of claims 1, 2, and 31, wherein the first strand is a variant comprising at least a first, a second, and a third substitution of the nucleotide sequence UGCUGCUGCUGCUGCUGCUGC (SEQ ID NO:867), wherein the first substitution generates the first mismatch and is at position 9 based on the numbering of UGCUGCUGCUGCUGCUGCUGC (SEQ ID NO:867), wherein the second substitution generates the second mismatch and the third substitution generates the third mismatch, and wherein the second substitution and the third substitution are from 1 to 7 bases 3′ of the first mismatch.

35. The double-stranded RNA of any one of claims 31-34, wherein the first strand comprises no more than 3 mismatches with the target CAG repeat region.

36. The double-stranded RNA of any one of claims 31-34, wherein the first strand comprises no more than 4 mismatches with the target CAG repeat region.

37. The double-stranded RNA of claim 31, wherein the first strand comprises a nucleotide sequence selected from:
i)
(SEQ ID NO: 325)
CUGCUGCUAAAGCUGCUGCUG (CUG_307);
ii)
(SEQ ID NO: 326)
CUGCUGCUAAUACUGCUGCUG (CUG_334);
iii)
(SEQ ID NO: 327)
CUGCUGCUAAUGAUGCUGCUG (CUG_361);
iv)
(SEQ ID NO: 328)
CUGCUGCUAAUGCAGCUGCUG (CUG_388);
v)
(SEQ ID NO: 329)
CUGCUGCUAAUGCUACUGCUG (CUG_415);
vi)
(SEQ ID NO: 330)
CUGCUGCUACAGAUGCUGCUG (CUG_631);
vii)
(SEQ ID NO: 331)
CUGCUGCUACAGCAGCUGCUG (CUG_658);
viii)
(SEQ ID NO: 332)
CUGCUGCUACAGCUGAUGCUG (CUG_712);
ix)
(SEQ ID NO: 336)
CUGCUGCUAAUGCUGAUGCUG (CUG_442);
x)
(SEQ ID NO: 337)
CUGCUGCUACAACUGCUGCUG (CUG_604);
and
xi)
(SEQ ID NO: 338)
CUGCUGCUACAGCUACUGCUG (CUG_685).

38. The double-stranded RNA of claim 1 or claim 2, wherein the first strand comprises a first mismatch to the target CAG repeat region, wherein the first mismatch is at position 9 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or SEQ ID NO:867 (UGCUGCUGCUGCUGCUGCUGC (SEQ ID NO:867), wherein the first strand comprises a second mismatch, a third mismatch, and a fourth mismatch to the target CAG repeat region, and wherein the second, third, and fourth mismatches are from 1 to 7 bases 3′ of the first mismatch.

39. The double-stranded RNA of any one of claims 1, 2, and 38, wherein the first strand is a variant comprising at least a first, a second, a third, and a fourth substitution of the nucleotide sequence CUGCUGCUGCUGCUGCUGCUG (SEQ ID NO:743), wherein the first substitution generates the first mismatch and is at position 9 based on the numbering of CUGCUGCUGCUGCUGCUGCUG (SEQ ID NO: 743), wherein the second substitution generates the second mismatch, the third substitution generates the third mismatch, and the fourth substitution generates the fourth mismatch, and wherein the second, third, and fourth substitutions are from 1 to 7 bases 3′ of the first mismatch.

40. The double-stranded RNA of any one of claims 1, 2, and 38, wherein the first strand is a variant comprising at least a first, a second, a third, and a fourth substitution of the nucleotide sequence GCUGCUGCUGCUGCUGCUGCU (SEQ ID NO:866), wherein the first substitution generates the first mismatch and is at position 9 based on the numbering of GCUGCUGCUGCUGCUGCUGCU (SEQ ID NO:866), wherein the second substitution generates the second mismatch, the third substitution generates the third mismatch, and the fourth substitution generates the fourth mismatch, and wherein the second, third, and fourth substitutions are from 1 to 7 bases 3′ of the first mismatch.

41. The double-stranded RNA of any one of claims 1, 2, and 38, wherein the first strand is a variant comprising at least a first, a second, a third, and a fourth substitution of the nucleotide sequence UGCUGCUGCUGCUGCUGCUGC (SEQ ID NO:867), wherein the first substitution generates the first mismatch and is at position 9 based on the numbering of UGCUGCUGCUGCUGCUGCUGC (SEQ ID NO:867), wherein the second substitution generates the second mismatch, the third substitution generates the third mismatch, and the fourth substitution generates the fourth mismatch, and wherein the second, third, and fourth substitutions are from 1 to 7 bases 3′ of the first mismatch.

42. The double-stranded RNA of any one of claims 38-41, wherein the first strand comprises no more than 4 mismatches with the target CAG repeat region.

43. The double-stranded RNA of claim 38, wherein the first strand comprises a nucleotide sequence selected from SEQ ID NOs:341-344.

44. The double-stranded RNA of claim 38, wherein the first strand comprises a nucleotide sequence selected from SEQ ID NOs:347-367.

45. The double-stranded RNA of claim 1 or claim 2, wherein the first strand comprises:
i) a first mismatch to the target CAG repeat region, wherein the first mismatch is at position 10 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)); and
ii) a second mismatch to the target CAG repeat region, wherein the second mismatch is from 1 to 6 bases 3′ of the first mismatch.

46. The double-stranded RNA of any one of claims 1, 2, or 45, wherein the first strand is a variant comprising at least a first and a second substitution of the nucleotide sequence CUGCUGCUGCUGCUGCUGCUG (SEQ ID NO:743), wherein the first substitution generates the first mismatch and is at position 10 based on the numbering of CUGCUGCUGCUGCUGCUGCUG (SEQ ID NO:743), and wherein the second substitution generates the second mismatch and is from 1 to 6 bases 3′ of the first mismatch.

47. The double-stranded RNA of any one of claims 1, 2, or 45, wherein the first strand is a variant comprising at least a first and a second substitution of the nucleotide sequence GCUGCUGCUGCUGCUGCUGCU (SEQ ID NO:866), wherein the first substitution generates the first mismatch and is at position 10 based on the numbering of GCUGCUGCUGCUGCUGCUGCU (SEQ ID NO:866), and wherein the second substitution generates the second mismatch and is from 1 to 6 bases 3′ of the first mismatch.

48. The double-stranded RNA of any one of claims 1, 2, or 45, wherein the first strand is a variant comprising at least a first and a second substitution of the nucleotide sequence UGCUGCUGCUGCUGCUGCUGC (SEQ ID NO:867), wherein the first substitution generates the first mismatch and is at position 10 based on the numbering of UGCUGCUGCUGCUGCUGCUGC (SEQ ID NO:867), and wherein the second substitution generates the second mismatch and is from 1 to 6 bases 3′ of the first mismatch.

49. The double-stranded RNA of any one of claims 45-48, wherein the first strand comprises no more than 2 mismatches with the target CAG repeat region.

50. The double-stranded RNA of any one of claims 45-48, wherein the first strand comprises no more than 3 mismatches with the target CAG repeat region.

51. The double-stranded RNA of any one of claims 45-48, wherein the first strand comprises no more than 4 mismatches with the target CAG repeat region.

52. The double-stranded RNA of claim 45, wherein the first strand comprises a nucleotide sequence selected from SEQ ID NOs:305-310.

53. The double-stranded RNA of claim 1 or claim 2, wherein the first mismatch is at position 10 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)), wherein the first strand comprises a second mismatch and a third mismatch to the target CAG repeat region, and wherein the second and third mismatches are from 1 to 6 bases 3′ of the first mismatch.

54. The double-stranded RNA of any one of claims 1, 2, or 53, wherein the first strand is a variant comprising at least a first, a second, and a third substitution of the nucleotide sequence CUGCUGCUGCUGCUGCUGCUG (SEQ ID NO:743), wherein the first substitution generates the first mismatch and is at position 10 based on the numbering of CUGCUGCUGCUGCUGCUGCUG (SEQ ID NO:743), wherein the second substitution generates the second mismatch and the third substitution generates the third mismatch, and wherein the second substitution and the third substitution are from 1 to 6 bases 3′ of the first mismatch.

55. The double-stranded RNA of any one of claims 1, 2, or 53, wherein the first strand is a variant comprising at least a first, a second, and a third substitution of the nucleotide sequence GCUGCUGCUGCUGCUGCUGCU (SEQ ID NO:866), wherein the first substitution generates the first mismatch and is at position 10 based on the numbering of GCUGCUGCUGCUGCUGCUGCU (SEQ ID NO:866), wherein the second substitution generates the second mismatch and the third substitution generates the third mismatch, and wherein the second substitution and the third substitution are from 1 to 6 bases 3′ of the first mismatch.

56. The double-stranded RNA of any one of claims 1, 2, or 53, wherein the first strand is a variant comprising at least a first, a second, and a third substitution of the nucleotide sequence UGCUGCUGCUGCUGCUGCUGC (SEQ ID NO:867), wherein the first substitution generates the first mismatch and is at position 10 based on the numbering UGCUGCUGCUGCUGCUGCUGC (SEQ ID NO:867), wherein the second substitution generates the second mismatch and the third substitution generates the third mismatch, and wherein the second substitution and the third substitution are from 1 to 6 bases 3′ of the first mismatch.

57. The double-stranded RNA of any one of claims 53-56, wherein the first strand comprises no more than 3 mismatches with the target CAG repeat region.

58. The double-stranded RNA of any one of claims 53-56, wherein the first strand comprises no more than 4 mismatches with the target CAG repeat region.

59. The double-stranded RNA of claim 53, wherein the first strand comprises a nucleotide sequence selected from:
i)
(SEQ ID NO: 333)
CUGCUGCUGAAGAUGCUGCUG (CUG_2116);
ii)
(SEQ ID NO: 334)
CUGCUGCUGAAGCAGCUGCUG (CUG_2143);
iii)
(SEQ ID NO: 335)
CUGCUGCUGAAGCUACUGCUG (CUG_2170);
iv)
(SEQ ID NO: 339)
CUGCUGCUGAAACUGCUGCUG (CUG_2089);
and
v)
(SEQ ID NO: 340)
CUGCUGCUGAAGCUGAUGCUG (CUG_2197).

60. The double-stranded RNA of claim 1 or claim 2, wherein the first strand comprises a first mismatch to the target CAG repeat region, wherein the first mismatch is at position 10 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)), wherein the first strand comprises a second mismatch, a third mismatch, and a fourth mismatch to the target CAG repeat region, and wherein the second, third, and fourth mismatches are from 1 to 6 bases 3′ of the first mismatch.

61. The double-stranded RNA of claim 1, 2, or 60, wherein the first strand is a variant comprising at least a first, a second, a third, and a fourth substitution of the nucleotide sequence CUGCUGCUGCUGCUGCUGCUG (SEQ ID NO:743), wherein the first substitution generates the first mismatch and is at position 10 based on the numbering of CUGCUGCUGCUGCUGCUGCUG (SEQ ID NO:743), wherein the second substitution generates the second mismatch, the third substitution generates the third mismatch, and the fourth substitution generates the fourth mismatch, and wherein the second, third, and fourth substitutions are from 1 to 6 bases 3′ of the first mismatch.

62. The double-stranded RNA of claim 1, 2, or 60, wherein the first strand is a variant comprising at least a first, a second, a third, and a fourth substitution of the nucleotide sequence GCUGCUGCUGCUGCUGCUGCU (SEQ ID NO:866), wherein the first substitution generates the first mismatch and is at position 10 based on the numbering of CUGCUGCUGCUGCUGCUGCUG (SEQ ID NO:866), wherein the second substitution generates the second mismatch, the third substitution generates the third mismatch, and the fourth substitution generates the fourth mismatch, and wherein the second, third, and fourth substitutions are from 1 to 6 bases 3′ of the first mismatch.

63. The double-stranded RNA of claim 1, 2, or 60, wherein the first strand is a variant comprising at least a first, a second, a third, and a fourth substitution of the nucleotide sequence UGCUGCUGCUGCUGCUGCUGC (SEQ ID NO:867), wherein the first substitution generates the first mismatch and is at position 10 based on the numbering of UGCUGCUGCUGCUGCUGCUGC (SEQ ID NO:867), wherein the second substitution generates the second mismatch, the third substitution generates the third mismatch, and the fourth substitution generates the fourth mismatch, and wherein the second, third, and fourth substitutions are from 1 to 6 bases 3′ of the first mismatch.

64. The double-stranded RNA of any one of claims 60-63, wherein the first strand comprises no more than 4 mismatches with the target CAG repeat region.

65. The double-stranded RNA of claim 60, wherein the first strand comprises a nucleotide sequence selected from SEQ ID NOs:345, 346, and 368-375.

66. The double-stranded RNA of claim 1 or claim 2, wherein the first strand comprises:
i) a first mismatch to the target CAG repeat region, wherein the first mismatch is at position 11 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)); and
ii) a second mismatch to the target CAG repeat region, wherein the second mismatch is from 1 to 5 bases 3′ of the first mismatch.

67. The double-stranded RNA of any one of claims 1, 2, and 66, wherein the first strand is a variant comprising at least a first and a second substitution of the nucleotide sequence CUGCUGCUGCUGCUGCUGCUG (SEQ ID NO:743), wherein the first substitution generates the first mismatch and is at position 11 based on the numbering of CUGCUGCUGCUGCUGCUGCUG (SEQ ID NO:743), and the second substitution generates the second mismatch and is from 1 to 5 bases 3′ of the first mismatch.

68. The double-stranded RNA of any one of claims 1, 2, and 66, wherein the first strand is a variant comprising at least a first and a second substitution of the nucleotide sequence GCUGCUGCUGCUGCUGCUGCU (SEQ ID NO:866), wherein the first substitution generates the first mismatch and is at position 11 based on the numbering of GCUGCUGCUGCUGCUGCUGCU (SEQ ID NO:866), and the second substitution generates the second mismatch and is from 1 to 5 bases 3′ of the first mismatch.

69. The double-stranded RNA of any one of claims 1, 2, and 66, wherein the first strand is a variant comprising at least a first and a second substitution of the nucleotide sequence UGCUGCUGCUGCUGCUGCUGC (SEQ ID NO:867), wherein the first substitution generates the first mismatch and is at position 11 based on the numbering of UGCUGCUGCUGCUGCUGCUGC (SEQ ID NO:867), and the second substitution generates the second mismatch and is from 1 to 5 bases 3′ of the first mismatch.

70. The double-stranded RNA of any one of claims 66-69, wherein the first strand comprises no more than 2 mismatches with the target CAG repeat region.

71. The double-stranded RNA of any one of claims 66-69, wherein the first strand comprises no more than 3 mismatches with the target CAG repeat region.

72. The double-stranded RNA of any one of claims 66-69, wherein the first strand comprises no more than 4 mismatches with the target CAG repeat region.

73. The double-stranded RNA of claim 66, wherein the first strand comprises a nucleotide sequence selected from:
i)
(SEQ ID NO: 311)
CUGCUGCUGCAACUGCUGCUG (CUG_217);
ii)
(SEQ ID NO: 312)
CUGCUGCUGCAGAUGCUGCUG (CUG_226);
iii)
(SEQ ID NO: 313)
CUGCUGCUGCAGCAGCUGCUG (CUG_235);
iv)
(SEQ ID NO: 314)
CUGCUGCUGCAGCUACUGCUG (CUG_244);
and
v)
(SEQ ID NO: 315)
CUGCUGCUGCAGCUGAUGCUG (CUG_253).

74. The double-stranded RNA of claim 66, wherein the first strand comprises a nucleotide sequence selected from SEQ ID NOs:793-803.

75. The double-stranded RNA of claim 1 or claim 2, wherein the first strand comprises a first mismatch to the target CAG repeat region, wherein the first mismatch is at position 11 based on the numbering of SEQ ID NO:743 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)); and wherein the first strand comprises a second mismatch and a third mismatch to the target CAG repeat region, and wherein the second and third mismatches are from 1 to 5 bases 3′ of the first mismatch.

76. The double-stranded RNA of any one of claims 1, 2, and 75, wherein the first strand is a variant comprising at least a first, a second, and a third substitution of the nucleotide sequence CUGCUGCUGCUGCUGCUGCUG (SEQ ID NO:743), wherein the first substitution generates the first mismatch and is at position 11 based on the numbering of CUGCUGCUGCUGCUGCUGCUG (SEQ ID NO:743), wherein the second substitution generates the second mismatch and the third substitution generates the third mismatch, and wherein the second substitution and the third substitution are from 1 to 5 bases 3′ of the first mismatch.

77. The double-stranded RNA of any one of claims 1, 2, and 75, wherein the first strand is a variant comprising at least a first, a second, and a third substitution of the nucleotide sequence GCUGCUGCUGCUGCUGCUGCU (SEQ ID NO:866), wherein the first substitution generates the first mismatch and is at position 11 based on the numbering of GCUGCUGCUGCUGCUGCUGCU (SEQ ID NO:866), wherein the second substitution generates the second mismatch and the third substitution generates the third mismatch, and wherein the second substitution and the third substitution are from 1 to 5 bases 3′ of the first mismatch.

78. The double-stranded RNA of any one of claims 1, 2, and 75, wherein the first strand is a variant comprising at least a first, a second, and a third substitution of the nucleotide sequence UGCUGCUGCUGCUGCUGCUGC (SEQ ID NO:867), wherein the first substitution generates the first mismatch and is at position 11 based on the numbering of UGCUGCUGCUGCUGCUGCUGC (SEQ ID NO:867), wherein the second substitution generates the second mismatch and the third substitution generates the third mismatch, and wherein the second substitution and the third substitution are from 1 to 5 bases 3′ of the first mismatch.

79. The double-stranded RNA of any one of claims 75-78, wherein the first strand comprises no more than 3 mismatches with the target CAG repeat region.

80. The double-stranded RNA of any one of claims 75-78, wherein the first strand comprises no more than 4 mismatches with the target CAG repeat region.

81. The double-stranded RNA of claim 1 or claim 2, wherein the first strand comprises a first mismatch to the target CAG repeat region, wherein the first mismatch is at position 11 (CUGCUGCUGCUGCUGCUGCUG (SEQ ID NO:743)), SEQ ID NO:866 (GCUGCUGCUGCUGCUGCUGCU (SEQ ID NO:866)), or (UGCUGCUGCUGCUGCUGCUGC (SEQ ID NO:867)), wherein the first strand comprises a second mismatch, a third mismatch, and a fourth mismatch to the target CAG repeat region, and wherein the second, third, and fourth mismatches are from 1 to 5 bases 3′ of the first mismatch.

82. The double-stranded RNA of any one of claims 1, 2, and 81, wherein the first strand is a variant comprising at least a first, a second, a third, and a fourth substitution of the nucleotide sequence CUGCUGCUGCUGCUGCUGCUG (SEQ ID NO:743), wherein the first substitution generates the first mismatch and is at position 11 based on the numbering of CUGCUGCUGCUGCUGCUGCUG (SEQ ID NO:743), wherein the second substitution generates the second mismatch, the third substitution generates the third mismatch, and the fourth substitution generates the fourth mismatch, and wherein the second, third, and fourth substitutions are from 1 to 5 bases 3′ of the first mismatch.

83. The double-stranded RNA of any one of claims 1, 2, and 81, wherein the first strand is a variant comprising at least a first, a second, a third, and a fourth substitution of the nucleotide sequence GCUGCUGCUGCUGCUGCUGCU (SEQ ID NO:866), wherein the first substitution generates the first mismatch and is at position 11 based on the numbering of GCUGCUGCUGCUGCUGCUGCU (SEQ ID NO:866), wherein the second substitution generates the second mismatch, the third substitution generates the third mismatch, and the fourth substitution generates the fourth mismatch, and wherein the second, third, and fourth substitutions are from 1 to 5 bases 3′ of the first mismatch.

84. The double-stranded RNA of any one of claims 1, 2, and 81, wherein the first strand is a variant comprising at least a first, a second, a third, and a fourth substitution of the nucleotide sequence UGCUGCUGCUGCUGCUGCUGC (SEQ ID NO:867), wherein the first substitution generates the first mismatch and is at position 11 based on the numbering of UGCUGCUGCUGCUGCUGCUGC (SEQ ID NO:867), wherein the second substitution generates the second mismatch, the third substitution generates the third mismatch, and the fourth substitution generates the fourth mismatch, and wherein the second, third, and fourth substitutions are from 1 to 5 bases 3′ of the first mismatch.

85. The double-stranded RNA of any one of claims 81-84, wherein the first strand comprises no more than 4 mismatches with the target CAG repeat region.

86. The double-stranded RNA of any one of claims 1-85, wherein the second strand is 100% complementary to the first strand.

87. The double-stranded RNA of any one of claims 1-85, wherein the second strand comprises from 1 to 10 mismatches, from 3 to 5 mismatches, from 4 to 7 mismatches, or from 5 to 10 mismatches, to the first strand.

88. The double-stranded RNA of any one of claims 1-85, wherein the double-stranded RNA has a length of from 18 bases to 25 nucleotides, from 19 to 25 nucleotides, from 19 to 23 nucleotides, or from 19 to 21 nucleotides.

89. The double-stranded RNA of any one of claims 1-85, wherein the double-stranded RNA has a length of from 21 nucleotides to 25 nucleotides.

90. A DNA molecule comprising a nucleotide sequence encoding the first strand as set forth in any one of claims 1-89, wherein the nucleotide sequence is operably linked to a promoter that is functional in a eukaryotic cell.

91. A recombinant nucleic acid comprising:
a1) the double-stranded RNA of any one of claims 1-89; and
b1) a microRNA scaffold comprising a 5′ flanking polynucleotide, a loop polynucleotide, and a 3′ flanking polynucleotide,
wherein the recombinant nucleic acid comprises:
i) the 5′ flanking polynucleotide;
ii) the first strand of the double-stranded RNA;
iii) the loop polynucleotide;
iv) the second strand of the double-stranded RNA; and
iv) the 3′ flanking polynucleotide;
wherein at least one of the 5′ flanking polynucleotide, the loop polynucleotide, and the 3′ flanking polynucleotide is heterologous to the first and/or the second strand of the double-stranded RNA; or
a2) the double-stranded RNA of any one of claims 1-89; and
b2) a microRNA scaffold comprising a 5′ flanking polynucleotide, a loop polynucleotide, and a 3′ flanking polynucleotide,
wherein the recombinant nucleic acid comprises:
i) the 5′ flanking polynucleotide;
ii) the second strand of the double-stranded RNA;
iii) the loop polynucleotide;
iv) the first strand of the double-stranded RNA; and
v) the 3′ flanking polynucleotide;
wherein at least one of the 5′ flanking polynucleotide, the loop polynucleotide, and the 3′ flanking polynucleotide is heterologous to the first and/or the second strand of the double-stranded RNA; or
a3) the double-stranded RNA of any one of claims 1-89; and
b3) a microRNA scaffold comprising a 5′ flanking polynucleotide and a 3′ flanking polynucleotide,
wherein the recombinant nucleic acid comprises:
i) the 5′ flanking polynucleotide;
ii) the first strand of the double-stranded RNA;
iii) the second strand of the double-stranded RNA; and
iv) the 3′ flanking polynucleotide;
wherein one or both of the 5′ flanking polynucleotide and the 3′ flanking polynucleotide is heterologous to the first and/or the second strand of the double-stranded RNA; or
a4) the double-stranded RNA of any one of claims 1-89; and
b4) a microRNA scaffold comprising a 5′ flanking polynucleotide and a 3′ flanking polynucleotide,
wherein the recombinant nucleic acid comprises:
i) the 5′ flanking polynucleotide;
ii) the second strand of the double-stranded RNA;
iii) the first strand of the double-stranded RNA; and
iv) the 3′ flanking polynucleotide;
wherein one or both of the 5′ flanking polynucleotide and the 3′ flanking polynucleotide is heterologous to the first and/or the second strand of the double-stranded RNA.

92. The recombinant nucleic acid of claim 91, wherein the recombinant nucleic acid comprises:
a) the 5′ flanking polynucleotide;
b) the first strand of the double-stranded RNA;
c) the loop polynucleotide;
d) the second strand of the double-stranded RNA; and
e) the 3′ flanking polynucleotide.

93. The recombinant nucleic acid of claim 91 or claim 92, wherein the 5′ flanking polynucleotide, the loop polynucleotide, and the 3′ flanking polynucleotide are derived from miR33.

94. The recombinant nucleic acid of claim 91, wherein the recombinant nucleic acid comprises:
a) the 5′ flanking polynucleotide;
b) the first strand of the double-stranded RNA;
c) the second strand of the double-stranded RNA; and
d) the 3′ flanking polynucleotide.

95. The recombinant nucleic acid of claim 91 or claim 94, wherein the 5′ flanking polynucleotide and the 3′ flanking polynucleotide are derived from miR451.

96. A DNA molecule comprising a nucleotide sequence encoding a recombinant nucleic acid according to any one of claims 91-95.

97. The DNA molecule of claim 96, wherein the 5′ flanking polynucleotide is encoded by the nucleotide sequence: tgcacacctcctggcgggcagctctg (SEQ ID NO:738).

98. The DNA molecule of claim 96 or claim 97, wherein the loop polynucleotide is encoded by the nucleotide sequence: tgttctggcaatacctg (SEQ ID NO:739).

99. The DNA molecule of any one of claims 96-98, wherein the 3′ flanking polynucleotide is encoded by the nucleotide sequence: gggaggcctgccctgactgcccac (SEQ ID NO:740).

100. The DNA molecule of any one of claims 96-99, comprising the nucleotide sequence set forth in any one of SEQ ID NOs:579-656.

101. The DNA molecule of claim 96, wherein the 5′ flanking polynucleotide is encoded by the nucleotide sequence acctactgactgccagggcacttgggaatggcaagg (SEQ ID NO:854).

102. The DNA molecule of claim 96 or claim 101, wherein the 3′ flanking polynucleotide is encoded by the nucleotide sequence tcttgctatacccagaaaacgtgccaggaagagaac (SEQ ID NO:855).

103. The DNA molecule of claim 96, wherein:
i) the 5′ flanking polynucleotide is encoded by the nucleotide sequence acctactgactgccagggcacttgggaatggcaagg (SEQ ID NO:854);
ii) the 3′ flanking polynucleotide is encoded by the nucleotide sequence tcttgctatacccagaaaacgtgccaggaagagaac (SEQ ID NO:855); and
iii) the first strand or the second strand is encoded by the nucleotide sequence set forth in any one of SEQ ID NOs:379-456.

104. The DNA molecule of claim 96, wherein the 5′ flanking polynucleotide is encoded by the nucleotide sequence set forth in SEQ ID NO:856.

105. The DNA molecule of claim 96 or claim 104, wherein the 3′ flanking polynucleotide is encoded by the nucleotide sequence set forth in SEQ ID NO:857.

106. The DNA molecule of claim 96, wherein:
i) the 5′ flanking polynucleotide is encoded by the nucleotide sequence set forth in SEQ ID NO:856;
ii) the 3′ flanking polynucleotide is encoded by the nucleotide sequence set forth in SEQ ID NO:857; and
iii) the first strand or the second strand is encoded by the nucleotide sequence set forth in any one of SEQ ID NOs:379-456.

107. The DNA molecule of claim 96, wherein the 5′ flanking polynucleotide is encoded by the nucleotide sequence set forth in SEQ ID NO:858.

108. The DNA molecule of claim 96 or claim 107, wherein the 3′ flanking polynucleotide is encoded by the nucleotide sequence set forth in SEQ ID NO:859.

109. The DNA molecule of claim 96, wherein:
i) the 5′ flanking polynucleotide is encoded by the nucleotide sequence set forth in SEQ ID NO:858;
ii) the 3′ flanking polynucleotide is encoded by the nucleotide sequence set forth in SEQ ID NO:859; and
iii) the first strand or the second strand is encoded by the nucleotide sequence set forth in any one of SEQ ID NOs:379-456.

110. A recombinant expression vector comprising the DNA molecule of any one of claims 96 to 109.

111. The recombinant expression vector of claim 110, wherein the nucleotide sequence is operably linked to a promoter that is functional in a eukaryotic cell.

112. The recombinant expression vector of claim 111, wherein the promoter is an RNA polymerase II promoter or an RNA polymerase III promoter.

113. The recombinant expression vector of claim 111 or claim 112, wherein the promoter is a CAG promoter, a CBA promoter, a CMV promoter, a U6 promoter, an EF1α promoter, or an H1 promoter.

114. The recombinant expression vector of any one of claims 110-113, wherein the recombinant expression vector comprises a 5′ adeno-associated virus (AAV) inverted terminal repeat (ITR) sequence and a 3′ AAV ITR sequence.

115. A recombinant expression vector comprising a nucleotide sequence encoding the recombinant nucleic acid of any one of claims 91-95.

116. The recombinant expression vector of claim 115, wherein the nucleotide sequence is operably linked to a promoter that is functional in a eukaryotic cell.

117. The recombinant expression vector of claim 116, wherein the promoter is an RNA polymerase II promoter or an RNA polymerase III promoter.

118. The recombinant expression vector of claim 116 or claim 117, wherein the promoter is a CAG promoter, a CBA promoter a CMV promoter, a U6 promoter, an EF1α promoter, or an H1 promoter.

119. The recombinant expression vector of any one of claims 115-118, wherein the recombinant expression vector comprises a 5′ adeno-associated virus (AAV) inverted terminal repeat (ITR) sequence and a 3′ AAV ITR sequence.

120. A delivery vehicle comprising the recombinant expression vector of any one of claims 110-119.

121. The delivery vehicle of claim 120, wherein the delivery vehicle a non-viral delivery vehicle.

122. The delivery vehicle of claim 121, wherein the delivery vehicle is a lipid nanoparticle.

123. The delivery vehicle of claim 120, wherein the delivery vehicle is a viral particle.

124. A viral particle comprising the recombinant expression vector of any one of claims 110-119.

125. The viral particle of claim 126, wherein the viral particle is an adeno-associated virus (AAV) particle.

126. The viral particle of claim 125, wherein the AAV particle comprises an AAV9 capsid.

127. The viral particle of claim 125, wherein the AAV particle comprises an AAV2 capsid.

128. A composition comprising:
a) the recombinant expression vector of any one of claims 110-119; and
b) a pharmaceutically acceptable excipient.

129. A composition comprising:
a) the delivery vehicle of any one of claims 120-123; and
b) a pharmaceutically acceptable excipient.

130. A composition comprising:
a) a viral particle comprising the recombinant expression vector of any one of claims 124-127; and
b) a pharmaceutically acceptable excipient.

131. A method for selectively reducing translation of a disease-associated CAG repeat-containing RNA in an individual having a CAG repeat expansion disorder, the method comprising administering to the individual an effective amount of the expression vector of any one of claims 110-119, delivery vehicle of any one of claims 120-123, the viral particle of any one of claims 124-127, or the pharmaceutical composition of any one of claims 128-130.

132. The method of claim 131, wherein the repeat expansion disorder is Huntington's disease, spinocerebellar ataxia type 1, spinocerebellar ataxia type 2, spinocerebellar ataxia type 3, spinocerebellar ataxia type 6, spinocerebellar ataxia type 7, spinocerebellar ataxia type 12, spinocerebellar ataxia type 17, spinal and bulbar muscular atrophy, dentatorubral pallidoluysian atrophy, or cleidocranial dysplasia.

133. The method of claim 131 or claim 132, wherein said administering comprises direct injection to the central nervous system of the individual.

134. The method of claim 133, wherein the direct injection is intracerebral ventricular injection, intraparenchymal injection, intrathecal injection, intrastriatal injection, intrathalamic injection, intracisternal magna injection, subpial injection, or any combination thereof.

135. The method of any one of claims 131-134, wherein said administering provides for a ratio of a polypeptide encoded by the non-disease-associated CAG repeat-containing RNA to a polypeptide encoded by disease-associated CAG repeat-containing RNA of greater than 1.0.

136. The method of any one of claims 131-134, wherein said administering provides for a ratio of a polypeptide encoded by non-disease-associated CAG repeat-containing RNA to a polypeptide encoded by disease-associated CAG repeat-containing RNA of from 1.1 to 1.8.

137. The method of any one of claims 131-134, wherein said administering provides for a ratio of a polypeptide encoded by non-disease-associated CAG repeat-containing RNA to a polypeptide encoded by disease-associated CAG repeat-containing RNA of greater than 1.8.
